(19) United States
(12) Patent Application Publication
(10) Pub. No.: US 2023/0131598 A1
(43) Pub. Date: Apr. 27, 2023

(54) COMBINATION TREATMENT FOR CANCER

(71) Applicant: GLAXOSMITHKLINE INTELLECTUAL PROPERTY DEVELOPMENT LIMITED, Brentford, Middlesex (GB)

(72) Inventors: Marc S. BALLAS, Collegeville, PA (US); Catherine E. ELLIS, Collegeville, PA (US); Steven HIRSCHFELD, Collegeville, PA (US)

(21) Appl. No.: 17/911,816

(22) PCT Filed: Apr. 12, 2021
(86) PCT No.: PCT/EP2021/059376

§ 371 Date: Sep. 15, 2022

Related U.S. Application Data
(60) Provisional application No. 63/009,565, filed on Apr. 14, 2020.
(60) Provisional application No. 63/110,101, filed on Nov. 5, 2020.

(51) Int. Cl. C07K 16/28; A61P 35/00

(52) U.S. Cl. CPC C07K 16/2818; A61P 35/00; A61K 2039/507

(57) ABSTRACT

The invention relates to a method of treating cancer, such as head and neck cancer (e.g. head and neck squamous cell carcinoma and oropharyngeal cancer), lung cancer (e.g. non small cell lung cancer), urothelial cancer, melanoma or cervical cancer, involving the combination of an ICOS binding protein (e.g. an anti-ICOS antibody) and a TIM-3 binding protein (e.g. an anti-TIM-3 antibody) and, optionally, a PD-1 binding protein (e.g. an anti-PD-1 antibody).

### SEQUENCE LISTING

The instant application contains a Sequence Listing which has been submitted electronically in ASCII format and is hereby incorporated by reference in its entirety. Said ASCII copy, created on Nov. 5, 2020, is named PB66868_WO_Seqlist.txt and is 62 kilobytes in size.

### FIELD OF THE INVENTION

The present invention relates to a method of treating cancer in a mammal and to combinations useful in such treatment. In particular, the present invention relates to an Inducible T-cell COStimulator (ICOS) binding protein in combination with a T Cell Immunoglobulin and Mucin Domain-3 (TIM-3) binding protein.

### BACKGROUND TO THE INVENTION

Effective treatment of hyperproliferative disorders, including cancer, is a continuing goal in the oncology field. Generally, cancer results from the deregulation of the normal processes that control cell division, differentiation and apoptotic cell death and is characterized by the proliferation of malignant cells which have the potential for unlimited growth, local expansion and systemic metastasis. Deregulation of normal processes includes abnormalities in signal transduction pathways and response to factors that differ from those found in normal cells.

Immunotherapies are one approach to treat hyperproliferative disorders. A major hurdle that scientists and clinicians have encountered in the development of various types of cancer immunotherapies has been to break tolerance to self-antigen (cancer) in order to mount a robust anti-tumor response leading to tumor regression. Unlike traditional development of small and large molecule agents that target the tumor, cancer immunotherapies may, among other things, target cells of the immune system that have the potential to generate a memory pool of effector cells to induce more durable effects and minimize recurrences.

Though there have been many recent advances in the treatment of cancer, there remains a need for more effective and/or enhanced treatment of an individual suffering the effects of cancer. The methods herein that relate to combining therapeutic approaches for enhancing anti-tumor immunity address this need.

### SUMMARY OF THE INVENTION

According to an aspect of the invention, there is provided a combination comprising: an ICOS binding protein comprising a heavy chain amino acid sequence comprising a CDRH1 of SEQ ID NO:1, a CDRH2 of SEQ ID NO:2, and a CDRH3 of SEQ ID NO:3, and a light chain amino acid sequence comprising a CDRL1 of SEQ ID NO:4, a CDRL2 of SEQ ID NO:5, and a CDRL3 of SEQ ID NO:6; and a TIM-3 binding protein comprising a heavy chain amino acid sequence comprising a CDRH1 of SEQ ID NO:13, a CDRH2 of SEQ ID NO:14, and a CDRH3 of SEQ ID NO:15, and a light chain amino acid sequence comprising a CDRL1 of SEQ ID NO:16, a CDRL2 of SEQ ID NO:17, and a CDRL3 of SEQ ID NO:18, for use in the treatment of a cancer.

According to a further aspect of the invention, there is provided a combination comprising: an ICOS binding protein comprising a heavy chain amino acid sequence at least about 90% identical to the amino acid sequence of SEQ ID NO:9 and a light chain amino acid sequence at least about 90% identical to the amino acid sequence of SEQ ID NO:10; and a TIM-3 binding protein comprising a heavy chain amino acid sequence at least about 90% identical to the amino acid sequence of SEQ ID NO:21 and a light chain amino acid sequence at least about 90% identical to the amino acid sequence of SEQ ID NO:22, for use in the treatment of a cancer.

According to a further aspect of the invention, there is provided an ICOS binding protein comprising a heavy chain amino acid sequence comprising a CDRH1 of SEQ ID NO:1, a CDRH2 of SEQ ID NO:2, and a CDRH3 of SEQ ID NO:3, and a light chain amino acid sequence comprising a CDRL1 of SEQ ID NO:4, a CDRL2 of SEQ ID NO:5, and a CDRL3 of SEQ ID NO:6, for use in treating cancer in a human, wherein the ICOS binding protein is to be administered in combination with a TIM-3 binding protein comprising a CDRH1 of SEQ ID NO:13, a CDRH2 of SEQ ID NO:14, and a CDRH3 of SEQ ID NO:15, and a light chain amino acid sequence comprising a CDRL1 of SEQ ID NO:16, a CDRL2 of SEQ ID NO:17, and a CDRL3 of SEQ ID NO:18.

According to a further aspect of the invention, there is provided a TIM-3 binding protein comprising a heavy chain amino acid sequence comprising a CDRH1 of SEQ ID NO:13, a CDRH2 of SEQ ID NO:14, and a CDRH3 of SEQ ID NO:15, and a light chain amino acid sequence comprising a CDRL1 of SEQ ID NO:16, a CDRL2 of SEQ ID NO:17, and a CDRL3 of SEQ ID NO:18, for use in treating cancer, wherein the TIM-3 binding protein is to be administered in combination with an ICOS binding protein comprising a CDRH1 of SEQ ID NO:1, a CDRH2 of SEQ ID NO:2, and a CDRH3 of SEQ ID NO:3, and a light chain amino acid sequence comprising a CDRL1 of SEQ ID NO:4, a CDRL2 of SEQ ID NO:5, and a CDRL3 of SEQ ID NO:6.

According to a further aspect of the invention, there is provided a method for the treatment of cancer in a subject in need thereof comprising administering a therapeutically effective amount of a combination comprising an ICOS binding protein comprising a heavy chain amino acid sequence comprising a CDRH1 of SEQ ID NO:1, a CDRH2 of SEQ ID NO:2, and a CDRH3 of SEQ ID NO:3, and a light chain amino acid sequence comprising a CDRL1 of SEQ ID NO:4, a CDRL2 of SEQ ID NO:5, and a CDRL3 of SEQ ID NO:6, and a TIM-3 binding protein comprising a heavy chain amino acid sequence comprising a CDRH1 of SEQ ID NO:13, a CDRH2 of SEQ ID NO:14, and a CDRH3 of SEQ ID NO:15, and a light chain amino acid sequence comprising a CDRL1 of SEQ ID NO:16, a CDRL2 of SEQ ID NO:17, and a CDRL3 of SEQ ID NO:18, to the subject.

According to a further aspect of the invention, there is provided a use of an ICOS binding protein comprising a heavy chain amino acid sequence comprising a CDRH1 of SEQ ID NO:1, a CDRH2 of SEQ ID NO:2, and a CDRH3 of SEQ ID NO:3, and a light chain amino acid sequence comprising a CDRL1 of SEQ ID NO:4, a CDRL2 of SEQ ID NO:5, and a CDRL3 of SEQ ID NO:6, in the manufacture of a medicament for use in the treatment of a cancer, wherein the medicament is to be administered in combination with a TIM-3 binding protein comprising a heavy chain amino acid sequence comprising a CDRH1 of SEQ ID NO:13, a CDRH2 of SEQ ID NO:14, and a CDRH3 of SEQ ID NO:15, and a light chain amino acid sequence comprising a CDRL1 of SEQ ID NO:16, a CDRL2 of SEQ ID NO:17, and a CDRL3 of SEQ ID NO:18.

According to a further aspect of the invention, there is provided a use of a TIM-3 binding protein comprising a heavy chain amino acid sequence comprising a CDRH1 of SEQ ID NO:13, a CDRH2 of SEQ ID NO:14, and a CDRH3 of SEQ ID NO:15, and a light chain amino acid sequence comprising a CDRL1 of SEQ ID NO:16, a CDRL2 of SEQ ID NO:17, and a CDRL3 of SEQ ID NO:18, in the manufacture of a medicament for use in the treatment of a cancer, wherein the medicament is to be administered in combination with an ICOS binding protein comprising a heavy chain amino acid sequence comprising a CDRH1 of SEQ ID NO:1, a CDRH2 of SEQ ID NO:2, and a CDRH3 of SEQ ID NO:3, and a light chain amino acid sequence comprising a CDRL1 of SEQ ID NO:4, a CDRL2 of SEQ ID NO:5, and a CDRL3 of SEQ ID NO:6.

According to a further aspect of the invention, there is provided a kit comprising:

(i) an ICOS binding protein comprising a heavy chain amino acid sequence comprising a CDRH1 of SEQ ID NO:1, a CDRH2 of SEQ ID NO:2, and a CDRH3 of SEQ ID NO:3, and a light chain amino acid sequence comprising a CDRL1 of SEQ ID NO:4, a CDRL2 of SEQ ID NO:5, and a CDRL3 of SEQ ID NO:6;

(ii) a TIM-3 binding protein comprising a heavy chain amino acid sequence comprising a CDRH1 of SEQ ID NO:13, a CDRH2 of SEQ ID NO:14, and a CDRH3 of SEQ ID NO:15, and a light chain amino acid sequence comprising a CDRL1 of SEQ ID NO:16, a CDRL2 of SEQ ID NO:17, and a CDRL3 of SEQ ID NO:18; and alternatively comprising

(iii) instructions for using (i) and (ii) in combination in the treatment of a cancer in a human.

### DESCRIPTION OF DRAWINGS/FIGURES

FIGS. 1A-1B Results from an in vivo efficacy study in a murine syngeneic tumor model (EMT-6) showing FIG. 1A) tumor volume growth and FIG. 1B) survival curves.

FIG. 2 Summary of study design described in Example 2.

FIG. 3 Modified Toxicity Probability Interval (mTPI) Dose Decision Rules. Columns provide the numbers of subjects treated at a dose level, and rows provide the corresponding numbers of subjects experiencing DLT (dose limiting toxicity). The entries in the table are dose-finding decisions (i.e. E, S, and D) representing escalating the dose, staying at the same dose, and de-escalating the dose, respectively. In addition, decision U indicates that the current dose level is unacceptable because of high toxicity and should be excluded from further investigation in the study.

FIGS. 4A-4B Time and Events table for Safety, Laboratory, Efficacy, Study Treatment Procedures as described in Example 2. The tables of FIG. 4A and FIG. 4B summarises assessment windows and sequencing of assessments and procedures.

FIGS. 5A-5B Time and Events table for Pharmacokinetics, Immunogenicity, Biomarker Assessments as described in Example 2. The tables of FIG. 5A and FIG. 5B summarises assessment windows and sequencing of assessments and procedures.

FIG. 6 Time and Events table for Patient Reported Outcome Assessments as described in Example 2. The table summarises assessment windows and sequencing of assessments and procedures.

### DETAILED DESCRIPTION OF THE INVENTION

### Definitions

“Antigen binding protein” (ABP) means a protein that binds an antigen, including antibodies or engineered molecules that function in similar ways to antibodies. Such alternative antibody formats include triabody, tetrabody, miniantibody, and a minibody. An ABP also includes antigen binding fragments of such antibodies or other molecules. Further, an ABP may comprise the V_{H} regions of the invention formatted into a full length antibody, a (Fab′)₂ fragment, a Fab fragment, a bi-specific or biparatopic molecule or equivalent thereof (such as scFv, bi- tri- or tetra-bodies, TANDABS etc.), when paired with an appropriate light chain. The ABP may comprise an antibody that is an IgG1, IgG2, IgG3, or IgG4; or IgM; IgA, IgE or IgD or a modified variant thereof. The constant domain of the antibody heavy chain may be selected accordingly. The light chain constant domain may be a kappa or lambda constant domain. The ABP may also be a chimeric antibody of the type described in WO86/01533, which comprises an antigen binding region and a non-immunoglobulin region. The terms “ABP”, “antigen binding protein”, “binding protein”, “antigen binding agent” and “binding agent” are used interchangeably herein. For example, disclosed herein are ICOS binding proteins and TIM-3 binding proteins.

“Antigen binding site” refers to a site on an antigen binding protein that is capable of specifically binding to an antigen, this may be a single variable domain, or it may be paired V_{H}/V_{L} domains as can be found on a standard antibody. Single-chain Fv (scFv) domains can also provide antigen-binding sites.

The term “antibody” is used herein in the broadest sense to refer to molecules comprising an immunoglobulin-like domain (for example IgG, IgM, IgA, IgD or IgE) and includes monoclonal, recombinant, polyclonal, chimeric, human, humanized, multispecific antibodies, including bispecific antibodies, and heteroconjugate antibodies; a single variable domain (e.g. V_{H}, V_{HH}, V_{L}, domain antibody (DAB)), antigen binding antibody fragments, Fab, F(ab′)₂, Fv, disulphide linked Fv, single chain Fv, disulphide-linked scFv, diabodies, TANDABS, etc. and modified versions of any of the foregoing (for a summary of alternative “antibody” formats see, e.g. Holliger and Hudson, Nature Biotechnology, 2005, Vol 23, No. 9, 1126-1136).

A “chimeric antibody” refers to a type of engineered antibody that contains a naturally-occurring variable region (light chain and heavy chains) derived from a donor antibody in association with light and heavy chain constant regions derived from an acceptor antibody.

A “humanized antibody” refers to a type of engineered antibody having its CDRs derived from a non-human donor immunoglobulin, the remaining immunoglobulin-derived parts of the molecule being derived from one or more human immunoglobulin(s). In addition, framework support residues may be altered to preserve binding affinity (see, e.g. Queen et al. Proc. Natl Acad Sci USA, 86:10029-10032 (1989), Hodgson et al. Bio/Technology, 9:421 (1991)). A suitable human acceptor antibody may be one selected from a conventional database, e.g. the KABAT database, Los Alamos database, and Swiss Protein database, by homology to the nucleotide and amino acid sequences of the donor antibody. A human antibody characterized by a homology to the framework regions of the donor antibody (on an amino acid basis) may be suitable to provide a heavy chain constant region and/or a heavy chain variable framework region for insertion of the donor CDRs. A suitable acceptor antibody capable of donating light chain constant or variable framework regions may be selected in a similar manner. It should be noted that the acceptor antibody heavy and light chains are not required to originate from the same acceptor antibody. The prior art describes several ways of producing such humanized antibodies—see, for example, EP-A-0239400 and EP-A-054951.

The term “fully human antibody” includes antibodies having variable and constant regions (if present) derived from human germline immunoglobulin sequences. The human sequence antibodies of the invention may include amino acid residues not encoded by human germline immunoglobulin sequences (e.g. mutations introduced by random or site-specific mutagenesis in vitro or by somatic mutation in vivo). Fully human antibodies comprise amino acid sequences encoded only by polynucleotides that are ultimately of human origin or amino acid sequences that are identical to such sequences. As meant herein, antibodies encoded by human immunoglobulin-encoding DNA inserted into a mouse genome produced in a transgenic mouse are fully human antibodies since they are encoded by DNA that is ultimately of human origin. In this situation, human immunoglobulin-encoding DNA can be rearranged (to encode an antibody) within the mouse, and somatic mutations may also occur. Antibodies encoded by originally human DNA that has undergone such changes in a mouse are fully human antibodies as meant herein. The use of such transgenic mice makes it possible to select fully human antibodies against a human antigen. As is understood in the art, fully human antibodies can be made using phage display technology wherein a human DNA library is inserted in phage for generation of antibodies comprising human germline DNA sequence.

The term, full, whole or intact antibody, used interchangeably herein, refers to a heterotetrameric glycoprotein with an approximate molecular weight of 150,000 daltons. An intact antibody is composed of two identical heavy chains (HCs) and two identical light chains (LCs) linked by covalent disulphide bonds. This H₂L₂ structure folds to form three functional domains comprising two antigen-binding fragments, known as ‘Fab’ fragments, and a ‘Fc’ crystallisable fragment. The Fab fragment is composed of the variable domain at the amino-terminus, variable heavy (V_{H}) or variable light (V_{L}), and the constant domain at the carboxyl terminus, CH1 (heavy) and CL (light). The Fc fragment is composed of two domains formed by dimerization of paired CH2 and CH3 regions. The Fc may elicit effector functions by binding to receptors on immune cells or by binding C1q, the first component of the classical complement pathway. The five classes of antibodies IgM, IgA, IgG, IgE and IgD are defined by distinct heavy chain amino acid sequences which are called μ, α, γ, ε and δ respectively, each heavy chain can pair with either a K or A light chain. The majority of antibodies in the serum belong to the IgG class, there are four isotypes of human IgG, IgG1, IgG2, IgG3 and IgG4, the sequences of which differ mainly in their hinge region.

Fully human antibodies can be obtained using a variety of methods, for example using yeast-based libraries or transgenic animals (e.g. mice) which are capable of producing repertoires of human antibodies. Yeast presenting human antibodies on their surface which bind to an antigen of interest can be selected using FACS (Fluorescence-Activated Cell Sorting) based methods or by capture on beads using labelled antigens. Transgenic animals that have been modified to express human immunoglobulin genes can be immunised with an antigen of interest and antigen-specific human antibodies isolated using B-cell sorting techniques. Human antibodies produced using these techniques can then be characterised for desired properties such as affinity, developability and selectivity.

Alternative antibody formats include alternative scaffolds in which the one or more CDRs of the antigen binding protein can be arranged onto a suitable non-immunoglobulin protein scaffold or skeleton, such as an affibody, a SpA scaffold, an LDL receptor class A domain, an avimer (see, e.g. U.S. Patent Application Publication Nos. 2005/0053973, 2005/0089932, 2005/0164301) or an EGF domain.

The term “domain” refers to a folded polypeptide structure that retains its tertiary structure independent of the rest of the polypeptide. Generally domains are responsible for discrete functional properties of polypeptides and in many cases may be added, removed or transferred to other polypeptides without loss of function of the remainder of the protein and/or of the domain.

The term “single variable domain” refers to a folded polypeptide domain comprising sequences characteristic of antibody variable domains. It therefore includes complete antibody variable domains such as V_{H}, V_{HH} and V_{L} and modified antibody variable domains, for example, in which one or more loops have been replaced by sequences which are not characteristic of antibody variable domains, or antibody variable domains which have been truncated or comprise N- or C-terminal extensions, as well as folded fragments of variable domains which retain at least the binding activity and specificity of the full-length domain. A single variable domain is capable of binding an antigen or epitope independently of a different variable region or domain. A “domain antibody” or “DAB” may be considered the same as a “single variable domain”. A single variable domain may be a human single variable domain, but also includes single variable domains from other species such as rodent, nurse shark and Camelid V_{HH} DABS. Camelid V_{HH} are immunoglobulin single variable domain polypeptides that are derived from species including camel, llama, alpaca, dromedary, and guanaco, which produce heavy chain antibodies naturally devoid of light chains. Such V_{HH} domains may be humanized according to standard techniques available in the art, and such domains are considered to be “single variable domains”. As used herein V_{H} includes camelid V_{HH} domains.

The terms “V_{H}” and “V_{L}” are used herein to refer to the heavy chain variable region and light chain variable region respectively of an antigen binding protein.

“CDRs” are defined as the complementarity determining region amino acid sequences of an antigen binding protein. These are the hypervariable regions of immunoglobulin heavy and light chains. There are three heavy chain and three light chain CDRs (or CDR regions) in the variable portion of an immunoglobulin. Thus, “CDRs” as used herein refers to all three heavy chain CDRs, all three light chain CDRs, all heavy and light chain CDRs, or at least two CDRs.

Throughout this specification, amino acid residues in variable domain sequences and variable domain regions within full length antigen binding sequences, e.g. within an antibody heavy chain sequence or antibody light chain sequence, are numbered according to the Kabat numbering convention. Similarly, the terms “CDR”, “CDRL1”, “CDRL2”, “CDRL3”, “CDRH1”, “CDRH2”, “CDRH3” used in the Examples follow the Kabat numbering convention. For further information, see Kabat et al. Sequences of Proteins of Immunological Interest, 5th Ed., U.S. Department of Health and Human Services, National Institutes of Health (1991).

It will be apparent to those skilled in the art that there are alternative numbering conventions for amino acid residues in variable domain sequences and full length antibody sequences. There are also alternative numbering conventions for CDR sequences, for example those set out in Chothia et al. (1989) Nature 342: 877-883. The structure and protein folding of the antigen binding protein may mean that other residues are considered part of the CDR sequence and would be understood to be so by a skilled person.

Other numbering conventions for CDR sequences available to a skilled person include “AbM” (University of Bath) and “contact” (University College London) methods. The minimum overlapping region using at least two of the Kabat, Chothia, AbM and contact methods can be determined to provide the “minimum binding unit”. The minimum binding unit may be a sub-portion of a CDR.

CDRs or minimum binding units may be modified by at least one amino acid substitution, deletion or addition, wherein the variant antigen binding protein substantially retains the biological characteristics of the unmodified protein, such as an antibody comprising SEQ ID NO:7 and SEQ ID NO:8.

CDRs or minimum binding units may be modified by at least one amino acid substitution, deletion or addition, wherein the variant antigen binding protein substantially retains the biological characteristics of the unmodified protein, such as an antibody comprising SEQ ID NO:7 and SEQ ID NO:8. It will be appreciated that each of CDR H1, H2, H3, L1, L2, L3 may be modified alone or in combination with any other CDR, in any permutation or combination. In one embodiment, a CDR is modified by the substitution, deletion or addition of up to 3 amino acids, for example 1 or 2 amino acids, for example 1 amino acid. Typically, the modification is a substitution, particularly a conservative substitution (referred herein also as a direct equivalent), for example as shown in Table 1 below.

TABLE 1

| Side chain | Members |
|---|---|
| Hydrophobic | Met, Ala, Val, Leu, Ile |
| Neutral hydrophilic | Cys, Ser, Thr |
| Acidic | Asp, Glu |
| Basic | Asn, Gln, His, Lys, Arg |
| Residues that influence chain orientation | Gly, Pro |
| Aromatic | Trp, Tyr, Phe |

“Percent identity” between a query amino acid sequence and a subject amino acid sequence is the “Identities” value, expressed as a percentage, that is calculated using a suitable algorithm or software, such as BLASTP, FASTA, DNASTAR Lasergene, GeneDoc, Bioedit, EMBOSS needle or EMBOSS infoalign, over the entire length of the query sequence after a pair-wise global sequence alignment has been performed using a suitable algorithm/software such as BLASTP, FASTA, ClustalW, MUSCLE, MAFFT, EMBOSS Needle, T-Coffee, and DNASTAR Lasergene. Importantly, a query amino acid sequence may be described by an amino acid sequence identified in one or more claims herein.

The query sequence may be 100% identical to the subject sequence, or it may include up to a certain integer number of amino acid or nucleotide alterations as compared to the subject sequence such that the % identity is less than 100%. For example, the query sequence is at least 50, 60, 70, 75, 80, 85, 90, 95, 96, 97, 98, or 99% identical to the subject sequence. Such alterations include at least one amino acid deletion, substitution (including conservative and non-conservative substitution), or insertion, and wherein said alterations may occur at the amino- or carboxy-terminal positions of the query sequence or anywhere between those terminal positions, interspersed either individually among the amino acids or nucleotides in the query sequence or in one or more contiguous groups within the query sequence.

The % identity may be determined across the entire length of the query sequence, including the CDRs. Alternatively, the % identity may exclude one or more or all of the CDRs, for example all of the CDRs are 100% identical to the subject sequence and the % identity variation is in the remaining portion of the query sequence, e.g. the framework sequence, so that the CDR sequences are fixed and intact.

The variant sequence substantially retains the biological characteristics of the unmodified protein, such as an agonist for ICOS.

An antigen binding fragment may be provided by means of arrangement of one or more CDRs on non-antibody protein scaffolds. “Protein Scaffold” as used herein includes but is not limited to an immunoglobulin (Ig) scaffold, for example an IgG scaffold, which may be a four chain or two chain antibody, or which may comprise only the Fc region of an antibody, or which may comprise one or more constant regions from an antibody, which constant regions may be of human or primate origin, or which may be an artificial chimera of human and primate constant regions.

The protein scaffold may be an Ig scaffold, for example an IgG, or IgA scaffold. The IgG scaffold may comprise some or all the domains of an antibody (i.e. CH1, CH2, CH3, V_{H}, V_{L}). The antigen binding protein may comprise an IgG scaffold selected from IgG1, IgG2, IgG3, IgG4 or IgG4PE. For example, the scaffold may be IgG1. The scaffold may consist of, or comprise, the Fc region of an antibody, or is a part thereof.

The subclass of an antibody in part determines secondary effector functions, such as complement activation or Fc receptor (FcR) binding and antibody dependent cell cytotoxicity (ADCC) (Huber et al. Nature 229(5284): 419-20 (1971); Brunhouse et al. Mol Immunol 16(11): 907-17 (1979)). In identifying the optimal type of antibody for a particular application, the effector functions of the antibodies can be taken into account. For example, hIgG1 antibodies have a relatively long half life, are very effective at fixing complement, and they bind to both FcγRI and FcγRII. In contrast, human IgG4 antibodies have a shorter half life, do not fix complement and have a lower affinity for the FcRs. Replacement of serine 228 with a proline (S228P) in the Fc region of IgG4 reduces heterogeneity observed with hIgG4 and extends the serum half life (Kabat et al. “Sequences of proteins of immunological interest” 5.sup.th Edition (1991); Angal et al. Mol Immunol 30(1): 105-8 (1993)). A second mutation that replaces leucine 235 with a glutamic acid (L235E) eliminates the residual FcR binding and complement binding activities (Alegre et al. J Immunol 148(11): 3461-8 (1992)). The numbering of the hIgG4 amino acids was derived from EU numbering reference: Edelman et al. Proc. Natl. Acad. USA, 63, 78-85 (1969). PMID: 5257969.

The term “donor antibody” refers to an antibody that contributes the amino acid sequences of its variable regions, CDRs, or other functional fragments or analogs thereof to a first immunoglobulin partner. The donor, therefore, provides the altered immunoglobulin coding region and resulting expressed altered antibody with the antigenic specificity and neutralising activity characteristic of the donor antibody.

The term “acceptor antibody” refers to an antibody that is heterologous to the donor antibody, which contributes all (or any portion) of the amino acid sequences encoding its heavy and/or light chain framework regions and/or its heavy and/or light chain constant regions to the first immunoglobulin partner. A human antibody may be the acceptor antibody.

Affinity, also referred to as “binding affinity”, is the strength of binding at a single interaction site, i.e. of one molecule, e.g. an antigen binding protein of the invention, to another molecule, e.g. its target antigen, at a single binding site. The binding affinity of an antigen binding protein to its target may be determined by equilibrium methods (e.g. enzyme-linked immunoabsorbent assay (ELISA) or radioimmunoassay (RIA)), or kinetics (e.g. BIACORE analysis).

Avidity, also referred to as functional affinity, is the cumulative strength of binding at multiple interaction sites, e.g. the sum total of the strength of binding of two molecules (or more, e.g. in the case of a bispecific or multispecific molecule) to one another at multiple sites, e.g. taking into account the valency of the interaction.

As used herein an “immuno-modulator” or “immuno-modulatory agent” refers to any substance including monoclonal antibodies that affects the immune system. In some embodiments, the immuno-modulator or immuno-modulatory agent upregulates an aspect of the immune system. Immuno-modulators can be used as anti-neoplastic agents for the treatment of cancer. For example, immuno-modulators include, but are not limited to, anti-PD-1 antibodies (e.g. dostarlimab, OPDIVO/nivolumab, KEYTRUDA/pembrolizumab, LIBTAYO/cemiplimab) and anti-ICOS antibodies.

As used herein the term “agonist” refers to an antigen binding protein including but not limited to an antibody, that upon contact with a co-signalling receptor causes one or more of the following (1) stimulates or activates the receptor, (2) enhances, increases or promotes, induces or prolongs an activity, function or presence of the receptor and/or (3) enhances, increases, promotes or induces the expression of the receptor. Agonist activity can be measured in vitro by various assays know in the art such as, but not limited to, measurement of cell signalling, cell proliferation, immune cell activation markers, cytokine production. Agonist activity can also be measured in vivo by various assays that measure surrogate end points such as, but not limited to the measurement of T cell proliferation or cytokine production. In one embodiment, the ICOS binding protein is an agonist ICOS binding protein.

As used herein the term “antagonist” refers to an antigen binding protein including but not limited to an antibody, that upon contact with a co-signalling receptor causes one or more of the following (1) attenuates, blocks or inactivates the receptor and/or blocks activation of a receptor by its natural ligand, (2) reduces, decreases or shortens the activity, function or presence of the receptor and/or (3) reduces, decreases, abrogates the expression of the receptor. Antagonist activity can be measured in vitro by various assays know in the art such as, but not limited to, measurement of an increase or decrease in cell signalling, cell proliferation, immune cell activation markers, cytokine production. Antagonist activity can also be measured in vivo by various assays that measure surrogate end points such as, but not limited to the measurement of T cell proliferation or cytokine production. In one embodiment, the PD-1 binding protein is an antagonist PD-1 binding protein.

By “isolated” it is intended that the molecule, such as an antigen binding protein or nucleic acid, is removed from the environment in which it may be found in nature. For example, the molecule may be purified away from substances with which it would normally exist in nature. For example, the mass of the molecule in a sample may be 95% of the total mass.

The term “expression vector” as used herein means an isolated nucleic acid which can be used to introduce a nucleic acid of interest into a cell, such as a eukaryotic cell or prokaryotic cell, or a cell free expression system where the nucleic acid sequence of interest is expressed as a peptide chain such as a protein. Such expression vectors may be, for example, cosmids, plasmids, viral sequences, transposons, and linear nucleic acids comprising a nucleic acid of interest. Once the expression vector is introduced into a cell or cell free expression system (e.g. reticulocyte lysate) the protein encoded by the nucleic acid of interest is produced by the transcription/translation machinery. Expression vectors within the scope of the disclosure may provide necessary elements for eukaryotic or prokaryotic expression and include viral promoter driven vectors, such as CMV promoter driven vectors, e.g. pcDNA3.1, pCEP4, and their derivatives, Baculovirus expression vectors, Drosophila expression vectors, and expression vectors that are driven by mammalian gene promoters, such as human Ig gene promoters. Other examples include prokaryotic expression vectors, such as T7 promoter driven vectors, e.g. pET41, lactose promoter driven vectors and arabinose gene promoter driven vectors. Those of ordinary skill in the art will recognize many other suitable expression vectors and expression systems.

The term “recombinant host cell” as used herein means a cell that comprises a nucleic acid sequence of interest that was isolated prior to its introduction into the cell. For example, the nucleic acid sequence of interest may be in an expression vector while the cell may be prokaryotic or eukaryotic. Exemplary eukaryotic cells are mammalian cells, such as but not limited to, COS-1, COS-7, HEK293, BHK21, CHO, BSC-1, HepG2, 653, SP2/0, NS0, 293, HeLa, myeloma, lymphoma cells or any derivative thereof. Most preferably, the eukaryotic cell is a HEK293, NS0, SP2/0, or CHO cell. E. coli is an exemplary prokaryotic cell. A recombinant cell according to the disclosure may be generated by transfection, cell fusion, immortalization, or other procedures well known in the art. A nucleic acid sequence of interest, such as an expression vector, transfected into a cell may be extrachromasomal or stably integrated into the chromosome of the cell.

As used herein, the term “effective dose” means that dose of a drug or pharmaceutical agent that will elicit the biological or medical response of a tissue, system, animal or human that is being sought, for instance, by a researcher or clinician. Furthermore, the term “therapeutically effective dose” means any dose that, as compared to a corresponding subject who has not received such dose, results in improved treatment, healing, prevention, or amelioration of a disease, disorder, or side effect, or a decrease in the rate of advancement of a disease or disorder. The term also includes within its scope doses effective to enhance normal physiological function. Therapeutically effective amounts and treatment regimes are generally determined empirically and may be dependent on factors, such as the age, weight, and health status of the patient and disease or disorder to be treated. Such factors are within the purview of the attending physician.

Ranges provided herein, of any type, include all values within a particular range described and values about an endpoint for a particular range.

### Combinations

The present invention relates to a combination comprising an ICOS binding protein and a TIM-3 binding protein for use in the treatment of a cancer, in particular in the treatment of a cancer in a human.

Therefore, according to a first aspect of the invention, there is provided a combination comprising: an ICOS binding protein comprising a heavy chain amino acid sequence comprising a CDRH1 of SEQ ID NO:1, a CDRH2 of SEQ ID NO:2, and a CDRH3 of SEQ ID NO:3, and a light chain amino acid sequence comprising a CDRL1 of SEQ ID NO:4, a CDRL2 of SEQ ID NO:5, and a CDRL3 of SEQ ID NO:6; and a TIM-3 binding protein comprising a heavy chain amino acid sequence comprising a CDRH1 of SEQ ID NO:13, a CDRH2 of SEQ ID NO:14, and a CDRH3 of SEQ ID NO:15, and a light chain amino acid sequence comprising a CDRL1 of SEQ ID NO:16, a CDRL2 of SEQ ID NO:17, and a CDRL3 of SEQ ID NO:18, for use in the treatment of a cancer.

In another aspect, there is provided a combination comprising: an ICOS binding protein comprising a heavy chain amino acid sequence at least about 90% identical to the amino acid sequence of SEQ ID NO:9 and a light chain amino acid sequence at least about 90% identical to the amino acid sequence of SEQ ID NO:10; and a TIM-3 binding protein comprising a heavy chain amino acid sequence at least about 90% identical to the amino acid sequence of SEQ ID NO:21 and a light chain amino acid sequence at least about 90% identical to the amino acid sequence of SEQ ID NO:22, for use in the treatment of a cancer.

In another aspect, there is provided an ICOS binding protein comprising a heavy chain amino acid sequence comprising a CDRH1 of SEQ ID NO:1, a CDRH2 of SEQ ID NO:2, and a CDRH3 of SEQ ID NO:3, and a light chain amino acid sequence comprising a CDRL1 of SEQ ID NO:4, a CDRL2 of SEQ ID NO:5, and a CDRL3 of SEQ ID NO:6, for use in treating cancer in a human, wherein the ICOS binding protein is to be administered in combination with a TIM-3 binding protein comprising a CDRH1 of SEQ ID NO:13, a CDRH2 of SEQ ID NO:14, and a CDRH3 of SEQ ID NO:15, and a light chain amino acid sequence comprising a CDRL1 of SEQ ID NO:16, a CDRL2 of SEQ ID NO:17, and a CDRL3 of SEQ ID NO:18.

In another aspect, there is provided a TIM-3 binding protein comprising a heavy chain amino acid sequence comprising a CDRH1 of SEQ ID NO:13, a CDRH2 of SEQ ID NO:14, and a CDRH3 of SEQ ID NO:15, and a light chain amino acid sequence comprising a CDRL1 of SEQ ID NO:16, a CDRL2 of SEQ ID NO:17, and a CDRL3 of SEQ ID NO:18, for use in treating cancer, wherein the TIM-3 binding protein is to be administered in combination with an ICOS binding protein comprising a CDRH1 of SEQ ID NO:1, a CDRH2 of SEQ ID NO:2, and a CDRH3 of SEQ ID NO:3, and a light chain amino acid sequence comprising a CDRL1 of SEQ ID NO:4, a CDRL2 of SEQ ID NO:5, and a CDRL3 of SEQ ID NO:6.

The term “combination” of the invention described herein refers to at least two therapeutic agents (i.e. antigen binding proteins). It will be understood that references to a “combination” include embodiments where the two therapeutic agents are administered concurrently (i.e. simultaneously) or sequentially. Therefore, the individual therapeutic agents of the combination of the invention, and pharmaceutical compositions comprising such therapeutic agents may be administered together or separately. When administered separately, this may occur simultaneously or sequentially in any order (by the same or by different routes of administration). Such sequential administration may be close in time or remote in time. The dose of a therapeutic agents of the invention or pharmaceutically acceptable salt thereof and the further therapeutically active agent(s) and the relative timings of administration will be selected in order to achieve the desired combined therapeutic effect.

The administration of the combinations of the invention may be advantageous over the individual therapeutic agents in that the combinations may provide one or more of the following improved properties when compared to the individual administration of a single therapeutic agent alone: i) a greater anticancer effect than the most active single agent, ii) synergistic or highly synergistic anticancer activity, iii) a dosing protocol that provides enhanced anticancer activity with reduced side effect profile, iv) a reduction in the toxic effect profile, v) an increase in the therapeutic window, and/or vi) an increase in the bioavailability of one or both of the therapeutic agents.

In one embodiment, each antigen binding protein in a combination is individually formulated into its own pharmaceutical composition and each of the pharmaceutical compositions are administered to treat cancer. In this embodiment, each of the pharmaceutical compositions may have the same or different carriers, diluents or excipients. For example, in one embodiment, a first pharmaceutical composition contains an ICOS binding protein, a second pharmaceutical composition contains a TIM-3 binding protein, and the first and second pharmaceutical compositions are both administered to treat cancer.

In one embodiment, each binding protein in the combination is formulated together into a single pharmaceutical composition and administered to treat cancer. For example, in one embodiment, a single pharmaceutical composition contains both an ICOS binding protein and a TIM-3 binding protein and is administered as a single pharmaceutical composition to treat cancer.

Combinations of the invention may additionally comprise a Programmed Death 1 (PD-1) binding protein. As described hereinbefore, this binding protein may be administered concurrently (i.e. simultaneously) or sequentially with other binding agents of the combination combination in any order or combination of administration. For example, in one embodiment, administration may comprise ICOS binding protein followed by PD-1 binding protein followed by TIM-3 binding protein. In an alternative embodiment, administration may comprise ICOS binding protein followed by TIM-3 binding protein followed by PD-1 binding protein. In yet another embodiment, administration may comprise a TIM-3 binding protein followed by an ICOS binding protein followed by a PD-1 binding protein. In another embodiment, administration may comprise a TIM-3 binding protein followed by a PD-1 binding protein followed by an ICOS binding protein. In another embodiment, administration may comprise a PD-1 binding protein followed by an ICOS binding protein followed by a TIM-3 binding protein. In yet another embodiment, administration may comprise a PD-1 binding protein followed by a TIM-3 binding protein followed by an ICOS binding protein. All aspects and embodiments described herein, may also be applied to combinations additionally comprising PD-1 binding agents.

Antigen Binding Proteins and Antibodies that Bind ICOS

Agents directed to ICOS in any of the aspects or embodiments of the present invention include a monoclonal antibody (mAb), or antigen binding fragment thereof, which specifically binds to ICOS. In some embodiments, the mAb to ICOS specifically binds to human ICOS. In one embodiment, the ICOS binding protein is a monoclonal antibody or antigen binding fragment thereof. The mAb may be a human antibody, a humanized antibody or a chimeric antibody, and may include a human constant region. The human constant region is selected from the group consisting of IgG1, IgG2, IgG3 and IgG4 constant regions, and in preferred embodiments, the human constant region is an IgG1 or IgG4 constant region. The antigen binding fragment may be selected from the group consisting of Fab, Fab′-SH, F(ab′)2, scFv and Fv fragments.

As used herein “ICOS” means any Inducible T-cell costimulator protein. Pseudonyms for ICOS (Inducible T-cell COStimulator) include AILIM; CD278; CVID1, JTT-1 or JTT-2, MGC39850, or 8F4. ICOS is a CD28-superfamily costimulatory molecule that is expressed on activated T cells. The protein encoded by this gene belongs to the CD28 and CTLA-4 cell-surface receptor family. It forms homodimers and plays an important role in cell-cell signaling, immune responses, and regulation of cell proliferation. The amino acid sequence of human ICOS (isoform 2) (Accession No.: UniProtKB-Q9Y6W8-2) is shown below as SEQ ID NO:11.

(SEQ ID NO: 11)
MKSGLWYFFLFCLRIKVLTGEINGSANYEMFIFHNGGVQILCKYPDIVQQ
FKMQLLKGGQILCDLTKTKGSGNTVSIKSLKFCHSQLSNNSVSFFLYNLD
HSHANYYFCNLSIFDPPPFKVTLTGGYLHIYESQLCCQLKFWLPIGCAAF
VVVCILGCILICWLTKKM

The amino acid sequence of human ICOS (isoform 1) (Accession No.: UniProtKB-Q9Y6W8-1) is shown below as SEQ ID NO:12.

(SEQ ID NO: 12)
MKSGLWYFFLFCLRIKVLTGEINGSANYEMFIFHNGGVQILCKYPDIVQQ
FKMQLLKGGQILCDLTKTKGSGNTVSIKSLKFCHSQLSNNSVSFFLYNLD
HSHANYYFCNLSIFDPPPFKVTLTGGYLHIYESQLCCQLKFWLPIGCAAF
VVVCILGCILICWLTKKKYSSSVHDPNGEYMFMRAVNTAKKSRLTDVTL

Activation of ICOS occurs through binding by ICOS-L (B7RP-1/B7-H2). Neither B7-1 nor B7-2 (ligands for CD28 and CTLA4) bind or activate ICOS. However, ICOS-L has been shown to bind weakly to both CD28 and CTLA-4 (Yao et al. “B7-H2 is a costimulatory ligand for CD28 in human”, Immunity, 34(5); 729-40 (2011)). Expression of ICOS appears to be restricted to T cells. ICOS expression levels vary between different T cell subsets and on T cell activation status. ICOS expression has been shown on resting TH17, T follicular helper (TFH) and regulatory T (Treg) cells; however, unlike CD28; it is not highly expressed on naïve T_{H}1 and T_{H}2 effector T cell populations (Paulos et al. “The inducible costimulator (ICOS) is critical for the development of human Th17 cells”, Sci Transl Med, 2(55); 55ra78 (2010)). ICOS expression is highly induced on CD4+ and CD8+ effector T cells following activation through TCR engagement (Wakamatsu et al. “Convergent and divergent effects of costimulatory molecules in conventional and regulatory CD4+ T cells”, Proc Natl Acad Sci USA, 110(3); 1023-8 (2013)). Co-stimulatory signalling through ICOS receptor only occurs in T cells receiving a concurrent TCR activation signal (Sharpe A H and Freeman G J. “The B7-CD28 Superfamily”, Nat. Rev Immunol, 2(2); 116-26 (2002)). In activated antigen specific T cells, ICOS regulates the production of both T_{H}1 and T_{H}2 cytokines including IFN-γ, TNF-α, IL-10, IL-4, IL-13 and others. ICOS also stimulates effector T cell proliferation, albeit to a lesser extent than CD28 (Sharpe A H and Freeman G J. “The B7-CD28 Superfamily”, Nat. Rev Immunol, 2(2); 116-26 (2002)).

By “agent directed to ICOS” is meant any chemical compound or biological molecule capable of binding to ICOS. In some embodiments, the agent directed to ICOS is an ICOS binding protein. In some other embodiments, the agent directed to ICOS is an ICOS agonist. In some embodiments, the ICOS binding protein is an agonist ICOS binding protein.

The term “ICOS binding protein” as used herein refers to antibodies and other protein constructs, such as domains, which are capable of binding to ICOS. In some instances, the ICOS is human ICOS. The term “ICOS binding protein” can be used interchangeably with “ICOS binding agent”, “ICOS antigen binding protein” or “ICOS antigen binding agent”. Thus, as is understood in the art, anti-ICOS antibodies and/or ICOS antigen binding proteins would be considered ICOS binding proteins. This definition does not include the natural cognate ligand or receptor. References to ICOS binding proteins includes antigen binding portions or fragments thereof. As used herein “antigen binding portion” of an ICOS binding protein would include any portion of the ICOS binding protein capable of binding to ICOS, including but not limited to, an antigen binding antibody fragment.

In one embodiment, the ICOS binding proteins of the present invention comprise any one or a combination of the following CDRs:

| (SEQ ID NO: 1) | |
|---|---|
| CDRH1: | DYAMH |
| (SEQ ID NO: 2) | |
| CDRH2: | LISIYSDHTNYNQKFQG |
| (SEQ ID NO: 3) | |
| CDRH3: | NNYGNYGWYFDV |
| (SEQ ID NO: 4) | |
| CDRL1: | SASSSVSYMH |
| (SEQ ID NO: 5) | |
| CDRL2: | DTSKLAS |
| (SEQ ID NO: 6) | |
| CDRL3: | FQGSGYPYT |

In one embodiment, the ICOS binding protein comprises a heavy chain variable region CDR1 (“CDRH1”) comprising an amino acid sequence with one or two amino acid variation(s) (“CDR variant”) to the amino acid sequence set forth in SEQ ID NO:1.

In one embodiment, the ICOS binding protein comprises a heavy chain variable region CDR2 (“CDRH2”) comprising an amino acid sequence with five or fewer, such as four or fewer, three or fewer, two or fewer, or one amino acid variation(s) (“CDR variant”) to the amino acid sequence set forth in SEQ ID NO:2. In a further embodiment, the CDRH2 comprises an amino acid sequence with one or two amino acid variation(s) to the amino acid sequence set forth in SEQ ID NO:2.

In one embodiment, the ICOS binding protein comprises a heavy chain variable region CDR3 (“CDRH3”) comprising an amino acid sequence with one or two amino acid variation(s) (“CDR variant”) to the amino acid sequence set forth in SEQ ID NO:3.

In one embodiment, the ICOS binding protein comprises a light chain variable region CDR1 (“CDRL1”) comprising an amino acid sequence with three or fewer, such as one or two amino acid variation(s) (“CDR variant”) to the amino acid sequence set forth in SEQ ID NO:4.

In one embodiment, the ICOS binding protein comprises a light chain variable region CDR2 (“CDRL2”) comprising an amino acid sequence with one or two amino acid variation(s) (“CDR variant”) to the amino acid sequence set forth in SEQ ID NO:5.

In one embodiment, the ICOS binding protein comprises a light chain variable region CDR3 (“CDRL3”) comprising an amino acid sequence with three or fewer, such as one or two amino acid variation(s) (“CDR variant”) to the amino acid sequence set forth in SEQ ID NO:6.

In one embodiment, the ICOS binding protein comprises a CDRH1 comprising an amino acid sequence with up to one amino acid variation to the amino acid sequence set forth in SEQ ID NO:1; a CDRH2 comprising an amino acid sequence with up to five amino acid variations to the amino acid sequence set forth in SEQ ID NO:2; a CDRH3 comprising an amino acid sequence with up to one amino acid variation to the amino acid sequence set forth in SEQ ID NO:3; a CDRL1 comprising an amino acid sequence with up to three amino acid variations to the amino acid sequence set forth in SEQ ID NO:4; a CDRL2 comprising an amino acid sequence with up to one amino acid variation to the amino acid sequence set forth in SEQ ID NO:5; and/or a CDRL3 comprising an amino acid sequence with up to three amino acid variations to the amino acid sequence set forth in SEQ ID NO:6.

In one embodiment of the present invention the ICOS binding protein comprises CDRH1 (SEQ ID NO:1), CDRH2 (SEQ ID NO:2), and CDRH3 (SEQ ID NO:3) in the heavy chain variable region having the amino acid sequence set forth in SEQ ID NO:7. ICOS binding proteins of the present invention comprising the humanized heavy chain variable region set forth in SEQ ID NO:7 are designated as “H2.” In some embodiments, the anti-ICOS antibodies of the present invention comprise a heavy chain variable region having at least 90% sequence identity to SEQ ID NO:7. Suitably, the ICOS binding proteins of the present invention may comprise a heavy chain variable region having about 85%, 86%, 87%, 88%, 89%, 90%, 91%, 92%, 93%, 94%, 95%, 96%, 97%, 98%, 99%, or 100% sequence identity to SEQ ID NO:7.

Humanized heavy chain (V_{H}) variable region (H2):
(SEQ ID NO: 7; underlined amino acid residues
correspond to the positions of CDRs).
QVQLVQSGAEVKKPGSSVKVSCKASGYTFTDYAMHWVRQAPGQGLEWMG
LISIYSDHTNYNQKFQGRVTITADKSTSTAYMELSSLRSEDTAVYYCGR
NNYGNYGWYFDVWGQGTTVTVSS

In one embodiment, the ICOS binding protein comprises a heavy chain variable region (“V_{H}”) comprising an amino acid sequence with at least about 90%, 91%, 92%, 93%, 94%, 95%, 96%, 97%, 98%, 99% or 100% sequence identity to the amino acid sequence set forth in SEQ ID NO:7. In one embodiment, the V_{H} comprises an amino acid sequence with at least one amino acid variation to the amino acid sequence set forth in SEQ ID NO:7, such as between 1 and 5, such as between 1 and 3, in particular up to 2 amino acid variations to the amino acid sequence set forth in SEQ ID NO:7.

In one embodiment of the present invention the ICOS binding protein comprises CDRL1 (SEQ ID NO:4), CDRL2 (SEQ ID NO:5), and CDRL3 (SEQ ID NO:6) in the light chain variable region having the amino acid sequence set forth in SEQ ID NO:8. ICOS binding proteins of the present invention comprising the humanized light chain variable region set forth in SEQ ID NO:8 are designated as “L5.” Thus, an ICOS binding protein of the present invention comprising the heavy chain variable region of SEQ ID NO:7 and the light chain variable region of SEQ ID NO:8 can be designated as H2L5 herein.

In some embodiments, the ICOS binding proteins of the present invention comprise a light chain variable region having at least 90% sequence identity to the amino acid sequence set forth in SEQ ID NO:8. Suitably, the ICOS binding proteins of the present invention may comprise a light chain variable region having about 85%, 86%, 87%, 88%, 89%, 90%, 91%, 92%, 93%, 94%, 95%, 96%, 97%, 98%, 99%, or 100% sequence identity to SEQ ID NO:8.

Humanized light chain (V_{L}) variable region (L5):
(SEQ ID NO: 8; underlined amino acid residues
correspond to the positions of CDRs).
EIVLTQSPATLSLSPGERATLSCSASSSVSYMHWYQQKPGQAPRLLIY
DTSKLASGIPARFSGSGSGTDYTLTISSLEPEDFAVYYC
FQGSGYPYTFGQGTKLEIK

In one embodiment, the ICOS binding protein comprises a light chain variable region (“V_{L}”) comprising an amino acid sequence with at least about 90%, 91%, 92%, 93%, 94%, 95%, 96%, 97%, 98%, 99% or 100% sequence identity to the amino acid sequence set forth in SEQ ID NO:8. In one embodiment, the V_{L} comprises an amino acid sequence with at least one amino acid variation to the amino acid sequence set forth in SEQ ID NO:8, such as between 1 and 5, such as between 1 and 3, in particular up to 2 amino acid variations to the amino acid sequence set forth in SEQ ID NO:8.

In one embodiment, the ICOS binding protein comprises a V_{H} domain comprising an amino acid sequence at least 90% identical to the amino acid sequence set forth in SEQ ID NO:7 and/or a V_{L} domain comprising an amino acid sequence at least 90% identical to the amino acid sequence as set forth in SEQ ID NO:8 wherein said ICOS binding protein specifically binds to human ICOS. In one embodiment, the ICOS binding protein comprises a V_{H} with the amino acid sequence set forth in SEQ ID NO:7; and a V_{L} with the amino acid sequence set forth in SEQ ID NO:8.

In one embodiment, the ICOS binding protein comprises a V_{H} comprising an amino acid sequence of SEQ ID NO:7 and a V_{L} comprising an amino acid sequence of SEQ ID NO:8

In one embodiment, the ICOS binding protein comprises a V_{H} comprising an amino acid sequence with at least about 90%, 91%, 92%, 93%, 94%, 95%, 96%, 97%, 98%, 99% or 100% sequence identity to the amino acid sequence set forth in SEQ ID NO:7; and a V_{L} comprising an amino acid sequence with at least about 90%, 91%, 92%, 93%, 94%, 95%, 96%, 97%, 98%, 99% or 100% sequence identity to the amino acid sequence set forth in SEQ ID NO:8.

In one embodiment, the ICOS binding protein is a humanized monoclonal antibody comprising a heavy chain (HC) amino acid sequence having at least 90%, 91%, 92,%, 93%, 94%, 95%, 96%, 97%, 98%, 99% or 100% sequence identity to the amino acid sequence set forth in SEQ ID NO:9.

(SEQ ID NO: 9)
QVQLVQSGAEVKKPGSSVKVSCKASGYTFTDYAMHWVRQAPGQGLEWMGL
ISIYSDHTNYNQKFQGRVTITADKSTSTAYMELSSLRSEDTAVYYCGRNN
YGNYGWYFDVWGQGTTVTVSSASTKGPSVFPLAPCSRSTSESTAALGCLV
KDYFPEPVTVSWNSGALTSGVHTFPAVLQSSGLYSLSSVVTVPSSSLGTK
TYTCNVDHKPSNTKVDKRVESKYGPPCPPCPAPEFEGGPSVFLFPPKPKD
TLMISRTPEVTCVVVDVSQEDPEVQFNWYVDGVEVHNAKTKPREEQFNST
YRVVSVLTVLHQDWLNGKEYKCKVSNKGLPSSIEKTISKAKGQPREPQVY
TLPPSQEEMTKNQVSLTCLVKGFYPSDIAVEWESNGQPENNYKTTPPVLD
SDGSFFLYSRLTVDKSRWQEGNVFSCSVMHEALHNHYTQKSLSLSLGK

In one embodiment, the HC comprises an amino acid sequence with at least one amino acid variation to the amino acid sequence set forth in SEQ ID NO:9, such as between 1 and 10, such as between 1 and 7, in particular up to 6 amino acid variations to the amino acid sequence set forth in SEQ ID NO:9. In a further embodiment, the HC comprises one, two, three, four, five, six or seven amino acid variations to the amino acid sequence set forth in SEQ ID NO:9.

In one embodiment, the ICOS binding protein is a humanized monoclonal antibody comprising a light chain (LC) amino acid sequence having at least 90%, 91%, 92,%, 93%, 94%, 95%, 96%, 97%, 98%, 99% or 100% sequence identity to the amino acid sequence set forth in SEQ ID NO:10.

(SEQ ID NO: 10)
EIVLTQSPATLSLSPGERATLSCSASSSVSYMHWYQQKPGQAPRLLIYDT
SKLASGIPARFSGSGSGTDYTLTISSLEPEDFAVYYCFQGSGYPYTFGQG
TKLEIKRTVAAPSVFIFPPSDEQLKSGTASVVCLLNNFYPREAKVQWKVD
NALQSGNSQESVTEQDSKDSTYSLSSTLTLSKADYEKHKVYACEVTHQGL
SSPVTKSFNRGEC

In one embodiment, the LC comprises an amino acid sequence with at least one amino acid variation to the amino acid sequence set forth in SEQ ID NO:10, such as between 1 and 10, such as between 1 and 5, in particular up to 3 amino acid variations to the amino acid sequence set forth in SEQ ID NO:10. In a further embodiment, the LC comprises one, two or three amino acid variations to the amino acid sequence set forth in SEQ ID NO:10.

In one embodiment, the ICOS binding protein comprises a HC comprising an amino acid sequence with at least about 90%, 91%, 92%, 93%, 94%, 95%, 96%, 97%, 98%, 99% or 100% sequence identity to the amino acid sequence set forth in SEQ ID NO:9; and a LC comprising an amino acid sequence with at least about 90%, 91%, 92%, 93%, 94%, 95%, 96%, 97%, 98%, 99% or 100% sequence identity to the amino acid sequence set forth in SEQ ID NO:10. Therefore, the antibody is an antibody with a heavy chain at least about 90% identical to the heavy chain amino acid sequence of SEQ ID NO:9 and/or with a light chain at least about 90% identical to the light chain amino acid sequence of SEQ ID NO:10.

In one embodiment, the ICOS binding protein comprises a heavy chain amino acid sequence at least about 90% identical to the amino acid sequence of SEQ ID NO:9 and/or a light chain amino acid sequence at least about 90% identical to the amino acid sequence of SEQ ID NO:10.

In one embodiment, the ICOS binding protein comprises a heavy chain sequence of SEQ ID NO:9 and a light chain sequence of SEQ ID NO:10.

In one embodiment there is provided an ICOS binding protein comprising a heavy chain constant region such that has reduced ADCC and/or complement activation or effector functionality. In one such embodiment the heavy chain constant region may comprise a naturally disabled constant region of IgG2 or IgG4 isotype or a mutated IgG1 constant region.

In one embodiment, the ICOS binding protein comprises an IgG4 Fc region comprising the amino acid substitutions S228P and L235E or functional equivalents thereof. In one embodiment, the ICOS binding protein comprises an IgG4 Fc region comprising the amino acid substitutions S229P and L236E. Such embodiments may have the designation IgG4PE. Thus, an ICOS binding protein having the heavy chain variable region H2 and the light chain variable region L5 and an IgG4PE Fc region will be designated as H2L5 IgG4PE or synonymously as H2L5 hIgG4PE.

In one embodiment, the ICOS binding protein is feladilimab. In one embodiment, the ICOS binding protein is H2L5. In one embodiment, the ICOS binding protein is H2L5 IgG4PE.

Antibodies to ICOS and methods of using in the treatment of disease are described, for instance, in WO2012131004, US20110243929, and US20160215059. US20160215059 is incorporated by reference herein. CDRs for murine antibodies to human ICOS having agonist activity are shown in PCT/EP2012/055735 (WO2012131004). Antibodies to ICOS are also disclosed in WO2008137915, WO2010056804, EP1374902, EP1374901, and EP1125585. Agonist antibodies to ICOS or ICOS binding proteins are disclosed in WO2012/13004, WO2014033327, WO2016120789, US20160215059, and US20160304610. Exemplary antibodies in US20160304610 include 37A10S713. In an embodiment, the ICOS binding protein is vopratelimab. Sequences of 37A10S713 are reproduced below as SEQ ID NOS:41-48.

37A10S713 V_{H} CDR1:
(SEQ ID NO: 41)
GFTFSDYWMD
37A10S713 V_{H} CDR2:
(SEQ ID NO: 42)
NIDEDGSITEYSPFVKG
37A10S713 V_{H} CDR3:
(SEQ ID NO: 43)
WGRFGFDS
37A10S713 V_{L} CDR1:
(SEQ ID NO: 44)
KSSQSLLSGSFNYLT
37A10S713 V_{L} CDR2:
(SEQ ID NO: 45)
YASTRHT
37A10S713 V_{L} CDR3:
(SEQ ID NO: 46)
HHHYNAPPT
37A10S713 heavy chain variable region:
(SEQ ID NO: 47)
EVQLVESGGLVQPGGSLRLSCAASGFTFSDYWMDWVRQAPGKGLVWVSNI
DEDGSITEYSPFVKGRFTISRDNAKNTLYLQMNSLRAEDTAVYYCTRWGR
FGFDSWGQGTLVTVSS
37A10S713 light chain variable region:
(SEQ ID NO: 48)
DIVMTQSPDSLAVSLGERATINCKSSQSLLSGSFNYLTWYQQKPGQPPKL
LIFYASTRHTGVPDRFSGSGSGTDFTLTISSLQAEDVAVYYCHHHYNAPP
TFGPGTKVDIK
In an embodiment, the ICOS binding protein is
vopratelimab. In one embodiment, the ICOS
binding protein is JTX-2011.
Exemplary antibodies in US2018/0289790 include
ICOS.33 IgG1f S267E. Sequences of ICOS.33 IgG1f
S267E are reproduced below as SEQ ID NOS: 49-50:
ICOS.33 IgG1f S267E heavy chain variable domain:
(SEQ ID NO: 49)
EVQLVESGGGLVKPGGSLRLSCAASGFTFSDYFMHWVRQAPGKGLEWVGV
IDTKSFNYATYYSDLVKGRFTISRDDSKNTLYLQMNSLKTEDTAVYYCTA
TIAVPYYFDYWGQGTLVTVSS
ICOS.33 IgG1f S267E light chain variable domain:
(SEQ ID NO: 50)
DIQMTQSPSSLSASVGDRVTITCQASQDISNYLSWYQQKPGKAPKLLIYY
TNLLAEGVPSRFSGSGSGTDFTFTISSLQPEDIATYYCQQYYNYRIHGPG
TKVDIK

In one embodiment, the ICOS binding protein is BMS-986226.

Exemplary antibodies in WO2018/029474 include STIM003. Sequences of STIM003 are reproduced below as SEQ ID NOS: 51-52.

STIM003 heavy chain variable domain:
(SEQ ID NO: 51)
EVQLVESGGGVVRPGGSLRLSCVASGVTFDDYGMSWVRQAPGKGLEWVSG
INWNGGDTDYSDSVKGRFTISRDNAKNSLYLQMNSLRAEDTALYYCARDF
YGSGSYYHVPFDYWGQGILVTVSS
STIM003 light chain variable domain:
(SEQ ID NO: 52)
EIVLTQSPGTLSLSPGERATLSCRASQSVSRSYLAWYQQKRGQAPRLLIY
GASSRATGIPDRFSGDGSGTDFTLSISRLEPEDFAVYYCHQYDMSPFTFG
PGTKVDIK

In one embodiment, the ICOS binding protein is KY1044.

Exemplary antibodies in WO2018/045110 include XENP23104. Sequences of the ICOS binding Fab side ([ICOS]_H0.66_L0) of XENP23104 are reproduced below as SEQ ID NOS: 53-60.

XENP23104 [ICOS]_H0.66_L0 heavy chain variable
domain:
QVQLVQSGAEVKKPGASVKVSCKASGYTFTGYYMHWVRQAPGQGLEWMG
WINPHSGETIYAQKFQGRVTMTRDTSISTAYMELSSLRSEDTAVYYCAR
TYYYDTSGYYHDAFDVWGQGTMVTVSS
(SEQ ID NO: 53; underlined amino acid residues
correspond to the positions of CDRs).
XENP23104 [ICOS]_H0.66_L0 V_{H} CDR1:
(SEQ ID NO: 54)
GYYMH
XENP23104 [ICOS]_H0.66_L0 V_{H} CDR2:
(SEQ ID NO: 55)
WINPHSGETIYAQKFQG
XENP23104 [ICOS]_H0.66_L0 V_{H} CDR3:
(SEQ ID NO: 56)
TYYYDTSGYYHDAFDV
XENP23104 [ICOS]_H0.66_L0 light chain variable
domain:
DIQMTQSPSSVSASVGDRVTITCRASQGISRLLAWYQQKPGKAPKLLIYV
ASSLQSGVPSRFSGSGSGTDFTLTISSLQPEDFATYYCQQANSFPWTFGQ
GTKVEIK (SEQ ID NO: 57; underlined amino acid
residues correspond to the positions of CDRs).
XENP23104 [ICOS]_H0.66_L0 V_{L} CDR1:
(SEQ ID NO: 58)
RASQGISRLLA
XENP23104 [ICOS]_H0.66_L0 V_{L} CDR2:
(SEQ ID NO: 59)
VASSLQS
XENP23104 [ICOS]_H0.66_L0 V_{L} CDR3:
(SEQ ID NO: 60)
QQANSFPWT

As used herein “ICOS-L” and “ICOS Ligand” are used interchangeably and refer to the membrane bound natural ligand of human ICOS. ICOS ligand is a protein that in humans is encoded by the ICOSLG gene. ICOSLG has also been designated as CD275 (cluster of differentiation 275). Pseudonyms for ICOS-L include B7RP-1 and B7-H2.

Antigen Binding Proteins and Antibodies that Bind TIM-3

Agents directed to TIM-3 in any of the aspects or embodiments of the present invention include a monoclonal antibody (mAb), or antigen binding fragment thereof, that specifically binds to TIM-3. In some embodiments, the mAb to TIM-3 specifically binds to human TIM-3. In one embodiment, the TIM-3 binding protein is a monoclonal antibody or antigen binding fragment thereof. The mAb may be a human antibody, a humanized antibody or a chimeric antibody, and may include a human constant region. The human constant region is selected from the group consisting of IgG1, IgG2, IgG3 and IgG4 constant regions, and in preferred embodiments, the human constant region is an IgG1 or IgG4 constant region. The antigen binding fragment may be selected from the group consisting of Fab, Fab′-SH, F(ab′)2, scFv and Fv fragments.

As used herein “TIM-3” refers to the T Cell Immunoglobulin and Mucin Domain-3, also known as Hepatitis A Virus Cellular Receptor 2 (HAVCR2). It is a Th1-specific cell surface protein that regulates macrophage activation and enhances the severity of experimental autoimmune encephalomyelitis in mice. TIM-3 is highly expressed on the surface of multiple immune cell types, including, for example, Th1 IFN-γ+ cells, Th17 cells, natural killer (NK) cells, monocytes, and tumor-associated dendritic cells (DCs) (see, e.g. WO 2018/129553 and references contained therein). TIM-3 also is highly expressed on “exhausted” or impaired CD8+ T-cells in a variety of chronic viral infections (e.g. HIV, HCV, and HBV) and in certain cancers (see, e.g. WO 2018/129553 and references contained therein).

Putative ligands for TIM-3 include phosphatidylserine (Nakayama et al. Blood, 113: 3821-3830 (2009)), galectin-9 (Zhu et al. Nat. Immunol., 6: 1245-1252 (2005)), high-mobility group protein 1 (HMGB1) (Chiba et al. Nat. Immunol., 13: 832-842 (2012)), and carcinoembryonic antigen cell adhesion molecule 1 (CEACAM1) (Huang et al. Nature, 517(7534): 386-90 (2015)).

TIM-3 functions to regulate various aspects of the immune response. The interaction of TIM-3 and galectin-9 (Gal-9) induces cell death and in vivo blockade of this interaction exacerbates autoimmunity and abrogates tolerance in experimental models, strongly suggesting that TIM-3 is a negative regulatory molecule. In contrast to its effect on T-cells, the TIM-3-Gal-9 interaction exhibits antimicrobial effects by promoting macrophage clearance of intracellular pathogens (see, e.g. Sakuishi et al., Trends in Immunology, 32(8): 345-349 (2011)). In vivo suppression of TIM-3 has been shown to enhance the pathological severity of experimental autoimmune encephalomyelitis (Manney et al. supra; and Anderson, A. C. and Anderson, D. E., Curr. Opin. Immunol., 18: 665-669 (2006)). Studies also suggest that dysregulation of the TIM-3-galectin-9 pathway could play a role in chronic autoimmune diseases, such as multiple sclerosis (Anderson and Anderson, supra). TIM-3 promotes clearance of apoptotic cells by binding phosphatidyl serine through its unique binding cleft (see, e.g. DeKruyff et al., J. Immunol., 184(4):1918-1930 (2010)).

The amino acid sequence of human TIM-3 (Accession No.: UniProtKB-Q8TDQ0) is shown below as SEQ ID NO:23.

(SEQ ID NO: 23)
MFSHLPFDCVLLLLLLLLTRSSEVEYRAEVGQNAYLPCFYTPAAPGNLV
PVCWGKGACPVFECGNWLRTDERDVNYWTSRYWLNGDFRKGDVSLTIEN
VTLADSGIYCCRIQIPGIMNDEKFNLKLVIKPAKVTPAPTRQRDFTAAF
PRMLTTRGHGPAETQTLGSLPDINLTQISTLANELRDSRLANDLRDSGA
TIRIGIYIGAGICAGLALALIFGALIFKWYSHSKEKIQNLSLISLANLP
PSGLANAVAEGIRSEENIYTIEENVYEVEEPNEYYCYVSSRQQPSQPLG
CRFAMP

By “agent directed to TIM-3” is meant any chemical compound or biological molecule capable of binding to TIM-3. In some embodiments, the agent directed to TIM-3 is a TIM-3 binding protein.

The term “TIM-3 binding protein” as used herein refers to antibodies and other protein constructs, such as domains, that are capable of binding to TIM-3. In some instances, the TIM-3 is human TIM-3. The term “TIM-3 binding protein” can be used interchangeably with “TIM-3 binding agent”, “TIM-3 antigen binding protein” or “TIM-3 antigen binding agent”. Thus, as is understood in the art, anti-TIM-3 antibodies and/or TIM-3 antigen binding proteins would be considered TIM-3 binding proteins. This definition does not include the natural cognate ligand or receptor. References to TIM-3 binding proteins includes antigen binding portions or fragments thereof. As used herein “antigen binding portion” of a TIM-3 binding protein would include any portion of the TIM-3 binding protein capable of binding to TIM-3, including but not limited to, an antigen binding antibody fragment.

In one embodiment, the TIM-3 binding proteins of the present invention comprise any one or a combination of the following CDRs:

CDRH1:
(SEQ ID NO: 13)
SYDMS
CDRH2:
(SEQ ID NO: 14)
TISGGGTYTYYQDSVKG
CDRH3:
(SEQ ID NO: 15)
MDY
CDRL1:
(SEQ ID NO: 16)
RASQSIRRYLN
CDRL2:
(SEQ ID NO: 17)
GASTLQS
CDRL3:
(SEQ ID NO: 18)
QQSHSAPLT

In one embodiment, the TIM-3 binding protein comprises a heavy chain variable region CDR1 (“CDRH1”) comprising an amino acid sequence with one or two amino acid variation(s) (“CDR variant”) to the amino acid sequence set forth in SEQ ID NO:13.

In one embodiment, the TIM-3 binding protein comprises a heavy chain variable region CDR2 (“CDRH2”) comprising an amino acid sequence with five or fewer, such as four or fewer, three or fewer, two or fewer, or one amino acid variation(s) (“CDR variant”) to the amino acid sequence set forth in SEQ ID NO:14. In a further embodiment, the CDRH2 comprises an amino acid sequence with one or two amino acid variation(s) to the amino acid sequence set forth in SEQ ID NO:14.

In one embodiment, the TIM-3 binding protein comprises a heavy chain variable region CDR3 (“CDRH3”) comprising an amino acid sequence with one amino acid variation (“CDR variant”) to the amino acid sequence set forth in SEQ ID NO:15.

In one embodiment, the TIM-3 binding protein comprises a light chain variable region CDR1 (“CDRL1”) comprising an amino acid sequence with three or fewer, such as one or two amino acid variation(s) (“CDR variant”) to the amino acid sequence set forth in SEQ ID NO:16.

In one embodiment, the TIM-3 binding protein comprises a light chain variable region CDR2 (“CDRL2”) comprising an amino acid sequence with one or two amino acid variation(s) (“CDR variant”) to the amino acid sequence set forth in SEQ ID NO:17.

In one embodiment, the TIM-3 binding protein comprises a light chain variable region CDR3 (“CDRL3”) comprising an amino acid sequence with three or fewer, such as one or two amino acid variation(s) (“CDR variant”) to the amino acid sequence set forth in SEQ ID NO:18.

In one embodiment, the TIM-3 binding protein comprises a CDRH1 comprising an amino acid sequence with up to one amino acid variation to the amino acid sequence set forth in SEQ ID NO:13; a CDRH2 comprising an amino acid sequence with up to five amino acid variations to the amino acid sequence set forth in SEQ ID NO:14; a CDRH3 comprising an amino acid sequence with up to one amino acid variation to the amino acid sequence set forth in SEQ ID NO:15; a CDRL1 comprising an amino acid sequence with up to three amino acid variations to the amino acid sequence set forth in SEQ ID NO:16; a CDRL2 comprising an amino acid sequence with up to one amino acid variation to the amino acid sequence set forth in SEQ ID NO:17; and/or a CDRL3 comprising an amino acid sequence with up to three amino acid variations to the amino acid sequence set forth in SEQ ID NO:18.

In one embodiment of the present invention the TIM-3 binding protein comprises CDRH1 (SEQ ID NO:13), CDRH2 (SEQ ID NO:14), and CDRH3 (SEQ ID NO:15) in the heavy chain variable region having the amino acid sequence set forth in SEQ ID NO:19. In some embodiments, the TIM-3 binding proteins of the present invention comprise a heavy chain variable region having at least 90% sequence identity to SEQ ID NO:19. Suitably, the TIM-3 binding proteins of the present invention may comprise a heavy chain variable region having about 85%, 86%, 87%, 88%, 89%, 90%, 91%, 92%, 93%, 94%, 95%, 96%, 97%, 98%, 99%, or 100% sequence identity to SEQ ID NO:19.

TIM-3 heavy chain (V_{H}) variable region:
(SEQ ID NO: 19)
EVQLLESGGGLVQPGGSLRLSCAAASGFTFSSYDMSWVRQAPGKGLDWVS
TISGGGTYTYYQDSVKGRFTISRDNSKNTL
YLQMNSLRAEDTA VYYCASMDYWGQGTTVTVSS

In one embodiment, the TIM-3 binding protein comprises a heavy chain variable region (“V_{H}”) comprising an amino acid sequence with at least about 90%, 91%, 92%, 93%, 94%, 95%, 96%, 97%, 98%, 99% or 100% sequence identity to the amino acid sequence set forth in SEQ ID NO:19. In one embodiment, the V_{H} comprises an amino acid sequence with at least one amino acid variation to the amino acid sequence set forth in SEQ ID NO:19, such as between 1 and 5, such as between 1 and 3, in particular up to 2 amino acid variations to the amino acid sequence set forth in SEQ ID NO:19.

In one embodiment of the present invention the TIM-3 binding protein comprises CDRL1 (SEQ ID NO:16), CDRL2 (SEQ ID NO:17), and CDRL3 (SEQ ID NO:18) in the light chain variable region having the amino acid sequence set forth in SEQ ID NO:20. In some embodiments, the TIM-3 binding proteins of the present invention comprise a light chain variable region having at least 90% sequence identity to the amino acid sequence set forth in SEQ ID NO:20. Suitably, the TIM-3 binding proteins of the present invention may comprise a light chain variable region having about 85%, 86%, 87%, 88%, 89%, 90%, 91%, 92%, 93%, 94%, 95%, 96%, 97%, 98%, 99%, or 100% sequence identity to SEQ ID NO:20.

TIM-3 light chain (V_{L}) variable region:
(SEQ ID NO: 20)
DIQMTQSPSSLSASVGDRVTITCRASQSIRRYLNWYHQKPGKAPKLLIYG
ASTLQSGVPSRFSGSGSGTDFTLTISSLQPEDFA
VYYCQQSHSAPLTFGGGTKVEIK

In one embodiment, the TIM-3 binding protein comprises a light chain variable region (“V_{L}”) comprising an amino acid sequence with at least about 90%, 91%, 92%, 93%, 94%, 95%, 96%, 97%, 98%, 99% or 100% sequence identity to the amino acid sequence set forth in SEQ ID NO:20. In one embodiment, the V_{L} comprises an amino acid sequence with at least one amino acid variation to the amino acid sequence set forth in SEQ ID NO:20, such as between 1 and 5, such as between 1 and 3, in particular up to 2 amino acid variations to the amino acid sequence set forth in SEQ ID NO:20.

In one embodiment, a TIM-3 binding protein comprises a V_{H} with the amino acid sequence set forth in SEQ ID NO:19; and a V_{L} with the amino acid sequence set forth in SEQ ID NO:20.

In one embodiment, the TIM-3 binding protein comprises a V_{H} comprising an amino acid sequence with at least about 90%, 91%, 92%, 93%, 94%, 95%, 96%, 97%, 98%, 99% or 100% sequence identity to the amino acid sequence set forth in SEQ ID NO:19; and a V_{L} comprising an amino acid sequence with at least about 90%, 91%, 92%, 93%, 94%, 95%, 96%, 97%, 98%, 99% or 100% sequence identity to the amino acid sequence set forth in SEQ ID NO:20.

In one embodiment, the TIM-3 binding protein is a monoclonal antibody comprising a heavy chain (HC) amino acid sequence having at least 90%, 91%, 92,%, 93%, 94%, 95%, 96%, 97%, 98%, 99% or 100% sequence identity to the amino acid sequence set forth in SEQ ID NO:21.

(SEQ ID NO: 21)
EVQLLESGGGLVQPGGSLRLSCAAASGFTFSSYDMSWVRQAPGKGLDWVS
TISGGGTYTYYQDSVKGRFTISRDNSKNTLYLQMNSLRAEDTAVYYCASM
DYWGQGTTVTVSSASTKGPSVFPLAPCSRSTSESTAALGCLVKDYFPEPV
TVSWNSGALTSGVHTFPAVLQSSGLYSLSSVVTVPSSSLGTKTYTCNVDH
KPSNTKVDKRVESKYGPPCPPCPAPEFLGGPSVFLFPPKPKDTLMISRTP
EVTCVWVDVSQEDPEVQFNWYVDGVEVHNAKTKPREEQFNSTYRVVSVLT
VLHQDWLNGKEYKCKVSNKGLPSSIEKTISKAKGQPREPQVYTLPPSQEE
MTKNQVSLTCLVKGFYPSDIAVEWESNGQPENNYKTTPPVLDSDGSFFLY
SRLTVDKSRWQEGNVFSCSVMHEALHNHYTQKSLSLSLGK

In one embodiment, the HC comprises an amino acid sequence with at least one amino acid variation to the amino acid sequence set forth in SEQ ID NO:21, such as between 1 and 10, such as between 1 and 7, in particular up to 6 amino acid variations to the amino acid sequence set forth in SEQ ID NO:21. In a further embodiment, the HC comprises one, two, three, four, five, six or seven amino acid variations to the amino acid sequence set forth in SEQ ID NO:21.

In one embodiment, the TIM-3 binding protein is a humanized monoclonal antibody comprising a light chain (LC) amino acid sequence having at least 90%, 91%, 92,%, 93%, 94%, 95%, 96%, 97%, 98%, 99% or 100% sequence identity to the amino acid sequence set forth in SEQ ID NO:22.

(SEQ ID NO: 22)
DIQMTQSPSSLSASVGDRVTITCRASQSIRRYLNWYHQKPGKAPKLLIYG
ASTLQSGVPSRFSGSGSGTDFTLTISSLQPEDFAVYYCQQSHSAPLTFGG
GTKVEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC

In one embodiment, the LC comprises an amino acid sequence with at least one amino acid variation to the amino acid sequence set forth in SEQ ID NO:22, such as between 1 and 10, such as between 1 and 5, in particular up to 3 amino acid variations to the amino acid sequence set forth in SEQ ID NO:22. In a further embodiment, the LC comprises one, two or three amino acid variations to the amino acid sequence set forth in SEQ ID NO:22.

In one embodiment, the TIM-3 binding protein comprises a HC comprising an amino acid sequence with at least about 90%, 91%, 92%, 93%, 94%, 95%, 96%, 97%, 98%, 99% or 100% sequence identity to the amino acid sequence set forth in SEQ ID NO:21; and a LC comprising an amino acid sequence with at least about 90%, 91%, 92%, 93%, 94%, 95%, 96%, 97%, 98%, 99% or 100% sequence identity to the amino acid sequence set forth in SEQ ID NO:22. Therefore, the antibody is an antibody with a heavy chain at least about 90% identical to the heavy chain amino acid sequence of SEQ ID NO:21 and/or with a light chain at least about 90% identical to the light chain amino acid sequence of SEQ ID NO:22.

In one embodiment, the TIM-3 binding protein comprises a heavy chain sequence of SEQ ID NO:21 and a light chain sequence of SEQ ID NO:22. In one embodiment, the antibody is cobolimab comprising a heavy chain sequence of SEQ ID NO:21 and a light chain sequence of SEQ ID NO:22.

Examples of mAbs that bind human TIM-3 are described in WO 2015/117002 and U.S. Pat. No. 9,605,070.

MBG453 is a human monoclonal antibody being developed by Novartis directed against the inhibitory T cell receptor TIM-3, with potential immune checkpoint inhibitory and antineoplastic activities. MBG453 binds to and blocks TIM-3 expressed on immune cells, including tumour infiltrating lymphocytes, resulting in the abrogation of T cell inhibition, activation of antigen-specific T lymphocytes and enhancement of cytotoxic T cell-mediated tumour cell lysis. This results in a reduction of tumour cell growth. MBG453 is currently being investigated in clinical trial NCT03946670 in 1L Myelodysplastic Syndrome (MDS) and clinical trial NCT04150029 in 1L acute myeloid leukaemia (AML).

LY3321367 is a human monoclonal antibody being developed by Eli Lilly directed against TIM-3 which has been shown to partially block the TIM-3/Gal-9 complex but not the TIM-3/CEACAM-1 complex. LY3321367 has also been shown to completely block phosphatidylserine (PS) binding to TIM-3 (Haidar et al. (2019) Cancer Res 79 (13 Supplement):Abstract No. 2753). LY3321367 is currently being investigated in patients with advanced relapsed or refractory solid tumours (NCT03099109 and NCT02791334).

Antigen Binding Proteins and Antibodies that Bind to PD-1

Agents directed to PD-1 in any of the aspects or embodiments of the present invention include a monoclonal antibody (mAb), or antigen binding fragment thereof, which specifically binds to PD-1. In one embodiment, the PD-1 binding protein is a monoclonal antibody or antigen binding fragment thereof. In some embodiments, the mAb to PD-1 specifically binds to human PD-1. The mAb may be a human antibody, a humanized antibody or a chimeric antibody, and may include a human constant region. The human constant region is selected from the group consisting of IgG1, IgG2, IgG3 and IgG4 constant regions, and in preferred embodiments, the human constant region is an IgG1 or IgG4 constant region. In a further embodiment, the PD-1 binding agent is an immunoglobulin G4 (IgG4) monoclonal antibody, in particular an IgG4 humanized monoclonal antibody. The antigen binding fragment may be selected from the group consisting of Fab, Fab′-SH, F(ab′)2, scFv and Fv fragments.

The protein Programmed Death 1 (PD-1) is an inhibitory member of the CD28 family of receptors, that also includes CD28, CTLA-4, ICOS and BTLA. PD-1 is expressed on activated B cells, T cells, and myeloid cells (Okazaki et al. (2002) Curr. Opin. Immunol 14:391779-82; Bennett et al. (2003) J Immunol 170:711-8) The initial members of the family, CD28 and ICOS, were discovered by functional effects on augmenting T cell proliferation following the addition of monoclonal antibodies (Hutloff et al. (1999) Nature 397:263-266; Hansen et al. (1980) Immunogenics 10:247-260). PD-1 was discovered through screening for differential expression in apototic cells (Ishida et al. (1992) EMBO J 11:3887-95). The other members of the family, CTLA-4, and BTLA were discovered through screening for differential expression in cytotoxic T lymphocytes and TH1 cells, respectively. CD28, ICOS and CTLA-4 all have an unpaired cysteine residue allowing for homodimerization. In contrast, PD-1 is suggested to exist as a monomer, lacking the unpaired cysteine residue characteristic in other CD28 family members. PD-1 antibodies and methods of using in treatment of disease are described in U.S. Pat. Nos. 7,595,048; 8,168,179; 8,728,474; 7,722,868; 8,008,449; 7,488,802; 7,521,051; 8,088,905; 8,168,757; and 8,354,509; and US Publication Nos.: US20110171220; US20110171215; and US20110271358. Combinations of CTLA-4 and PD-1 antibodies are described in U.S. Pat. No. 9,084,776.

The agent directed to PD-1 is a PD-1 antagonist and blocks binding of PD-L1 expressed on a cancer cell to PD-1 expressed on an immune cell (T cell, B cell or NKT cell) and may also block binding of PD-L2 expressed on a cancer cell to the immune-cell expressed PD-1. Alternative names or synonyms for PD-1 and its ligands include: PDCD1, PD1, CD279 and SLEB2 for PD-1; PDCD1L1, PDL1, B7H1, B7-4, CD274 and B7-H for PD-L1; and PDCD1L2, PDL2, B7-DC, Btdc and CD273 for PD-L2. Human PD-1 amino acid sequences can be found in NCBI Locus No.: NP_005009. The amino acid sequence in NCBI Locus No.: NP_005009 is reproduced below:

(SEQ ID NO: 38)
MQIPQAPWPVVWAVLQLGWRPGWFLDSPDRPWNPPTFSPALLWVTEGDNA
TFTCSFSNTSESFVLNWYRMSPSNQTDKLAAFPEDRSQPGQDCRFRVTQL
PNGRDFHMSVVRARRNDSGTYLCGAISLAPKAQIKESLRAELRVTERRAE
VPTAHPSPSPRPAGQFQTLVVGVVGGLLGSLVLLVWLAVICSRAARGTIG
ARRTGQPLKEDPSAVPVFSVDYGELDFQWREKTPEPPVPCVPEQTEYATI
VFPSGMGTSSPARRGSADGPRSAQPLRPEDGHCSWPL

Human PD-L1 and PD-L2 amino acid sequences can be found in NCBI Locus No.: NP_054862 and NP_079515, respectively.

The amino acid sequence in NCBI Locus No.:
NP_054862 is reproduced below:
(SEQ ID NO: 39)
MRIFAVFIFMTYWHLLNAFTVTVPKDLYVVEYGSNMTIECKFPVEKQLD
LAALIVYWEMEDKNIIQFVHGEEDLKVQHSSYRQRARLLKDQLSLGNAA
LQITDVKLQDAGVYRCMISYGGADYKRITVKVNAPYNKINQRILVVDPV
TSEHELTCQAEGYPKAEVIWTSSDHQVLSGKTTTTNSKREEKLFNVTST
LRINTTTNEIFYCTFRRLDPEENHTAELVIPELPLAHPPNERTHLVILG
AILLCLGVALTFIFRLRKGRMMDVKKCGIQDTNSKKQSDTHLEET
The amino acid sequence in NCBI Locus No.:
NP_079515 is reproduced below:
(SEQ ID NO: 40)
MIFLLLMLSLELQLHQIAALFTVTVPKELYIIEHGSNVTLECNFDTGSH
VNLGAITASLQKVENDTSPHRERATLLEEQLPLGKASFHIPQVQVRDEG
QYQCIIIYGVAWDYKYLTLKVKASYRKINTHILKVPETDEVELTCQATG
YPLAEVSWPNVSVPANTSHSRTPEGLYQVTSVLRLKPPPGRNFSCVFWN
THVRELTLASIDLQSQMEPRTHPTWLLHIFIPFCIIAFIFIATVIALRK
QLCQKLYSSKDTTKRPVTTTKREVNSAI

As used herein, an “agent directed to PD-1” or “agent directed to PD1” means any chemical compound or biological molecule capable of binding to PD-1. In some embodiments, the agent directed to PD-1 is a PD-1 binding protein. In some embodiments, the agent directed to PD-1 is a PD-1 antagonist. In some embodiments, the PD-1 binding protein is an antagonist PD-1 binding protein.

The term “PD-1 binding protein” or “PD1 binding protein” as used herein refers to antibodies and other protein constructs, such as domains, that are capable of binding to PD-1. In some instances, the PD-1 is human PD-1. The term “PD-1 binding protein” can be used interchangeably with “PD-1 binding agent”, “PD-1 antigen binding protein” or “PD-1 antigen binding agent”. Thus, as is understood in the art, anti-PD-1 antibodies and/or PD-1 antigen binding proteins would be considered PD-1 binding proteins. This definition does not include the natural cognate ligand or receptor. References to PD-1 binding proteins includes antigen binding portions or fragments thereof. As used herein “antigen binding portion” of a PD-1 binding protein would include any portion of the PD-1 binding protein capable of binding to PD-1, including but not limited to, an antigen binding antibody fragment.

In one embodiment, the PD-1 binding proteins of the present invention comprise any one or a combination of the following CDRs:

CDRH1:
(SEQ ID NO: 24)
SYDMS
CDRH2:
(SEQ ID NO: 25)
CDRH3:PYYAMDY
(SEQ ID NO: 26)
TISGGGSYTYYQDSVKG
CDRL1:
(SEQ ID NO: 27)
KASQDVGTAVA
CDRL2:
(SEQ ID NO: 28)
WASTLHT
CDRL3:
(SEQ ID NO: 29)
QHYSSYPWT

In one embodiment, the PD-1 binding protein comprises a heavy chain variable region CDR1 (“CDRH1”) comprising an amino acid sequence with one or two amino acid variation(s) (“CDR variant”) to the amino acid sequence set forth in SEQ ID NO:24.

In one embodiment, the PD-1 binding protein comprises a heavy chain variable region CDR2 (“CDRH2”) comprising an amino acid sequence with five or fewer, such as four or fewer, three or fewer, two or fewer, or one amino acid variation(s) (“CDR variant”) to the amino acid sequence set forth in SEQ ID NO:25. In a further embodiment, the CDRH2 comprises an amino acid sequence with one or two amino acid variation(s) to the amino acid sequence set forth in SEQ ID NO:25.

In one embodiment, the PD-1 binding protein comprises a heavy chain variable region CDR3 (“CDRH3”) comprising an amino acid sequence with one or two amino acid variation(s) (“CDR variant”) to the amino acid sequence set forth in SEQ ID NO:26.

In one embodiment, the PD-1 binding protein comprises a light chain variable region CDR1 (“CDRL1”) comprising an amino acid sequence with three or fewer, such as one or two amino acid variation(s) (“CDR variant”) to the amino acid sequence set forth in SEQ ID NO:27.

In one embodiment, the PD-1 binding protein comprises a light chain variable region CDR2 (“CDRL2”) comprising an amino acid sequence with one or two amino acid variation(s) (“CDR variant”) to the amino acid sequence set forth in SEQ ID NO:28.

In one embodiment, the PD-1 binding protein comprises a light chain variable region CDR3 (“CDRL3”) comprising an amino acid sequence with three or fewer, such as one or two amino acid variation(s) (“CDR variant”) to the amino acid sequence set forth in SEQ ID NO:29. In a particular embodiment, the CDRL3 comprises an amino acid sequence with one amino acid variation to the amino acid sequence set forth in SEQ ID NO:29. In a further embodiment, the variant CDRL3 comprises the amino acid sequence set forth in SEQ ID NO:37.

In one embodiment, the PD-1 binding protein comprises a CDRH1 comprising an amino acid sequence with up to one amino acid variation to the amino acid sequence set forth in SEQ ID NO:24; a CDRH2 comprising an amino acid sequence with up to five amino acid variations to the amino acid sequence set forth in SEQ ID NO:25; a CDRH3 comprising an amino acid sequence with up to one amino acid variation to the amino acid sequence set forth in SEQ ID NO:26; a CDRL1 comprising an amino acid sequence with up to three amino acid variations to the amino acid sequence set forth in SEQ ID NO:27; a CDRL2 comprising an amino acid sequence with up to one amino acid variation to the amino acid sequence set forth in SEQ ID NO:28; and/or a CDRL3 comprising an amino acid sequence with up to three amino acid variations to the amino acid sequence set forth in SEQ ID NO:29.

In one embodiment of the present invention the PD-1 binding protein comprises CDRH1 (SEQ ID NO:24), CDRH2 (SEQ ID NO:25), and CDRH3 (SEQ ID NO:26) in the heavy chain variable region having the amino acid sequence set forth in SEQ ID NO:30. In some embodiments, the anti-PD-1 antibodies of the present invention comprise a heavy chain variable region having at least 90% sequence identity to SEQ ID NO:30. Suitably, the PD-1 binding proteins of the present invention may comprise a heavy chain variable region having about 85%, 86%, 87%, 88%, 89%, 90%, 91%, 92%, 93%, 94%, 95%, 96%, 97%, 98%, 99%, or 100% sequence identity to SEQ ID NO:30.

PD-1 heavy chain (V_{H}) variable region:
(SEQ ID NO: 30)
EVQLLESGGGLVQPGGSLRLSCAASGFTFSSYDMSWVRQAPGKGLEWSTI
SGGGSYTYYQDSVKGRFTISRDNSKNTLYLQMNSLRAEDTAVYYCASPYY
AMDYWGQGTTVTVSS

In one embodiment, the PD-1 binding protein comprises a heavy chain variable region (“V_{H}”) comprising an amino acid sequence with at least about 90%, 91%, 92%, 93%, 94%, 95%, 96%, 97%, 98%, 99% or 100% sequence identity to the amino acid sequence set forth in SEQ ID NO:30. In one embodiment, the V_{H} comprises an amino acid sequence with at least one amino acid variation to the amino acid sequence set forth in SEQ ID NO:30, such as between 1 and 5, such as between 1 and 3, in particular up to 2 amino acid variations to the amino acid sequence set forth in SEQ ID NO:30.

In one embodiment of the present invention the PD-1 binding protein comprises CDRL1 (SEQ ID NO:27), CDRL2 (SEQ ID NO:28), and CDRL3 (SEQ ID NO:29) in the light chain variable region having the amino acid sequence set forth in SEQ ID NO:31. In one embodiment, a PD-1 binding protein of the present invention comprises the heavy chain variable region of SEQ ID NO:30 and the light chain variable region of SEQ ID NO:31.

In some embodiments, the PD-1 binding proteins of the present invention comprise a light chain variable region having at least 90% sequence identity to the amino acid sequence set forth in SEQ ID NO:31. Suitably, the PD-1 binding proteins of the present invention may comprise a light chain variable region having about 85%, 86%, 87%, 88%, 89%, 90%, 91%, 92%, 93%, 94%, 95%, 96%, 97%, 98%, 99%, or 100% sequence identity to SEQ ID NO:31.

PD-1 light chain (V_{L}) variable region:
(SEQ ID NO: 31)
DIQLTQSPSFLSAYVGDRVTITCKASQDVGTAVAWYQQKPGKAPKLLIY
WASTLHTGVPSRFSGSGSGTEFTLTISSLQPEDFATYYCQHYSSYPWTF
GQGTKLEIK

In one embodiment, the PD-1 binding protein comprises a light chain variable region (“V_{L}”) comprising an amino acid sequence with at least about 90%, 91%, 92%, 93%, 94%, 95%, 96%, 97%, 98%, 99% or 100% sequence identity to the amino acid sequence set forth in SEQ ID NO:31. In one embodiment, the V_{L} comprises an amino acid sequence with at least one amino acid variation to the amino acid sequence set forth in SEQ ID NO:31, such as between 1 and 5, such as between 1 and 3, in particular up to 2 amino acid variations to the amino acid sequence set forth in SEQ ID NO:31.

In one embodiment, the PD-1 binding protein comprises a V_{H} comprising an amino acid sequence with at least about 90%, 91%, 92%, 93%, 94%, 95%, 96%, 97%, 98%, 99% or 100% sequence identity to the amino acid sequence set forth in SEQ ID NO:30; and a V_{L} comprising an amino acid sequence with at least about 90%, 91%, 92%, 93%, 94%, 95%, 96%, 97%, 98%, 99% or 100% sequence identity to the amino acid sequence set forth in SEQ ID NO:31. In one embodiment, the PD-1 binding protein comprises a V_{H} at least about 90% identical to the amino acid sequence of SEQ ID NO:30 and/or a V_{L} at least about 90% identical to the amino acid sequence of SEQ ID NO:31.

In one embodiment, a PD-1 binding protein comprises a V_{H} with the amino acid sequence set forth in SEQ ID NO:30, and a V_{L} with the amino acid sequence set forth in SEQ ID NO:31.

In one embodiment, the PD-1 binding protein is a monoclonal antibody comprising a heavy chain (HC) amino acid sequence having at least 90%, 91%, 92,%, 93%, 94%, 95%, 96%, 97%, 98%, 99% or 100% sequence identity to the amino acid sequence set forth in SEQ ID NO:32.

(SEQ ID NO: 32)
EVQLLESGGGLVQPGGSLRLSCAASGFTFSSYDMSWVRQAPGKGLEWVS
TISGGGSYTYYQDSVKGRFTISRDNSKNTLYLQMNSLRAEDTAVYYCAS
PYYAMDYWGQGTTVTVSSASTKGPSVFPLAPCSRSTSESTAALGCLVKD
YFPEPVTVSWNSGALTSGVHTFPAVLQSSGLYSLSSVVTVPSSSLGTKT
YTCNVDHKPSNTKVDKRVESKYGPPCPPCPAPEFLGGPSVFLFPPKPKD
TLMISRTPEVTCVVVDVSQEDPEVQFNWYVDGVEVHNAKTKPREEQFNS
TYRVVSVLTVLHQDWLNGKEYKCKVSNKGLPSSIEKTISKAKGQPREPQ
VYTLPPSQEEMTKNQVSLTCLVKGFYPSDIAVEWESNGQPENNYKTTPP
VLDSDGSFFLYSRLTVDKSRWQEGNVFSCSVMHEALHNHYTQKSLSLSL
GK

In one embodiment, the HC comprises an amino acid sequence with at least one amino acid variation to the amino acid sequence set forth in SEQ ID NO:32, such as between 1 and 10, such as between 1 and 7, in particular up to 6 amino acid variations to the amino acid sequence set forth in SEQ ID NO:32. In a further embodiment, the HC comprises one, two, three, four, five, six or seven amino acid variations to the amino acid sequence set forth in SEQ ID NO:32.

In one embodiment, the HC chain comprises a variation at position 380 and/or 385 of SEQ ID NO:32. The asparagine residues at these positions may be modified, e.g. by deamidation (conversion of a asparagine (N) residue into an aspartate (D) residue). Therefore, in one embodiment, the HC comprises an amino acid sequence of SEQ ID NO:34 (N380D), SEQ ID NO:35 (N385D) or SEQ ID NO:36 (N380D and N385D).

In one embodiment, the PD-1 binding protein is a monoclonal antibody comprising a light chain (LC) amino acid sequence having at least 90%, 91%, 92,%, 93%, 94%, 95%, 96%, 97%, 98%, 99% or 100% sequence identity to the amino acid sequence set forth in SEQ ID NO:33.

(SEQ ID NO: 33)
DIQLTQSPSFLSAYVGDRVTITCKASQDVGTAVAWYQQKPGKAPKLLIYW
ASTLHTGVPSRFSGSGSGTEFTLTISSLQPEDFATYYCQHYSSYPWTFGQ
GTKLEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC

In one embodiment, the LC comprises an amino acid sequence with at least one amino acid variation to the amino acid sequence set forth in SEQ ID NO:33, such as between 1 and 10, such as between 1 and 5, in particular up to 3 amino acid variations to the amino acid sequence set forth in SEQ ID NO:33. In a further embodiment, the LC comprises one, two or three amino acid variations to the amino acid sequence set forth in SEQ ID NO:33.

In one embodiment, the PD-1 binding protein comprises a HC comprising an amino acid sequence with at least about 90%, 91%, 92%, 93%, 94%, 95%, 96%, 97%, 98%, 99% or 100% sequence identity to the amino acid sequence set forth in SEQ ID NO:32; and a LC comprising an amino acid sequence with at least about 90%, 91%, 92%, 93%, 94%, 95%, 96%, 97%, 98%, 99% or 100% sequence identity to the amino acid sequence set forth in SEQ ID NO:33. Therefore, the antibody is an antibody with a heavy chain at least about 90% identical to the heavy chain amino acid sequence of SEQ ID NO:32 and/or with a light chain at least about 90% identical to the light chain amino acid sequence of SEQ ID NO:33.

In one embodiment, the PD-1 binding protein comprises a heavy chain amino acid sequence at least about 90% identical to the amino acid sequence of SEQ ID NO:32 and/or a light chain amino acid sequence at least about 90% identical to the amino acid sequence of SEQ ID NO:33.

In one embodiment, the PD-1 binding protein comprises a heavy chain sequence of SEQ ID NO:32 and a light chain sequence of SEQ ID NO:33. In one embodiment, the antibody is dostarlimab comprising a heavy chain sequence of SEQ ID NO:32 and a light chain sequence of SEQ ID NO:33.

Examples of mAbs that bind to human PD-1 are described in U.S. Pat. Nos. 8,552,154; 8,008,449; 7,521,051; 7,488,802; and WO2004072286, WO2004056875 and WO2004004771.

Other PD-1 binding proteins include an immunoadhesin that specifically binds to PD-1, and preferably specifically binds to human PD-1, e.g. a fusion protein containing the extracellular or PD-1 binding portion of PD-L1 or PD-L2 fused to a constant region such as an Fc region of an immunoglobulin molecule. Examples of immunoadhesin molecules that specifically bind to PD-1 are described in WO2010027827 and WO2011066342. Specific fusion proteins useful as the PD-1 antagonist in the treatment method, medicaments and uses of the present invention include AMP-224 (also known as B7-DCIg), which is a PD-L2-FC fusion protein and binds to human PD-1.

OPDIVO/nivolumab is a fully human monoclonal antibody marketed by Bristol Myers Squibb directed against the negative immunoregulatory human cell surface receptor PD-1 (programmed death-1 or programmed cell death-1/PCD-1) with immunopotentiation activity. As an aspect of its function, nivolumab binds to and blocks the activation of PD-1, an Ig superfamily transmembrane protein, by its ligands PD-L1 and/or PD-L2, resulting in the activation of T-cells and cell-mediated immune responses against tumor cells or pathogens. Activated PD-1 negatively regulates T-cell activation and effector function through the suppression of P13k/Akt pathway activation. Other names for nivolumab include: BMS-936558, MDX-1106, and ONO-4538. The amino acid sequence for nivolumab and methods of using and making are disclosed in U.S. Pat. No. 8,008,449.

LIBTAYO/cemiplimab-rwlc is an anti-PD-1 antibody for treatment of cancer, including advanced cutaneous squamous cell carcinoma.

### Methods of Treatment

The antigen binding proteins described herein may also be used in methods of treatment. It will be appreciated by those skilled in the art that references herein to treatment refer to the treatment of established conditions. However, compositions of the invention may, depending on the condition, also be useful in the prevention of certain diseases. The antigen binding proteins described herein can be used in an effective amount for therapeutic, prophylactic or preventative treatment. A therapeutically effective amount of the antigen binding proteins described herein is an amount effective to ameliorate or reduce one or more symptoms of, or to prevent or cure, the disease.

In one aspect, there is provided a method of treating cancer in a human in need thereof, the method comprising administering to the human an ICOS binding protein. In another aspect, there is provided an ICOS binding protein for use in treating cancer. In a further aspect, there is provided use of an ICOS binding protein in the manufacture of a medicament for treating cancer. There is disclosed a pharmaceutical kit comprising an ICOS binding protein.

In one aspect, there is provided a method of treating cancer in a human in need thereof, the method comprising administering to the human a TIM-3 binding protein. In another aspect, there is provided a TIM-3 binding protein for use in treating cancer. In a further aspect, there is provided use of a TIM-3 binding protein in the manufacture of a medicament for treating cancer. There is disclosed a pharmaceutical kit comprising a TIM-3 binding protein.

In one embodiment, the binding proteins are administered simultaneously/concurrently. In an alternative embodiment, the binding proteins are administered sequentially (e.g. a first regimen administered prior to administration of any doses of a second regimen).

In one aspect, there is provided a method of treating cancer in a human in need thereof, the method comprising administering to the human an ICOS binding protein and a TIM-3 binding protein. In a further aspect, there is provided an ICOS binding protein and a TIM-3 binding protein for concurrent or sequential use in treating cancer. In another aspect, there is provided an ICOS binding protein for use in treating cancer is provided, wherein the ICOS binding protein is to be administered concurrently or sequentially with a TIM-3 binding protein. In one aspect, there is provided the use of an ICOS binding protein in the manufacture of a medicament for treating cancer, wherein the ICOS binding protein is to be administered concurrently or sequentially with a TIM-3 binding protein. In another aspect, there is provided a pharmaceutical kit comprising an ICOS binding protein and a TIM-3 binding protein.

In one embodiment the ICOS binding protein comprises a V_{H} domain comprising an amino acid sequence at least 90% identical to the amino acid sequence set forth in SEQ ID NO:7 and/or a V_{L} domain comprising an amino acid sequence at least 90% identical to the amino acid sequence as set forth in SEQ ID NO:8 wherein said ICOS binding protein specifically binds to human ICOS. In one embodiment, the ICOS binding protein comprises one or more of: CDRH1 as set forth in SEQ ID NO:1; CDRH2 as set forth in SEQ ID NO:2; CDRH3 as set forth in SEQ ID NO:3; CDRL1 as set forth in SEQ ID NO:4; CDRL2 as set forth in SEQ ID NO:5 and/or CDRL3 as set forth in SEQ ID NO:6 or a direct equivalent of each CDR wherein a direct equivalent has no more than two amino acid substitutions in said CDR. In one embodiment, the ICOS binding protein comprises a heavy chain variable region comprising one or more of SEQ ID NO:1; SEQ ID NO:2; and SEQ ID NO:3 and wherein said ICOS binding protein comprises a light chain variable region comprising one or more of SEQ ID NO:4; SEQ ID NO:5, and SEQ ID NO:6. In one embodiment, the ICOS binding protein comprises a heavy chain variable region comprising SEQ ID NO:1; SEQ ID NO:2; and SEQ ID NO:3 and wherein said ICOS binding protein comprises a light chain variable region comprising SEQ ID NO:4; SEQ ID NO:5, and SEQ ID NO:6. In one embodiment, the ICOS binding protein comprises a V_{H} domain comprising the amino acid sequence set forth in SEQ ID NO:7 and a V_{L} domain comprising the amino acid sequence as set forth in SEQ ID NO:8. In one embodiment, the ICOS binding protein comprises a heavy chain comprising the amino acid sequence set forth in SEQ ID NO:9 and a light chain comprising the amino acid sequence as set forth in SEQ ID NO:10.

In one embodiment the TIM-3 binding protein comprises a V_{H} domain comprising an amino acid sequence at least 90% identical to the amino acid sequence set forth in SEQ ID NO:19 and/or a V_{L} domain comprising an amino acid sequence at least 90% identical to the amino acid sequence as set forth in SEQ ID NO:20 wherein said TIM-3 binding protein specifically binds to human TIM-3. In one embodiment, the TIM-3 binding protein comprises one or more of: CDRH1 as set forth in SEQ ID NO:13; CDRH2 as set forth in SEQ ID NO:14; CDRH3 as set forth in SEQ ID NO:15; CDRL1 as set forth in SEQ ID NO:16; CDRL2 as set forth in SEQ ID NO:17 and/or CDRL3 as set forth in SEQ ID NO:18 or a direct equivalent of each CDR wherein a direct equivalent has no more than two amino acid substitutions in said CDR. In one embodiment, the TIM-3 binding protein comprises a heavy chain variable region comprising one or more of SEQ ID NO:13; SEQ ID NO:14; and SEQ ID NO:15 and wherein said TIM-3 binding protein comprises a light chain variable region comprising one or more of SEQ ID NO:16; SEQ ID NO:17, and SEQ ID NO:18. In one embodiment, the TIM-3 binding protein comprises a heavy chain variable region comprising SEQ ID NO:13; SEQ ID NO:14; and SEQ ID NO:15 and wherein said TIM-3 binding protein comprises a light chain variable region comprising SEQ ID NO:16; SEQ ID NO:17, and SEQ ID NO:18. In one embodiment, the TIM-3 binding protein comprises a V_{H} domain comprising the amino acid sequence set forth in SEQ ID NO:19 and a V_{L} domain comprising the amino acid sequence as set forth in SEQ ID NO:20. In one embodiment, the TIM-3 binding protein comprises a heavy chain comprising the amino acid sequence set forth in SEQ ID NO:21 and a light chain comprising the amino acid sequence as set forth in SEQ ID NO:22.

The methods of the invention may additionally comprise PD-1. Therefore, in one aspect, there is provided a method of treating cancer in a human in need thereof, the method comprising administering to the human an ICOS binding protein, a TIM-3 binding protein and a PD-1 binding protein. In a further aspect, there is provided an ICOS binding protein, a TIM-3 binding protein and a PD-1 binding protein for concurrent or sequential use in treating cancer. In another aspect, there is provided an ICOS binding protein for use in treating cancer, wherein the ICOS binding protein is to be administered concurrently or sequentially with a TIM-3 binding protein and a PD-1 binding protein. In one aspect, there is provided the use of an ICOS binding protein in the manufacture of a medicament for treating cancer, wherein the ICOS binding protein is to be administered concurrently or sequentially with a TIM-3 binding protein and a PD-1 binding protein. In another aspect, there is provided a TIM-3 binding protein for use in treating cancer, wherein the TIM-3 binding protein is to be administered concurrently or sequentially with an ICOS binding protein and a PD-1 binding protein. In one aspect, there is provided the use of a TIM-3 binding protein in the manufacture of a medicament for treating cancer, wherein the TIM-3 binding protein is to be administered concurrently or sequentially with an ICOS binding protein and a PD-1 binding protein. In another aspect, there is provided a pharmaceutical kit comprising an ICOS binding protein, a TIM-3 binding protein and a PD-1 binding protein. All aspects and embodiments described hereinbefore also apply to combinations where PD-1 binding proteins are also used.

### Dosage

In one aspect, the method comprises administering a therapeutically effective amount of a combinations as described herein (i.e. comprising an ICOS binding protein and a TIM-3 binding protein, and optionally a PD-1 binding protein) to a subject in need thereof.

In some embodiments, a therapeutically effective dose of the ICOS binding protein is a dose of about 0.01-1000 mg (e.g. a dose about 0.01 mg; a dose about 0.08 mg, a dose about 0.1 mg; a dose about 0.24 mg; a dose about 0.8 mg; a dose about 1 mg; a dose about 2.4 mg; a dose about 7.2 mg; a dose about 8 mg; a dose about 10 mg; a dose about 20 mg; a dose about 24 mg; a dose about 30 mg; a dose about 40 mg; a dose about 48 mg; a dose about 50 mg; a dose about 60 mg; a dose about 70 mg; a dose about 72 mg; a dose about 80 mg; a dose about 90 mg; a dose about 100 mg; a dose about 160 mg; a dose about 200 mg; a dose about 240 mg; a dose about 300 mg; a dose about 320 mg; a dose about 400 mg; a dose about 480 mg; a dose about 500 mg; a dose about 600 mg; a dose about 700 mg; a dose about 720 mg; a dose about 800 mg; a dose about 900 mg; or a dose about 1000 mg).

In some embodiments, a therapeutically effective dose of the ICOS binding protein is a dose of about 0.001 mg/kg to 10 mg/kg. In some embodiments, a therapeutically effective dose is about 0.001 mg/kg. In some embodiments, a therapeutically effective dose is about 0.003 mg/kg. In some embodiments, a therapeutically effective dose is about 0.01 mg/kg. In some embodiments, a therapeutically effective dose is about 0.03 mg/kg. In some embodiments, a therapeutically effective dose is about 0.1 mg/kg. In some embodiments, a therapeutically effective dose is about 0.3 mg/kg. In some embodiments, a therapeutically effective dose is about 0.6 mg/kg. In some embodiments, a therapeutically effective dose is about 1 mg/kg. In some embodiments, a therapeutically effective dose is about 2 mg/kg. In some embodiments, a therapeutically effective dose is about 3 mg/kg. In some embodiments, a therapeutically effective dose is about 4 mg/kg; about 5 mg/kg; about 6 mg/kg; about 7 mg/kg; about 8 mg/kg; about 9 mg/kg or about 10 mg/kg. In some embodiments, a therapeutically effective dose is a dose about 500 mg. In some embodiments, a therapeutically effective dose is about 800 mg. In some embodiments, a therapeutically effective dose is about 1000 mg.

In some embodiments, a therapeutically effective dose of the TIM-3 binding protein is a dose of about 0.01-5000 mg (e.g. a dose about 0.01 mg; a dose about 0.1 mg; a dose about 1 mg; a dose about 10 mg; a dose about 20 mg; a dose about 30 mg; a dose about 40 mg; a dose about 50 mg; a dose about 60 mg; a dose about 70 mg; a dose about 80 mg; a dose about 90 mg; a dose about 100 mg; a dose about 200 mg; a dose about 300 mg; a dose about 400 mg; a dose about 500 mg; a dose about 600 mg; a dose about 700 mg; a dose about 800 mg; a dose about 900 mg; a dose about 1000 mg; a dose about 1100 mg; a dose about 1200 mg; a dose about 1300 mg; a dose about 1400 mg; a dose about 1500 mg; a dose about 1600 mg; a dose about 1700 mg; a dose about 1800 mg; a dose about 1900 mg; a dose about 2000 mg; a dose about 2100 mg; a dose about 2200 mg; a dose about 2300 mg; a dose about 2400 mg, a dose about 2500 mg; a dose about 3000 mg; a dose about 4000 mg; or a dose about 5000 mg), such as a dose of about 5-5000 mg. In some embodiments, the therapeutically effective dose of the TIM-3 binding protein is about 100 mg, 300 mg or 900 mg. In some embodiments, the therapeutically effective dose of the TIM-3 binding protein is 300 mg. In some embodiments, a therapeutically effective dose is about 0.001 mg/kg. In some embodiments, a therapeutically effective dose is about 0.003 mg/kg. In some embodiments, a therapeutically effective dose is about 0.01 mg/kg. In some embodiments, a therapeutically effective dose is about 0.03 mg/kg. In some embodiments, a therapeutically effective dose is about 0.1 mg/kg. In some embodiments, a therapeutically effective dose is about 0.3 mg/kg. In some embodiments, a therapeutically effective dose is about 1 mg/kg. In some embodiments, a therapeutically effective dose of the TIM-3 binding protein is about 1.25 mg/kg. In some embodiments, a therapeutically effective dose is about 2 mg/kg. In some embodiments, a therapeutically effective dose is about 3 mg/kg. In some embodiments, a therapeutically effective dose of the TIM-3 binding protein is about 3.75 mg/kg. In some embodiments, a therapeutically effective dose is about 10 mg/kg. In some embodiments, a therapeutically effective dose of the TIM-3 binding protein is about 11.25 mg/kg.

In some embodiments, a therapeutically effective dose of the PD-1 binding protein is a dose of about 0.01-5000 mg (e.g. a dose about 0.01 mg; a dose about 0.1 mg; a dose about 1 mg; a dose about 10 mg; a dose about 20 mg; a dose about 30 mg; a dose about 40 mg; a dose about 50 mg; a dose about 60 mg; a dose about 70 mg; a dose about 80 mg; a dose about 90 mg; a dose about 100 mg; a dose about 200 mg; a dose about 300 mg; a dose about 400 mg; a dose about 500 mg; a dose about 600 mg; a dose about 700 mg; a dose about 800 mg; a dose about 900 mg; a dose about 1000 mg; a dose about 1100 mg; a dose about 1200 mg; a dose about 1300 mg; a dose about 1400 mg; a dose about 1500 mg; a dose about 1600 mg; a dose about 1700 mg; a dose about 1800 mg; a dose about 1900 mg; a dose about 2000 mg; a dose about 2100 mg; a dose about 2200 mg; a dose about 2300 mg or a dose about 2400 mg; a dose about 2500 mg; a dose about 3000 mg; a dose about 4000 mg or a dose about 5000 mg). In some embodiments, a therapeutically effective dose is about 0.001 mg/kg. In some embodiments, a therapeutically effective dose is about 0.003 mg/kg. In some embodiments, a therapeutically effective dose is about 0.01 mg/kg. In some embodiments, a therapeutically effective dose is about 0.03 mg/kg. In some embodiments, a therapeutically effective dose is about 0.1 mg/kg. In some embodiments, a therapeutically effective dose is about 0.3 mg/kg. In some embodiments, a therapeutically effective dose is about 1 mg/kg. In some embodiments, a therapeutically effective dose is about 2 mg/kg. In some embodiments, a therapeutically effective dose is about 3 mg/kg. In some embodiments, a therapeutically effective dose is about 10 mg/kg. In some embodiments, a therapeutically effective dose is a dose about 500 mg. In some embodiments, a therapeutically effective dose is about 800 mg. In some embodiments, a therapeutically effective dose is about 1000 mg.

In one embodiment, the combination is administered once every 2-6 weeks (e.g. 2, 3 or 4 weeks, in particular 3 weeks). In one embodiment, the combination is administered for once every 3 weeks. In one embodiment, the combination is administered for once every 6 weeks. In one embodiment, the combination is administered for once every 3 weeks for 2-6 dosing cycles (e.g. the first 3, 4, or 5 dosing cycles, in particular, the first 4 dosing cycles).

If desired, the effective daily dose of a (therapeutic) combination may be administered as two, three, four, five, six or more doses administered separately at appropriate intervals throughout the day, optionally, in unit dosage forms.

The present disclosure provides methods of treating cancer comprising administering to a patient in need of treatment one or both of the binding proteins in the combination at a first dose at a first interval for a first period; and administering to the patient one or both of the binding proteins in the combination at a second dose at a second interval for a second period. There may be a rest period between the first and second periods in which one or both of the binding proteins in the combination are not administered to the patient. In some embodiments, there is a rest period between the first period and second period. In some embodiments, the rest period is between 1 and 30 days. In some embodiments, the rest period is 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or 31 days. In some embodiments, the rest period is 1 week, 2 weeks, 3 weeks, 4 weeks, 5 weeks, 6 weeks, 7 weeks, 9 weeks, 10 weeks, 11 weeks, 12 weeks, 13 weeks, 14 weeks or 15 weeks.

In some embodiments, the first dose and second dose are the same. In some embodiments, the first dose and the second dose are 300 mg. In some embodiments, the first dose and second dose are different. In some embodiments, the first dose is about 500 mg and the second dose is 1000 mg.

In some embodiments, the first interval and second interval are the same. In some embodiments, the first interval and the second interval are once every three weeks. In some embodiments, the first interval and the second interval are once every six weeks.

In some embodiments, the first interval and the second interval are different. In some embodiments, the first interval is once every three weeks and the second interval is once every six weeks. In some embodiments, the combination is administered at the first dose of 24 mg once every three weeks for the first period of 2-6 dosing cycles (e.g. the first 3, 4, or 5 dosing cycles, in particular, the first 4 dosing cycles), and at the second dose of 80 mg once every six weeks until therapy is discontinued (e.g. due to disease progression, an adverse event, or as determined by a physician). In some embodiments, the combination is administered at the first dose of 24 mg once every three weeks for the first three dosing cycles, and at the second dose of 80 mg once every six weeks or more until therapy is discontinued (e.g. due to disease progression, an adverse event, or as determined by a physician). In some embodiments, the combination is administered at the first dose of 24 mg once every three weeks for the first four dosing cycles, and at the second dose of 80 mg once every six weeks or more until therapy is discontinued (e.g. due to disease progression, an adverse event, or as determined by a physician). In some embodiments, the combination is administered at the first dose of 24 mg once every three weeks for the first five dosing cycles, and at the second dose of 80 mg once every six weeks or more until therapy is discontinued (e.g. due to disease progression, an adverse event, or as determined by a physician). In some embodiments, combination is administered at the first dose of 48 mg once every three weeks for the first period of 2-6 dosing cycles (e.g. the first 3, 4, or 5 dosing cycles, in particular, the first 4 dosing cycles), and at the second dose of 160 mg once every six weeks until therapy is discontinued (e.g. due to disease progression, an adverse event, or as determined by a physician). In some embodiments, the combination is administered at the first dose of 48 mg once every three weeks for the first three dosing cycles, and at the second dose of 160 mg once every six weeks or more until therapy is discontinued (e.g. due to disease progression, an adverse event, or as determined by a physician). In some embodiments, the combination is administered at the first dose of 48 mg once every three weeks for the first four dosing cycles, and at the second dose of 160 mg once every six weeks or more until therapy is discontinued (e.g. due to disease progression, an adverse event, or as determined by a physician). In some embodiments, the combination is administered at the first dose of 48 mg once every three weeks for the first five dosing cycles, and at the second dose of 160 mg once every six weeks or more until therapy is discontinued (e.g. due to disease progression, an adverse event, or as determined by a physician). In some embodiments, the second dose is administered once every six weeks.

In some embodiments, the combination is administered at an administration interval (or treatment cycle) of once a week (Q1W), once every 2 weeks (Q2W), once every 3 weeks (Q3W), once every 4 weeks (Q4W), once every 5 weeks (Q5W), or once every 6 weeks (Q6W). In some embodiments, the combination is administered at an administration interval (or treatment cycle) of once a week (Q1W). In some embodiments, the combination is administered at an administration interval (or treatment cycle) of once every 2 weeks (Q2W). In some embodiments, the combination is administered at an administration interval (or treatment cycle) of once every three weeks (Q3W). In some embodiments, the combination is administered at an administration interval (or treatment cycle) of once every 4 weeks (Q4W). In some embodiments, the combination is administered at an administration interval (or treatment cycle) of once every 5 weeks (Q5W). In some embodiments, the combination is administered at an administration interval (or treatment cycle) of once every 6 weeks (Q6W). In some embodiments, the combination is administered for a period of at least about 2, 3, 4, 5, 6, 7, 8, 9, 10, 11, 12, 13, 14, 15, 16, 17, 18, 19, 20 weeks, or more. In some embodiments, the combination is administered on the first day of a treatment cycle or within 1, 2, or 3 days of the first day of a treatment cycle.

In some embodiments, the combination described herein is administered according to dosing regimens demonstrated to achieve a clinical benefit for the patient. In some embodiments, a clinical benefit is stable disease (“SD”), a partial response (“PR”) and/or a complete response (“CR”). In some embodiments, a clinical benefit is stable disease (“SD”). In some embodiments, a clinical benefit is a partial response (“PR”). In some embodiments, a clinical benefit is a complete response (“CR”). In some embodiments, PR or CR is determined in accordance with Response Evaluation Criteria in Solid Tumors (RECIST). In some embodiments, the combination is administered for a longer period to maintain clinical benefit.

In one aspect there is provided a method of treating cancer in a human, the method comprising administering to the human an ICOS binding protein (or antigen binding portion thereof) at a dose of about 0.08 mg to about 240 mg and administering to the human a TIM-3 binding protein (or antigen binding portion thereof). In one embodiment, the ICOS binding protein is administered at a dose of 0.08 mg, 0.24 mg, 0.8 mg, 2.4 mg, 8 mg, 24 mg, 48 mg, 80 mg, 160 mg or 240 mg in particular 24 mg, 48 mg, 80 mg or 160 mg. In one aspect there is provided a method of treating cancer in a human, the method comprising administering to the human a TIM-3 binding protein (or antigen binding portion thereof) at a dose of about 5 mg to about 5000 mg and administering to the human an ICOS binding protein (or antigen binding portion thereof). In one embodiment, the TIM-3 binding protein is administered at a dose of 100 mg, 300 mg or 900 mg. In one embodiment, the TIM-3 binding protein is administered at a dose of 300 mg. In one aspect there is provided a method of treating cancer in a human, the method comprising administering to the human an ICOS binding protein (or antigen binding portion thereof) at a dose of about 0.08 mg to about 240 mg and administering to the human a TIM-3 binding protein and a PD-1 binding protein (or antigen binding portion thereof). In one embodiment, the TIM-3 binding protein is administered at a dose of 100 mg, 300 mg or 900 mg. In one embodiment, the TIM-3 binding protein is administered at a dose of 300 mg. In one embodiment, the PD-1 binding protein is administered at a dose of 500 mg or 1000 mg.

In one embodiment, there is a method of treating cancer in a human, the method comprising administering to the human an ICOS binding protein at a dose of about 0.08 mg to about 240 mg and administering to the human a TIM-3 binding protein at a dose of about 100 mg to about 1000 mg. In another embodiment, there is a method of treating cancer in a human, the method comprising administering to the human an ICOS binding protein at a dose of about 0.08 mg to about 240 mg and administering to the human a TIM-3 binding protein at a dose of about 5 mg to about 5000 mg and a PD-1 binding protein at a dose of about 100 mg to about 2000 mg. In one embodiment, the ICOS binding protein is administered at a dose of 24 mg, 48 mg, 80 mg or 160 mg and the TIM-3 binding protein is administered at a dose of 100 mg, 300 mg or 900 mg. In a further embodiment, the ICOS binding protein is administered at a dose of 24 mg, 48 mg, 80 mg or 160 mg and the TIM-3 binding protein is administered at a dose of 300 mg. In one embodiment, the ICOS binding protein is administered at a dose of 24 mg, 48 mg, 80 mg or 160 mg, the TIM-3 binding protein is administered at a dose of 100 mg, 300 mg or 900 mg and the PD-1 binding protein is administered at a dose of 500 mg or 1000 mg. In further embodiment, the ICOS binding protein is administered at a dose of 24 mg, 48 mg, 80 mg or 160 mg, the TIM-3 binding protein is administered at a dose of 300 mg and the PD-1 binding protein is administered at a dose of 500 mg or 1000 mg.

In one aspect, there is provided an ICOS binding protein and a TIM-3 binding protein for concurrent (i.e. simultaneous) or sequential use in treating cancer, wherein the ICOS binding protein is to be administered at a dose of about 0.08 mg to about 240 mg. In one embodiment, the ICOS binding protein is administered at a dose of 0.08 mg, 0.24 mg, 0.8 mg, 2.4 mg, 8 mg, 24 mg, 48 mg, 80 mg, 160 mg or 240 mg, in particular 24 mg, 48 mg, 80 mg or 160 mg. In one aspect, there is provided an ICOS binding protein and a TIM-3 binding protein for concurrent (i.e. simultaneous) or sequential use in treating cancer, wherein the TIM-3 binding protein is to be administered at a dose of about 100 mg to about 1000 mg. In one embodiment, the TIM-3 binding protein is administered at a dose of 100 mg, 300 mg or 900 mg. In one embodiment, the TIM-3 binding protein is administered at a dose of 300 mg. In one aspect, there is provided an ICOS binding protein, a TIM-3 binding protein and a PD-1 binding protein for concurrent (i.e. simultaneous) or sequential use in treating cancer, wherein the PD-1 binding protein is to be administered at a dose of about 100 mg to about 2000 mg. In one embodiment, the PD-1 binding protein is administered at a dose of 500 mg or 1000 mg.

In one embodiment, there is provided an ICOS binding protein and a TIM-3 binding protein for concurrent or sequential use in treating cancer, wherein the ICOS binding protein is to be administered at a dose of about 0.08 mg to about 240 mg and the TIM-3 binding protein is to be administered at a dose of about 100 mg to about 1000 mg. In another embodiment, the ICOS binding protein is administered at a dose of 24 mg, 48 mg, 80 mg or 160 mg, and the TIM-3 binding protein is administered at a dose of 100 mg, 300 mg or 900 mg. In a further embodiment, the ICOS binding protein is administered at a dose of 24 mg, 48 mg, 80 mg or 160 mg, and the TIM-3 binding protein is administered at a dose of 300 mg. In another embodiment, there is provided an ICOS binding protein, a PD-1 binding protein and a TIM-3 binding protein for concurrent or sequential use in treating cancer, wherein the ICOS binding protein is to be administered at a dose of about 0.08 mg to about 240 mg, the PD-1 binding protein is to be administered at a dose of about 100 mg to about 2000 mg and the TIM-3 binding protein is to be administered at a dose of about 5 mg to about 5000 mg. In another embodiment, the ICOS binding protein is administered at a dose of 24 mg, 48 mg, 80 mg or 160 mg, the PD-1 binding protein is administered at a dose of 500 mg or 1000 mg and the TIM-3 binding protein is to be administered at a dose of 100 mg, 300 mg or 900 mg. In one embodiment, the ICOS binding protein is administered at a dose of 24 mg, 48 mg, 80 mg or 160 mg, the PD-1 binding protein is administered at a dose of 500 mg or 1000 mg and the TIM-3 binding protein is to be administered at a dose of 300 mg.

In another aspect, an ICOS binding protein for use in treating cancer is provided, wherein the ICOS binding protein is to be administered at a dose of about 0.08 mg to about 240 mg and is to be administered concurrently (i.e. simultaneously) or sequentially with a TIM-3 binding protein. In one embodiment, the ICOS binding protein is administered at a dose of 8 mg, 24 mg, 48 mg, 80 mg or 160 mg. In another aspect, a TIM-3 binding protein for use in treating cancer is provided, wherein the TIM-3 binding protein is to be administered at a dose of about 100 mg to about 1000 mg and is to be administered concurrently (i.e. simultaneously) or sequentially with an ICOS binding protein. In one embodiment, the TIM-3 binding protein is administered at a dose of 100 mg, 300 mg or 900 mg. In a further embodiment, the TIM-3 binding protein is administered at a dose of 300 mg. In one embodiment, the ICOS binding protein is to be administered at a dose of about 0.08 mg to about 240 mg and is to be administered concurrently or sequentially with a TIM-3 binding protein at a dose of about 100 mg to about 1000 mg. In another embodiment, the ICOS binding protein is administered at a dose of 24 mg, 48 mg, 80 mg or 160 mg, and the TIM-3 binding protein is administered at a dose of 100 mg, 300 mg or 900 mg. In a further embodiment, the ICOS binding protein is administered at a dose of 24 mg, 48 mg, 80 mg or 160 mg, and the TIM-3 binding protein is administered at a dose of 300 mg.

In another aspect, an ICOS binding protein for use in treating cancer is provided, wherein the ICOS binding protein is to be administered at a dose of about 0.08 mg to about 240 mg and is to be administered concurrently (i.e. simultaneously) or sequentially with a PD-1 binding protein and a TIM-3 binding protein. In one embodiment, the ICOS binding protein is administered at a dose of 8 mg, 24 mg, 48 mg, 80 mg, 160 mg or 240 mg. In another aspect, a TIM-3 binding protein for use in treating cancer is provided, wherein the TIM-3 binding protein is to be administered at a dose of about 100 mg to about 1000 mg and is to be administered concurrently (i.e. simultaneously) or sequentially with an ICOS binding protein and a PD-1 binding protein. In one embodiment, the TIM-3 binding protein is administered at a dose of 100 mg, 300 mg or 900 mg. In one embodiment, the TIM-3 binding protein is administered at a dose of 300 mg. In one embodiment, the ICOS binding protein is to be administered at a dose of about 0.08 mg to about 240 mg and is to be administered concurrently or sequentially with a PD-1 binding protein at a dose of about 100 mg to about 2000 mg and a TIM-3 binding protein at a dose of about 5 mg to about 5000 mg. In another embodiment, the ICOS binding protein is administered at a dose of 24 mg, 48 mg, 80 mg or 160 mg, the PD-1 binding protein is administered at a dose of 500 mg or 1000 mg and the TIM-3 binding protein is administered at a dose of 100 mg, 300 mg or 900 mg. In a further embodiment, the ICOS binding protein is administered at a dose of 24 mg, 48 mg, 80 mg or 160 mg, the PD-1 binding protein is administered at a dose of 500 mg or 1000 mg and the TIM-3 binding protein is administered at a dose of 300 mg.

In another aspect, there is provided use of an ICOS binding protein in the manufacture of a medicament for treating cancer, wherein the ICOS binding protein is to be administered at a dose of about 0.08 mg to about 240 mg and is to be administered concurrently or sequentially with a TIM-3 binding protein. In one embodiment, the ICOS binding protein is administered at a dose of 8 mg, 24 mg, 48 mg, 80 mg or 160 mg. In another aspect, there is provided use of a TIM-3 binding protein in the manufacture of a medicament for treating cancer, wherein the TIM-3 binding protein is to be administered at a dose of about 100 mg to about 1000 mg and is to be administered concurrently or sequentially with an ICOS binding protein. In one embodiment, the TIM-3 binding protein is administered at a dose of 100 mg, 300 mg or 900 mg. In one embodiment, there is a use of an ICOS binding protein in the manufacture of a medicament for treating cancer, wherein the ICOS binding protein is to be administered at a dose of about 0.08 mg to about 240 mg and is to be administered concurrently or sequentially with a TIM-3 binding protein at a dose of about 100 mg to about 1000 mg. In another embodiment, the ICOS binding protein is administered at a dose of 24 mg, 48 mg, 80 mg or 160 mg, and the TIM-3 binding protein is administered at a dose of 100 mg, 300 mg or 900 mg. In a further embodiment, the ICOS binding protein is administered at a dose of 24 mg, 48 mg, 80 mg or 160 mg, and the TIM-3 binding protein is administered at a dose of 300 mg.

In another aspect, there is provided use of an ICOS binding protein in the manufacture of a medicament for treating cancer, wherein the ICOS binding protein is to be administered at a dose of about 0.08 mg to about 240 mg and is to be administered concurrently or sequentially with a PD-1 binding protein and a TIM-3 binding protein. In one embodiment, the ICOS binding protein is administered at a dose of 8 mg, 24 mg, 48 mg, 80 mg, 160 mg or 240 mg. In another aspect, there is provided use of a TIM-3 binding protein in the manufacture of a medicament for treating cancer, wherein the TIM-3 binding protein is to be administered at a dose of about 100 mg to about 1000 mg and is to be administered concurrently or sequentially with an ICOS binding protein and a PD-1 binding protein. In one embodiment, the TIM-3 binding protein is administered at a dose of 100 mg, 300 mg or 900 mg. In another embodiment, the TIM-3 binding protein is administered at a dose of 300 mg. In one embodiment, there is a use of an ICOS binding protein in the manufacture of a medicament for treating cancer, wherein the ICOS binding protein is to be administered at a dose of about 0.08 mg to about 240 mg and is to be administered concurrently or sequentially with a PD-1 binding protein at a dose of about 100 mg to about 2000 mg and a TIM-3 binding protein at a dose of about 5 mg to about 5000 mg. In another embodiment, the ICOS binding protein is administered at a dose of 24 mg, 48 mg, 80 mg or 160 mg, the PD-1 binding protein is administered at a dose of 500 mg or 1000 mg and the TIM-3 binding protein is administered at a dose of 100 mg, 300 mg or 900 mg. In a further embodiment, the ICOS binding protein is administered at a dose of 24 mg, 48 mg, 80 mg or 160 mg, the PD-1 binding protein is administered at a dose of 500 mg or 1000 mg and the TIM-3 binding protein is administered at a dose of 300 mg.

In one aspect, there is provided a pharmaceutical kit comprising about 0.08 mg to about 240 mg of an ICOS binding protein and a TIM-3 binding protein. In a further embodiment, the pharmaceutical kit comprises about 8 mg, about 24 mg, about 48 mg, about 80 mg or about 160 mg of the ICOS binding protein. In one embodiment, the pharmaceutical kit comprises about 100 mg to about 1000 mg of the TIM-3 binding protein. In a further embodiment, the pharmaceutical kit comprises about 100 mg, about 300 mg or about 900 mg of the TIM-3 binding protein. In one embodiment, the pharmaceutical kit comprises about 300 mg of the TIM-3 binding protein. In one embodiment, the TIM-3 binding protein is cobolimab.

In one aspect, there is provided a pharmaceutical kit comprising about 100 mg to about 1000 mg of a TIM-3 binding protein and an ICOS binding protein. In one embodiment, the pharmaceutical kit comprises about 0.08 mg to about 240 mg of the ICOS binding protein. In a further embodiment, the pharmaceutical kit comprises about 24 mg or about 48 mg of the ICOS binding protein. In a further embodiment, the pharmaceutical kit comprises about 80 mg or about 160 mg of the ICOS binding protein.

In one aspect, there is provided a pharmaceutical kit comprising about 0.08 mg to about 240 mg of an ICOS binding protein, a PD-1 binding protein and a TIM-3 binding protein. In a further embodiment, the pharmaceutical kit comprises about 8 mg, about 24 mg, about 48 mg, about 80 mg or about 160 mg of the ICOS binding protein. In one embodiment, the pharmaceutical kit comprises about 100 mg to about 2000 mg of the PD-1 binding protein. In a further embodiment, the pharmaceutical kit comprises about 500 mg or about 1000 mg of the PD-1 binding protein. In one embodiment, the PD-1 binding protein is dostarlimab. In one embodiment, the pharmaceutical kit comprises about 5 mg to about 5000 mg of the TIM-3 binding protein. In a further embodiment, the pharmaceutical kit comprises about 100 mg, about 300 mg or about 900 mg of the TIM-3 binding protein. In one embodiment, the pharmaceutical kit comprises about 300 mg of the TIM-3 binding protein. In one embodiment, the TIM-3 binding protein is cobolimab.

In one aspect, there is provided a pharmaceutical kit comprising about 100 mg to about 1000 mg of a TIM-3 binding protein, an ICOS binding protein and a PD-1 binding protein. In one embodiment, the pharmaceutical kit comprises about 0.08 mg to about 240 mg of the ICOS binding protein. In a further embodiment, the pharmaceutical kit comprises about 24 mg or about 48 mg of the ICOS binding protein. In an further embodiment, the pharmaceutical kit comprises about 80 mg or about 160 mg of the ICOS binding protein.

In one embodiment, the pharmaceutical kit comprises the ICOS binding protein at a concentration of 10 mg/mL. In one embodiment, the pharmaceutical kit comprises the TIM-3 binding protein at a concentration of about 5 mg/mL to about 100 mg/mL. In a further embodiment, the pharmaceutical kit comprises the TIM-3 binding protein at a concentration of 10 mg/mL to 40 mg/mL. In one embodiment, the TIM-3 binding protein is at a concentration of 20 mg/mL. In one embodiment, the pharmaceutical kit comprises the PD-1 binding protein at a concentration of about 20 mg/mL to about 125 mg/mL. In a further embodiment, the pharmaceutical kit comprises the PD-1 binding protein at a concentration of 20 mg/mL to 50 mg/mL. In one embodiment, the PD-1 binding protein is at a concentration of 20 mg/mL. In another embodiment, the PD-1 binding protein is at a concentration of 50 mg/mL.

In another aspect, there is provided a pharmaceutical formulation comprising an ICOS binding protein at a concentration of 10 mg/mL. In another aspect, there is provided a pharmaceutical formulation comprising a TIM-3 binding protein at a concentration of about 5 mg/mL to about 100 mg/mL. In a further embodiment, the pharmaceutical formulation comprises a TIM-3 binding protein at a concentration of 10 mg/mL to 40 mg/mL. In one embodiment, the TIM-3 binding protein is at a concentration of 20 mg/mL. Thus, in one embodiment, the pharmaceutical formulation comprises an ICOS binding protein at a concentration of 10 mg/ml and a TIM-3 binding protein at a concentration of about 5 mg/mL to about 100 mg/mL. In a further embodiment, the pharmaceutical formulation comprises an ICOS binding protein at a concentration of 10 mg/ml and a TIM-3 binding protein at a concentration of 10 mg/mL to 40 mg/mL. In one embodiment, the pharmaceutical formulation comprises an ICOS binding protein at a concentration of 10 mg/ml and a TIM-3 binding protein at a concentration of 20 mg/mL. In another aspect, there is provided a pharmaceutical formulation comprising a PD-1 binding protein at a concentration of about 20 mg/mL to about 125 mg/mL. In a further embodiment, the pharmaceutical formulation comprises a PD-1 binding protein at a concentration of 20 mg/mL to 50 mg/mL. In one embodiment, the PD-1 binding protein is at a concentration of 20 mg/mL. In another embodiment, the PD-1 binding protein is at a concentration of 50 mg/mL. Thus, in one embodiment, the pharmaceutical formulation comprises an ICOS binding protein at a concentration of 10 mg/ml, a TIM-3 binding protein at a concentration of about 5 mg/mL to about 100 mg/mL and a PD-1 binding protein at a concentration of about 20 mg/mL to about 125 mg/mL. In a further embodiment, the pharmaceutical formulation comprises an ICOS binding protein at a concentration of 10 mg/ml, a TIM-3 binding protein at a concentration of 10 mg/mL to 40 mg/mL and a PD-1 binding protein at a concentration of 20 mg/mL to 50 mg/mL. In one embodiment, the pharmaceutical formulation comprises an ICOS binding protein at a concentration of 10 mg/ml, a TIM-3 binding protein at a concentration of 20 mg/mL and a PD-1 binding protein at a concentration of 20 mg/mL. In another embodiment, the pharmaceutical formulation comprises an ICOS binding protein at a concentration of 10 mg/ml, a TIM-3 binding protein at a concentration of 20 mg/mL and a PD-1 binding protein at a concentration of 50 mg/mL.

In some embodiments, the ICOS binding protein is administered at a dose of about 0.08-800 mg (e.g. a dose about 0.08 mg; a dose about 0.24 mg; a dose about 0.8 mg; a dose about 2.4 mg; a dose about 8 mg; a dose about 16 mg; a dose about 24 mg; a dose about 32 mg; a dose about 40 mg; a dose about 48 mg; a dose about 56 mg; a dose about 64 mg; a dose about 72 mg; a dose about 80 mg; a dose about 88 mg; a dose about 96 mg; a dose about 100 mg; a dose about 160 mg; a dose about 200 mg; a dose about 240 mg; a dose about 300 mg; a dose about 400 mg; a dose about 500 mg; a dose about 600 mg; a dose about 700 mg or a dose about 800 mg). In some embodiments, the ICOS binding protein is administered at a dose of about 0.08-240 mg. In further embodiments, the ICOS binding protein is administered at a dose of about 0.001-10 mg/kg (e.g. a dose about 0.001 mg/kg, a dose about 0.003 mg/kg, a dose about 0.01 mg/kg, a dose about 0.03 mg/kg, a dose about 0.1 mg/kg, a dose about 0.3 mg/kg, a dose about 0.6 mg/kg, a dose about 1.0 mg/kg, a dose about 2.0 mg/kg, a dose about 3.0 mg/kg, a dose about 6 mg/kg or a dose about 10 mg/kg). In some embodiments, the ICOS binding protein is administered at a dose of about 0.001-3 mg/kg. In some embodiments, the ICOS binding protein is administered at a dose of about 0.3 mg/kg. In some embodiments, the ICOS binding protein is administered at a dose of about 1 mg/kg. In some embodiments, the ICOS binding protein is administered at a dose of about 3 mg/kg. In some embodiments, the ICOS binding protein is administered at a dose of about 24 mg. In some embodiments, the ICOS binding protein is administered at a dose of about 48 mg. In some embodiments, the ICOS binding protein is administered at a dose of about 72 mg. In some embodiments, the ICOS binding protein is administered at a dose of about 80 mg. In some embodiments, the ICOS protein is administered at a dose of about 96 mg. In some embodiments, the ICOS protein is administered at a dose of about 120 mg. In some embodiments, the ICOS protein is administered at a dose of about 148 mg. In some embodiments, the ICOS binding protein is administered at a dose of about 160 mg. In some embodiments, the ICOS binding protein is administered at a dose of about 240 mg. In some embodiments, the ICOS protein is administered at a dose of about 320 mg. In some embodiments, the ICOS protein is administered at a dose of about 480 mg.

In one embodiment, the dose of the ICOS binding protein is in the range of about 0.08 mg to about 800 mg. In another embodiment, the dose of the ICOS binding protein is in the range of about 0.8 mg to about 240 mg.

In another embodiment, the dose of the ICOS binding protein is in the range of about 8 mg to about 80 mg. In another embodiment, the dose of the ICOS binding protein is about 0.08 mg, about 0.24 mg, about 0.48 mg, about 0.8 mg, about 1.6 mg, about 2.4 mg, about 8 mg, about 24 mg, about 48 mg, about 80 mg, about 160 mg or about 240 mg. In one embodiment, the dose of ICOS binding protein is about 24 mg, about 48 mg, about 80 mg or about 160 mg. In one embodiment, the dose of the ICOS binding protein is at least about 24 mg. In one embodiment, the dose of the ICOS binding protein is at least about 48 mg.

In one embodiment, the ICOS binding protein is administered once every 2-6 weeks (e.g. 2, 3 or 4 weeks, in particular 3 weeks). In one embodiment the ICOS binding protein is administered for once every 3 weeks for 2-6 dosing cycles (e.g. the first 3, 4, or 5 dosing cycles, in particular, the first 4 dosing cycles).

In one embodiment, the ICOS binding protein is vopratelimab. In one embodiment, vopratelimab is administered at 0.03 mg/kg, 0.3 mg/kg or 0.1 mg/kg. In another embodiment, the dosing amount and interval between doses of vopratelimab is pulsatile. In one embodiment, vopratelimab is administered every 3 weeks.

In some embodiments, the TIM-3 binding protein is administered at a dose of about 5-5000 mg (e.g. a dose about 5 mg; a dose about 10 mg; a dose about 50 mg; a dose about 100 mg; a dose about 200 mg; a dose about 300 mg; a dose about 400 mg; a dose about 500 mg; a dose about 600 mg; a dose about 700 mg; a dose about 800 mg; a dose about 900 mg; a dose about 1000 mg; a dose about 1100 mg; a dose about 1200 mg; a dose about 1300 mg; a dose about 1400 mg; a dose about 1500 mg; a dose about 2000 mg; a dose about 3000 mg; a dose about 4000 mg; or a dose about 5000 mg). In some embodiments, the TIM-3 binding protein is administered at a dose of about 100 mg, 300 mg or 900 mg. In some embodiments, the TIM-3 binding protein is administered at a dose of about 300 mg. In some embodiments, the TIM-3 binding protein is administered at a dose of about 1.25 mg/kg. In some embodiments, the TIM-3 binding protein is administered at a dose of about 3.75 mg/kg. In some embodiments, the TIM-3 binding protein is administered at a dose of about 11.25 mg/kg.

In one embodiment, the TIM-3 binding protein is administered once every 2-6 weeks (e.g. 2, 3 or 4 weeks, in particular 3 weeks). In one embodiment the TIM-3 binding protein is administered once every 3 weeks. In one embodiment the TIM-3 binding protein is administered once every 3 weeks for 2-6 dosing cycles (e.g. the first 3, 4, or 5 dosing cycles, in particular, the first 4 dosing cycles).

In one embodiment, the TIM-3 binding protein is administered at a dose of about 100 mg every 3 weeks. In one embodiment, the TIM-3 binding protein is administered at a dose of about 300 mg every 3 weeks. In one embodiment, the TIM-3 binding protein is administered at a dose of about 900 mg every 3 weeks. In some embodiments, the TIM-3 binding protein is administered at a dose of about 800 mg to about 1500 mg (e.g. about 800 mg, about 900 mg, about 1000 mg, about 1100 mg, about 1200 mg, about 1300 mg, about 1400 mg or about 1500 mg) every 4 weeks. In some embodiments, the TIM-3 binding protein is administered at a dose of about 800 mg to about 1500 mg (e.g. about 800 mg, about 900 mg, about 1000 mg, about 1100 mg, about 1200 mg, about 1300 mg, about 1400 mg or about 1500 mg) every 6 weeks. In some embodiments, the TIM-3 binding protein is administered at a dose of about 800 mg to about 1500 mg (e.g. about 800 mg, about 900 mg, about 1000 mg, about 1100 mg, about 1200 mg, about 1300 mg, about 1400 mg or about 1500 mg) every 8 weeks.

In one embodiment, the TIM-3 binding protein is cobolimab. In one embodiment, cobolimab is administered at a dose of 100 mg, 300 mg or 900 mg every 3 weeks. In one embodiment, cobolimab is administered at a dose of 300 mg every 3 weeks.

In one embodiment, the TIM-3 binding protein is MBG453. In one embodiment, MBG453 is administered at a dose of 80-1200 mg every two weeks or every four weeks. In another embodiment, MBG453 is administered at a dose of 800 mg every four weeks.

In one embodiment, the TIM-3 binding protein is LY3321367. In one embodiment, LY3321367 is administered at a dose of 3-1200 mg every two weeks. In another embodiment, LY3321367 is administered at a dose of 70-1200 mg every two weeks. In another embodiment, LY3321367 is administered at a dose of 1200 mg every two weeks.

In some embodiments, the PD-1 binding protein is administered at a dose of about 100-2000 mg (e.g. a dose about 100 mg; a dose about 200 mg; a dose about 300 mg; a dose about 400 mg; a dose about 500 mg; a dose about 600 mg; a dose about 700 mg; a dose about 800 mg; a dose about 900 mg; a dose about 1000 mg; a dose about 1100 mg; a dose about 1200 mg; a dose about 1300 mg; a dose about 1400 mg; a dose about 1500 mg; a dose about 1600 mg; a dose about 1700 mg; a dose about 1800 mg; a dose about 1900 mg; or a dose about 2000 mg). In some embodiments, the PD-1 binding protein is administered at a dose of about 1 mg/kg. In some embodiments, the PD-1 binding protein is administered at a dose of about 3 mg/kg. In some embodiments, the PD-1 binding protein is administered at a dose of about 6.25 mg/kg. In some embodiments, the PD-1 binding protein is administered at a dose of about 10 mg/kg. In some embodiments, the PD-1 binding protein is administered at a dose of about 12.5 mg/kg. In some embodiments, the PD-1 binding protein is administered at a dose of about 500 mg. In some embodiments, the PD-1 binding protein is administered at a dose of about 800 mg. In some embodiments, the PD-1 binding protein is administered at a dose of about 1000 mg.

In one embodiment, the PD-1 binding protein is administered once every 2-6 weeks (e.g. 2, 3 or 4 weeks, in particular 3 weeks). In one embodiment the PD-1 binding protein is administered for once every 3 weeks for 2-6 dosing cycles (e.g. the first 3, 4, or 5 dosing cycles, in particular, the first 4 dosing cycles).

In one embodiment, the PD-1 binding protein is administered at a dose of about 500 mg every 3 weeks. In one embodiment, the PD-1 binding protein is administered at a dose of about 1000 mg every 6 weeks. In one embodiment, the PD-1 binding protein is administered at a first dose of about 500 mg once every 3 weeks (Q3W) for 4 cycles, followed by a second dose of about 1000 mg once every 6 weeks (Q6W). In one embodiment, the PD-1 binding protein is administered at a dose of about 240 mg every 3 weeks. In one embodiment, the PD-1 binding protein is administered at a dose of about 350 mg every 3 weeks. In one embodiment, the PD-1 binding protein is administered at a dose of about 840 mg every 2 weeks, about 1200 mg every 3 weeks or about 1680 mg every 4 weeks. In one embodiment, the PD-1 binding protein is administered at a dose of about 800 mg every 2 weeks. In one embodiment, the PD-1 binding protein is administered at a dose of about 10 mg/kg every 2 weeks. In one embodiment, the PD-1 binding protein is administered at a dose of about 6.25 mg/kg every 3 weeks. In one embodiment, the PD-1 binding protein is administered at a dose of about 12.5 mg/kg every 6 weeks. In a further embodiment, the PD-1 binding protein is administered at a first dose of about 6.25 mg/kg once every 3 weeks (Q3W) for 4 cycles, followed by a second dose of about 12.5 mg/kg once every 6 weeks (Q6W).

In one embodiment, the PD-1 binding protein is dostarlimab. In one embodiment, dostarlimab is administered at a dose of 500 mg every 3 weeks. In one embodiment, dostarlimab is administered at a dose of 1000 mg every 6 weeks. In one embodiment, dostarlimab is administered at a dose of 6.25 mg/kg every 3 weeks. In one embodiment, dostarlimab is administered at a dose of 12.5 mg/kg every 6 weeks.

In one embodiment, the PD-1 binding protein is nivolumab. In one embodiment, nivolumab is administered at a dose of 240 mg every 3 weeks. In one embodiment, nivolumab is administered at a dose of 3 mg/kg every 3 weeks.

In one embodiment, the PD-1 binding protein is cemiplimab. In one embodiment, the PD-1 binding protein is cemiplimab. In one embodiment, cemiplimab is administered at a dose of 350 mg every 3 weeks. In one embodiment, cemiplimab is administered at a dose of 4.375 mg/kg every 3 weeks.

In one embodiment, the PD-1 binding protein is atezolizumab. In one embodiment, atezolizumab is administered at a dose of 840 mg every 2 weeks, 1200 mg every 3 weeks or 1680 mg every 4 weeks. In one embodiment, atezolizumab is administered at a dose of 10.5 mg/kg every 2 weeks, 15 mg/kg every 3 weeks or 21 mg/kg every 4 weeks.

In one embodiment, the PD-1 binding protein is avelumab. In one embodiment, avelumab is administered at a dose of 800 mg every 2 weeks. In one embodiment, avelumab is administered at a dose of 10 mg/kg every 2 weeks.

In one embodiment, the PD-1 binding protein is durvalumab. In one embodiment, durvalumab is administered at a dose of 800 mg every 2 weeks. In one embodiment, durvalumab is administered at a dose of 10 mg/kg every 2 weeks.

Fixed doses may be tested assuming a typical median weight of 80 kg.

Therapeutic monoclonal antibodies are often dosed based on body-size due to the concept that this reduces inter-subject variability in drug exposure. However, body-weight dependency of PK parameters does not always explain the observed variability in the exposure of monoclonal antibodies (Zhao et al. Annals of Oncology. (2017) 28:2002-2008). The advantage of body-weight based versus fixed dosing in the study provided in the Examples was evaluated through population PK modelling and simulation efforts. A preliminary population PK model was developed from monotherapy dose escalation (data up to doses of 1 mg/kg; n=19 subjects).

Simulations were performed by considering body weight distribution in the simulations were based on the observed distribution in the preliminary dataset. At the 5th percentile of body weight (40-47 kg), there was a 70-100% increase in median steady-state AUC(0-); H2L5 hIgG4PE exposures higher than these increases have been evaluated in the current Phase 1 study with the 3 mg/kg dose regimen. At the 95th percentile of body weight (107-118 kg), there was a 23-32% decrease in median steady-state AUC (0-) as compared to the median 80 kg exposure providing adequate receptor occupancy (RO) with the minimal lowering of exposure. A similar outcome is expected for steady-state Cmax and trough concentrations between body weight-based and fixed dosing.

Overall, these preliminary population PK simulations indicate that using fixed dosing would result in a similar range of exposures as that of body weight-based dosing. Also, fixed dosing offers the advantage of reduced dosing errors, reduced drug wastage, shorten preparation time, and improve ease of administration. Thus, switching to a fixed dose based on a reference body weight of 80 kg is reasonable and appropriate.

It is to be understood that where mg/kg is used, this is mg/kg of body weight. In one embodiment, the dose of the ICOS binding protein is between about 0.001 mg/kg to about 3.0 mg/kg. In another embodiment, the dose of the ICOS binding protein is about 0.001 mg/kg, about 0.003 mg/kg, about 0.01 mg/kg, about 0.03 mg/kg, about 0.1 mg/kg, about 0.3 mg/kg, about 0.6 mg/kg, about 1.0 mg/kg, about 2.0 mg/kg, about 3.0 mg/kg, or about 10 mg/kg. In one embodiment, the dose of ICOS binding protein is about 0.3 mg/kg. In another embodiment, the dose of the ICOS binding protein is at least 3.0 mg/kg. In one embodiment, the dose of the ICOS binding protein is in the range of about 0.001 mg/kg to about 10 mg/kg. In one embodiment, the dose of the ICOS binding protein is about 0.1 mg/kg to about 1.0 mg/kg. In one embodiment, the dose of the ICOS binding protein is about 0.1 mg/kg. In one embodiment, the dose of the ICOS binding protein is at least 0.1 mg/kg. In another embodiment, the dose of the ICOS binding protein is about 0.3 mg/kg. In another embodiment, the dose of the ICOS binding protein is about 1 mg/kg. In one embodiment, the dose of the ICOS binding protein is about 2 mg/kg. In one embodiment, the dose of the ICOS binding protein is about 3 mg/kg. In one embodiment, a fixed dose of ICOS binding protein may be administered, assuming a typical median weight of 80 kg.

In one embodiment, the dose of ICOS binding protein is increased during the treatment regimen. In one embodiment an initial dose of about 0.001 mg/kg, about 0.003 mg/kg, about 0.01 mg/kg, about 0.03 mg/kg, about 0.1 mg/kg, about 0.3 mg/kg, about 1.0 mg/kg is increased to about 0.003 mg/kg, about 0.01 mg/kg, about 0.03 mg/kg, about 0.1 mg/kg, about 0.3 mg/kg, about 1.0 mg/kg, about 2.0 mg/kg, about 3.0 mg/kg or at least 3.0 mg/kg. In one embodiment, an initial dose of 0.1 mg/kg is increased to 1 mg/kg. In one embodiment, an initial dose of 0.3 mg/kg is increased to 1 mg/kg. In one embodiment, the initial dose of 0.6 mg/kg is increased to 2 mg/kg.

In one embodiment, the ICOS binding protein is administered at 0.1 mg/kg×3 doses then 1 mg/kg. In one embodiment, the ICOS binding protein is administered at about 0.001 mg/kg, about 0.003 mg/kg, about 0.01 mg/kg, about 0.03 mg/kg, about 0.1 mg/kg, about 0.3 mg/kg, about 1.0 mg/kg, or about 3.0 mg/kg then increased to about 0.01 mg/kg, about 0.03 mg/kg, about 0.1 mg/kg, about 0.3 mg/kg, about 1.0 mg/kg, about 3.0 mg/kg or about 10 mg/kg.

In one embodiment, the dose of the TIM-3 binding protein is between about 0.0625 mg/kg to about 62.5 mg/kg. In another embodiment, the dose of the TIM-3 binding protein is about 1.25 mg/kg, about 3.75 mg/kg or about 11.25 mg/kg. In another embodiment, the dose of the TIM-3 binding protein is about 3.75 mg/kg. In one embodiment, the dose of the TIM-3 binding protein is in the range of about 1.25 mg/kg to about 11.25 mg/kg. In one embodiment, a fixed dose of TIM-3 binding protein may be administered, assuming a typical median weight of 80 kg.

In one embodiment, the dose of TIM-3 binding protein is increased during the treatment regimen. In one embodiment, an initial dose of about 1.25 mg/kg is increased to about 11.25 mg/kg. In one embodiment, an initial dose of about 1.25 mg/kg is increased to about 3.75 mg/kg. In one embodiment, an initial dose of about 3.75 mg/kg is increased to about 11.25 mg/kg. In one embodiment, an initial dose of about 1.25 mg/kg is increased to about 3.75 mg/kg and subsequently increased to about 11.25 mg/kg.

In one embodiment, the dose of the PD-1 binding protein is between about 1.25 mg/kg to about 25.0 mg/kg. In another embodiment, the dose of the PD-1 binding protein is about 1.25 mg/kg, about 6.25 mg/kg, about 12.5 mg/kg, about 18.75 mg/kg, or about 25.0 mg/kg. In another embodiment, the dose of the PD-1 binding protein is at least 6.25 mg/kg. In one embodiment, the dose of the PD-1 binding protein is in the range of about 6.25 mg/kg to about 12.5 mg/kg. In one embodiment, the dose of the PD-1 binding protein is about 6.25 mg/kg. In another embodiment, the dose of the PD-1 binding protein is about 12.5 mg/kg. In one embodiment, a fixed dose of PD-1 binding protein may be administered, assuming a typical median weight of 80 kg.

In one embodiment, the dose of PD-1 binding protein is increased during the treatment regimen. In one embodiment an initial dose of about 6.25 mg/kg is increased to about 12.5 mg/kg.

In one embodiment, the ICOS binding protein is administered once every 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 31 days, 32 days, 33 days, 34 days, 35 days, 36 days, 37 days, 38 days, 39 days, 40 days, 41 days, or 42 days. In one embodiment, the TIM-3 binding protein is administered once every 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 31 days, 32 days, 33 days, 34 days, 35 days, 36 days, 37 days, 38 days, 39 days, 40 days, 41 days, or 42 days.

In one embodiment, the ICOS binding protein is administered once every week, once every two weeks, once every three weeks, once every four weeks, once every five weeks or once every six weeks. In one embodiment, the ICOS binding protein is administered once every three weeks. In one embodiment, the ICOS binding protein is administered once every six weeks. In one embodiment, the ICOS binding protein is administered once every three weeks or once every six weeks until disease progression. In one embodiment, the ICOS binding protein is administered once every three weeks for 35 cycles.

In one embodiment, the TIM-3 binding protein is administered once every week, once every two weeks, once every three weeks, once every four weeks, once every five weeks or once every six weeks. In one embodiment, the TIM-3 binding protein is administered once every three weeks. In one embodiment, the TIM-3 binding protein is administered once every six weeks. In one embodiment, the TIM-3 binding protein is administered once every three weeks or once every six weeks until disease progression. In one embodiment, the TIM-3 binding protein is administered once every three weeks for 35 cycles.

In one embodiment, the PD-1 binding protein is administered once every week, once every two weeks, once every three weeks, once every four weeks, once every five weeks or once every six weeks. In one embodiment, the PD-1 binding protein is administered once every three weeks. In one embodiment, the PD-1 binding protein is administered once every six weeks. In one embodiment, the PD-1 binding protein is administered once every three weeks or once every six weeks until disease progression. In one embodiment, the PD-1 binding protein is administered once every three weeks for 35 cycles.

In one embodiment, the ICOS binding protein, TIM-3 binding protein and/or PD-1 binding protein is administered every three weeks up to 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40 cycles. In one embodiment, the ICOS binding protein, TIM-3 binding protein and/or PD-1 binding protein is administered every three weeks up to 35 cycles. In one embodiment, the ICOS binding protein, TIM-3 binding protein and/or PD-1 binding protein is administered every six weeks up to 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40 cycles. In one embodiment, the ICOS binding protein, TIM-3 binding protein and/or PD-1 binding protein is administered every six weeks up to 35 cycles.

The individual components of the combinations disclosed herein may be administered either in separate or combined form (e.g. as pharmaceutical formulations) by any convenient route.

For some therapeutic agents (i.e. binding proteins), suitable routes include oral, rectal, nasal, topical (including buccal and sublingual), vaginal, and parenteral (including subcutaneous, intramuscular, intravenous, intradermal, intrathecal, and epidural). It will be appreciated that the preferred route may vary with, for example, the condition of the recipient of the combination and the cancer to be treated. It will also be appreciated that each of the agents administered may be administered by the same or different routes and that the therapeutic agents may be formulated together or in separate pharmaceutical compositions.

In one embodiment, one or more binding agents of a combination of the invention are administered intravenously. In a further embodiment, the one or more binding agents of a combination of the invention are administered by intravenous infusion. In another embodiment, one or more therapeutic agents of a combination of the invention are administered intratumorally. In another embodiment, one or more binding agents of a combination of the invention are administered orally. In another embodiment, one or more binding agents of a combination of the invention are administered systemically, e.g. intravenously, and one or more other therapeutic agents of a combination of the invention are administered intratumorally. In another embodiment, all of the therapeutic agents of a combination of the invention are administered systemically, e.g. intravenously. In an alternative embodiment, all of the therapeutic agents of the combination of the invention are administered intratumorally. In any of the embodiments, e.g. in this paragraph, the therapeutic agents of the invention may be administered as one or more pharmaceutical compositions.

In one embodiment, the ICOS binding protein is administered via intravenous (IV) infusion. In one embodiment, the TIM-3 binding protein is administered via IV infusion. In one embodiment, the PD-1 binding protein is administered via IV infusion.

In one embodiment, the therapeutic agent (e.g. the ICOS binding protein, the PD-1 binding protein or the TIM-3 binding protein) is administered via IV infusion over 30 minutes, 60 minutes or 90 minutes. In one embodiment, the therapeutic agent is administered via IV infusion over 30 minutes. In one embodiment, the ICOS binding protein is administered via IV infusion over 30 minutes.

In one embodiment, where two or more therapeutic agents are administered concurrently via IV infusion, the second therapeutic agent is administered via IV infusion at least 30 minutes and no longer than one hour following the end of infusion (EOI) of the first therapeutic agent. Where a third therapeutic agent is administered concurrently with the first and second, the third therapeutic agent is administered via IV infusion at least 30 minutes and no longer than one hour following the end of infusion of the second therapeutic agent. In one embodiment, the ICOS binding protein is administered first, followed by the TIM-3 binding protein. In one embodiment, the ICOS binding protein is administered first, followed by the PD-1 binding protein and then followed by the TIM-3 binding protein. In one embodiment, the ICOS binding protein is administered first at a dose of 24 mg Q3W via IV infusion. The PD-1 binding protein is administered at a dose of 500 mg Q3W via IV infusion at least 30 minutes and no longer than one hour following end of infusion of the ICOS binding protein. The TIM-3 binding protein is administered at a dose of 300 mg Q3W via IV infusion at least 30 minutes and no longer than one hour following end of infusion of the PD-1 binding protein. In one embodiment the ICOS binding protein is H2L5 IgG4PE. In one embodiment, the PD-1 binding protein is dostarlimab. In one embodiment, the TIM-3 binding protein is cobolimab.

In one embodiment, the ICOS binding protein is administered via IV infusion at a dose of about 0.08 mg, about 0.24 mg, about 0.48 mg, about 0.8 mg, about 1.6 mg, about 2.4 mg, about 8.0 mg, about 24 mg, about 48 mg, about 80 mg, about 160 mg or about 240 mg every three weeks. In one embodiment, the ICOS binding protein is administered at a dose of 24 mg or 80 mg via IV infusion every three weeks. In one embodiment, the ICOS binding protein is administered at a dose of 0.3 mg/kg or 1 mg/kg via IV infusion every three weeks. In one embodiment, the ICOS binding protein is administered via IV infusion at a dose of about 8 mg, about 24 mg, about 48 mg, about 80 mg, about 160 mg or about 240 mg every six weeks. In one embodiment, the ICOS binding protein is administered at a dose of 48 mg or 160 mg via IV infusion every six weeks. In one embodiment, the ICOS binding protein is administered at a dose of 0.6 mg/kg or 2 mg/kg via IV infusion every six weeks.

In one embodiment, the TIM-3 binding protein is administered via IV infusion at a dose of about 10 mg, about 20 mg, about 30 mg, about 40 mg, about 50 mg, about 60 mg, about 70 mg, about 80 mg, about 90 mg, about 100 mg, about 150 mg, about 200 mg, about 300 mg, about 400 mg, about 500 mg, about 600 mg, about 700 mg, about 800 mg, about 900 mg, about 1000 mg, about 2000 mg, about 3000 mg, about 4000 mg or about 5000 mg every three weeks. In one embodiment, the TIM-3 binding protein is administered at a dose of 100 mg via IV infusion every three weeks. In one embodiment, the TIM-3 binding protein is administered at a dose of about 1.25 mg/kg via IV infusion every three weeks. In one embodiment, the TIM-3 binding protein is administered at a dose of 300 mg via IV infusion every three weeks. In one embodiment, the TIM-3 binding protein is administered at a dose of about 3.75 mg/kg via IV infusion every three weeks. In one embodiment, the TIM-3 binding protein is administered at a dose of 900 mg via IV infusion every three weeks. In one embodiment, the TIM-3 binding protein is administered at a dose of about 11.25 mg/kg via IV infusion every three weeks. In one embodiment, the TIM-3 binding protein is administered via IV infusion at a dose of about 100 mg, about 300 mg or about 900 mg every six weeks. In one embodiment, the TIM-3 binding protein is administered at a dose of about 1.25 mg/kg, about 3.75 mg/kg or about 11.25 mg/kg via IV infusion every six weeks.

In one embodiment, the PD-1 binding protein is administered via IV infusion at a dose of about 100 mg, about 200 mg, about 300 mg, about 400 mg, about 500 mg, about 600 mg, about 700 mg, about 800 mg, about 900 mg, or about 1000 mg every three weeks. In one embodiment, the PD-1 binding protein is administered at a dose of 500 mg via IV infusion every three weeks. In one embodiment, the PD-1 binding protein is administered at a dose of about 6.25 mg/kg via IV infusion every three weeks. In one embodiment, the PD-1 binding protein is administered via IV infusion at a dose of about 500 mg, about 600 mg, about 700 mg, about 800 mg, about 900 mg, about 1000 mg, about 1100 mg, about 1200 mg, about 1300 mg, about 1400 mg or about 1500 mg every six weeks. In one embodiment, the PD-1 binding protein is administered at a dose of 1000 mg via IV infusion every six weeks. In one embodiment, the ICOS binding protein is administered at a dose of about 12.5 mg/kg via IV infusion every six weeks.

In one embodiment, the ICOS binding protein is administered at a dose of 0.3 mg/kg via IV infusion every three weeks and the TIM-3 binding protein is administered at a dose of 100 mg via IV infusion every three weeks. In one embodiment, the ICOS binding protein is administered at a dose of 24 mg via IV infusion every three weeks and the TIM-3 binding protein is administered at a dose of 100 mg via IV infusion every three weeks. In one embodiment, the ICOS binding protein is administered at a dose of 0.3 mg/kg via IV infusion every three weeks and the TIM-3 binding protein is administered at a dose of 300 mg via IV infusion every three weeks. In one embodiment, the ICOS binding protein is administered at a dose of 24 mg via IV infusion every three weeks and the TIM-3 binding protein is administered at a dose of 300 mg via IV infusion every three weeks. In one embodiment, the ICOS binding protein is administered at a dose of 0.3 mg/kg via IV infusion every three weeks and the TIM-3 binding protein is administered at a dose of 900 mg via IV infusion every three weeks. In one embodiment, the ICOS binding protein is administered at a dose of 24 mg via IV infusion every three weeks and the TIM-3 binding protein is administered at a dose of 900 mg via IV infusion every three weeks. In one embodiment, the ICOS binding protein is administered at a dose of 1.0 mg/kg via IV infusion every three weeks and the TIM-3 binding protein is administered at a dose of 100 mg via IV infusion every three weeks. In one embodiment, the ICOS binding protein is administered at a dose of 80 mg via IV infusion every three weeks and the TIM-3 binding protein is administered at a dose of 100 mg via IV infusion every three weeks. In one embodiment, the ICOS binding protein is administered at a dose of 1.0 mg/kg via IV infusion every three weeks and the TIM-3 binding protein is administered at a dose of 300 mg via IV infusion every three weeks. In one embodiment, the ICOS binding protein is administered at a dose of 80 mg via IV infusion every three weeks and the TIM-3 binding protein is administered at a dose of 300 mg via IV infusion every three weeks. In one embodiment, the ICOS binding protein is administered at a dose of 1.0 mg/kg via IV infusion every three weeks and the TIM-3 binding protein is administered at a dose of 900 mg via IV infusion every three weeks. In one embodiment, the ICOS binding protein is administered at a dose of 80 mg via IV infusion every three weeks and the TIM-3 binding protein is administered at a dose of 900 mg via IV infusion every three weeks.

In one embodiment, the ICOS binding protein is administered at a dose of 0.6 mg/kg via IV infusion every six weeks and the TIM-3 binding protein is administered at a dose of 100 mg via IV infusion every three weeks. In one embodiment, the ICOS binding protein is administered at a dose of 48 mg via IV infusion every six weeks and the TIM-3 binding protein is administered at a dose of 100 mg via IV infusion every three weeks. In one embodiment, the ICOS binding protein is administered at a dose of 0.6 mg/kg via IV infusion every six weeks and the TIM-3 binding protein is administered at a dose of 300 mg via IV infusion every three weeks. In one embodiment, the ICOS binding protein is administered at a dose of 48 mg via IV infusion every six weeks and the TIM-3 binding protein is administered at a dose of 300 mg via IV infusion every three weeks. In one embodiment, the ICOS binding protein is administered at a dose of 0.6 mg/kg via IV infusion every six weeks and the TIM-3 binding protein is administered at a dose of 900 mg via IV infusion every three weeks. In one embodiment, the ICOS binding protein is administered at a dose of 48 mg via IV infusion every six weeks and the TIM-3 binding protein is administered at a dose of 900 mg via IV infusion every three weeks. In one embodiment, the ICOS binding protein is administered at a dose of 2.0 mg/kg via IV infusion every six weeks and the TIM-3 binding protein is administered at a dose of 100 mg via IV infusion every three weeks. In one embodiment, the ICOS binding protein is administered at a dose of 160 mg via IV infusion every six weeks and the TIM-3 binding protein is administered at a dose of 100 mg via IV infusion every three weeks. In one embodiment, the ICOS binding protein is administered at a dose of 2.0 mg/kg via IV infusion every six weeks and the TIM-3 binding protein is administered at a dose of 300 mg via IV infusion every three weeks. In one embodiment, the ICOS binding protein is administered at a dose of 160 mg via IV infusion every six weeks and the TIM-3 binding protein is administered at a dose of 300 mg via IV infusion every three weeks. In one embodiment, the ICOS binding protein is administered at a dose of 2.0 mg/kg via IV infusion every six weeks and the TIM-3 binding protein is administered at a dose of 900 mg via IV infusion every three weeks. In one embodiment, the ICOS binding protein is administered at a dose of 160 mg via IV infusion every six weeks and the TIM-3 binding protein is administered at a dose of 900 mg via IV infusion every three weeks.

In one embodiment, the TIM-3 binding protein is administered once every three weeks. In one embodiment, the TIM-3 binding protein is cobolimab. In one embodiment, 100 mg of cobolimab is administered via IV infusion every 3 weeks. In one embodiment, 300 mg of cobolimab is administered via IV infusion every 3 weeks. In one embodiment, 900 mg of cobolimab is administered via IV infusion every 3 weeks. In one embodiment, 100 mg of cobolimab is administered via IV infusion every 3 weeks for four dosing cycles and then 100 mg, 300 mg or 900 mg every 6 weeks thereafter (i.e. until disease progression). In one embodiment, 300 mg of cobolimab is administered via IV infusion every 3 weeks for four dosing cycles and then 300 mg or 900 mg every 6 weeks thereafter. In one embodiment, 900 mg of cobolimab is administered via IV infusion every 3 weeks for four dosing cycles and then 900 mg every 6 weeks thereafter.

In one embodiment, the ICOS binding protein is administered at a dose of 0.3 mg/kg via IV infusion every three weeks, the PD-1 binding protein is administered at a dose of 500 mg via IV infusion every three weeks and the TIM-3 binding protein is administered at a dose of 100 mg via IV infusion every three weeks. In one embodiment, the ICOS binding protein is administered at a dose of 24 mg via IV infusion every three weeks, the PD-1 binding protein is administered at a dose of 500 mg via IV infusion every three weeks and the TIM-3 binding protein is administered at a dose of 100 mg via IV infusion every three weeks. In one embodiment, the ICOS binding protein is administered at a dose of 0.3 mg/kg via IV infusion every three weeks, the PD-1 binding protein is administered at a dose of 500 mg via IV infusion every three weeks and the TIM-3 binding protein is administered at a dose of 300 mg via IV infusion every three weeks. In one embodiment, the ICOS binding protein is administered at a dose of 24 mg via IV infusion every three weeks, the PD-1 binding protein is administered at a dose of 500 mg via IV infusion every three weeks and the TIM-3 binding protein is administered at a dose of 300 mg via IV infusion every three weeks. In one embodiment, the ICOS binding protein is administered at a dose of 0.3 mg/kg via IV infusion every three weeks, the PD-1 binding protein is administered at a dose of 500 mg via IV infusion every three weeks and the TIM-3 binding protein is administered at a dose of 900 mg via IV infusion every three weeks. In one embodiment, the ICOS binding protein is administered at a dose of 24 mg via IV infusion every three weeks, the PD-1 binding protein is administered at a dose of 500 mg via IV infusion every three weeks and the TIM-3 binding protein is administered at a dose of 900 mg via IV infusion every three weeks. In one embodiment, the ICOS binding protein is administered at a dose of 1.0 mg/kg via IV infusion every three weeks, the PD-1 binding protein is administered at a dose of 500 mg via IV infusion every three weeks and the TIM-3 binding protein is administered at a dose of 100 mg via IV infusion every three weeks. In one embodiment, the ICOS binding protein is administered at a dose of 80 mg via IV infusion every three weeks, the PD-1 binding protein is administered at a dose of 500 mg via IV infusion every three weeks and the TIM-3 binding protein is administered at a dose of 100 mg via IV infusion every three weeks. In one embodiment, the ICOS binding protein is administered at a dose of 1.0 mg/kg via IV infusion every three weeks, the PD-1 binding protein is administered at a dose of 500 mg via IV infusion every three weeks and the TIM-3 binding protein is administered at a dose of 300 mg via IV infusion every three weeks. In one embodiment, the ICOS binding protein is administered at a dose of 80 mg via IV infusion every three weeks, the PD-1 binding protein is administered at a dose of 500 mg via IV infusion every three weeks and the TIM-3 binding protein is administered at a dose of 300 mg via IV infusion every three weeks. In one embodiment, the ICOS binding protein is administered at a dose of 1.0 mg/kg via IV infusion every three weeks, the PD-1 binding protein is administered at a dose of 500 mg via IV infusion every three weeks and the TIM-3 binding protein is administered at a dose of 900 mg via IV infusion every three weeks. In one embodiment, the ICOS binding protein is administered at a dose of 80 mg via IV infusion every three weeks, the PD-1 binding protein is administered at a dose of 500 mg via IV infusion every three weeks and the TIM-3 binding protein is administered at a dose of 900 mg via IV infusion every three weeks.

In one embodiment, the ICOS binding protein is administered at a dose of 0.6 mg/kg via IV infusion every six weeks, the PD-1 binding protein is administered at a dose of 1000 mg via IV infusion every six weeks and the TIM-3 binding protein is administered at a dose of 100 mg via IV infusion every three weeks. In one embodiment, the ICOS binding protein is administered at a dose of 48 mg via IV infusion every six weeks, the PD-1 binding protein is administered at a dose of 1000 mg via IV infusion every six weeks and the TIM-3 binding protein is administered at a dose of 100 mg via IV infusion every three weeks. In one embodiment, the ICOS binding protein is administered at a dose of 0.6 mg/kg via IV infusion every six weeks, the PD-1 binding protein is administered at a dose of 1000 mg via IV infusion every six weeks and the TIM-3 binding protein is administered at a dose of 300 mg via IV infusion every three weeks. In one embodiment, the ICOS binding protein is administered at a dose of 48 mg via IV infusion every six weeks, the PD-1 binding protein is administered at a dose of 1000 mg via IV infusion every six weeks and the TIM-3 binding protein is administered at a dose of 300 mg via IV infusion every three weeks. In one embodiment, the ICOS binding protein is administered at a dose of 0.6 mg/kg via IV infusion every six weeks, the PD-1 binding protein is administered at a dose of 1000 mg via IV infusion every six weeks and the TIM-3 binding protein is administered at a dose of 900 mg via IV infusion every three weeks. In one embodiment, the ICOS binding protein is administered at a dose of 48 mg via IV infusion every six weeks, the PD-1 binding protein is administered at a dose of 1000 mg via IV infusion every six weeks and the TIM-3 binding protein is administered at a dose of 900 mg via IV infusion every three weeks. In one embodiment, the ICOS binding protein is administered at a dose of 2.0 mg/kg via IV infusion every six weeks, the PD-1 binding protein is administered at a dose of 1000 mg via IV infusion every six weeks and the TIM-3 binding protein is administered at a dose of 100 mg via IV infusion every three weeks. In one embodiment, the ICOS binding protein is administered at a dose of 160 mg via IV infusion every six weeks, the PD-1 binding protein is administered at a dose of 1000 mg via IV infusion every six weeks and the TIM-3 binding protein is administered at a dose of 100 mg via IV infusion every three weeks. In one embodiment, the ICOS binding protein is administered at a dose of 2.0 mg/kg via IV infusion every six weeks, the PD-1 binding protein is administered at a dose of 1000 mg via IV infusion every six weeks and the TIM-3 binding protein is administered at a dose of 300 mg via IV infusion every three weeks. In one embodiment, the ICOS binding protein is administered at a dose of 160 mg via IV infusion every six weeks, the PD-1 binding protein is administered at a dose of 1000 mg via IV infusion every six weeks and the TIM-3 binding protein is administered at a dose of 300 mg via IV infusion every three weeks. In one embodiment, the ICOS binding protein is administered at a dose of 2.0 mg/kg via IV infusion every six weeks, the PD-1 binding protein is administered at a dose of 1000 mg via IV infusion every six weeks and the TIM-3 binding protein is administered at a dose of 900 mg via IV infusion every three weeks. In one embodiment, the ICOS binding protein is administered at a dose of 160 mg via IV infusion every six weeks, the PD-1 binding protein is administered at a dose of 1000 mg via IV infusion every six weeks and the TIM-3 binding protein is administered at a dose of 900 mg via IV infusion every three weeks. In one embodiment, the ICOS binding protein is H2L5 IgG4PE. In one embodiment, the PD-1 binding protein is dostarlimab. In one embodiment, the TIM-3 binding protein is cobolimab.

In one embodiment, the PD-1 binding protein is administered once every three weeks. In one embodiment, the PD-1 binding protein is dostarlimab. In one embodiment, 500 mg of dostarlimab is administered via IV infusion every 3 weeks. In one embodiment, 500 mg of dostarlimab is administered via IV infusion every 3 weeks for four dosing cycles and then 1000 mg every 6 weeks thereafter (i.e. until disease progression). In a further embodiment, 6.25 mg/kg of dostarlimab is administered via IV infusion every 3 weeks. In one embodiment, 6.25 mg/kg of dostarlimab is administered via IV infusion every 3 weeks for four dosing cycles and then 12.5 mg/kg every 6 weeks thereafter.

In some embodiments, the patient is first administered the ICOS binding protein as a monotherapy regimen and then the ICOS binding protein with the TIM-3 binding protein as a combination therapy regimen. In some embodiments, the patient is first administered the TIM-3 binding protein as a monotherapy regimen and then the ICOS binding protein with the TIM-3 binding protein as a combination therapy regimen. In some embodiments, the patient is first administered the ICOS binding protein as a monotherapy regimen and then the ICOS binding protein with the PD-1 binding protein and TIM-3 binding protein as a combination therapy regimen. In some embodiments, the patient is first administered the TIM-3 binding protein as a monotherapy regimen and then the TIM-3 binding protein with the ICOS binding protein and PD-1 binding protein as a combination therapy regimen.

In some embodiments, the patient is first administered the ICOS binding protein at a dose of about 0.08 mg to about 800 mg as a monotherapy regimen and then the ICOS binding protein at a dose of about 0.08 mg to about 800 mg with the TIM-3 binding protein at a dose of 5 mg to 5000 mg as a combination therapy regimen. In one embodiment, the patient is first administered the ICOS binding protein at a dose of about 24 mg, about 48 mg, about 80 mg or about 160 mg as a monotherapy regimen and then the ICOS binding protein at a dose of about about 24 mg, about 48 mg, about 80 mg or about 160 mg with the TIM-3 binding protein at a dose of 5 mg to 5000 mg as a combination therapy regimen. In one embodiment, the patient is first administered the ICOS binding protein at a dose of 24 mg as a monotherapy regimen and then the ICOS binding protein at a dose of 24 mg with the TIM-3 binding protein at a dose of 100 mg as a combination therapy regimen. In one embodiment, the patient is first administered the ICOS binding protein at a dose of 24 mg as a monotherapy regimen and then the ICOS binding protein at a dose of 24 mg with the TIM-3 binding protein at a dose of 300 mg as a combination therapy regimen. In one embodiment, the patient is first administered the ICOS binding protein at a dose of 24 mg as a monotherapy regimen and then the ICOS binding protein at a dose of 24 mg with the TIM-3 binding protein at a dose of 900 mg as a combination therapy regimen. In one embodiment, the patient is first administered the ICOS binding protein at a dose of 80 mg as a monotherapy regimen and then the ICOS binding protein at a dose of 80 mg with the TIM-3 binding protein at a dose of 100 mg as a combination therapy regimen. In one embodiment, the patient is first administered the ICOS binding protein at a dose of 80 mg as a monotherapy regimen and then the ICOS binding protein at a dose of 80 mg with the TIM-3 binding protein at a dose of 300 mg as a combination therapy regimen. In one embodiment, the patient is first administered the ICOS binding protein at a dose of 80 mg as a monotherapy regimen and then the ICOS binding protein at a dose of 80 mg with the TIM-3 binding protein at a dose of 900 mg as a combination therapy regimen.

In some embodiments, the patient is first administered the ICOS binding protein at a dose of about 0.08 mg to about 800 mg as a monotherapy regimen and then the ICOS binding protein at a dose of about 0.08 mg to about 800 mg with the PD-1 binding protein at a dose of 100 mg to 2000 mg and the TIM-3 binding protein at a dose of 5 mg to 5000 mg as a combination therapy regimen. In one embodiment, the patient is first administered the ICOS binding protein at a dose of about 24 mg, about 48 mg, about 80 mg or about 160 mg as a monotherapy regimen and then the ICOS binding protein at a dose of about 24 mg, about 48 mg, about 80 mg or about 160 mg with the PD-1 binding protein at a dose of 100 mg to 2000 mg and the TIM-3 binding protein at a dose of 5 mg to 5000 mg as a combination therapy regimen. In one embodiment, the patient is first administered the ICOS binding protein at a dose of 24 mg as a monotherapy regimen and then the ICOS binding protein at a dose of 24 mg with the PD-1 binding protein at a dose of 500 mg and the TIM-3 binding protein at a dose of 100 mg as a combination therapy regimen. In one embodiment, the patient is first administered the ICOS binding protein at a dose of 24 mg as a monotherapy regimen and then the ICOS binding protein at a dose of 24 mg with the PD-1 binding protein at a dose of 500 mg and the TIM-3 binding protein at a dose of 300 mg as a combination therapy regimen. In one embodiment, the patient is first administered the ICOS binding protein at a dose of 24 mg as a monotherapy regimen and then the ICOS binding protein at a dose of 24 mg with the PD-1 binding protein at a dose of 500 mg and the TIM-3 binding protein at a dose of 900 mg as a combination therapy regimen. In one embodiment, the patient is first administered the ICOS binding protein at a dose of 80 mg as a monotherapy regimen and then the ICOS binding protein at a dose of 80 mg with the PD-1 binding protein at a dose of 500 mg and the TIM-3 binding protein at a dose of 100 mg as a combination therapy regimen. In one embodiment, the patient is first administered the ICOS binding protein at a dose of 80 mg as a monotherapy regimen and then the ICOS binding protein at a dose of 80 mg with the PD-1 binding protein at a dose of 500 mg and the TIM-3 binding protein at a dose of 300 mg as a combination therapy regimen. In one embodiment, the patient is first administered the ICOS binding protein at a dose of 80 mg as a monotherapy regimen and then the ICOS binding protein at a dose of 80 mg with the PD-1 binding protein at a dose of 500 mg and the TIM-3 binding protein at a dose of 900 mg as a combination therapy regimen. In one embodiment, the patient is first administered the ICOS binding protein at a dose of 24 mg as a monotherapy regimen and then the ICOS binding protein at a dose of 24 mg with the PD-1 binding protein at a dose of 1000 mg and the TIM-3 binding protein at a dose of 100 mg as a combination therapy regimen. In one embodiment, the patient is first administered the ICOS binding protein at a dose of 24 mg as a monotherapy regimen and then the ICOS binding protein at a dose of 24 mg with the PD-1 binding protein at a dose of 1000 mg and the TIM-3 binding protein at a dose of 300 mg as a combination therapy regimen. In one embodiment, the patient is first administered the ICOS binding protein at a dose of 24 mg as a monotherapy regimen and then the ICOS binding protein at a dose of 24 mg with the PD-1 binding protein at a dose of 1000 mg and the TIM-3 binding protein at a dose of 900 mg as a combination therapy regimen. In one embodiment, the patient is first administered the ICOS binding protein at a dose of 80 mg as a monotherapy regimen and then the ICOS binding protein at a dose of 80 mg with the PD-1 binding protein at a dose of 1000 mg and the TIM-3 binding protein at a dose of 100 mg as a combination therapy regimen. In one embodiment, the patient is first administered the ICOS binding protein at a dose of 80 mg as a monotherapy regimen and then the ICOS binding protein at a dose of 80 mg with the PD-1 binding protein at a dose of 1000 mg and the TIM-3 binding protein at a dose of 300 mg as a combination therapy regimen. In one embodiment, the patient is first administered the ICOS binding protein at a dose of 80 mg as a monotherapy regimen and then the ICOS binding protein at a dose of 80 mg with the PD-1 binding protein at a dose of 1000 mg and the TIM-3 binding protein at a dose of 900 mg as a combination therapy regimen.

In a further embodiment, the patient is first administered the ICOS binding protein at a dose of 24 mg as a monotherapy regimen every 3 weeks for up to 1, 2, 3, 4, 5, 6, 7, 8, 9, 10, 11, 12, 13 or 14 cycles and then the ICOS binding protein at a dose of 24 mg with the TIM-3 binding protein at a dose of 100 mg as a combination therapy regimen every 3 weeks for up to 1, 2, 3, 4, 5, 6, 7, 8, 9, 10, 11, 12, 13 or 14 cycles. In a further embodiment, the patient is first administered the ICOS binding protein at a dose of 24 mg as a monotherapy regimen every 3 weeks for up to 1, 2, 3, 4, 5, 6, 7, 8, 9, 10, 11, 12, 13 or 14 cycles and then the ICOS binding protein at a dose of 24 mg with the TIM-3 binding protein at a dose of 300 mg as a combination therapy regimen every 3 weeks for up to 1, 2, 3, 4, 5, 6, 7, 8, 9, 10, 11, 12, 13 or 14 cycles. In a further embodiment, the patient is first administered the ICOS binding protein at a dose of 24 mg as a monotherapy regimen every 3 weeks for up to 1, 2, 3, 4, 5, 6, 7, 8, 9, 10, 11, 12, 13 or 14 cycles and then the ICOS binding protein at a dose of 24 mg with the TIM-3 binding protein at a dose of 900 mg as a combination therapy regimen every 3 weeks for up to 1, 2, 3, 4, 5, 6, 7, 8, 9, 10, 11, 12, 13 or 14 cycles. In a further embodiment, the patient is first administered the ICOS binding protein at a dose of 80 mg as a monotherapy regimen every 3 weeks for up to 1, 2, 3, 4, 5, 6, 7, 8, 9, 10, 11, 12, 13 or 14 cycles and then the ICOS binding protein at a dose of 80 mg with the TIM-3 binding protein at a dose of 100 mg as a combination therapy regimen every 3 weeks for up to 1, 2, 3, 4, 5, 6, 7, 8, 9, 10, 11, 12, 13 or 14 cycles. In a further embodiment, the patient is first administered the ICOS binding protein at a dose of 80 mg as a monotherapy regimen every 3 weeks for up to 1, 2, 3, 4, 5, 6, 7, 8, 9, 10, 11, 12, 13 or 14 cycles and then the ICOS binding protein at a dose of 80 mg with the TIM-3 binding protein at a dose of 300 mg as a combination therapy regimen every 3 weeks for up to 1, 2, 3, 4, 5, 6, 7, 8, 9, 10, 11, 12, 13 or 14 cycles. In a further embodiment, the patient is first administered the ICOS binding protein at a dose of 80 mg as a monotherapy regimen every 3 weeks for up to 1, 2, 3, 4, 5, 6, 7, 8, 9, 10, 11, 12, 13 or 14 cycles and then the ICOS binding protein at a dose of 80 mg with the TIM-3 binding protein at a dose of 900 mg as a combination therapy regimen every 3 weeks for up to 1, 2, 3, 4, 5, 6, 7, 8, 9, 10, 11, 12, 13 or 14 cycles.

In a further embodiment, the patient is first administered the ICOS binding protein at a dose of 24 mg as a monotherapy regimen every 3 weeks for up to 1, 2, 3, 4, 5, 6, 7, 8, 9, 10, 11, 12, 13 or 14 cycles and then the ICOS binding protein at a dose of 24 mg with the PD-1 binding protein at a dose of 500 mg and the TIM-3 binding protein at a dose of 100 mg as a combination therapy regimen every 3 weeks for up to 1, 2, 3, 4, 5, 6, 7, 8, 9, 10, 11, 12, 13 or 14 cycles. In a further embodiment, the patient is first administered the ICOS binding protein at a dose of 24 mg as a monotherapy regimen every 3 weeks for up to 1, 2, 3, 4, 5, 6, 7, 8, 9, 10, 11, 12, 13 or 14 cycles and then the ICOS binding protein at a dose of 24 mg with the PD-1 binding protein at a dose of 500 mg and the TIM-3 binding protein at a dose of 300 mg as a combination therapy regimen every 3 weeks for up to 1, 2, 3, 4, 5, 6, 7, 8, 9, 10, 11, 12, 13 or 14 cycles. In a further embodiment, the patient is first administered the ICOS binding protein at a dose of 24 mg as a monotherapy regimen every 3 weeks for up to 1, 2, 3, 4, 5, 6, 7, 8, 9, 10, 11, 12, 13 or 14 cycles and then the ICOS binding protein at a dose of 24 mg with the PD-1 binding protein at a dose of 500 mg and the TIM-3 binding protein at a dose of 900 mg as a combination therapy regimen every 3 weeks for up to 1, 2, 3, 4, 5, 6, 7, 8, 9, 10, 11, 12, 13 or 14 cycles. In a further embodiment, the patient is first administered the ICOS binding protein at a dose of 80 mg as a monotherapy regimen every 3 weeks for up to 1, 2, 3, 4, 5, 6, 7, 8, 9, 10, 11, 12, 13 or 14 cycles and then the ICOS binding protein at a dose of 80 mg with the PD-1 binding protein at a dose of 500 mg and the TIM-3 binding protein at a dose of 100 mg as a combination therapy regimen every 3 weeks for up to 1, 2, 3, 4, 5, 6, 7, 8, 9, 10, 11, 12, 13 or 14 cycles. In a further embodiment, the patient is first administered the ICOS binding protein at a dose of 80 mg as a monotherapy regimen every 3 weeks for up to 1, 2, 3, 4, 5, 6, 7, 8, 9, 10, 11, 12, 13 or 14 cycles and then the ICOS binding protein at a dose of 80 mg with the PD-1 binding protein at a dose of 500 mg and the TIM-3 binding protein at a dose of 300 mg as a combination therapy regimen every 3 weeks for up to 1, 2, 3, 4, 5, 6, 7, 8, 9, 10, 11, 12, 13 or 14 cycles. In a further embodiment, the patient is first administered the ICOS binding protein at a dose of 80 mg as a monotherapy regimen every 3 weeks for up to 1, 2, 3, 4, 5, 6, 7, 8, 9, 10, 11, 12, 13 or 14 cycles and then the ICOS binding protein at a dose of 80 mg with the PD-1 binding protein at a dose of 500 mg and the TIM-3 binding protein at a dose of 900 mg as a combination therapy regimen every 3 weeks for up to 1, 2, 3, 4, 5, 6, 7, 8, 9, 10, 11, 12, 13 or 14 cycles.

In a further embodiment, the patient is first administered the ICOS binding protein at a dose of 48 mg as a monotherapy regimen every 6 weeks for up to 1, 2, 3, 4, 5, 6, 7, 8, 9, 10, 11, 12, 13 or 14 cycles and then the ICOS binding protein at a dose of 48 mg with the PD-1 binding protein at a dose of 1000 mg and the TIM-3 binding protein at a dose of 100 mg as a combination therapy regimen every 6 weeks for up to 1, 2, 3, 4, 5, 6, 7, 8, 9, 10, 11, 12, 13 or 14 cycles. In a further embodiment, the patient is first administered the ICOS binding protein at a dose of 48 mg as a monotherapy regimen every 6 weeks for up to 1, 2, 3, 4, 5, 6, 7, 8, 9, 10, 11, 12, 13 or 14 cycles and then the ICOS binding protein at a dose of 48 mg with the PD-1 binding protein at a dose of 1000 mg and the TIM-3 binding protein at a dose of 300 mg as a combination therapy regimen every 6 weeks for up to 1, 2, 3, 4, 5, 6, 7, 8, 9, 10, 11, 12, 13 or 14 cycles. In a further embodiment, the patient is first administered the ICOS binding protein at a dose of 48 mg as a monotherapy regimen every 6 weeks for up to 1, 2, 3, 4, 5, 6, 7, 8, 9, 10, 11, 12, 13 or 14 cycles and then the ICOS binding protein at a dose of 48 mg with the PD-1 binding protein at a dose of 1000 mg and the TIM-3 binding protein at a dose of 900 mg as a combination therapy regimen every 6 weeks for up to 1, 2, 3, 4, 5, 6, 7, 8, 9, 10, 11, 12, 13 or 14 cycles. In a further embodiment, the patient is first administered the ICOS binding protein at a dose of 160 mg as a monotherapy regimen every 6 weeks for up to 1, 2, 3, 4, 5, 6, 7, 8, 9, 10, 11, 12, 13 or 14 cycles and then the ICOS binding protein at a dose of 160 mg with the PD-1 binding protein at a dose of 1000 mg and the TIM-3 binding protein at a dose of 100 mg as a combination therapy regimen every 6 weeks for up to 1, 2, 3, 4, 5, 6, 7, 8, 9, 10, 11, 12, 13 or 14 cycles. In a further embodiment, the patient is first administered the ICOS binding protein at a dose of 160 mg as a monotherapy regimen every 6 weeks for up to 1, 2, 3, 4, 5, 6, 7, 8, 9, 10, 11, 12, 13 or 14 cycles and then the ICOS binding protein at a dose of 160 mg with the PD-1 binding protein at a dose of 1000 mg and the TIM-3 binding protein at a dose of 300 mg as a combination therapy regimen every 6 weeks for up to 1, 2, 3, 4, 5, 6, 7, 8, 9, 10, 11, 12, 13 or 14 cycles. In a further embodiment, the patient is first administered the ICOS binding protein at a dose of 160 mg as a monotherapy regimen every 6 weeks for up to 1, 2, 3, 4, 5, 6, 7, 8, 9, 10, 11, 12, 13 or 14 cycles and then the ICOS binding protein at a dose of 160 mg with the PD-1 binding protein at a dose of 1000 mg and the TIM-3 binding protein at a dose of 900 mg as a combination therapy regimen every 6 weeks for up to 1, 2, 3, 4, 5, 6, 7, 8, 9, 10, 11, 12, 13 or 14 cycles.

In some embodiments, the patient is first administered the ICOS binding protein at a dose of about 0.001 mg/kg to about 10 mg/kg as a monotherapy regimen and then the ICOS binding protein at a dose of about 0.001 mg/kg to about 10 mg/kg with the TIM-3 binding protein at a dose of 0.0625 mg/kg to 62.5 mg/kg as a combination therapy regimen. In one embodiment, the patient is first administered the ICOS binding protein at a dose of 0.3 mg/kg as a monotherapy regimen and then the ICOS binding protein at a dose of 0.3 mg/kg with the TIM-3 binding protein at a dose of 1.25 mg/kg as a combination therapy regimen. In one embodiment, the patient is first administered the ICOS binding protein at a dose of 0.3 mg/kg as a monotherapy regimen and then the ICOS binding protein at a dose of 0.3 mg/kg with the TIM-3 binding protein at a dose of 3.75 mg/kg as a combination therapy regimen. In one embodiment, the patient is first administered the ICOS binding protein at a dose of 0.3 mg/kg as a monotherapy regimen and then the ICOS binding protein at a dose of 0.3 mg/kg with the TIM-3 binding protein at a dose of 11.25 mg/kg as a combination therapy regimen. In one embodiment, the patient is first administered the ICOS binding protein at a dose of 1 mg/kg as a monotherapy regimen and then the ICOS binding protein at a dose of 1 mg/kg with the TIM-3 binding protein at a dose of 1.25 mg/kg as a combination therapy regimen. In one embodiment, the patient is first administered the ICOS binding protein at a dose of 1 mg/kg as a monotherapy regimen and then the ICOS binding protein at a dose of 1 mg/kg with the TIM-3 binding protein at a dose of 3.75 mg/kg as a combination therapy regimen. In one embodiment, the patient is first administered the ICOS binding protein at a dose of 1 mg/kg as a monotherapy regimen and then the ICOS binding protein at a dose of 1 mg/kg with the TIM-3 binding protein at a dose of 11.25 mg/kg as a combination therapy regimen.

In some embodiments, the patient is first administered the ICOS binding protein at a dose of about 0.001 mg/kg to about 10 mg/kg as a monotherapy regimen and then the ICOS binding protein at a dose of about 0.001 mg/kg to about 10 mg/kg with the PD-1 binding protein at a dose of 1.25 mg/kg to 25 mg/kg and the TIM-3 binding protein at a dose of 0.0625 mg/kg to 62.5 mg/kg as a combination therapy regimen. In one embodiment, the patient is first administered the ICOS binding protein at a dose of 0.3 mg/kg as a monotherapy regimen and then the ICOS binding protein at a dose of 0.3 mg/kg with the PD-1 binding protein at a dose of 6.25 mg/kg and the TIM-3 binding protein at a dose of 1.25 mg/kg as a combination therapy regimen. In one embodiment, the patient is first administered the ICOS binding protein at a dose of 0.3 mg/kg as a monotherapy regimen and then the ICOS binding protein at a dose of 0.3 mg/kg with the PD-1 binding protein at a dose of 6.25 mg/kg and the TIM-3 binding protein at a dose of 3.75 mg/kg as a combination therapy regimen. In one embodiment, the patient is first administered the ICOS binding protein at a dose of 0.3 mg/kg as a monotherapy regimen and then the ICOS binding protein at a dose of 0.3 mg/kg with the PD-1 binding protein at a dose of 6.25 mg/kg and the TIM-3 binding protein at a dose of 11.25 mg/kg as a combination therapy regimen. In one embodiment, the patient is first administered the ICOS binding protein at a dose of 1 mg/kg as a monotherapy regimen and then the ICOS binding protein at a dose of 1 mg/kg with the PD-1 binding protein at a dose of 6.25 mg/kg and the TIM-3 binding protein at a dose of 1.25 mg/kg as a combination therapy regimen. In one embodiment, the patient is first administered the ICOS binding protein at a dose of 1 mg/kg as a monotherapy regimen and then the ICOS binding protein at a dose of 1 mg/kg with the PD-1 binding protein at a dose of 6.25 mg/kg and the TIM-3 binding protein at a dose of 3.75 mg/kg as a combination therapy regimen. In one embodiment, the patient is first administered the ICOS binding protein at a dose of 1 mg/kg as a monotherapy regimen and then the ICOS binding protein at a dose of 1 mg/kg with the PD-1 binding protein at a dose of 6.25 mg/kg and the TIM-3 binding protein at a dose of 11.25 mg/kg as a combination therapy regimen. In one embodiment, the patient is first administered the ICOS binding protein at a dose of 0.3 mg/kg as a monotherapy regimen and then the ICOS binding protein at a dose of 0.3 mg/kg with the PD-1 binding protein at a dose of 12.5 mg/kg and the TIM-3 binding protein at a dose of 1.25 mg/kg as a combination therapy regimen. In one embodiment, the patient is first administered the ICOS binding protein at a dose of 0.3 mg/kg as a monotherapy regimen and then the ICOS binding protein at a dose of 0.3 mg/kg with the PD-1 binding protein at a dose of 12.5 mg/kg and the TIM-3 binding protein at a dose of 3.75 mg/kg as a combination therapy regimen. In one embodiment, the patient is first administered the ICOS binding protein at a dose of 0.3 mg/kg as a monotherapy regimen and then the ICOS binding protein at a dose of 0.3 mg/kg with the PD-1 binding protein at a dose of 12.5 mg/kg and the TIM-3 binding protein at a dose of 11.25 mg/kg as a combination therapy regimen. In one embodiment, the patient is first administered the ICOS binding protein at a dose of 1 mg/kg as a monotherapy regimen and then the ICOS binding protein at a dose of 1 mg/kg with the PD-1 binding protein at a dose of 12.5 mg/kg and the TIM-3 binding protein at a dose of 1.25 mg/kg as a combination therapy regimen. In one embodiment, the patient is first administered the ICOS binding protein at a dose of 1 mg/kg as a monotherapy regimen and then the ICOS binding protein at a dose of 1 mg/kg with the PD-1 binding protein at a dose of 12.5 mg/kg and the TIM-3 binding protein at a dose of 3.75 mg/kg as a combination therapy regimen. In one embodiment, the patient is first administered the ICOS binding protein at a dose of 1 mg/kg as a monotherapy regimen and then the ICOS binding protein at a dose of 1 mg/kg with the PD-1 binding protein at a dose of 12.5 mg/kg and the TIM-3 binding protein at a dose of 11.25 mg/kg as a combination therapy regimen. In a further embodiment, the patient is first administered the ICOS binding protein at a dose of 0.3 mg/kg as a monotherapy regimen every 3 weeks for up to 1, 2, 3, 4, 5, 6, 7, 8, 9, 10, 11, 12, 13 or 14 cycles and then the ICOS binding protein at a dose of 0.3 mg/kg with the TIM-3 binding protein at a dose of 1.25 mg/kg as a combination therapy regimen every 3 weeks for up to 1, 2, 3, 4, 5, 6, 7, 8, 9, 10, 11, 12, 13 or 14 cycles. In a further embodiment, the patient is first administered the ICOS binding protein at a dose of 0.3 mg/kg as a monotherapy regimen every 3 weeks for up to 1, 2, 3, 4, 5, 6, 7, 8, 9, 10, 11, 12, 13 or 14 cycles and then the ICOS binding protein at a dose of 0.3 mg/kg with the TIM-3 binding protein at a dose of 3.75 mg/kg as a combination therapy regimen every 3 weeks for up to 1, 2, 3, 4, 5, 6, 7, 8, 9, 10, 11, 12, 13 or 14 cycles. In a further embodiment, the patient is first administered the ICOS binding protein at a dose of 0.3 mg/kg as a monotherapy regimen every 3 weeks for up to 1, 2, 3, 4, 5, 6, 7, 8, 9, 10, 11, 12, 13 or 14 cycles and then the ICOS binding protein at a dose of 0.3 mg/kg with the TIM-3 binding protein at a dose of 11.25 mg/kg as a combination therapy regimen every 3 weeks for up to 1, 2, 3, 4, 5, 6, 7, 8, 9, 10, 11, 12, 13 or 14 cycles.

In a further embodiment, the patient is first administered the ICOS binding protein at a dose of 1.0 mg/kg as a monotherapy regimen every 3 weeks for up to 1, 2, 3, 4, 5, 6, 7, 8, 9, 10, 11, 12, 13 or 14 cycles and then the ICOS binding protein at a dose of 1.0 mg/kg with the TIM-3 binding protein at a dose of 1.25 mg/kg as a combination therapy regimen every 3 weeks for up to 1, 2, 3, 4, 5, 6, 7, 8, 9, 10, 11, 12, 13 or 14 cycles. In a further embodiment, the patient is first administered the ICOS binding protein at a dose of 1.0 mg/kg as a monotherapy regimen every 3 weeks for up to 1, 2, 3, 4, 5, 6, 7, 8, 9, 10, 11, 12, 13 or 14 cycles and then the ICOS binding protein at a dose of 1.0 mg/kg with the TIM-3 binding protein at a dose of 3.75 mg/kg as a combination therapy regimen every 3 weeks for up to 1, 2, 3, 4, 5, 6, 7, 8, 9, 10, 11, 12, 13 or 14 cycles. In a further embodiment, the patient is first administered the ICOS binding protein at a dose of 1.0 mg/kg as a monotherapy regimen every 3 weeks for up to 1, 2, 3, 4, 5, 6, 7, 8, 9, 10, 11, 12, 13 or 14 cycles and then the ICOS binding protein at a dose of 1.0 mg/kg with the TIM-3 binding protein at a dose of 11.25 mg/kg as a combination therapy regimen every 3 weeks for up to 1, 2, 3, 4, 5, 6, 7, 8, 9, 10, 11, 12, 13 or 14 cycles.

In a further embodiment, the patient is first administered the ICOS binding protein at a dose of 0.3 mg/kg as a monotherapy regimen every 3 weeks for up to 1, 2, 3, 4, 5, 6, 7, 8, 9, 10, 11, 12, 13 or 14 cycles and then the ICOS binding protein at a dose of 0.3 mg/kg with the PD-1 binding protein at a dose of 6.25 mg/kg and the TIM-3 binding protein at a dose of 1.25 mg/kg as a combination therapy regimen every 3 weeks for up to 1, 2, 3, 4, 5, 6, 7, 8, 9, 10, 11, 12, 13 or 14 cycles. In a further embodiment, the patient is first administered the ICOS binding protein at a dose of 0.3 mg/kg as a monotherapy regimen every 3 weeks for up to 1, 2, 3, 4, 5, 6, 7, 8, 9, 10, 11, 12, 13 or 14 cycles and then the ICOS binding protein at a dose of 0.3 mg/kg with the PD-1 binding protein at a dose of 6.25 mg/kg and the TIM-3 binding protein at a dose of 3.75 mg/kg as a combination therapy regimen every 3 weeks for up to 1, 2, 3, 4, 5, 6, 7, 8, 9, 10, 11, 12, 13 or 14 cycles. In a further embodiment, the patient is first administered the ICOS binding protein at a dose of 0.3 mg/kg as a monotherapy regimen every 3 weeks for up to 1, 2, 3, 4, 5, 6, 7, 8, 9, 10, 11, 12, 13 or 14 cycles and then the ICOS binding protein at a dose of 0.3 mg/kg with the PD-1 binding protein at a dose of 6.25 mg/kg and the TIM-3 binding protein at a dose of 11.25 mg/kg as a combination therapy regimen every 3 weeks for up to 1, 2, 3, 4, 5, 6, 7, 8, 9, 10, 11, 12, 13 or 14 cycles. In a further embodiment, the patient is first administered the ICOS binding protein at a dose of 1.0 mg/kg as a monotherapy regimen every 3 weeks for up to 1, 2, 3, 4, 5, 6, 7, 8, 9, 10, 11, 12, 13 or 14 cycles and then the ICOS binding protein at a dose of 1.0 mg/kg with the PD-1 binding protein at a dose of 6.25 mg/kg and the TIM-3 binding protein at a dose of 1.25 mg/kg as a combination therapy regimen every 3 weeks for up to 1, 2, 3, 4, 5, 6, 7, 8, 9, 10, 11, 12, 13 or 14 cycles. In a further embodiment, the patient is first administered the ICOS binding protein at a dose of 1.0 mg/kg as a monotherapy regimen every 3 weeks for up to 1, 2, 3, 4, 5, 6, 7, 8, 9, 10, 11, 12, 13 or 14 cycles and then the ICOS binding protein at a dose of 1.0 mg/kg with the PD-1 binding protein at a dose of 6.25 mg/kg and the TIM-3 binding protein at a dose of 3.75 mg/kg as a combination therapy regimen every 3 weeks for up to 1, 2, 3, 4, 5, 6, 7, 8, 9, 10, 11, 12, 13 or 14 cycles. In a further embodiment, the patient is first administered the ICOS binding protein at a dose of 1.0 mg/kg as a monotherapy regimen every 3 weeks for up to 1, 2, 3, 4, 5, 6, 7, 8, 9, 10, 11, 12, 13 or 14 cycles and then the ICOS binding protein at a dose of 1.0 mg/kg with the PD-1 binding protein at a dose of 6.25 mg/kg and the TIM-3 binding protein at a dose of 11.25 mg/kg as a combination therapy regimen every 3 weeks for up to 1, 2, 3, 4, 5, 6, 7, 8, 9, 10, 11, 12, 13 or 14 cycles.

In a further embodiment, the patient is first administered the ICOS binding protein at a dose of 0.6 mg/kg as a monotherapy regimen every 6 weeks for up to 1, 2, 3, 4, 5, 6, 7, 8, 9, 10, 11, 12, 13 or 14 cycles and then the ICOS binding protein at a dose of 0.6 mg/kg with the PD-1 binding protein at a dose of 12.5 mg/kg and the TIM-3 binding protein at a dose of 1.25 mg/kg as a combination therapy regimen every 6 weeks for up to 1, 2, 3, 4, 5, 6, 7, 8, 9, 10, 11, 12, 13 or 14 cycles. In a further embodiment, the patient is first administered the ICOS binding protein at a dose of 0.6 mg/kg as a monotherapy regimen every 6 weeks for up to 1, 2, 3, 4, 5, 6, 7, 8, 9, 10, 11, 12, 13 or 14 cycles and then the ICOS binding protein at a dose of 0.6 mg/kg with the PD-1 binding protein at a dose of 12.5 mg/kg and the TIM-3 binding protein at a dose of 3.75 mg/kg as a combination therapy regimen every 6 weeks for up to 1, 2, 3, 4, 5, 6, 7, 8, 9, 10, 11, 12, 13 or 14 cycles. In a further embodiment, the patient is first administered the ICOS binding protein at a dose of 0.6 mg/kg as a monotherapy regimen every 6 weeks for up to 1, 2, 3, 4, 5, 6, 7, 8, 9, 10, 11, 12, 13 or 14 cycles and then the ICOS binding protein at a dose of 0.6 mg/kg with the PD-1 binding protein at a dose of 12.5 mg/kg and the TIM-3 binding protein at a dose of 11.25 mg/kg as a combination therapy regimen every 6 weeks for up to 1, 2, 3, 4, 5, 6, 7, 8, 9, 10, 11, 12, 13 or 14 cycles. In a further embodiment, the patient is first administered the ICOS binding protein at a dose of 2.0 mg/kg as a monotherapy regimen every 6 weeks for up to 1, 2, 3, 4, 5, 6, 7, 8, 9, 10, 11, 12, 13 or 14 cycles and then the ICOS binding protein at a dose of 2.0 mg/kg with the PD-1 binding protein at a dose of 12.5 mg/kg and the TIM-3 binding protein at a dose of 1.25 mg/kg as a combination therapy regimen every 6 weeks for up to 1, 2, 3, 4, 5, 6, 7, 8, 9, 10, 11, 12, 13 or 14 cycles. In a further embodiment, the patient is first administered the ICOS binding protein at a dose of 2.0 mg/kg as a monotherapy regimen every 6 weeks for up to 1, 2, 3, 4, 5, 6, 7, 8, 9, 10, 11, 12, 13 or 14 cycles and then the ICOS binding protein at a dose of 2.0 mg/kg with the PD-1 binding protein at a dose of 12.5 mg/kg and the TIM-3 binding protein at a dose of 3.75 mg/kg as a combination therapy regimen every 6 weeks for up to 1, 2, 3, 4, 5, 6, 7, 8, 9, 10, 11, 12, 13 or 14 cycles. In a further embodiment, the patient is first administered the ICOS binding protein at a dose of 2.0 mg/kg as a monotherapy regimen every 6 weeks for up to 1, 2, 3, 4, 5, 6, 7, 8, 9, 10, 11, 12, 13 or 14 cycles and then the ICOS binding protein at a dose of 2.0 mg/kg with the PD-1 binding protein at a dose of 12.5 mg/kg and the TIM-3 binding protein at a dose of 11.25 mg/kg as a combination therapy regimen every 6 weeks for up to 1, 2, 3, 4, 5, 6, 7, 8, 9, 10, 11, 12, 13 or 14 cycles.

In some embodiments, the patient is first administered the TIM-3 binding protein at a dose of 5 mg to 5000 mg as a monotherapy regimen and then the TIM-3 binding protein at a dose of 5 mg to 5000 mg with the ICOS binding protein at a dose of about 0.08 mg to about 800 mg as a combination therapy regimen. In one embodiment, the patient is first administered the TIM-3 binding protein at a dose of 5 mg to 5000 mg and then the TIM-3 binding protein at a dose of 5 mg to 5000 mg with the ICOS binding protein at a dose of about 24 mg, about 48 mg, about 80 mg or about 160 mg as a combination therapy regimen. In one embodiment, the patient is first administered the TIM-3 binding protein at a dose of 100 mg and then the TIM-3 binding protein at a dose of 100 mg with the ICOS binding protein at a dose of 24 mg as a combination therapy regimen. In one embodiment, the patient is first administered the TIM-3 binding protein at a dose of 300 mg and then the TIM-3 binding protein at a dose of 300 mg with the ICOS binding protein at a dose of 24 mg as a combination therapy regimen. In one embodiment, the patient is first administered the TIM-3 binding protein at a dose of 900 mg and then the TIM-3 binding protein at a dose of 900 mg with the ICOS binding protein at a dose of 24 mg as a combination therapy regimen. In one embodiment, the patient is first administered the TIM-3 binding protein at a dose of 100 mg and then the TIM-3 binding protein at a dose of 100 mg with the ICOS binding protein at a dose of 80 mg as a combination therapy regimen. In one embodiment, the patient is first administered the TIM-3 binding protein at a dose of 300 mg and then the TIM-3 binding protein at a dose of 300 mg with the ICOS binding protein at a dose of 80 mg as a combination therapy regimen. In one embodiment, the patient is first administered the TIM-3 binding protein at a dose of 900 mg and then the TIM-3 binding protein at a dose of 900 mg with the ICOS binding protein at a dose of 80 mg as a combination therapy regimen.

In some embodiments, the patient is first administered the TIM-3 binding protein at a dose of 5 mg to 5000 mg as a monotherapy regimen and then the TIM-3 binding protein at a dose of 5 mg to 5000 mg with the ICOS binding protein at a dose of about 0.08 mg to about 800 mg and the PD-1 binding protein at a dose of about 100 mg to about 2000 mg as a combination therapy regimen. In one embodiment, the patient is first administered the TIM-3 binding protein at a dose of 5 mg to 5000 mg and then the TIM-3 binding protein at a dose of 5 mg to 5000 mg with the ICOS binding protein at a dose of about 8 mg, about 24 mg, about 48 mg, about 80 mg, about 160 mg or about 240 mg and the PD-1 binding protein at a dose about 100 mg to about 2000 mg as a combination therapy regimen. In one embodiment, the patient is first administered the TIM-3 binding protein at a dose of 100 mg and then the TIM-3 binding protein at a dose of 300 mg with the ICOS binding protein at a dose of 24 mg and the PD-1 binding protein at a dose of 500 mg as a combination therapy regimen. In one embodiment, the patient is first administered the TIM-3 binding protein at a dose of 300 mg and then the TIM-3 binding protein at a dose of 300 mg with the ICOS binding protein at a dose of 24 mg and the PD-1 binding protein at a dose of 500 mg as a combination therapy regimen. In one embodiment, the patient is first administered the TIM-3 binding protein at a dose of 900 mg and then the TIM-3 binding protein at a dose of 900 mg with the ICOS binding protein at a dose of 24 mg and the PD-1 binding protein at a dose of 500 mg as a combination therapy regimen. In one embodiment, the patient is first administered the TIM-3 binding protein at a dose of 100 mg and then the TIM-3 binding protein at a dose of 100 mg with the PD-1 binding protein at a dose of 500 mg and the ICOS binding protein at a dose of 80 mg as a combination therapy regimen. In one embodiment, the patient is first administered the TIM-3 binding protein at a dose of 300 mg and then the TIM-3 binding protein at a dose of 300 mg with the PD-1 binding protein at a dose of 500 mg and the ICOS binding protein at a dose of 80 mg as a combination therapy regimen. In one embodiment, the patient is first administered the TIM-3 binding protein at a dose of 900 mg and then the TIM-3 binding protein at a dose of 900 mg with the PD-1 binding protein at a dose of 500 mg and the ICOS binding protein at a dose of 80 mg as a combination therapy regimen. In one embodiment, the patient is first administered the TIM-3 binding protein at a dose of 100 mg and then the TIM-3 binding protein at a dose of 100 mg with the ICOS binding protein at a dose of 24 mg and the PD-1 binding protein at a dose of 1000 mg as a combination therapy regimen. In one embodiment, the patient is first administered the TIM-3 binding protein at a dose of 300 mg and then the TIM-3 binding protein at a dose of 300 mg with the ICOS binding protein at a dose of 24 mg and the PD-1 binding protein at a dose of 1000 mg as a combination therapy regimen. In one embodiment, the patient is first administered the TIM-3 binding protein at a dose of 900 mg and then the TIM-3 binding protein at a dose of 900 mg with the ICOS binding protein at a dose of 24 mg and the PD-1 binding protein at a dose of 1000 mg as a combination therapy regimen. In one embodiment, the patient is first administered the TIM-3 binding protein at a dose of 100 mg and then the TIM-3 binding protein at a dose of 100 mg with the ICOS binding protein at a dose of 80 mg and the PD-1 binding protein at a dose of 1000 mg as a combination therapy regimen. In one embodiment, the patient is first administered the TIM-3 binding protein at a dose of 300 mg and then the TIM-3 binding protein at a dose of 300 mg with the ICOS binding protein at a dose of 80 mg and the PD-1 binding protein at a dose of 1000 mg as a combination therapy regimen. In one embodiment, the patient is first administered the TIM-3 binding protein at a dose of 900 mg and then the TIM-3 binding protein at a dose of 900 mg with the ICOS binding protein at a dose of 80 mg and the PD-1 binding protein at a dose of 1000 mg as a combination therapy regimen.

In a further embodiment, the patient is first administered the TIM-3 binding protein at a dose of 100 mg as a monotherapy regimen every 3 weeks for up to 1, 2, 3, 4, 5, 6, 7, 8, 9, 10, 11, 12, 13 or 14 cycles and then the TIM-3 binding protein at a dose of 100 mg with the ICOS binding protein at a dose of 24 mg as a combination therapy regimen every 3 weeks for up to 1, 2, 3, 4, 5, 6, 7, 8, 9, 10, 11, 12, 13 or 14 cycles. In a further embodiment, the patient is first administered the TIM-3 binding protein at a dose of 300 mg as a monotherapy regimen every 3 weeks for up to 1, 2, 3, 4, 5, 6, 7, 8, 9, 10, 11, 12, 13 or 14 cycles and then the TIM-3 binding protein at a dose of 300 mg with the ICOS binding protein at a dose of 24 mg as a combination therapy regimen every 3 weeks for up to 1, 2, 3, 4, 5, 6, 7, 8, 9, 10, 11, 12, 13 or 14 cycles. In a further embodiment, the patient is first administered the TIM-3 binding protein at a dose of 900 mg as a monotherapy regimen every 3 weeks for up to 1, 2, 3, 4, 5, 6, 7, 8, 9, 10, 11, 12, 13 or 14 cycles and then the TIM-3 binding protein at a dose of 900 mg with the ICOS binding protein at a dose of 24 mg as a combination therapy regimen every 3 weeks for up to 1, 2, 3, 4, 5, 6, 7, 8, 9, 10, 11, 12, 13 or 14 cycles.

In a further embodiment, the patient is first administered the TIM-3 binding protein at a dose of 100 mg as a monotherapy regimen every 3 weeks for up to 1, 2, 3, 4, 5, 6, 7, 8, 9, 10, 11, 12, 13 or 14 cycles and then the TIM-3 binding protein at a dose of 100 mg with the ICOS binding protein at a dose of 24 mg and the PD-1 binding protein at a dose of 500 mg as a combination therapy regimen every 3 weeks for up to 1, 2, 3, 4, 5, 6, 7, 8, 9, 10, 11, 12, 13 or 14 cycles. In a further embodiment, the patient is first administered the TIM-3 binding protein at a dose of 300 mg as a monotherapy regimen every 3 weeks for up to 1, 2, 3, 4, 5, 6, 7, 8, 9, 10, 11, 12, 13 or 14 cycles and then the TIM-3 binding protein at a dose of 300 mg with the ICOS binding protein at a dose of 24 mg and the PD-1 binding protein at a dose of 500 mg as a combination therapy regimen every 3 weeks for up to 1, 2, 3, 4, 5, 6, 7, 8, 9, 10, 11, 12, 13 or 14 cycles. In a further embodiment, the patient is first administered the TIM-3 binding protein at a dose of 900 mg as a monotherapy regimen every 3 weeks for up to 1, 2, 3, 4, 5, 6, 7, 8, 9, 10, 11, 12, 13 or 14 cycles and then the TIM-3 binding protein at a dose of 900 mg with the ICOS binding protein at a dose of 24 mg and the PD-1 binding protein at a dose of 500 mg as a combination therapy regimen every 3 weeks for up to 1, 2, 3, 4, 5, 6, 7, 8, 9, 10, 11, 12, 13 or 14 cycles. In a further embodiment, the patient is first administered the TIM-3 binding protein at a dose of 100 mg as a monotherapy regimen every 3 weeks for up to 1, 2, 3, 4, 5, 6, 7, 8, 9, 10, 11, 12, 13 or 14 cycles and then the TIM-3 binding protein at a dose of 100 mg with the ICOS binding protein at a dose of 80 mg and the PD-1 binding protein at a dose of 500 mg as a combination therapy regimen every 3 weeks for up to 1, 2, 3, 4, 5, 6, 7, 8, 9, 10, 11, 12, 13 or 14 cycles. In a further embodiment, the patient is first administered the TIM-3 binding protein at a dose of 300 mg as a monotherapy regimen every 3 weeks for up to 1, 2, 3, 4, 5, 6, 7, 8, 9, 10, 11, 12, 13 or 14 cycles and then the TIM-3 binding protein at a dose of 300 mg with the ICOS binding protein at a dose of 80 mg and the PD-1 binding protein at a dose of 500 mg as a combination therapy regimen every 3 weeks for up to 1, 2, 3, 4, 5, 6, 7, 8, 9, 10, 11, 12, 13 or 14 cycles. In a further embodiment, the patient is first administered the TIM-3 binding protein at a dose of 900 mg as a monotherapy regimen every 3 weeks for up to 1, 2, 3, 4, 5, 6, 7, 8, 9, 10, 11, 12, 13 or 14 cycles and then the TIM-3 binding protein at a dose of 900 mg with the ICOS binding protein at a dose of 80 mg and the PD-1 binding protein at a dose of 500 mg as a combination therapy regimen every 3 weeks for up to 1, 2, 3, 4, 5, 6, 7, 8, 9, 10, 11, 12, 13 or 14 cycles.

In a further embodiment, the patient is first administered the TIM-3 binding protein at a dose of 100 mg as a monotherapy regimen every 3 weeks for up to 1, 2, 3, 4, 5, 6, 7, 8, 9, 10, 11, 12, 13 or 14 cycles and then the TIM-3 binding protein at a dose of 100 mg with the ICOS binding protein at a dose of 80 mg as a combination therapy regimen every 3 weeks for up to 1, 2, 3, 4, 5, 6, 7, 8, 9, 10, 11, 12, 13 or 14 cycles. In a further embodiment, the patient is first administered the TIM-3 binding protein at a dose of 300 mg as a monotherapy regimen every 3 weeks for up to 1, 2, 3, 4, 5, 6, 7, 8, 9, 10, 11, 12, 13 or 14 cycles and then the TIM-3 binding protein at a dose of 300 mg with the ICOS binding protein at a dose of 80 mg as a combination therapy regimen every 3 weeks for up to 1, 2, 3, 4, 5, 6, 7, 8, 9, 10, 11, 12, 13 or 14 cycles. In a further embodiment, the patient is first administered the TIM-3 binding protein at a dose of 900 mg as a monotherapy regimen every 3 weeks for up to 1, 2, 3, 4, 5, 6, 7, 8, 9, 10, 11, 12, 13 or 14 cycles and then the TIM-3 binding protein at a dose of 900 mg with the ICOS binding protein at a dose of 80 mg as a combination therapy regimen every 3 weeks for up to 1, 2, 3, 4, 5, 6, 7, 8, 9, 10, 11, 12, 13 or 14 cycles.

In a further embodiment, the patient is first administered the TIM-3 binding protein at a dose of 100 mg as a monotherapy regimen every 6 weeks for up to 1, 2, 3, 4, 5, 6, 7, 8, 9, 10, 11, 12, 13 or 14 cycles and then the TIM-3 binding protein at a dose of 100 mg with the ICOS binding protein at a dose of 48 mg with the PD-1 binding protein at a dose of 1000 mg as a combination therapy regimen every 6 weeks for up to 1, 2, 3, 4, 5, 6, 7, 8, 9, 10, 11, 12, 13 or 14 cycles. In a further embodiment, the patient is first administered the TIM-3 binding protein at a dose of 300 mg as a monotherapy regimen every 6 weeks for up to 1, 2, 3, 4, 5, 6, 7, 8, 9, 10, 11, 12, 13 or 14 cycles and then the TIM-3 binding protein at a dose of 300 mg with the ICOS binding protein at a dose of 48 mg with the PD-1 binding protein at a dose of 1000 mg as a combination therapy regimen every 6 weeks for up to 1, 2, 3, 4, 5, 6, 7, 8, 9, 10, 11, 12, 13 or 14 cycles. In a further embodiment, the patient is first administered the TIM-3 binding protein at a dose of 900 mg as a monotherapy regimen every 6 weeks for up to 1, 2, 3, 4, 5, 6, 7, 8, 9, 10, 11, 12, 13 or 14 cycles and then the TIM-3 binding protein at a dose of 900 mg with the ICOS binding protein at a dose of 48 mg with the PD-1 binding protein at a dose of 1000 mg as a combination therapy regimen every 6 weeks for up to 1, 2, 3, 4, 5, 6, 7, 8, 9, 10, 11, 12, 13 or 14 cycles. In a further embodiment, the patient is first administered the TIM-3 binding protein at a dose of 100 mg as a monotherapy regimen every 6 weeks for up to 1, 2, 3, 4, 5, 6, 7, 8, 9, 10, 11, 12, 13 or 14 cycles and then the TIM-3 binding protein at a dose of 100 mg with the ICOS binding protein at a dose of 160 mg and the PD-1 binding protein at a dose of 1000 mg as a combination therapy regimen every 6 weeks for up to 1, 2, 3, 4, 5, 6, 7, 8, 9, 10, 11, 12, 13 or 14 cycles. In a further embodiment, the patient is first administered the TIM-3 binding protein at a dose of 300 mg as a monotherapy regimen every 6 weeks for up to 1, 2, 3, 4, 5, 6, 7, 8, 9, 10, 11, 12, 13 or 14 cycles and then the TIM-3 binding protein at a dose of 300 mg with the ICOS binding protein at a dose of 160 mg and the PD-1 binding protein at a dose of 1000 mg as a combination therapy regimen every 6 weeks for up to 1, 2, 3, 4, 5, 6, 7, 8, 9, 10, 11, 12, 13 or 14 cycles. In a further embodiment, the patient is first administered the TIM-3 binding protein at a dose of 900 mg as a monotherapy regimen every 6 weeks for up to 1, 2, 3, 4, 5, 6, 7, 8, 9, 10, 11, 12, 13 or 14 cycles and then the TIM-3 binding protein at a dose of 900 mg with the ICOS binding protein at a dose of 160 mg and the PD-1 binding protein at a dose of 1000 mg as a combination therapy regimen every 6 weeks for up to 1, 2, 3, 4, 5, 6, 7, 8, 9, 10, 11, 12, 13 or 14 cycles.

In some embodiments, the patient is first administered the TIM-3 binding protein at a dose of 0.0625 mg/kg to 62.5 mg/kg as a monotherapy regimen and then the TIM-3 binding protein at a dose of 0.0625 mg/kg to 62.5 mg/kg with the ICOS binding protein at a dose of about 0.001 mg/kg to about 10 mg/kg as a combination therapy regimen. In one embodiment, the patient is first administered the TIM-3 binding protein at a dose of 1.25 mg/kg as a monotherapy regimen and then the TIM-3 binding protein at a dose of 1.25 mg/kg with the ICOS binding protein at a dose of 0.3 mg/kg as a combination therapy regimen. In one embodiment, the patient is first administered the TIM-3 binding protein at a dose of 3.75 mg/kg as a monotherapy regimen and then the TIM-3 binding protein at a dose of 3.75 mg/kg with the ICOS binding protein at a dose of 0.3 mg/kg as a combination therapy regimen. In one embodiment, the patient is first administered the TIM-3 binding protein at a dose of 11.25 mg/kg as a monotherapy regimen and then the TIM-3 binding protein at a dose of 11.25 mg/kg with the ICOS binding protein at a dose of 0.3 mg/kg as a combination therapy regimen. In one embodiment, the patient is first administered the TIM-3 binding protein at a dose of 1.25 mg/kg as a monotherapy regimen and then the TIM-3 binding protein at a dose of 1.25 mg/kg with the ICOS binding protein at a dose of 1 mg/kg as a combination therapy regimen. In one embodiment, the patient is first administered the TIM-3 binding protein at a dose of 3.75 mg/kg as a monotherapy regimen and then the TIM-3 binding protein at a dose of 3.75 mg/kg with the ICOS binding protein at a dose of 1 mg/kg as a combination therapy regimen. In one embodiment, the patient is first administered the TIM-3 binding protein at a dose of 11.25 mg/kg as a monotherapy regimen and then the TIM-3 binding protein at a dose of 11.25 mg/kg with the ICOS binding protein at a dose of 1 mg/kg as a combination therapy regimen.

In some embodiments, the patient is first administered the TIM-3 binding protein at a dose of 0.0625 mg/kg to 62.5 mg/kg as a monotherapy regimen and then the TIM-3 binding protein at a dose of 0.0625 mg/kg to 62.5 mg/kg with the ICOS binding protein at a dose of about 0.001 mg/kg to about 10 mg/kg and the PD-1 binding protein at a dose of 1.25 mg/kg to 25 mg/kg as a combination therapy regimen. In one embodiment, the patient is first administered the TIM-3 binding protein at a dose of 1.25 mg/kg as a monotherapy regimen and then the TIM-3 binding protein at a dose of 1.25 mg/kg with the ICOS binding protein at a dose of 0.3 mg/kg and the PD-1 binding protein at a dose of 6.25 mg/kg as a combination therapy regimen. In one embodiment, the patient is first administered the TIM-3 binding protein at a dose of 3.75 mg/kg as a monotherapy regimen and then the TIM-3 binding protein at a dose of 3.75 mg/kg with the ICOS binding protein at a dose of 0.3 mg/kg and the PD-1 binding protein at a dose of 6.25 mg/kg as a combination therapy regimen. In one embodiment, the patient is first administered the TIM-3 binding protein at a dose of 11.25 mg/kg as a monotherapy regimen and then the TIM-3 binding protein at a dose of 11.25 mg/kg with the ICOS binding protein at a dose of 0.3 mg/kg and the PD-1 binding protein at a dose of 6.25 mg/kg as a combination therapy regimen. In one embodiment, the patient is first administered the TIM-3 binding protein at a dose of 1.25 mg/kg as a monotherapy regimen and then the TIM-3 binding protein at a dose of 1.25 mg/kg with the ICOS binding protein at a dose of 1 mg/kg and the PD-1 binding protein at a dose of 6.25 mg/kg as a combination therapy regimen. In one embodiment, the patient is first administered the TIM-3 binding protein at a dose of 3.75 mg/kg as a monotherapy regimen and then the TIM-3 binding protein at a dose of 3.75 mg/kg with the ICOS binding protein at a dose of 1 mg/kg and the PD-1 binding protein at a dose of 6.25 mg/kg as a combination therapy regimen. In one embodiment, the patient is first administered the TIM-3 binding protein at a dose of 11.25 mg/kg as a monotherapy regimen and then the TIM-3 binding protein at a dose of 11.25 mg/kg with the ICOS binding protein at a dose of 1 mg/kg and the PD-1 binding protein at a dose of 6.25 mg/kg as a combination therapy regimen. In one embodiment, the patient is first administered the TIM-3 binding protein at a dose of 1.25 mg/kg as a monotherapy regimen and then the TIM-3 binding protein at a dose of 1.25 mg/kg with the ICOS binding protein at a dose of 0.3 mg/kg and the PD-1 binding protein at a dose of 12.5 mg/kg as a combination therapy regimen. In one embodiment, the patient is first administered the TIM-3 binding protein at a dose of 3.75 mg/kg as a monotherapy regimen and then the TIM-3 binding protein at a dose of 3.75 mg/kg with the ICOS binding protein at a dose of 0.3 mg/kg and the PD-1 binding protein at a dose of 12.5 mg/kg as a combination therapy regimen. In one embodiment, the patient is first administered the TIM-3 binding protein at a dose of 11.25 mg/kg as a monotherapy regimen and then the TIM-3 binding protein at a dose of 11.25 mg/kg with the ICOS binding protein at a dose of 0.3 mg/kg and the PD-1 binding protein at a dose of 12.5 mg/kg as a combination therapy regimen. In one embodiment, the patient is first administered the TIM-3 binding protein at a dose of 1.25 mg/kg as a monotherapy regimen and then the TIM-3 binding protein at a dose of 1.25 mg/kg with the ICOS binding protein at a dose of 1 mg/kg and the PD-1 binding protein at a dose of 12.5 mg/kg as a combination therapy regimen. In one embodiment, the patient is first administered the TIM-3 binding protein at a dose of 3.75 mg/kg as a monotherapy regimen and then the TIM-3 binding protein at a dose of 3.75 mg/kg with the ICOS binding protein at a dose of 1 mg/kg and the PD-1 binding protein at a dose of 12.5 mg/kg as a combination therapy regimen. In one embodiment, the patient is first administered the TIM-3 binding protein at a dose of 11.25 mg/kg as a monotherapy regimen and then the TIM-3 binding protein at a dose of 11.25 mg/kg with the ICOS binding protein at a dose of 1 mg/kg and the PD-1 binding protein at a dose of 12.5 mg/kg as a combination therapy regimen.

In a further embodiment, the patient is first administered the TIM-3 binding protein at a dose of 1.25 mg/kg as a monotherapy regimen every 3 weeks for up to 1, 2, 3, 4, 5, 6, 7, 8, 9, 10, 11, 12, 13 or 14 cycles and then the TIM-3 binding protein at a dose of 1.25 mg/kg with the ICOS binding protein at a dose of 0.3 mg/kg as a combination therapy regimen every 3 weeks for up to 1, 2, 3, 4, 5, 6, 7, 8, 9, 10, 11, 12, 13 or 14 cycles. In a further embodiment, the patient is first administered the TIM-3 binding protein at a dose of 3.75 mg/kg as a monotherapy regimen every 3 weeks for up to 1, 2, 3, 4, 5, 6, 7, 8, 9, 10, 11, 12, 13 or 14 cycles and then the TIM-3 binding protein at a dose of 3.75 mg/kg with the ICOS binding protein at a dose of 0.3 mg/kg as a combination therapy regimen every 3 weeks for up to 1, 2, 3, 4, 5, 6, 7, 8, 9, 10, 11, 12, 13 or 14 cycles. In a further embodiment, the patient is first administered the TIM-3 binding protein at a dose of 11.25 mg/kg as a monotherapy regimen every 3 weeks for up to 1, 2, 3, 4, 5, 6, 7, 8, 9, 10, 11, 12, 13 or 14 cycles and then the TIM-3 binding protein at a dose of 11.25 mg/kg with the ICOS binding protein at a dose of 0.3 mg/kg as a combination therapy regimen every 3 weeks for up to 1, 2, 3, 4, 5, 6, 7, 8, 9, 10, 11, 12, 13 or 14 cycles.

In a further embodiment, the patient is first administered the TIM-3 binding protein at a dose of 1.25 mg/kg as a monotherapy regimen every 3 weeks for up to 1, 2, 3, 4, 5, 6, 7, 8, 9, 10, 11, 12, 13 or 14 cycles and then the TIM-3 binding protein at a dose of 1.25 mg/kg with the ICOS binding protein at a dose of 1 mg/kg as a combination therapy regimen every 3 weeks for up to 1, 2, 3, 4, 5, 6, 7, 8, 9, 10, 11, 12, 13 or 14 cycles. In a further embodiment, the patient is first administered the TIM-3 binding protein at a dose of 3.75 mg/kg as a monotherapy regimen every 3 weeks for up to 1, 2, 3, 4, 5, 6, 7, 8, 9, 10, 11, 12, 13 or 14 cycles and then the TIM-3 binding protein at a dose of 3.75 mg/kg with the ICOS binding protein at a dose of 1 mg/kg as a combination therapy regimen every 3 weeks for up to 1, 2, 3, 4, 5, 6, 7, 8, 9, 10, 11, 12, 13 or 14 cycles. In a further embodiment, the patient is first administered the TIM-3 binding protein at a dose of 11.25 mg/kg as a monotherapy regimen every 3 weeks for up to 1, 2, 3, 4, 5, 6, 7, 8, 9, 10, 11, 12, 13 or 14 cycles and then the TIM-3 binding protein at a dose of 11.25 mg/kg with the ICOS binding protein at a dose of 1 mg/kg as a combination therapy regimen every 3 weeks for up to 1, 2, 3, 4, 5, 6, 7, 8, 9, 10, 11, 12, 13 or 14 cycles.

In a further embodiment, the patient is first administered the TIM-3 binding protein at a dose of 1.25 mg/kg as a monotherapy regimen every 3 weeks for up to 1, 2, 3, 4, 5, 6, 7, 8, 9, 10, 11, 12, 13 or 14 cycles and then the TIM-3 binding protein at a dose of 1.25 mg/kg with the ICOS binding protein at a dose of 0.3 mg/kg and the PD-1 binding protein at a dose of 6.25 mg/kg as a combination therapy regimen every 3 weeks for up to 1, 2, 3, 4, 5, 6, 7, 8, 9, 10, 11, 12, 13 or 14 cycles. In a further embodiment, the patient is first administered the TIM-3 binding protein at a dose of 3.75 mg/kg as a monotherapy regimen every 3 weeks for up to 1, 2, 3, 4, 5, 6, 7, 8, 9, 10, 11, 12, 13 or 14 cycles and then the TIM-3 binding protein at a dose of 3.75 mg/kg with the ICOS binding protein at a dose of 0.3 mg/kg and the PD-1 binding protein at a dose of 6.25 mg/kg as a combination therapy regimen every 3 weeks for up to 1, 2, 3, 4, 5, 6, 7, 8, 9, 10, 11, 12, 13 or 14 cycles. In a further embodiment, the patient is first administered the TIM-3 binding protein at a dose of 11.25 mg/kg as a monotherapy regimen every 3 weeks for up to 1, 2, 3, 4, 5, 6, 7, 8, 9, 10, 11, 12, 13 or 14 cycles and then the TIM-3 binding protein at a dose of 11.25 mg/kg with the ICOS binding protein at a dose of 0.3 mg/kg and the PD-1 binding protein at a dose of 6.25 mg/kg as a combination therapy regimen every 3 weeks for up to 1, 2, 3, 4, 5, 6, 7, 8, 9, 10, 11, 12, 13 or 14 cycles. In a further embodiment, the patient is first administered the TIM-3 binding protein at a dose of 1.25 mg/kg as a monotherapy regimen every 3 weeks for up to 1, 2, 3, 4, 5, 6, 7, 8, 9, 10, 11, 12, 13 or 14 cycles and then the TIM-3 binding protein at a dose of 1.25 mg/kg with the ICOS binding protein at a dose of 1 mg/kg and the PD-1 binding protein at a dose of 6.25 mg/kg as a combination therapy regimen every 3 weeks for up to 1, 2, 3, 4, 5, 6, 7, 8, 9, 10, 11, 12, 13 or 14 cycles. In a further embodiment, the patient is first administered the TIM-3 binding protein at a dose of 3.75 mg/kg as a monotherapy regimen every 3 weeks for up to 1, 2, 3, 4, 5, 6, 7, 8, 9, 10, 11, 12, 13 or 14 cycles and then the TIM-3 binding protein at a dose of 3.75 mg/kg with the ICOS binding protein at a dose of 1 mg/kg and the PD-1 binding protein at a dose of 6.25 mg/kg as a combination therapy regimen every 3 weeks for up to 1, 2, 3, 4, 5, 6, 7, 8, 9, 10, 11, 12, 13 or 14 cycles. In a further embodiment, the patient is first administered the TIM-3 binding protein at a dose of 11.25 mg/kg as a monotherapy regimen every 3 weeks for up to 1, 2, 3, 4, 5, 6, 7, 8, 9, 10, 11, 12, 13 or 14 cycles and then the TIM-3 binding protein at a dose of 11.25 mg/kg with the ICOS binding protein at a dose of 1 mg/kg and the PD-1 binding protein at a dose of 6.25 mg/kg as a combination therapy regimen every 3 weeks for up to 1, 2, 3, 4, 5, 6, 7, 8, 9, 10, 11, 12, 13 or 14 cycles.

In a further embodiment, the patient is first administered the TIM-3 binding protein at a dose of 1.25 mg/kg as a monotherapy regimen every 6 weeks for up to 1, 2, 3, 4, 5, 6, 7, 8, 9, 10, 11, 12, 13 or 14 cycles and then the TIM-3 binding protein at a dose of 1.25 mg/kg with the ICOS binding protein at a dose of 0.6 mg/kg and the PD-1 binding protein at a dose of 12.5 mg/kg as a combination therapy regimen every 6 weeks for up to 1, 2, 3, 4, 5, 6, 7, 8, 9, 10, 11, 12, 13 or 14 cycles. In a further embodiment, the patient is first administered the TIM-3 binding protein at a dose of 3.75 mg/kg as a monotherapy regimen every 6 weeks for up to 1, 2, 3, 4, 5, 6, 7, 8, 9, 10, 11, 12, 13 or 14 cycles and then the TIM-3 binding protein at a dose of 3.75 mg/kg with the ICOS binding protein at a dose of 0.6 mg/kg and the PD-1 binding protein at a dose of 12.5 mg/kg as a combination therapy regimen every 6 weeks for up to 1, 2, 3, 4, 5, 6, 7, 8, 9, 10, 11, 12, 13 or 14 cycles. In a further embodiment, the patient is first administered the TIM-3 binding protein at a dose of 11.25 mg/kg as a monotherapy regimen every 6 weeks for up to 1, 2, 3, 4, 5, 6, 7, 8, 9, 10, 11, 12, 13 or 14 cycles and then the TIM-3 binding protein at a dose of 11.25 mg/kg with the ICOS binding protein at a dose of 0.6 mg/kg and the PD-1 binding protein at a dose of 12.5 mg/kg as a combination therapy regimen every 6 weeks for up to 1, 2, 3, 4, 5, 6, 7, 8, 9, 10, 11, 12, 13 or 14 cycles. In a further embodiment, the patient is first administered the TIM-3 binding protein at a dose of 1.25 mg/kg as a monotherapy regimen every 6 weeks for up to 1, 2, 3, 4, 5, 6, 7, 8, 9, 10, 11, 12, 13 or 14 cycles and then the TIM-3 binding protein at a dose of 1.25 mg/kg with the ICOS binding protein at a dose of 2 mg/kg and the PD-1 binding protein at a dose of 12.5 mg/kg as a combination therapy regimen every 6 weeks for up to 1, 2, 3, 4, 5, 6, 7, 8, 9, 10, 11, 12, 13 or 14 cycles. In a further embodiment, the patient is first administered the TIM-3 binding protein at a dose of 3.75 mg/kg as a monotherapy regimen every 6 weeks for up to 1, 2, 3, 4, 5, 6, 7, 8, 9, 10, 11, 12, 13 or 14 cycles and then the TIM-3 binding protein at a dose of 3.75 mg/kg with the ICOS binding protein at a dose of 2 mg/kg and the PD-1 binding protein at a dose of 12.5 mg/kg as a combination therapy regimen every 6 weeks for up to 1, 2, 3, 4, 5, 6, 7, 8, 9, 10, 11, 12, 13 or 14 cycles. In a further embodiment, the patient is first administered the TIM-3 binding protein at a dose of 11.25 mg/kg as a monotherapy regimen every 6 weeks for up to 1, 2, 3, 4, 5, 6, 7, 8, 9, 10, 11, 12, 13 or 14 cycles and then the TIM-3 binding protein at a dose of 11.25 mg/kg with the ICOS binding protein at a dose of 2 mg/kg and the PD-1 binding protein at a dose of 12.5 mg/kg as a combination therapy regimen every 6 weeks for up to 1, 2, 3, 4, 5, 6, 7, 8, 9, 10, 11, 12, 13 or 14 cycles.

It will be understood that between first administration to the patient of an ICOS binding protein or a TIM-3 binding protein as a monotherapy and the administration of the ICOS binding protein and TIM-3 binding protein as a combination therapy as described herein, a period of no treatment or no administration may be performed, such as for a defined number of cycles. For example, after first administration with a monotherapy, the patient may be administered no treatment for 1 cycle or 2 cycles of 3 weeks, 6 weeks or 12 weeks before being administered a combination therapy as described herein. Thus, in one embodiment, the patient is first administered an ICOS binding protein as a monotherapy as described herein, then administered no treatment for 1 cycle or 2 cycles of 3 weeks, 6 weeks or 12 weeks, before the patient is administered an ICOS binding protein with a TIM-3 binding protein as a combination therapy as described herein. In one embodiment, the patient is first administered a TIM-3 binding protein as a monotherapy as described herein, then administered no treatment for 1 cycle or 2 cycles of 3 weeks, 6 weeks or 12 weeks, before the patient is administered a TIM-3 binding protein with an ICOS binding protein as a combination therapy as described herein.

In one aspect, there is provided a method of treating cancer in a human in need thereof, the method comprising administering to the human an ICOS binding protein at a dose of about 0.08 mg to about 240 mg and administering to the human a TIM-3 binding protein, wherein the ICOS binding protein comprises a V_{H} domain comprising an amino acid sequence at least 90% identical to the amino acid sequence set forth in SEQ ID NO:7 and/or a V_{L} domain comprising an amino acid sequence at least 90% identical to the amino acid sequence as set forth in SEQ ID NO:8 wherein said ICOS binding protein specifically binds to human ICOS. In another aspect, there is provided a method of treating cancer in a human in need thereof, the method comprising administering to the human an ICOS binding protein at a dose of about 0.08 mg to about 240 mg and administering to the human a PD-1 binding protein and a TIM-3 binding protein, wherein the ICOS binding protein comprises a V_{H} domain comprising an amino acid sequence at least 90% identical to the amino acid sequence set forth in SEQ ID NO:7 and/or a V_{L} domain comprising an amino acid sequence at least 90% identical to the amino acid sequence as set forth in SEQ ID NO:8 wherein said ICOS binding protein specifically binds to human ICOS. In one embodiment, the ICOS binding protein is administered at a dose of about 24 mg to about 160 mg, wherein the ICOS binding protein comprises a V_{H} domain comprising an amino acid sequence at least 90% identical to the amino acid sequence set forth in SEQ ID NO:7 and/or a V_{L} domain comprising an amino acid sequence at least 90% identical to the amino acid sequence as set forth in SEQ ID NO:8 wherein said ICOS binding protein specifically binds to human ICOS. In one embodiment, the ICOS binding protein is administered at a dose of 24 mg, 48 mg, 80 mg or 160 mg. In another embodiment, the ICOS binding protein is administered at a dose of 24 mg, 48 mg, 80 mg or 160 mg, and the TIM-3 binding protein is administered at a dose of 100 mg, 300 mg or 900 mg. In a further embodiment, the ICOS binding protein is administered at a dose of 24 mg, 48 mg, 80 mg or 160 mg, the PD-1 binding protein is administered at a dose of 500 mg or 1000 mg and the TIM-3 binding protein is administered at a dose of 100 mg, 300 mg or 900 mg. In one embodiment, the TIM-3 binding protein is cobolimab. In one embodiment, the PD-1 binding protein is dostarlimab. In one embodiment, the ICOS binding protein comprises one or more of: CDRH1 as set forth in SEQ ID NO:1; CDRH2 as set forth in SEQ ID NO:2; CDRH3 as set forth in SEQ ID NO:3; CDRL1 as set forth in SEQ ID NO:4; CDRL2 as set forth in SEQ ID NO:5 and/or CDRL3 as set forth in SEQ ID NO:6 or a direct equivalent of each CDR wherein a direct equivalent has no more than two amino acid substitutions in said CDR. In one embodiment, the ICOS binding protein comprises a heavy chain variable region comprising one or more of SEQ ID NO:1; SEQ ID NO:2; and SEQ ID NO:3 and wherein said ICOS binding protein comprises a light chain variable region comprising one or more of SEQ ID NO:4; SEQ ID NO:5, and SEQ ID NO:6. In one embodiment, the ICOS binding protein comprises a heavy chain variable region comprising SEQ ID NO:1; SEQ ID NO:2; and SEQ ID NO:3 and wherein said ICOS binding protein comprises a light chain variable region comprising SEQ ID NO:4; SEQ ID NO:5, and SEQ ID NO:6. In one embodiment, the ICOS binding protein comprises a V_{H} domain comprising the amino acid sequence set forth in SEQ ID NO:7 and a V_{L} domain comprising the amino acid sequence as set forth in SEQ ID NO:8. In one embodiment, the ICOS binding protein comprises a heavy chain comprising the amino acid sequence set forth in SEQ ID NO:9 and a light chain comprising the amino acid sequence as set forth in SEQ ID NO:10.

In one aspect, there is provided a method of treating cancer in a human in need thereof, the method comprising administering to the human a TIM-3 binding protein at a dose of about 5 mg to about 5000 mg and administering to the human an ICOS binding protein, wherein the TIM-3 binding protein comprises a V_{H} domain comprising an amino acid sequence at least 90% identical to the amino acid sequence set forth in SEQ ID NO:19 and/or a V_{L} domain comprising an amino acid sequence at least 90% identical to the amino acid sequence as set forth in SEQ ID NO:20 wherein said TIM-3 binding protein specifically binds to human TIM-3. In another aspect, there is provided a method of treating cancer in a human in need thereof, the method comprising administering to the human a TIM-3 binding protein at a dose of about 5 mg to about 5000 mg and administering to the human an ICOS binding protein and a PD-1 binding protein, wherein the TIM-3 binding protein comprises a V_{H} domain comprising an amino acid sequence at least 90% identical to the amino acid sequence set forth in SEQ ID NO:19 and/or a V_{L} domain comprising an amino acid sequence at least 90% identical to the amino acid sequence as set forth in SEQ ID NO:20 wherein said TIM-3 binding protein specifically binds to human TIM-3. In one embodiment, the TIM-3 binding protein is administered at a dose of about 100 mg to about 900 mg, wherein the TIM-3 binding protein comprises a V_{H} domain comprising an amino acid sequence at least 90% identical to the amino acid sequence set forth in SEQ ID NO:19 and/or a V_{L} domain comprising an amino acid sequence at least 90% identical to the amino acid sequence as set forth in SEQ ID NO:20 wherein said TIM-3 binding protein specifically binds to human TIM-3. In one embodiment, the TIM-3 binding protein is administered at a dose of 100 mg, 300 mg or 900 mg. In another embodiment, the ICOS binding protein is administered at a dose of 24 mg, 48 mg, 80 mg or 160 mg, and the TIM-3 binding protein is administered at a dose of 100 mg, 300 mg or 900 mg. In a further embodiment, the ICOS binding protein is administered at a dose of 24 mg, 48 mg, 80 mg or 160 mg, the PD-1 binding protein is administered at a dose of 500 mg or 1000 mg and the TIM-3 binding protein is administered at a dose of 100 mg, 300 mg or 900 mg. In one embodiment, the TIM-3 binding protein comprises one or more of: CDRH1 as set forth in SEQ ID NO:13; CDRH2 as set forth in SEQ ID NO:14; CDRH3 as set forth in SEQ ID NO:15; CDRL1 as set forth in SEQ ID NO:16; CDRL2 as set forth in SEQ ID NO:17 and/or CDRL3 as set forth in SEQ ID NO:18 or a direct equivalent of each CDR wherein a direct equivalent has no more than two amino acid substitutions in said CDR. In one embodiment, the TIM-3 binding protein comprises a heavy chain variable region comprising one or more of SEQ ID NO:13; SEQ ID NO:14; and SEQ ID NO:15 and wherein said TIM-3 binding protein comprises a light chain variable region comprising one or more of SEQ ID NO:16; SEQ ID NO:17, and SEQ ID NO:18. In one embodiment, the TIM-3 binding protein comprises a heavy chain variable region comprising SEQ ID NO:13; SEQ ID NO:14; and SEQ ID NO:15 and wherein said TIM-3 binding protein comprises a light chain variable region comprising SEQ ID NO:16; SEQ ID NO:17, and SEQ ID NO:18. In one embodiment, the TIM-3 binding protein comprises a V_{H} domain comprising the amino acid sequence set forth in SEQ ID NO:19 and a V_{L} domain comprising the amino acid sequence as set forth in SEQ ID NO:20. In one embodiment, the TIM-3 binding protein comprises a heavy chain comprising the amino acid sequence set forth in SEQ ID NO:21 and a light chain comprising the amino acid sequence as set forth in SEQ ID NO:22. In one embodiment, the TIM-3 binding protein is cobolimab.

In one aspect, there is provided an ICOS binding protein and a TIM-3 binding protein for concurrent or sequential use in treating cancer, wherein the ICOS binding protein is to be administered at a dose of about 0.08 mg to about 240 mg and the TIM-3 binding protein is to be administered at a dose of about 5 mg to about 5000 mg, wherein the ICOS binding protein comprises a V_{H} domain comprising an amino acid sequence at least 90% identical to the amino acid sequence set forth in SEQ ID NO:7 and/or a V_{L} domain comprising an amino acid sequence at least 90% identical to the amino acid sequence as set forth in SEQ ID NO:8 wherein said ICOS binding protein specifically binds to human ICOS. In another aspect, there is provided an ICOS binding protein, a PD-1 binding protein and a TIM-3 binding protein for concurrent or sequential use in treating cancer, wherein the ICOS binding protein is to be administered at a dose of about 0.08 mg to about 240 mg, the PD-1 binding protein is to be administered at a dose of about 100 mg to about 2000 mg and the TIM-3 binding protein is to be administered at a dose of about 5 mg to about 5000 mg, wherein the ICOS binding protein comprises a V_{H} domain comprising an amino acid sequence at least 90% identical to the amino acid sequence set forth in SEQ ID NO:7 and/or a V_{L} domain comprising an amino acid sequence at least 90% identical to the amino acid sequence as set forth in SEQ ID NO:8 wherein said ICOS binding protein specifically binds to human ICOS. In one embodiment, the ICOS binding protein is to be administered at a dose of about 24 mg to about 160 mg and the TIM-3 binding protein is to be administered at a dose of about 100 mg to about 900 mg, wherein the ICOS binding protein comprises a V_{H} domain comprising an amino acid sequence at least 90% identical to the amino acid sequence set forth in SEQ ID NO:7 and/or a V_{L} domain comprising an amino acid sequence at least 90% identical to the amino acid sequence as set forth in SEQ ID NO:8 wherein said ICOS binding protein specifically binds to human ICOS. In one embodiment, the ICOS binding protein is administered at a dose of 24 mg, 48 mg, 80 mg or 160 mg. In another embodiment, the ICOS binding protein is administered at a dose of 24 mg, 48 mg, 80 mg or 160 mg, and the TIM-3 binding protein is administered at a dose of 100 mg, 300 mg or 900 mg. In a further embodiment, the ICOS binding protein is administered at a dose of 24 mg, 48 mg, 80 mg or 160 mg, the PD-1 binding protein is administered at a dose of 500 mg or 1000 mg and the TIM-3 binding protein is administered at a dose of 100 mg, 300 mg or 900 mg. In one embodiment, the TIM-3 binding protein is cobolimab. In one embodiment, the PD-1 binding protein is dostarlimab. In one embodiment, the ICOS binding protein comprises one or more of: CDRH1 as set forth in SEQ ID NO:1; CDRH2 as set forth in SEQ ID NO:2; CDRH3 as set forth in SEQ ID NO:3; CDRL1 as set forth in SEQ ID NO:4; CDRL2 as set forth in SEQ ID NO:5 and/or CDRL3 as set forth in SEQ ID NO:6 or a direct equivalent of each CDR wherein a direct equivalent has no more than two amino acid substitutions in said CDR. In one embodiment, the ICOS binding protein comprises a heavy chain variable region comprising one or more of SEQ ID NO:1; SEQ ID NO:2; and SEQ ID NO:3 and wherein said ICOS binding protein comprises a light chain variable region comprising one or more of SEQ ID NO:4; SEQ ID NO:5, and SEQ ID NO:6. In one embodiment, the ICOS binding protein comprises a heavy chain variable region comprising SEQ ID NO:1; SEQ ID NO:2; and SEQ ID NO:3 and wherein said ICOS binding protein comprises a light chain variable region comprising SEQ ID NO:4; SEQ ID NO:5, and SEQ ID NO:6. In one embodiment, the ICOS binding protein comprises a V_{H} domain comprising the amino acid sequence set forth in SEQ ID NO:7 and a V_{L} domain comprising the amino acid sequence as set forth in SEQ ID NO:8. In one embodiment, the ICOS binding protein comprises a heavy chain comprising the amino acid sequence set forth in SEQ ID NO:9 and a light chain comprising the amino acid sequence as set forth in SEQ ID NO:10.

In one aspect, there is provided a TIM-3 binding protein and an ICOS binding protein for concurrent or sequential use in treating cancer, wherein the TIM-3 binding protein is to be administered at a dose of about 5 mg to about 5000 mg and the ICOS binding protein is to be administered at a dose of about 0.08 mg to about 240 mg, wherein the TIM-3 binding protein comprises a V_{H} domain comprising an amino acid sequence at least 90% identical to the amino acid sequence set forth in SEQ ID NO:19 and/or a V_{L} domain comprising an amino acid sequence at least 90% identical to the amino acid sequence as set forth in SEQ ID NO:20 wherein said TIM-3 binding protein specifically binds to human TIM-3. In another aspect, there is provided a TIM-3 binding protein, an ICOS binding protein and a PD-1 binding protein for concurrent or sequential use in treating cancer, wherein the TIM-3 binding protein is to be administered at a dose of about 5 mg to about 5000 mg, the ICOS binding protein is to be administered at a dose of about 0.08 mg to about 240 mg and the PD-1 binding protein is to be administered at a dose of about 100 mg to about 2000 mg, wherein the TIM-3 binding protein comprises a V_{H} domain comprising an amino acid sequence at least 90% identical to the amino acid sequence set forth in SEQ ID NO:19 and/or a V_{L} domain comprising an amino acid sequence at least 90% identical to the amino acid sequence as set forth in SEQ ID NO:20 wherein said TIM-3 binding protein specifically binds to human TIM-3. In one embodiment, the TIM-3 binding protein is to be administered at a dose of about 100 mg to about 900 mg and the ICOS binding protein is to be administered at a dose of about 24 mg to about 160 mg, wherein the TIM-3 binding protein comprises a V_{H} domain comprising an amino acid sequence at least 90% identical to the amino acid sequence set forth in SEQ ID NO:19 and/or a V_{L} domain comprising an amino acid sequence at least 90% identical to the amino acid sequence as set forth in SEQ ID NO:20 wherein said TIM-3 binding protein specifically binds to human TIM-3. In one embodiment, the TIM-3 binding protein is to be administered at a dose of about 5 mg to about 5000 mg, the ICOS binding protein is to be administered at a dose of about 8 mg to about 160 mg and the PD-1 binding protein is to be administered at a dose of about 100 mg to about 2000 mg, wherein the TIM-3 binding protein comprises a V_{H} domain comprising an amino acid sequence at least 90% identical to the amino acid sequence set forth in SEQ ID NO:19 and/or a V_{L} domain comprising an amino acid sequence at least 90% identical to the amino acid sequence as set forth in SEQ ID NO:20 wherein said TIM-3 binding protein specifically binds to human TIM-3. In one embodiment, the TIM-3 binding protein is administered at a dose of 100 mg, 300 mg or 900 mg. In another embodiment, the ICOS binding protein is administered at a dose of 24 mg, 48 mg, 80 mg or 160 mg, and the TIM-3 binding protein is administered at a dose of 100 mg, 300 mg or 900 mg. In a further embodiment, the ICOS binding protein is administered at a dose of 24 mg, 48 mg, 80 mg or 160 mg, the PD-1 binding protein is administered at a dose of 500 mg or 1000 mg and the TIM-3 binding protein is administered at a dose of 100 mg, 300 mg or 900 mg. In one embodiment, the TIM-3 binding protein comprises one or more of: CDRH1 as set forth in SEQ ID NO:13; CDRH2 as set forth in SEQ ID NO:14; CDRH3 as set forth in SEQ ID NO:15; CDRL1 as set forth in SEQ ID NO:16; CDRL2 as set forth in SEQ ID NO:17 and/or CDRL3 as set forth in SEQ ID NO:18 or a direct equivalent of each CDR wherein a direct equivalent has no more than two amino acid substitutions in said CDR. In one embodiment, the TIM-3 binding protein comprises a heavy chain variable region comprising one or more of SEQ ID NO:13; SEQ ID NO:14; and SEQ ID NO:15 and wherein said TIM-3 binding protein comprises a light chain variable region comprising one or more of SEQ ID NO:16; SEQ ID NO:17, and SEQ ID NO:18. In one embodiment, the TIM-3 binding protein comprises a heavy chain variable region comprising SEQ ID NO:13; SEQ ID NO:14; and SEQ ID NO:15 and wherein said TIM-3 binding protein comprises a light chain variable region comprising SEQ ID NO:16; SEQ ID NO:17, and SEQ ID NO:18. In one embodiment, the TIM-3 binding protein comprises a V_{H} domain comprising the amino acid sequence set forth in SEQ ID NO:19 and a V_{L} domain comprising the amino acid sequence as set forth in SEQ ID NO:20. In one embodiment, the TIM-3 binding protein comprises a heavy chain comprising the amino acid sequence set forth in SEQ ID NO:21 and a light chain comprising the amino acid sequence as set forth in SEQ ID NO:22. In one embodiment, the TIM-3 binding protein is cobolimab.

In another aspect, an ICOS binding protein for use in treating cancer is provided, wherein the ICOS binding protein is to be administered at a dose of about 0.08 mg to about 240 mg and is to be administered concurrently or sequentially with a TIM-3 binding protein, wherein the ICOS binding protein comprises a V_{H} domain comprising an amino acid sequence at least 90% identical to the amino acid sequence set forth in SEQ ID NO:7 and/or a V_{L} domain comprising an amino acid sequence at least 90% identical to the amino acid sequence as set forth in SEQ ID NO:8, wherein said ICOS binding protein specifically binds to human ICOS. In a further aspect, an ICOS binding protein for use in treating cancer is provided, wherein the ICOS binding protein is to be administered at a dose of about 0.08 mg to about 240 mg and is to be administered concurrently or sequentially with a PD-1 binding protein and a TIM-3 binding protein, wherein the ICOS binding protein comprises a V_{H} domain comprising an amino acid sequence at least 90% identical to the amino acid sequence set forth in SEQ ID NO:7 and/or a V_{L} domain comprising an amino acid sequence at least 90% identical to the amino acid sequence as set forth in SEQ ID NO:8, wherein said ICOS binding protein specifically binds to human ICOS. In one embodiment, the ICOS binding protein is to be administered at a dose of about 24 mg to about 160 mg and is to be administered concurrently or sequentially with a TIM-3 binding protein, wherein the ICOS binding protein comprises a V_{H} domain comprising an amino acid sequence at least 90% identical to the amino acid sequence set forth in SEQ ID NO:7 and/or a V_{L} domain comprising an amino acid sequence at least 90% identical to the amino acid sequence as set forth in SEQ ID NO:8 wherein said ICOS binding protein specifically binds to human ICOS. In a further embodiment, the ICOS binding protein is to be administered at a dose of about 24 mg to about 160 mg and is to be administered concurrently or sequentially with a PD-1 binding protein and a TIM-3 binding protein, wherein the ICOS binding protein comprises a V_{H} domain comprising an amino acid sequence at least 90% identical to the amino acid sequence set forth in SEQ ID NO:7 and/or a V_{L} domain comprising an amino acid sequence at least 90% identical to the amino acid sequence as set forth in SEQ ID NO:8 wherein said ICOS binding protein specifically binds to human ICOS. In one embodiment, the ICOS binding protein is administered at a dose of 24 mg, 48 mg, 80 mg or 160 mg. In another embodiment, the ICOS binding protein is administered at a dose of 24 mg, 48 mg, 80 mg or 160 mg and the TIM-3 binding protein is administered at a dose of 100 mg. 300 mg or 900 mg. In yet further embodiment, the ICOS binding protein is administered at a dose of 24 mg, 48 mg, 80 mg or 160 mg, the PD-1 binding protein is administered at a dose of 500 mg or 1000 mg and the TIM-3 binding protein is administered at a dose of 100 mg, 300 mg or 900 mg. In one embodiment, the TIM-3 binding protein is cobolimab. In one embodiment, the PD-1 binding protein is dostarlimab. In one embodiment, the ICOS binding protein comprises one or more of: CDRH1 as set forth in SEQ ID NO:1; CDRH2 as set forth in SEQ ID NO:2; CDRH3 as set forth in SEQ ID NO:3; CDRL1 as set forth in SEQ ID NO:4; CDRL2 as set forth in SEQ ID NO:5 and/or CDRL3 as set forth in SEQ ID NO:6 or a direct equivalent of each CDR wherein a direct equivalent has no more than two amino acid substitutions in said CDR. In one embodiment, the ICOS binding protein comprises a heavy chain variable region comprising one or more of SEQ ID NO:1; SEQ ID NO:2; and SEQ ID NO:3 and wherein said ICOS binding protein comprises a light chain variable region comprising one or more of SEQ ID NO:4; SEQ ID NO:5, and SEQ ID NO:6. In one embodiment, the ICOS binding protein comprises a heavy chain variable region comprising SEQ ID NO:1; SEQ ID NO:2; and SEQ ID NO:3 and wherein said ICOS binding protein comprises a light chain variable region comprising SEQ ID NO:4; SEQ ID NO:5, and SEQ ID NO:6. In one embodiment, the ICOS binding protein comprises a V_{H} domain comprising the amino acid sequence set forth in SEQ ID NO:7 and a V_{L} domain comprising the amino acid sequence as set forth in SEQ ID NO:8. In one embodiment, the ICOS binding protein comprises a heavy chain comprising the amino acid sequence set forth in SEQ ID NO:9 and a light chain comprising the amino acid sequence as set forth in SEQ ID NO:10.

In another aspect, a TIM-3 binding protein for use in treating cancer is provided, wherein the TIM-3 binding protein is to be administered at a dose of about 5 mg to about 5000 mg and is to be administered concurrently or sequentially with an ICOS binding protein, wherein the TIM-3 binding protein comprises a V_{H} domain comprising an amino acid sequence at least 90% identical to the amino acid sequence set forth in SEQ ID NO:19 and/or a V_{L} domain comprising an amino acid sequence at least 90% identical to the amino acid sequence as set forth in SEQ ID NO:20, wherein said TIM-3 binding protein specifically binds to human TIM-3. In a further aspect, a TIM-3 binding protein for use in treating cancer is provided, wherein the TIM-3 binding protein is to be administered at a dose of about 5 mg to about 5000 mg and is to be administered concurrently or sequentially with an ICOS binding protein and a PD-1 binding protein, wherein the TIM-3 binding protein comprises a V_{H} domain comprising an amino acid sequence at least 90% identical to the amino acid sequence set forth in SEQ ID NO:19 and/or a V_{L} domain comprising an amino acid sequence at least 90% identical to the amino acid sequence as set forth in SEQ ID NO:20, wherein said TIM-3 binding protein specifically binds to human TIM-3. In one embodiment, the TIM-3 binding protein is to be administered at a dose of about 100 mg to about 900 mg and is to be administered concurrently or sequentially with an ICOS binding protein, wherein the TIM-3 binding protein comprises a V_{H} domain comprising an amino acid sequence at least 90% identical to the amino acid sequence set forth in SEQ ID NO:19 and/or a V_{L} domain comprising an amino acid sequence at least 90% identical to the amino acid sequence as set forth in SEQ ID NO:20 wherein said TIM-3 binding protein specifically binds to human TIM-3. In a further embodiment, the TIM-3 binding protein is to be administered at a dose of about 100 mg to about 900 mg and is to be administered concurrently or sequentially with an ICOS binding protein and a PD-1 binding protein, wherein the TIM-3 binding protein comprises a V_{H} domain comprising an amino acid sequence at least 90% identical to the amino acid sequence set forth in SEQ ID NO:19 and/or a V_{L} domain comprising an amino acid sequence at least 90% identical to the amino acid sequence as set forth in SEQ ID NO:20 wherein said TIM-3 binding protein specifically binds to human TIM-3. In one embodiment, the TIM-3 binding protein is administered at a dose of 100 mg, 300 mg or 900 mg. In another embodiment, the ICOS binding protein is administered at a dose of 24 mg, 48 mg, 80 mg or 160 mg and the TIM-3 binding protein is administered at a dose of 100 mg, 300 mg or 900 mg. In another embodiment, the ICOS binding protein is administered at a dose of 24 mg, 48 mg, 80 mg or 160 mg, the PD-1 binding protein is administered at a dose of 500 mg or 1000 mg and the TIM-3 binding protein is administered at a dose of 100 mg, 300 mg or 900 mg. In one embodiment, the TIM-3 binding protein comprises one or more of: CDRH1 as set forth in SEQ ID NO:13; CDRH2 as set forth in SEQ ID NO:14; CDRH3 as set forth in SEQ ID NO:15; CDRL1 as set forth in SEQ ID NO:16; CDRL2 as set forth in SEQ ID NO:17 and/or CDRL3 as set forth in SEQ ID NO:18 or a direct equivalent of each CDR wherein a direct equivalent has no more than two amino acid substitutions in said CDR. In one embodiment, the TIM-3 binding protein comprises a heavy chain variable region comprising one or more of SEQ ID NO:13; SEQ ID NO:14; and SEQ ID NO:15 and wherein said TIM-3 binding protein comprises a light chain variable region comprising one or more of SEQ ID NO:16; SEQ ID NO:17, and SEQ ID NO:18. In one embodiment, the TIM-3 binding protein comprises a heavy chain variable region comprising SEQ ID NO:13; SEQ ID NO:14; and SEQ ID NO:15 and wherein said TIM-3 binding protein comprises a light chain variable region comprising SEQ ID NO:16; SEQ ID NO:17, and SEQ ID NO:18. In one embodiment, the TIM-3 binding protein comprises a V_{H} domain comprising the amino acid sequence set forth in SEQ ID NO:19 and a V_{L} domain comprising the amino acid sequence as set forth in SEQ ID NO:20. In one embodiment, the TIM-3 binding protein comprises a heavy chain comprising the amino acid sequence set forth in SEQ ID NO:21 and a light chain comprising the amino acid sequence as set forth in SEQ ID NO:22. In one embodiment, the TIM-3 binding protein is cobolimab.

In another aspect, there is provided use of an ICOS binding protein in the manufacture of a medicament for treating cancer, wherein the ICOS binding protein is to be administered at a dose of about 0.08 mg to about 240 mg and is to be administered concurrently or sequentially with a TIM-3 binding protein, wherein the ICOS binding protein comprises a V_{H} domain comprising an amino acid sequence at least 90% identical to the amino acid sequence set forth in SEQ ID NO:7 and/or a V_{L} domain comprising an amino acid sequence at least 90% identical to the amino acid sequence as set forth in SEQ ID NO:8 wherein said ICOS binding protein specifically binds to human ICOS. In a further aspect, there is provided use of an ICOS binding protein in the manufacture of a medicament for treating cancer, wherein the ICOS binding protein is to be administered at a dose of about 0.08 mg to about 240 mg and is to be administered concurrently or sequentially with a PD-1 binding protein and a TIM-3 binding protein, wherein the ICOS binding protein comprises a V_{H} domain comprising an amino acid sequence at least 90% identical to the amino acid sequence set forth in SEQ ID NO:7 and/or a V_{L} domain comprising an amino acid sequence at least 90% identical to the amino acid sequence as set forth in SEQ ID NO:8 wherein said ICOS binding protein specifically binds to human ICOS. In one embodiment, the ICOS binding protein is to be administered at a dose of about 24 mg to about 160 mg and is to be administered concurrently or sequentially with a TIM-3 binding protein, wherein the ICOS binding protein comprises a V_{H} domain comprising an amino acid sequence at least 90% identical to the amino acid sequence set forth in SEQ ID NO:7 and/or a V_{L} domain comprising an amino acid sequence at least 90% identical to the amino acid sequence as set forth in SEQ ID NO:8 wherein said ICOS binding protein specifically binds to human ICOS. In another embodiment, the ICOS binding protein is to be administered at a dose of about 24 mg to about 160 mg and is to be administered concurrently or sequentially with a PD-1 binding protein and a TIM-3 binding protein, wherein the ICOS binding protein comprises a V_{H} domain comprising an amino acid sequence at least 90% identical to the amino acid sequence set forth in SEQ ID NO:7 and/or a V_{L} domain comprising an amino acid sequence at least 90% identical to the amino acid sequence as set forth in SEQ ID NO:8 wherein said ICOS binding protein specifically binds to human ICOS. In one embodiment, the ICOS binding protein is administered at a dose of 24 mg, 48 mg, 80 mg or 160 mg. In one embodiment, the ICOS binding protein is administered at a dose of 24 mg, 48 mg, 80 mg or 160 mg, and the TIM-3 binding protein is administered at a dose of 100 mg, 300 mg or 900 mg. In one embodiment, the ICOS binding protein is administered at a dose of 24 mg, 48 mg, 80 mg or 160 mg, the PD-1 binding protein is administered at a dose of 500 mg or 1000 mg and the TIM-3 binding protein is administered at a dose of 100 mg, 300 mg or 900 mg. In one embodiment, the TIM-3 binding protein is cobolimab. In one embodiment, the PD-1 binding protein is dostarlimab. In one embodiment, the ICOS binding protein comprises one or more of: CDRH1 as set forth in SEQ ID NO:1; CDRH2 as set forth in SEQ ID NO:2; CDRH3 as set forth in SEQ ID NO:3; CDRL1 as set forth in SEQ ID NO:4; CDRL2 as set forth in SEQ ID NO:5 and/or CDRL3 as set forth in SEQ ID NO:6 or a direct equivalent of each CDR wherein a direct equivalent has no more than two amino acid substitutions in said CDR. In one embodiment, the ICOS binding protein comprises a heavy chain variable region comprising one or more of SEQ ID NO:1; SEQ ID NO:2; and SEQ ID NO:3 and wherein said ICOS binding protein comprises a light chain variable region comprising one or more of SEQ ID NO:4; SEQ ID NO:5, and SEQ ID NO:6. In one embodiment, the ICOS binding protein comprises a heavy chain variable region comprising SEQ ID NO:1; SEQ ID NO:2; and SEQ ID NO:3 and wherein said ICOS binding protein comprises a light chain variable region comprising SEQ ID NO:4; SEQ ID NO:5, and SEQ ID NO:6. In one embodiment, the ICOS binding protein comprises a V_{H} domain comprising the amino acid sequence set forth in SEQ ID NO:7 and a V_{L} domain comprising the amino acid sequence as set forth in SEQ ID NO:8. In one embodiment, the ICOS binding protein comprises a heavy chain comprising the amino acid sequence set forth in SEQ ID NO:9 and a light chain comprising the amino acid sequence as set forth in SEQ ID NO:10.

In another aspect, there is provided use of a TIM-3 binding protein in the manufacture of a medicament for treating cancer, wherein the TIM-3 binding protein is to be administered at a dose of about 5 mg to about 5000 mg and is to be administered concurrently or sequentially with an ICOS binding protein, wherein the TIM-3 binding protein comprises a V_{H} domain comprising an amino acid sequence at least 90% identical to the amino acid sequence set forth in SEQ ID NO:19 and/or a V_{L} domain comprising an amino acid sequence at least 90% identical to the amino acid sequence as set forth in SEQ ID NO:20 wherein said TIM-3 binding protein specifically binds to human TIM-3. In a further aspect, there is provided use of a TIM-3 binding protein in the manufacture of a medicament for treating cancer, wherein the TIM-3 binding protein is to be administered at a dose of about 5 mg to about 5000 mg and is to be administered concurrently or sequentially with an ICOS binding protein and a PD-1 binding protein, wherein the TIM-3 binding protein comprises a V_{H} domain comprising an amino acid sequence at least 90% identical to the amino acid sequence set forth in SEQ ID NO:19 and/or a V_{L} domain comprising an amino acid sequence at least 90% identical to the amino acid sequence as set forth in SEQ ID NO:20 wherein said TIM-3 binding protein specifically binds to human TIM-3. In one embodiment, the TIM-3 binding protein is to be administered at a dose of about 100 mg to about 900 mg and is to be administered concurrently or sequentially with an ICOS binding protein, wherein the TIM-3 binding protein comprises a V_{H} domain comprising an amino acid sequence at least 90% identical to the amino acid sequence set forth in SEQ ID NO:19 and/or a V_{L} domain comprising an amino acid sequence at least 90% identical to the amino acid sequence as set forth in SEQ ID NO:20 wherein said TIM-3 binding protein specifically binds to human TIM-3. In another embodiment, the TIM-3 binding protein is to be administered at a dose of about 100 mg to about 900 mg and is to be administered concurrently or sequentially with an ICOS binding protein and a PD-1 binding protein, wherein the TIM-3 binding protein comprises a V_{H} domain comprising an amino acid sequence at least 90% identical to the amino acid sequence set forth in SEQ ID NO:19 and/or a V_{L} domain comprising an amino acid sequence at least 90% identical to the amino acid sequence as set forth in SEQ ID NO:20 wherein said TIM-3 binding protein specifically binds to human TIM-3. In one embodiment, the TIM-3 binding protein is administered at a dose of 100 mg, 300 mg or 900 mg. In one embodiment, the ICOS binding protein is administered at a dose of 24 mg, 48 mg, 80 mg or 160 mg, and the TIM-3 binding protein is administered at a dose of 100 mg, 300 mg or 900 mg. In a further embodiment, the ICOS binding protein is administered at a dose of 24 mg, 48 mg, 80 mg or 160 mg, the PD-1 binding protein is administered at a dose of 500 mg or 1000 mg and the TIM-3 binding protein is administered at a dose of 100 mg, 300 mg or 900 mg. In one embodiment, the TIM-3 binding protein comprises one or more of: CDRH1 as set forth in SEQ ID NO:13; CDRH2 as set forth in SEQ ID NO:14; CDRH3 as set forth in SEQ ID NO:15; CDRL1 as set forth in SEQ ID NO:16; CDRL2 as set forth in SEQ ID NO:17 and/or CDRL3 as set forth in SEQ ID NO:18 or a direct equivalent of each CDR wherein a direct equivalent has no more than two amino acid substitutions in said CDR. In one embodiment, the TIM-3 binding protein comprises a heavy chain variable region comprising one or more of SEQ ID NO:13; SEQ ID NO:14; and SEQ ID NO:15 and wherein said TIM-3 binding protein comprises a light chain variable region comprising one or more of SEQ ID NO:16; SEQ ID NO:17, and SEQ ID NO:18. In one embodiment, the TIM-3 binding protein comprises a heavy chain variable region comprising SEQ ID NO:13; SEQ ID NO:14; and SEQ ID NO:15 and wherein said TIM-3 binding protein comprises a light chain variable region comprising SEQ ID NO:16; SEQ ID NO:17, and SEQ ID NO:18. In one embodiment, the TIM-3 binding protein comprises a V_{H} domain comprising the amino acid sequence set forth in SEQ ID NO:19 and a V_{L} domain comprising the amino acid sequence as set forth in SEQ ID NO:20. In one embodiment, the TIM-3 binding protein comprises a heavy chain comprising the amino acid sequence set forth in SEQ ID NO:22 and a light chain comprising the amino acid sequence as set forth in SEQ ID NO:21. In one embodiment, the TIM-3 binding protein is cobolimab.

In one aspect, there is provided a pharmaceutical kit comprising about 0.08 mg to about 240 mg of an ICOS binding protein, and a TIM-3 binding protein, wherein the ICOS binding protein comprises a V_{H} domain comprising an amino acid sequence at least 90% identical to the amino acid sequence set forth in SEQ ID NO:7 and/or a V_{L} domain comprising an amino acid sequence at least 90% identical to the amino acid sequence as set forth in SEQ ID NO:8 wherein said ICOS binding protein specifically binds to human ICOS. In another aspect, there is provided a pharmaceutical kit comprising about 0.08 mg to about 240 mg of an ICOS binding protein, a PD-1 binding protein and a TIM-3 binding protein, wherein the ICOS binding protein comprises a V_{H} domain comprising an amino acid sequence at least 90% identical to the amino acid sequence set forth in SEQ ID NO:7 and/or a V_{L} domain comprising an amino acid sequence at least 90% identical to the amino acid sequence as set forth in SEQ ID NO:8 wherein said ICOS binding protein specifically binds to human ICOS. In one embodiment, the kit comprises 24 mg, 48 mg, 80 mg or 160 mg of an ICOS binding protein. In one embodiment, the kit comprises about 5 mg to about 5000 mg of a TIM-3 binding protein. In one embodiment, the kit comprises 100 mg, 300 mg or 900 mg of a TIM-3 binding protein. In one embodiment, the kit comprises about 100 mg to about 2000 mg of a PD-1 binding protein. In one embodiment, the kit comprises 500 mg or 1000 mg of a PD-1 binding protein. In another embodiment, the kit comprises 24 mg, 48 mg, 80 mg or 160 mg of an ICOS binding protein, and 100 mg, 300 mg or 900 mg of a TIM-3 binding protein. In another embodiment, the kit comprises 24 mg, 48 mg, 80 mg or 160 mg of an ICOS binding protein, 500 mg or 1000 mg of a PD-1 binding protein and 100 mg, 300 mg or 900 mg of a TIM-3 binding protein. In one embodiment, the TIM-3 binding protein is cobolimab. In one embodiment, the PD-1 binding protein is dostarlimab. In one embodiment, the ICOS binding protein comprises one or more of: CDRH1 as set forth in SEQ ID NO:1; CDRH2 as set forth in SEQ ID NO:2; CDRH3 as set forth in SEQ ID NO:3; CDRL1 as set forth in SEQ ID NO:4; CDRL2 as set forth in SEQ ID NO:5 and/or CDRL3 as set forth in SEQ ID NO:6 or a direct equivalent of each CDR wherein a direct equivalent has no more than two amino acid substitutions in said CDR. In one embodiment, the ICOS binding protein comprises a heavy chain variable region comprising one or more of SEQ ID NO:1; SEQ ID NO:2; and SEQ ID NO:3 and wherein said ICOS binding protein comprises a light chain variable region comprising one or more of SEQ ID NO:4; SEQ ID NO:5, and SEQ ID NO:6. In one embodiment, the ICOS binding protein comprises a heavy chain variable region comprising SEQ ID NO:1; SEQ ID NO:2; and SEQ ID NO:3 and wherein said ICOS binding protein comprises a light chain variable region comprising SEQ ID NO:4; SEQ ID NO:5, and SEQ ID NO:6. In one embodiment, the ICOS binding protein comprises a V_{H} domain comprising the amino acid sequence set forth in SEQ ID NO:7 and a V_{L} domain comprising the amino acid sequence as set forth in SEQ ID NO:8. In one embodiment, the ICOS binding protein comprises a heavy chain comprising the amino acid sequence set forth in SEQ ID NO:9 and a light chain comprising the amino acid sequence as set forth in SEQ ID NO:10.

In one aspect, there is provided a pharmaceutical kit comprising about 5 mg to about 5000 mg of a TIM-3 binding protein, and an ICOS binding protein, wherein the TIM-3 binding protein comprises a V_{H} domain comprising an amino acid sequence at least 90% identical to the amino acid sequence set forth in SEQ ID NO:19 and/or a V_{L} domain comprising an amino acid sequence at least 90% identical to the amino acid sequence as set forth in SEQ ID NO:20 wherein said TIM-3 binding protein specifically binds to human TIM-3. In another aspect, there is provided a pharmaceutical kit comprising about 5 mg to about 5000 mg of a TIM-3 binding protein, an ICOS binding protein and a PD-1 binding protein, wherein the TIM-3 binding protein comprises a V_{H} domain comprising an amino acid sequence at least 90% identical to the amino acid sequence set forth in SEQ ID NO:19 and/or a V_{L} domain comprising an amino acid sequence at least 90% identical to the amino acid sequence as set forth in SEQ ID NO:20 wherein said TIM-3 binding protein specifically binds to human TIM-3. In one embodiment, the kit comprises about 0.08 mg to about 240 mg of an ICOS binding protein. In one embodiment, the kit comprises 24 mg, 48 mg, 80 mg or 160 mg of an ICOS binding protein. In one embodiment, the kit comprises 100 mg, 300 mg or 900 mg of a TIM-3 binding protein. In one embodiment, the kit comprises 500 mg or 1000 mg of a PD-1 binding protein. In another embodiment, the kit comprises 24 mg, 48 mg, 80 mg or 160 mg of an ICOS binding protein, and 100 mg, 300 mg or 900 mg of a TIM-3 binding protein. In another embodiment, the kit comprises 24 mg, 48 mg, 80 mg or 160 mg of an ICOS binding protein, 500 mg or 1000 mg of a PD-1 binding protein and 100 mg, 300 mg or 900 mg of a TIM-3 binding protein. In one embodiment, the TIM-3 binding protein comprises one or more of: CDRH1 as set forth in SEQ ID NO:13; CDRH2 as set forth in SEQ ID NO:14; CDRH3 as set forth in SEQ ID NO:15; CDRL1 as set forth in SEQ ID NO:16; CDRL2 as set forth in SEQ ID NO:17 and/or CDRL3 as set forth in SEQ ID NO:18 or a direct equivalent of each CDR wherein a direct equivalent has no more than two amino acid substitutions in said CDR. In one embodiment, the TIM-3 binding protein comprises a heavy chain variable region comprising one or more of SEQ ID NO:13; SEQ ID NO:14; and SEQ ID NO:15 and wherein said TIM-3 binding protein comprises a light chain variable region comprising one or more of SEQ ID NO:16; SEQ ID NO:17, and SEQ ID NO:18. In one embodiment, the TIM-3 binding protein comprises a heavy chain variable region comprising SEQ ID NO:13; SEQ ID NO:14; and SEQ ID NO:15 and wherein said TIM-3 binding protein comprises a light chain variable region comprising SEQ ID NO:16; SEQ ID NO:17, and SEQ ID NO:18. In one embodiment, the TIM-3 binding protein comprises a V_{H} domain comprising the amino acid sequence set forth in SEQ ID NO:19 and a V_{L} domain comprising the amino acid sequence as set forth in SEQ ID NO:20. In one embodiment, the TIM-3 binding protein comprises a heavy chain comprising the amino acid sequence set forth in SEQ ID NO:21 and a light chain comprising the amino acid sequence as set forth in SEQ ID NO:22. In one embodiment, the TIM-3 binding protein is cobolimab.

In one aspect, there is provided a pharmaceutical kit comprising about 100 mg to about 2000 mg of a PD-1 binding protein, an ICOS binding protein and a TIM-3 binding protein, wherein the PD-1 binding protein comprises a V_{H} domain comprising an amino acid sequence at least 90% identical to the amino acid sequence set forth in SEQ ID NO:30 and/or a V_{L} domain comprising an amino acid sequence at least 90% identical to the amino acid sequence as set forth in SEQ ID NO:31 wherein said PD-1 binding protein specifically binds to human PD-1. In one embodiment, the kit comprises 500 mg or 1000 mg of PD-1 binding protein. In one embodiment, the kit comprises about 0.08 mg to about 240 mg of an ICOS binding protein. In one embodiment, the kit comprises 24 mg, 48 mg, 80 mg or 160 mg of an ICOS binding protein. In one embodiment, the kit comprises 100 mg, 300 mg or 900 mg of a TIM-3 binding protein. In another embodiment, the kit comprises 100 mg, 300 mg or 900 mg of a TIM-3 binding protein, 24 mg, 48 mg, 80 mg or 160 mg of an ICOS binding protein and 500 mg or 1000 mg of a PD-1 binding protein. In one embodiment, the PD-1 binding protein comprises one or more of: CDRH1 as set forth in SEQ ID NO:24; CDRH2 as set forth in SEQ ID NO:25; CDRH3 as set forth in SEQ ID NO:26; CDRL1 as set forth in SEQ ID NO:27; CDRL2 as set forth in SEQ ID NO:28 and/or CDRL3 as set forth in SEQ ID NO:29 or a direct equivalent of each CDR wherein a direct equivalent has no more than two amino acid substitutions in said CDR. In one embodiment, the PD-1 binding protein comprises a heavy chain variable region comprising one or more of SEQ ID NO:24; SEQ ID NO:25; and SEQ ID NO:26 and wherein said PD-1 binding protein comprises a light chain variable region comprising one or more of SEQ ID NO:27; SEQ ID NO:28, and SEQ ID NO:29. In one embodiment, the PD-1 binding protein comprises a heavy chain variable region comprising SEQ ID NO:24; SEQ ID NO:25; and SEQ ID NO:26 and wherein said PD-1 binding protein comprises a light chain variable region comprising SEQ ID NO:27; SEQ ID NO:28, and SEQ ID NO:29. In one embodiment, the PD-1 binding protein comprises a V_{H} domain comprising the amino acid sequence set forth in SEQ ID NO:30 and a V_{L} domain comprising the amino acid sequence as set forth in SEQ ID NO:31. In one embodiment, the PD-1 binding protein comprises a heavy chain comprising the amino acid sequence set forth in SEQ ID NO:32 and a light chain comprising the amino acid sequence as set forth in SEQ ID NO:33. In one embodiment, the PD-1 binding protein is dostarlimab.

In one aspect, there is provided a method of treating cancer, the method comprising administering to a subject (e.g. a human) an ICOS binding protein at a dose wherein the median plasma concentration of the ICOS binding protein is between 100 μg/ml and 0.1 μg/ml for at least 7 days after the first dose. In one aspect, there is provided a method of treating cancer, the method comprising administering to a subject (e.g. a human) a TIM-3 binding protein at a dose wherein the median plasma concentration of the TIM-3 binding protein is between 120 μg/ml and 0.1 μg/ml for at least 7 days after the first dose.

In one aspect, there is provided an ICOS binding protein for use in the treatment of cancer, wherein ICOS binding protein is administered at a dose wherein the median plasma concentration of the ICOS binding protein is between 100 μg/ml and 0.1 μg/ml for at least 7 days after the first dose.

In another aspect, there is provided use of an ICOS binding protein in the manufacture of a medicament for treating cancer, wherein the ICOS binding protein is administered at a dose wherein the median plasma concentration of the ICOS binding protein is between 100 μg/ml and 0.1 μg/ml for at least 7 days after the first dose.

In one embodiment, the ICOS binding protein is administered at a dose wherein the median plasma concentration of the ICOS binding protein is between 100 μg/ml, 10 μg/ml, 1 μg/ml or 0.1 μg/ml and 10 μg/ml, 1 μg/ml or 0.1 μg/ml for at least 1, 2.5, 4.5, 7, 14 or 21 days after the first dose.

In one embodiment, the ICOS binding protein is administered at a dose wherein the median plasma concentration of the ICOS binding protein is between 100 μg/ml, 90 μg/ml, 80 μg/ml, 70 μg/ml, 60 μg/ml, 50 μg/ml, 40 μg/ml, 30 μg/ml, 20 μg/ml, 10 μg/ml, 9 μg/ml, 8 μg/ml, 7 μg/ml, 6 μg/ml, 5 μg/ml, 4 μg/ml, 3 μg/ml, 2 μg/ml, 1 μg/ml, 0.9 μg/ml, 0.8 μg/ml, 0.7 μg/ml, 0.6 μg/ml, 0.5 μg/ml, 0.4 μg/ml, 0.3 μg/ml or 0.2 μg/ml and 90 μg/ml, 80 μg/ml, 70 μg/ml, 60 μg/ml, 50 μg/ml, 40 μg/ml, 30 μg/ml, 20 μg/ml, 10 μg/ml, 9 μg/ml, 8 μg/ml, 7 μg/ml, 6 μg/ml, 5 μg/ml, 4 μg/ml, 3 μg/ml, 2 μg/ml, 1 μg/ml, 0.9 μg/ml, 0.8 μg/ml, 0.7 μg/ml, 0.6 μg/ml, 0.5 μg/ml, 0.4 μg/ml, 0.3 μg/ml, 0.2 μg/ml or 0.1 μg/ml, for at least 1, 2, 2.5, 3, 4, 4.5, 5, 6, 7, 8, 9, 10, 11, 12, 13, 14, 15, 16, 17, 18, 19, 20 or 21 days after the first dose.

In one embodiment, the TIM-3 binding protein is administered at a dose wherein the median plasma concentration of the TIM-3 binding protein is between 120 μg/ml, 110 μg/ml, 100 μg/ml, 90 μg/ml, 80 μg/ml, 70 μg/ml, 60 μg/ml, 50 μg/ml, 40 μg/ml, 30 μg/ml, 20 μg/ml, 10 μg/ml, 9 μg/ml, 8 μg/ml, 7 μg/ml, 6 μg/ml, 5 μg/ml, 4 μg/ml, 3 μg/ml, 2 μg/ml, 1 μg/ml, 0.9 μg/ml, 0.8 μg/ml, 0.7 μg/ml, 0.6 μg/ml, 0.5 μg/ml, 0.4 μg/ml, 0.3 μg/ml or 0.2 μg/ml and 120 μg/ml, 110 μg/ml, 100 μg/ml, 90 μg/ml, 80 μg/ml, 70 μg/ml, 60 μg/ml, 50 μg/ml, 40 μg/ml, 30 μg/ml, 20 μg/ml, 10 μg/ml, 9 μg/ml, 8 μg/ml, 7 μg/ml, 6 μg/ml, 5 μg/ml, 4 μg/ml, 3 μg/ml, 2 μg/ml, 1 μg/ml, 0.9 μg/ml, 0.8 μg/ml, 0.7 μg/ml, 0.6 μg/ml, 0.5 μg/ml, 0.4 μg/ml, 0.3 μg/ml, 0.2 μg/ml or 0.1 μg/ml, for at least 1, 2, 2.5, 3, 4, 4.5, 5, 6, 7, 8, 9, 10, 11, 12, 13, 14, 15, 16, 17, 18, 19, 20 or 21 days after the first dose.

In one embodiment, the human is administered an ICOS binding protein at a dose wherein the median plasma concentration of the ICOS binding protein is between 10 μg/ml and 1 μg/ml at 21 days after the first dose. In one embodiment, the human is administered an ICOS binding protein at a dose wherein the median plasma concentration of the ICOS binding protein is between 10 μg/ml and 0.1 μg/ml at 21 days after the first dose.

In one embodiment, the human is administered an ICOS binding protein at a dose wherein the median plasma concentration of the ICOS binding protein is between 100 μg/ml and 1 μg/ml at 21 days after the first dose. In one embodiment, the human is administered an ICOS binding protein at a dose wherein the median plasma concentration of the ICOS binding protein is between 100 μg/ml and 10 μg/ml at 21 days after the first dose.

In one aspect, there is provided a method of treating cancer, the method comprising administering to a subject (e.g. a human) an ICOS binding protein at a dose wherein ICOS receptor saturation or occupancy in the subject is at or above around 50% for at least 7 days after the first dose.

In one aspect, there is provided an ICOS binding protein for use in the treatment of cancer, wherein the ICOS binding protein is administered to a subject (e.g. a human) at a dose wherein ICOS receptor saturation or occupancy in the subject is at or above around 50% for at least 7 days after the first dose.

In another aspect, there is provided use of an ICOS binding protein in the manufacture of a medicament for treating cancer, wherein the ICOS binding protein is administered to a human at a dose wherein ICOS receptor saturation or occupancy in the human is at or above around 50% for at least 7 days after first dose.

In one embodiment, the human is administered an ICOS binding protein at a dose wherein ICOS receptor saturation or occupancy in the human is at or above around 50%, 55%, 60%, 65%, 70%, 75%, 80%, 85%, 90% or 95% for at least 1, 2, 3, 4, 5, 6, 7, 8, 9, 10, 11, 12, 13, 14, 15, 16, 17, 18, 19, 20 or 21 days after first dose.

In one aspect, there is provided a method of treating cancer, the method comprising administering to a subject (e.g. a human) an ICOS binding protein at a dose wherein peripheral CD4⁺ or CD8⁺ T cell receptor occupancy is at or above 50% for at least 7 days after the first dose.

In one aspect, there is provided an ICOS binding protein for use in the treatment of cancer, wherein the ICOS binding protein is administered to a human at a dose wherein peripheral CD4⁺ or CD8⁺ T cell receptor occupancy is at or above 50% for at least 7 days after the first dose.

In another aspect, there is provided use of an ICOS binding protein in the manufacture of a medicament for treating cancer, wherein the ICOS binding protein is administered to a human at a dose wherein peripheral CD4⁺ or CD8⁺ T cell receptor occupancy is at or above 50% for at least 7 days after the first dose.

Peak CD4⁺ Receptor Occupancy (RO) corresponds to the ICOS binding protein maximum plasma concentration. Peak CD8⁺ Receptor Occupancy (RO) corresponds to the ICOS binding protein maximum plasma concentration.

In one embodiment, the ICOS binding protein is administered at a dose wherein peripheral CD4⁺ or CD8⁺ T cell receptor occupancy is at or above around 50%, 55%, 60%, 65%, 70%, 75%, 80%, 85%, 90% or 95% for at least 1, 2, 3, 4, 5, 6, 7, 8, 9, 10, 11, 12, 13, 14, 15, 16, 17, 18, 19, 20 or 21 days after the first dose.

In one embodiment, the ICOS binding protein is administered at a dose wherein peripheral CD4⁺ or CD8⁺ T cell receptor occupancy is at or above around 60%, for at least 21 days after the first dose. In one embodiment, the ICOS binding protein is administered at a dose wherein peripheral CD4⁺ or CD8⁺ T cell receptor occupancy is at or above around 70%, for at least 21 days after the first dose. In one embodiment, the ICOS binding protein is administered at a dose wherein peripheral CD4⁺ or CD8⁺ T cell receptor occupancy is at or above around 80%, for at least 21 days after the first dose. In one embodiment, the ICOS binding protein is administered at a dose wherein peripheral CD4⁺ or CD8⁺ T cell receptor occupancy is at or above around 90%, for at least 21 days after the first dose.

In one aspect, there is provided a pharmaceutical composition comprising an ICOS binding protein, wherein said composition provides an Area Under the Curve (AUC) value of 37 mg/mL×day to 255 mg/mL×day of the ICOS binding protein after a single dose. In one embodiment, said composition further provides a TIM-3 binding protein. In one embodiment, said composition provides an AUC value of 62 mg/mL×day to 220 mg/mL×day of the ICOS binding protein after a single dose.

In one embodiment, diterpenoids, such as paclitaxel, nab-paclitaxel or docetaxel; vinca alkaloids, such as vinblastine, vincristine, or vinorelbine; platinum coordination complexes, such as cisplatin or carboplatin; nitrogen mustards such as cyclophosphamide, melphalan, or chlorambucil; alkyl sulfonates such as busulfan; nitrosoureas such as carmustine; triazenes such as dacarbazine; actinomycins such as dactinomycin; anthrocyclins such as daunorubicin or doxorubicin; bleomycins; epipodophyllotoxins such as etoposide or teniposide; antimetabolite anti-neoplastic agents such as fluorouracil, pemetrexed, methotrexate, cytarabine, mecaptopurine, thioguanine, or gemcitabine; methotrexate; camptothecins such as irinotecan or topotecan; rituximab; ofatumumab; trastuzumab; cetuximab; bexarotene; sorafenib; erbB inhibitors such as lapatinib, erlotinib or gefitinib; pertuzumab; ipilimumab; tremelimumab; nivolumab; pembrolizumab; FOLFOX; capecitabine; FOLFIRI; bevacizumab; atezolizumab; selicrelumab; obinotuzumab or any combinations thereof is/are further administered concurrently or sequentially with the ICOS binding protein and/or TIM-3 binding protein and/or the PD-1 binding protein.

In one embodiment, chemotherapy is further administered concurrently or sequentially with ICOS binding protein and/or the TIM-3 binding protein and/or PD-1 binding protein. In one embodiment, chemotherapy is further administered concurrently or sequentially with ICOS binding protein and the TIM-3 binding protein. In one embodiment, the chemotherapy is platinum-based chemotherapy. In one embodiment, the chemotherapy is platinum-based chemotherapy and fluorouracil. In one embodiment, the platinum-based chemotherapy is paclitaxel, nab-paclitaxel, docetaxel, cisplatin, carboplatin or any combination thereof. In one embodiment, the platinum-based chemotherapy is fluorouracil, cisplatin, carboplatin or any combination thereof. In one embodiment, chemotherapy is a platinum doublet of cisplatin or carboplatin with any one of pemetrexed, paclitaxel (or nab-paclitaxel), gemcitabine, or fluorouracil. In one embodiment, chemotherapy is further administered concurrently or sequentially with ICOS binding protein and the TIM-3 binding protein to TIM-3 binding protein naïve patients.

In one embodiment, the ICOS binding protein, TIM-3 binding protein and chemotherapy are administered every 3 weeks for 6 cycles and then the ICOS binding protein and TIM-3 binding protein is administered every 3 weeks for 35 cycles.

In one embodiment, the ICOS binding protein and the TIM-3 binding protein are administered concurrently or sequentially to TIM-3 positive patients.

In one embodiment, radiotherapy is further administered concurrently or sequentially with ICOS binding protein and/or the TIM-3 binding protein. In one embodiment, radiotherapy is further administered concurrently or sequentially with ICOS binding protein and/or the Tim-3 binding protein and/or the PD-1 binding protein. In some embodiments, the radiotherapy is selected from the group consisting of systemic radiation therapy, external beam radiation therapy, image-guided radiation therapy, tomotherapy, stereotactic radio surgery, stereotactic body radiation therapy, and proton therapy. In some embodiments, the radiotherapy comprises external-beam radiation therapy, internal radiation therapy (brachytherapy), or systemic radiation therapy. See, e.g., Amini et al., Radiat Oncol. “Stereotactic body radiation therapy (SBRT) for lung cancer patients previously treated with conventional radiotherapy: a review” 9:210 (2014); Baker et al., Radiat Oncol. “A critical review of recent developments in radiotherapy for non-small cell lung cancer” 11(1):115 (2016); Ko et al., Clin Cancer Res “The Integration of Radiotherapy with Immunotherapy for the Treatment of Non-Small Cell Lung Cancer” (24) (23) 5792-5806; and, Yamoah et al., Int J Radiat Oncol Biol Phys “Radiotherapy Intensification for Solid Tumors: A Systematic Review of Randomized Trials” 93(4): 737-745 (2015).

In some embodiments, the radiotherapy comprises external-beam radiation therapy, and the external bean radiation therapy comprises intensity-modulated radiation therapy (IMRT), image-guided radiation therapy (IGRT), tomotherapy, stereotactic radiosurgery, stereotactic body radiation therapy, proton therapy, or other charged particle beams.

In some embodiments, the radiotherapy comprises stereotactic body radiation therapy.

### Cancer

Combinations and methods of the invention may be used in the treatment of cancer. By the term “treating” and grammatical variations thereof as used herein, is meant therapeutic therapy. In reference to a particular condition, treating means: (1) to ameliorate, or lessen the severity of, the condition of one or more of the biological manifestations of the condition, (2) to interfere with (a) one or more points in the biological cascade that leads to or is responsible for the condition or (b) one or more of the biological manifestations of the condition, (3) to alleviate one or more of the symptoms or signs, effects or side effects associated with the condition or treatment thereof, (4) to slow the progression of the condition, that is to say prolong survival, or one or more of the biological manifestations of the condition and/or (5) to cure said condition or one or more of the biological manifestations of the condition by eliminating or reducing to undetectable levels one or more of the biological manifestations of the condition for a period of time considered to be a state of remission for that manifestation without additional treatment over the period of remission. One skilled in the art will understand the duration of time considered to be remission for a particular disease or condition. Prophylactic therapy is also contemplated thereby. The skilled artisan will appreciate that “prevention” is not an absolute term. In medicine, “prevention” is understood to refer to the prophylactic administration of a drug to substantially diminish the likelihood or severity of a condition or biological manifestation thereof, or to delay the onset of such condition or biological manifestation thereof. Prophylactic therapy is appropriate, for example, when a subject is considered at high risk for developing cancer, such as when a subject has a strong family history of cancer or when a subject has been exposed to a carcinogen.

As used herein, the terms “cancer”, “neoplasm”, “malignancy”, and “tumor” are used interchangeably and, in either the singular or plural form, refer to cells that have undergone a malignant transformation that makes them pathological to the host organism. Primary cancer cells can be readily distinguished from non-cancerous cells by well-established techniques, particularly histological examination. The definition of a cancer cell, as used herein, includes not only a primary cancer cell, but any cell derived from a cancer cell ancestor. This includes metastasized cancer cells, and in vitro cultures and cell lines derived from cancer cells. When referring to a type of cancer that normally manifests as a solid tumor, a “clinically detectable” tumor is one that is detectable on the basis of tumor mass; e.g. by procedures such as computed tomography (CT) scan, magnetic resonance imaging (MRI), X-ray, ultrasound or palpation on physical examination, and/or which is detectable because of the expression of one or more cancer-specific antigens in a sample obtainable from a patient.

In one aspect, the invention relates to a method for treating or lessening the severity of a cancer. In one embodiment, the cancer is selected from: brain cancer, glioblastomas, glioma (such as diffuse intrinsic pontine glioma), Bannayan-Zonana syndrome, Cowden disease, Lhermitte-Duclos disease, breast cancer (e.g. inflammatory breast cancer), Wilm's tumor, ependymoma, medulloblastoma, cardiac tumors, colon cancer, colorectal cancer, head and neck cancer (e.g. squamous cell carcinoma of the head and neck, cancer of the mouth (i.e. oral cancer), salivary gland cancer, buccal cancer, pharyngeal cancer, oropharyngeal cancer, nasopharangeal cancer, hypopharyngeal cancer, laryngeal cancer), eye cancer (e.g. retinoblastoma), lung cancer (e.g. non-small cell lung cancer, small cell cancer), liver cancer (i.e. hepatocellular cancer), skin cancer (e.g. basal cell carcinoma, merkel cell carcinoma, squamous cell carcinoma), melanoma, ovarian cancer, pancreatic cancer, bile duct cancer, gallbladder cancer, prostate cancer, sarcoma (e.g. soft tissue sarcoma, Ewing's sarcoma, Kaposi sarcoma, rhabdomyosarcoma), bone cancer, osteosarcoma, giant cell tumor of bone, thyroid cancer, parathyroid cancer, thymoma, blood cancer (which may be broadly categorised as leukemias, lymphomas or myelomas, and include examples such as lymphoblastic T-cell leukemia, chronic myelogenous leukemia, chronic lymphocytic leukemia, hairy-cell leukemia, acute lymphoblastic leukemia, acute myelogenous leukemia, chronic neutrophilic leukemia, acute lymphoblastic T-cell leukemia, plasmacytoma, immunoblastic large cell leukemia, mantle cell leukemia, multiple myeloma megakaryoblastic leukemia, multiple myeloma, acute megakaryocytic leukemia, promyelocytic leukemia, erythroleukemia, malignant lymphoma, Hodgkin's lymphoma, non-Hodgkin's lymphoma, lymphoblastic T cell lymphoma, Burkitt's lymphoma, and follicular lymphoma), neuroblastoma, pituitary tumor, adrenocortical cancer, anal cancer (i.e. rectal cancer), bladder cancer, urothelial cancer, urethral cancer, vaginal cancer, vulvar cancer, cervical cancer, endometrial cancer, uterine cancer, fallopian tube cancer, renal cancer (i.e. kidney cancer, e.g. renal cell carcinoma), mesothelioma (e.g. malignant pleural mesothelioma), esophageal cancer (e.g. esophageal squamous cell carcinoma), gastric cancer (i.e. stomach cancer), gastroinstestinal carcinoid tumor, GIST (gastrointestinal stromal tumor), appendicial cancer, penile cancer, testicular cancer, germ cell tumors.

In one embodiment, the cancer exhibits microsatellite instability (MSI). Microsatellite instability (“MSI”) is or comprises a change that in the DNA of certain cells (such as tumor cells) in which the number of repeats of microsatellites (short, repeated sequences of DNA) is different than the number of repeats that was contained in the DNA from which it was inherited. Microsatellite instability arises from a failure to repair replication-associated errors due to a defective DNA mismatch repair (MMR) system. This failure allows persistence of mismatch mutations all over the genome, but especially in regions of repetitive DNA known as microsatellites, leading to increased mutational load. It has been demonstrated that at least some tumors characterized by MSI-H have improved responses to certain anti-PD-1 agents (Le et al. (2015) N. Engl. J. Med. 372(26):2509-2520; Westdorp et al. (2016) Cancer Immunol. Immunother. 65(10): 1249-1259).

In some embodiments, a cancer has a microsatellite instability status of high microsatellite instability (e.g. MSI-H status). In some embodiments, a cancer has a microsatellite instability status of low microsatellite instability (e.g. MSI-L status). In some embodiments, a cancer has a microsatellite instability status of microsatellite stable (e.g. MSS status). In some embodiments microsatellite instability status is assessed by a next generation sequencing (NGS)-based assay, an immunohistochemistry (IHC)-based assay, and/or a PCR-based assay. In some embodiments, microsatellite instability is detected by NGS. In some embodiments, microsatellite instability is detected by IHC. In some embodiments, microsatellite instability is detected by PCR.

In some embodiments, the cancer is associated with a high tumor mutation burden (TMB). In some embodiments, the cancer is associated with high TMB and MSI-H. In some embodiments, the cancer is associated with high TMB and MSI-L or MSS. In some embodiments, the cancer is endometrial cancer associated with high TMB. In some related embodiments, the endometrial cancer is associated with high TMB and MSI-H. In some related embodiments, the endometrial cancer is associated with high TMB and MSI-L or MSS.

In some embodiments, a cancer is a mismatch repair deficient (dMMR) cancer. Microsatellite instability may arise from a failure to repair replication-associated errors due to a defective DNA mismatch repair (MMR) system. This failure allows persistence of mismatch mutations all over the genome, but especially in regions of repetitive DNA known as microsatellites, leading to increased mutational load that may improve responses to certain therapeutic agents.

In some embodiments, a cancer is a hypermutated cancer. In some embodiments, a cancer harbors a mutation in polymerase epsilon (POLE). In some embodiments, a cancer harbors a mutation in polymerase delta (POLD).

In some embodiments, a cancer is endometrial cancer (e.g. MSI-H or MSS/MSI-L endometrial cancer). In some embodiments, a cancer is a MSI-H cancer comprising a mutation in POLE or POLD (e.g. a MSI-H non-endometrial cancer comprising a mutation in POLE or POLD).

In some embodiments, the cancer is an advanced cancer. In some embodiments, the cancer is a metastatic cancer. In some embodiments, the cancer is a recurrent cancer (e.g. a recurrent gynecological cancer such as recurrent epithelial ovarian cancer, recurrent fallopian tube cancer, recurrent primary peritoneal cancer, or recurrent endometrial cancer). In one embodiment, the cancer is recurrent or advanced.

In one embodiment, the cancer is selected from: appendiceal cancer, bladder cancer, breast cancer, cervical cancer, colorectal cancer, endometrial cancer, esophageal cancer (in particular esophageal squamous cell carcinoma), fallopian tube cancer, gastric cancer, glioma (such as diffuse intrinsic pontine glioma), head and neck cancer (in particular head and neck squamous cell carcinoma and oropharyngeal cancer), leukemia (in particular acute lymphoblastic leukemia, acute myeloid leukemia) lung cancer (in particular non small cell lung cancer), lymphoma (in particular Hodgkin's lymphoma, non-Hodgkin's lymphoma), melanoma, mesothelioma (in particular malignant pleural mesothelioma), Merkel cell carcinoma, neuroblastoma, oral cancer, osteosarcoma, ovarian cancer, prostate cancer, renal cancer, salivary gland tumor, sarcoma (in particular Ewing's sarcoma or rhabdomyosarcoma) squamous cell carcinoma, soft tissue sarcoma, thymoma, thyroid cancer, urothelial cancer, uterine cancer, vaginal cancer, vulvar cancer or Wilms tumor. In a further embodiment, the cancer is selected from: appendiceal cancer, bladder cancer, cervical cancer, colorectal cancer, esophageal cancer, head and neck cancer, melanoma, mesothelioma, non-small-cell lung cancer, prostate cancer and urothelial cancer. In a further embodiment, the cancer is selected from cervical cancer, endometrial cancer, head and neck cancer (in particular head and neck squamous cell carcinoma and oropharyngeal cancer), lung cancer (in particular non small cell lung cancer), lymphoma (in particular non-Hodgkin's lymphoma), melanoma, oral cancer, thyroid cancer, urothelial cancer or uterine cancer. In another embodiment, the cancer is selected from head and neck cancer (in particular head and neck squamous cell carcinoma and oropharyngeal cancer), lung cancer (in particular non small cell lung cancer), urothelial cancer, melanoma or cervical cancer.

In one embodiment, the human has a solid tumor. In one embodiment, the solid tumor is advanced solid tumor. In one embodiment, the cancer is selected from head and neck cancer, squamous cell carcinoma of the head and neck (SCCHN or HNSCC), gastric cancer, melanoma, renal cell carcinoma (RCC), esophageal cancer, non-small cell lung carcinoma, prostate cancer, colorectal cancer, ovarian cancer and pancreatic cancer. In one embodiment, the cancer is selected from the group consisting of: colorectal cancer, cervical cancer, bladder cancer, urothelial cancer, head and neck cancer, melanoma, mesothelioma, non-small cell lung carcinoma, prostate cancer, esophageal cancer, and esophageal squamous cell carcinoma. In one aspect the human has one or more of the following: SCCHN, colorectal cancer, esophageal cancer, cervical cancer, bladder cancer, breast cancer, head and neck cancer, ovarian cancer, melanoma, renal cell carcinoma (RCC), esophageal squamous cell carcinoma, non-small cell lung carcinoma, mesothelioma (e.g. pleural malignant mesothelioma), and prostate cancer.

In another aspect the human has a liquid tumor such as diffuse large B cell lymphoma (DLBCL), multiple myeloma, chronic lymphoblastic leukemia, follicular lymphoma, acute myeloid leukemia and chronic myelogenous leukemia.

In one embodiment, the cancer is head and neck cancer. In one embodiment, the cancer is HNSCC. Squamous cell carcinoma is a cancer that arises from particular cells called squamous cells. Squamous cells are found in the outer layer of skin and in the mucous membranes, which are the moist tissues that line body cavities such as the airways and intestines. Head and neck squamous cell carcinoma (HNSCC) develops in the mucous membranes of the mouth, nose, and throat. HNSCC is also known as SCCHN and squamous cell carcinoma of the head and neck.

HNSCC can occur in the mouth (oral cavity), the middle part of the throat near the mouth (oropharynx), the space behind the nose (nasal cavity and paranasal sinuses), the upper part of the throat near the nasal cavity (nasopharynx), the voicebox (larynx), or the lower part of the throat near the larynx (hypopharynx). Depending on the location, the cancer can cause abnormal patches or open sores (ulcers) in the mouth and throat, unusual bleeding or pain in the mouth, sinus congestion that does not clear, sore throat, earache, pain when swallowing or difficulty swallowing, a hoarse voice, difficulty breathing, or enlarged lymph nodes.

HNSCC can metastasize to other parts of the body, such as the lymph nodes, lungs or liver.

Tobacco use and alcohol consumption are the two most important risk factors for the development of HNSCC, and their contributions to risk are synergistic. In addition, the human papillomavirus (HPV), especially HPV-16, is now a well-established independent risk factor. Patients with HNSCC have a relatively poor prognosis. Recurrent/metastatic (R/M) HNSCC is especially challenging, regardless of human papillomavirus (HPV) status, and currently, few effective treatment options are available in the art. HPV-negative HNSCC is associated with a locoregional relapse rate of 19-35% and a distant metastatic rate of 14-22% following standard of care, compared with rates of 9-18% and 5-12%, respectively, for HPV-positive HNSCC. The median overall survival for patients with R/M disease is 10-13 months in the setting of first-line chemotherapy and 6 months in the second-line setting. The current standard of care is platinum-based doublet chemotherapy with or without cetuximab. Second-line standard of care options include cetuximab, methotrexate, and taxanes. All of these chemotherapeutic agents are associated with significant side effects, and only 10-13% of patients respond to treatment. HNSCC regressions from existing systemic therapies are transient and do not add significantly increased longevity, and virtually all patients succumb to their malignancy.

In one embodiment, the cancer is head and neck cancer. In one embodiment the cancer is head and neck squamous cell carcinoma (HNSCC). In one embodiment, the cancer is recurrent/metastatic (R/M) HNSCC. In one embodiment, the cancer is recurring/refractory (R/R) HNSCC. In one embodiment, the cancer is HPV-negative or HPV-positive HNSCC. In one embodiment, the cancer is a locally advanced HNSCC.

In one embodiment, the head and neck cancer is oropharyngeal cancer. In one embodiment, the head and neck cancer is an oral cancer (i.e. a mouth cancer).

In one embodiment, the cancer is lung cancer. In some embodiments, the lung cancer is a squamous cell carcinoma of the lung. In some embodiments, the lung cancer is small cell lung cancer (SCLC). In some embodiments, the lung cancer is non-small cell lung cancer (NSCLC), such as squamous NSCLC. In some embodiments, the lung cancer is an ALK-translocated lung cancer (e.g. ALK-translocated NSCLC). In some embodiments, the cancer is NSCLC with an identified ALK translocation. In some embodiments, the lung cancer is an EGFR-mutant lung cancer (e.g. EGFR-mutant NSCLC). In some embodiments, the cancer is NSCLC with an identified EGFR mutation.

In one embodiment, the cancer is melanoma. In some embodiments, the melanoma is an advanced melanoma. In some embodiments, the melanoma is a metastatic melanoma. In some embodiments, the melanoma is a MSI-H melanoma. In some embodiments, the melanoma is a MSS melanoma. In some embodiments, the melanoma is a POLE-mutant melanoma. In some embodiments, the melanoma is a POLD-mutant melanoma. In some embodiments, the melanoma is a high TMB melanoma.

In one embodiment, the cancer is colorectal cancer. In some embodiments, the colorectal cancer is an advanced colorectal cancer. In some embodiments, the colorectal cancer is a metastatic colorectal cancer. In some embodiments, the colorectal cancer is a MSI-H colorectal cancer. In some embodiments, the colorectal cancer is a MSS colorectal cancer. In some embodiments, the colorectal cancer is a POLE-mutant colorectal cancer. In some embodiments, the colorectal cancer is a POLD-mutant colorectal cancer. In some embodiments, the colorectal cancer is a high TMB colorectal cancer.

In some embodiments, the cancer is a gynecologic cancer (i.e. a cancer of the female reproductive system such as ovarian cancer, fallopian tube cancer, cervical cancer, vaginal cancer, vulvar cancer, uterine cancer, or primary peritoneal cancer, or breast cancer). In some embodiments, cancers of the female reproductive system include, but are not limited to, ovarian cancer, cancer of the fallopian tube(s), peritoneal cancer, and breast cancer.

In some embodiments, the cancer is ovarian cancer (e.g. serous or clear cell ovarian cancer). In some embodiments, the cancer is fallopian tube cancer (e.g. serous or clear cell fallopian tube cancer). In some embodiments, the cancer is primary peritoneal cancer (e.g. serous or clear cell primary peritoneal cancer).

In some embodiments, the ovarian cancer is an epithelial carcinoma. Epithelial carcinomas make up 85% to 90% of ovarian cancers. While historically considered to start on the surface of the ovary, new evidence suggests at least some ovarian cancer begins in special cells in a part of the fallopian tube. The fallopian tubes are small ducts that link a woman's ovaries to her uterus that are a part of a woman's reproductive system. In a normal female reproductive system, there are two fallopian tubes, one located on each side of the uterus. Cancer cells that begin in the fallopian tube may go to the surface of the ovary early on. The term “ovarian cancer” is often used to describe epithelial cancers that begin in the ovary, in the fallopian tube, and from the lining of the abdominal cavity, call the peritoneum. In some embodiments, the cancer is or comprises a germ cell tumor. Germ cell tumors are a type of ovarian cancer develops in the egg-producing cells of the ovaries. In some embodiments, a cancer is or comprises a stromal tumor. Stromal tumors develop in the connective tissue cells that hold the ovaries together, which sometimes is the tissue that makes female hormones called estrogen. In some embodiments, the cancer is or comprises a granulosa cell tumor.

Granulosa cell tumors may secrete estrogen resulting in unusual vaginal bleeding at the time of diagnosis. In some embodiments, a gynecologic cancer is associated with homologous recombination repair deficiency/homologous repair deficiency (HRD) and/or BRCA1/2 mutation(s). In some embodiments, a gynecologic cancer is platinum-sensitive. In some embodiments, a gynecologic cancer has responded to a platinum-based therapy. In some embodiments, a gynecologic cancer has developed resistance to a platinum-based therapy. In some embodiments, a gynecologic cancer has at one time shown a partial or complete response to platinum-based therapy (e.g. a partial or complete response to the last platinum-based therapy or to the penultimate platinum-based therapy). In some embodiments, a gynecologic cancer is now resistant to platinum-based therapy.

In some embodiments, the cancer is breast cancer. Usually breast cancer either begins in the cells of the milk producing glands, known as the lobules, or in the ducts. Less commonly breast cancer can begin in the stromal tissues. These include the fatty and fibrous connective tissues of the breast. Over time the breast cancer cells can invade nearby tissues such the underarm lymph nodes or the lungs in a process known as metastasis. The stage of a breast cancer, the size of the tumor and its rate of growth are all factors which determine the type of treatment that is offered. Treatment options include surgery to remove the tumor, drug treatment which includes chemotherapy and hormonal therapy, radiation therapy and immunotherapy. The prognosis and survival rate varies widely; the five year relative survival rates vary from 98% to 23% depending on the type of breast cancer that occurs. Breast cancer is the second most common cancer in the world with approximately 1.7 million new cases in 2012 and the fifth most common cause of death from cancer, with approximately 521,000 deaths. Of these cases, approximately 15% are triple-negative, which do not express the estrogen receptor, progesterone receptor (PR) or HER2. In some embodiments, triple negative breast cancer (TNBC) is characterized as breast cancer cells that are estrogen receptor expression negative (<1% of cells), progesterone receptor expression negative (<1% of cells), and HER2-negative.

In some embodiments, the cancer is estrogen receptor (ER)-positive breast cancer, ER-negative breast cancer, PR-positive breast cancer, PR-negative breast cancer, HER2-positive breast cancer, HER2-negative breast cancer, BRCA1/2-positive breast cancer, BRCA1/2-negative cancer, or TNBC. In some embodiments, the breast cancer is a metastatic breast cancer. In some embodiments, the breast cancer is an advanced breast cancer. In some embodiments, the cancer is a stage II, stage III or stage IV breast cancer. In some embodiments, the cancer is a stage IV breast cancer. In some embodiments, the breast cancer is a triple negative breast cancer.

In one embodiment, the cancer is endometrial cancer. Endometrial carcinoma is the most common cancer of the female genital, tract accounting for 10-20 per 100,000 person-years. The annual number of new cases of endometrial cancer (EC) is estimated at about 325 thousand worldwide. Further, EC is the most commonly occurring cancer in post-menopausal women. About 53% of endometrial cancer cases occur in developed countries. In 2015, approximately 55,000 cases of EC were diagnosed in the U.S. and no targeted therapies are currently approved for use in EC. There is a need for agents and regimens that improve survival for advanced and recurrent EC in 1L and 2L settings. Approximately 10,170 people are predicted to die from EC in the U.S. in 2016. The most common histologic form is endometrioid adenocarcinoma, representing about 75-80% of diagnosed cases. Other histologic forms include uterine papillary serous (less than 10%), clear cell 4%, mucinous 1%, squamous less than 1% and mixed about 10%.

From the pathogenetic point of view, EC falls into two different types, so-called types I and II. Type I tumors are low-grade and estrogen-related endometrioid carcinomas (EEC) while type II are non-endometrioid (NEEC) (mainly serous and clear cell) carcinomas. The World Health Organization has updated the pathologic classification of EC, recognizing nine different subtypes of EC, but EEC and serous carcinoma (SC) account for the vast majority of cases. EECs are estrogen-related carcinomas, which occur in perimenopausal patients, and are preceded by precursor lesions (endometrial hyperplasia/endometrioid intraepithelial neoplasia). Microscopically, lowgrade EEC (EEC 1-2) contains tubular glands, somewhat resembling the proliferative endometrium, with architectural complexity with fusion of the glands and cribriform pattern. High-grade EEC shows solid pattern of growth. In contrast, SC occurs in postmenopausal patients in absence of hyperestrogenism. At the microscope, SC shows thick, fibrotic or edematous papillae with prominent stratification of tumor cells, cellular budding, and anaplastic cells with large, eosinophilic cytoplasms. The vast majority of EEC are low grade tumors (grades 1 and 2), and are associated with good prognosis when they are restricted to the uterus. Grade 3 EEC (EEC3) is an aggressive tumor, with increased frequency of lymph node metastasis. SCs are very aggressive, unrelated to estrogen stimulation, mainly occurring in older women. EEC 3 and SC are considered high-grade tumors. SC and EEC3 have been compared using the surveillance, epidemiology and End Results (SEER) program data from 1988 to 2001. They represented 10% and 15% of EC respectively, but accounted for 39% and 27% of cancer death respectively. Endometrial cancers can also be classified into four molecular subgroups: (1) ultramutated/POLE-mutant; (2) hypermutated MSI+ (e.g., MSI-H or MSI-L); (3) copy number low/micro satellite stable (MSS); and (4) copy number high/serous-like. Approximately 28% of cases are MSI-high. (Murali, Lancet Oncol. (2014). In some embodiments, the patient has a mismatch repair deficient subset of 2L endometrial cancer. In some embodiments, the endometrial cancer is metastatic endometrial cancer. In some embodiments, the patient has a MSS endometrial cancer. In some embodiments, the patient has a MSI-H endometrial cancer.

In one embodiment, the cancer is cervical cancer. In some embodiments, the cervical cancer is an advanced cervical cancer. In some embodiments, the cervical cancer is a metastatic cervical cancer. In some embodiments, the cervical cancer is a MSI-H cervical cancer. In some embodiments, the cervical cancer is a MSS cervical cancer. In some embodiments, the cervical cancer is a POLE-mutant cervical cancer. In some embodiments, the cervical cancer is a POLD-mutant cervical cancer. In some embodiments, the cervical cancer is a high TMB cervical cancer.

In one embodiment, the cancer is uterine cancer. In some embodiments, the uterine cancer is an advanced uterine cancer. In some embodiments, the uterine cancer is a metastatic uterine cancer. In some embodiments, the uterine cancer is a MSI-H uterine cancer. In some embodiments, the uterine cancer is a MSS uterine cancer. In some embodiments, the uterine cancer is a POLE-mutant uterine cancer. In some embodiments, the uterine cancer is a POLD-mutant uterine cancer. In some embodiments, the uterine cancer is a high TMB uterine cancer.

In one embodiment, the cancer is urothelial cancer. In some embodiments, the urothelial cancer is an advanced urothelial cancer. In some embodiments, the urothelial cancer is a metastatic urothelial cancer. In some embodiments, the urothelial cancer is a MSI-H urothelial cancer. In some embodiments, the urothelial cancer is a MSS urothelial cancer. In some embodiments, the urothelial cancer is a POLE-mutant urothelial cancer. In some embodiments, the urothelial cancer is a POLD-mutant urothelial cancer. In some embodiments, the urothelial cancer is a high TMB urothelial cancer.

In one embodiment, the cancer is thyroid cancer. In some embodiments, the thyroid cancer is an advanced thyroid cancer. In some embodiments, the thyroid cancer is a metastatic thyroid cancer. In some embodiments, the thyroid cancer is a MSI-H thyroid cancer. In some embodiments, the thyroid cancer is a MSS thyroid cancer. In some embodiments, the thyroid cancer is a POLE-mutant thyroid cancer. In some embodiments, the thyroid cancer is a POLD-mutant thyroid cancer. In some embodiments, the thyroid cancer is a high TMB thyroid cancer.

Tumors may be a hematopoietic (or hematologic or hematological or blood-related) cancer, for example, cancers derived from blood cells or immune cells, which may be referred to as “liquid tumors”. Specific examples of clinical conditions based on hematologic tumors include leukemias such as chronic myelocytic leukemia, acute myelocytic leukemia, chronic lymphocytic leukemia and acute lymphocytic leukemia; plasma cell malignancies such as multiple myeloma, monoclonal gammopathy of undetermined (or unknown or unclear) significance (MGUS) and Waldenstrom's macroglobulinemia; lymphomas such as non-Hodgkin's lymphoma, Hodgkin's lymphoma, and the like.

The cancer may be any cancer in which an abnormal number of blast cells or unwanted cell proliferation is present or that is diagnosed as a hematological cancer, including both lymphoid and myeloid malignancies. Myeloid malignancies include, but are not limited to, acute myeloid (or myelocytic or myelogenous or myeloblastic) leukemia (undifferentiated or differentiated), acute promyeloid (or promyelocytic or promyelogenous or promyeloblastic) leukemia, acute myelomonocytic (or myelomonoblastic) leukemia, acute monocytic (or monoblastic) leukemia, erythroleukemia and megakaryocytic (or megakaryoblastic) leukemia. These leukemias may be referred together as acute myeloid (or myelocytic or myelogenous) leukemia. Myeloid malignancies also include myeloproliferative disorders (MPD) which include, but are not limited to, chronic myelogenous (or myeloid or myelocytic) leukemia (CML), chronic myelomonocytic leukemia (CMML), essential thrombocythemia (or thrombocytosis), and polcythemia vera (PCV). Myeloid malignancies also include myelodysplasia (or myelodysplastic syndrome or MDS), which may be referred to as refractory anemia (RA), refractory anemia with excess blasts (RAEB), and refractory anemia with excess blasts in transformation (RAEBT); as well as myelofibrosis (MFS) with or without agnogenic myeloid metaplasia.

In one embodiment, the cancer is non-Hodgkin's lymphoma. Hematopoietic cancers also include lymphoid malignancies, which may affect the lymph nodes, spleens, bone marrow, peripheral blood, and/or extranodal sites. Lymphoid cancers include B-cell malignancies, which include, but are not limited to, B-cell non-Hodgkin's lymphomas (B-NHLs). B-NHLs may be indolent (or low-grade), intermediate-grade (or aggressive) or high-grade (very aggressive). Indolent B cell lymphomas include follicular lymphoma (FL); small lymphocytic lymphoma (SLL); marginal zone lymphoma (MZL) including nodal MZL, extranodal MZL, splenic MZL and splenic MZL with villous lymphocytes; lymphoplasmacytic lymphoma (LPL); and mucosa-associated-lymphoid tissue (MALT or extranodal marginal zone) lymphoma. Intermediate-grade B-NHLs include mantle cell lymphoma (MCL) with or without leukemic involvement, diffuse large B cell lymphoma (DLBCL), follicular large cell (or grade 3 or grade 3B) lymphoma, and primary mediastinal lymphoma (PML). High-grade B-NHLs include Burkitt's lymphoma (BL), Burkitt-like lymphoma, small non-cleaved cell lymphoma (SNCCL) and lymphoblastic lymphoma. Other B-NHLs include immunoblastic lymphoma (or immunocytoma), primary effusion lymphoma, HIV associated (or AIDS related) lymphomas, and post-transplant lymphoproliferative disorder (PTLD) or lymphoma. B-cell malignancies also include, but are not limited to, chronic lymphocytic leukemia (CLL), prolymphocytic leukemia (PLL), Waldenstrom's macroglobulinemia (WM), hairy cell leukemia (HCL), large granular lymphocyte (LGL) leukemia, acute lymphoid (or lymphocytic or lymphoblastic) leukemia, and Castleman's disease. NHL may also include T-cell non-Hodgkin's lymphomas (T-NHLs), which include, but are not limited to T-cell non-Hodgkin's lymphoma not otherwise specified (NOS), peripheral T-cell lymphoma (PTCL), anaplastic large cell lymphoma (ALCL), angioimmunoblastic lymphoid disorder (AILD), nasal natural killer (NK) cell/T-cell lymphoma, gamma/delta lymphoma, cutaneous T cell lymphoma, mycosis fungoides, and Sezary syndrome.

Hematopoietic cancers also include Hodgkin's lymphoma (or disease) including classical Hodgkin's lymphoma, nodular sclerosing Hodgkin's lymphoma, mixed cellularity Hodgkin's lymphoma, lymphocyte predominant (LP) Hodgkin's lymphoma, nodular LP Hodgkin's lymphoma, and lymphocyte depleted Hodgkin's lymphoma. Hematopoietic cancers also include plasma cell diseases or cancers such as multiple myeloma (MM) including smoldering MM, monoclonal gammopathy of undetermined (or unknown or unclear) significance (MGUS), plasmacytoma (bone, extramedullary), lymphoplasmacytic lymphoma (LPL), Waldenstrom's Macroglobulinemia, plasma cell leukemia, and primary amyloidosis (AL). Hematopoietic cancers may also include other cancers of additional hematopoietic cells, including polymorphonuclear leukocytes (or neutrophils), basophils, eosinophils, dendritic cells, platelets, erythrocytes and natural killer cells. Tissues which include hematopoietic cells referred herein to as “hematopoietic cell tissues” include bone marrow; peripheral blood; thymus; and peripheral lymphoid tissues, such as spleen, lymph nodes, lymphoid tissues associated with mucosa (such as the gut-associated lymphoid tissues), tonsils, Peyer's patches and appendix, and lymphoid tissues associated with other mucosa, for example, the bronchial linings.

In one embodiment, the treatment is first-line or second line treatment of HNSCC. In one embodiment, the treatment is first-line or second line treatment of recurrent/metastatic HNSCC. In one embodiment the treatment is first line treatment of recurrent/metastatic (1L R/M) HNSCC.

In some embodiments, the treatment of cancer is first-line treatment of cancer. In one embodiment, the treatment of cancer is second-line treatment of cancer. In some embodiments, the treatment is third-line treatment of cancer. In some embodiments, the treatment is fourth-line treatment of cancer. In some embodiments, the treatment is fifth-line treatment of cancer.

In some embodiments, prior treatment to said second-line, third-line, fourth-line or fifth-line treatment of cancer comprises one or more of radiotherapy, chemotherapy, surgery or radiochemotherapy.

In one embodiment, the prior treatment comprises treatment with diterpenoids, such as paclitaxel, nab-paclitaxel or docetaxel; vinca alkaloids, such as vinblastine, vincristine, or vinorelbine; platinum coordination complexes, such as cisplatin or carboplatin; nitrogen mustards such as cyclophosphamide, melphalan, or chlorambucil; alkyl sulfonates such as busulfan; nitrosoureas such as carmustine; triazenes such as dacarbazine; actinomycins such as dactinomycin; anthrocyclins such as daunorubicin or doxorubicin; bleomycins; epipodophyllotoxins such as etoposide or teniposide; antimetabolite anti-neoplastic agents such as fluorouracil, methotrexate, cytarabine, mecaptopurine, thioguanine, or gemcitabine; methotrexate; camptothecins such as irinotecan or topotecan; rituximab; ofatumumab; trastuzumab; cetuximab; bexarotene; sorafenib; erbB inhibitors such as lapatinib, erlotinib or gefitinib; pertuzumab; ipilimumab; nivolumab; FOLFOX; capecitabine; FOLFIRI; bevacizumab; atezolizumab; selicrelumab; obinotuzumab or any combinations thereof. In one embodiment, prior treatment to said second line treatment, third-line, fourth-line or fifth-line treatment of cancer comprises ipilimumab and nivolumab. In one embodiment, prior treatment to said second line treatment, third-line, fourth-line or fifth-line treatment of cancer comprises FOLFOX, capecitabine, FOLFIRI/bevacizumab and atezolizumab/selicrelumab. In one embodiment, prior treatment to said second line treatment, third-line, fourth-line or fifth-line treatment of cancer comprises carboplatin/Nab-paclitaxel. In one embodiment, prior treatment to said second line treatment, third-line, fourth-line or fifth-line treatment of cancer comprises nivolumab and electrochemotherapy. In one embodiment, prior treatment to said second line treatment, third-line, fourth-line or fifth-line treatment of cancer comprises radiotherapy, cisplatin and carboplatin/paclitaxel.

In one embodiment, the treatment is first-line or second line treatment of head and neck cancer (in particular head and neck squamous cell carcinoma and oropharyngeal cancer). In one embodiment, the treatment is first-line or second line treatment of recurrent/metastatic HNSCC. In one embodiment the treatment is first line treatment of recurrent/metastatic (1L R/M) HNSCC.

In some embodiments, the treatment results in one or more of increased tumor infiltrating lymphocytes including cytotoxic T cells, helper T cell and NK cells, increased T cells, increased granzyme B+ cells, reduced proliferating tumor cells and increased activated T cells as compared to levels prior to treatment (e.g. baseline level). Activated T cells may be observed by greater OX40 and human leukocyte antigen DR expression.

In one embodiment, the methods of the present invention further comprise administering at least one neo-plastic agent or cancer adjuvant to said human. The methods of the present invention may also be employed with other therapeutic methods of cancer treatment.

Typically, any anti-neoplastic agent or cancer adjuvant that has activity versus a tumor, such as a susceptible tumor being treated may be co-administered in the treatment of cancer in the present invention. Examples of such agents can be found in Cancer Principles and Practice of Oncology by V. T. Devita, T. S. Lawrence, and S. A. Rosenberg (editors), 10^{th} edition (Dec. 5, 2014), Lippincott Williams & Wilkins Publishers.

In one embodiment, the human has previously been treated with one or more different cancer treatment modalities. In some embodiments, at least some of the patients in the cancer patient population have previously been treated with one or more therapies, such as surgery, radiotherapy, chemotherapy or immunotherapy. In some embodiments, at least some of the patients in the cancer patient population have previously been treated with chemotherapy (e.g. platinum-based chemotherapy). For example, a patient who has received two lines of cancer treatment can be identified as a 2L cancer patient (e.g. a 2L NSCLC patient). In some embodiments, a patient has received two lines or more lines of cancer treatment (e.g. a 2L+ cancer patient such as a 2L+ endometrial cancer patient). In some embodiments, a patient has not been previously treated with an antibody therapy, such as an anti-PD-1 therapy. In some embodiments, a patient previously received at least one line of cancer treatment (e.g. a patient previously received at least one line or at least two lines of cancer treatment). In some embodiments, a patient previously received at least one line of treatment for metastatic cancer (e.g. a patient previously received one or two lines of treatment for metastatic cancer). In some embodiments, a subject is resistant to treatment with an agent that inhibits PD-1. In some embodiments, a subject is refractory to treatment with an agent that inhibits PD-1. In some embodiments, a method described herein sensitizes the subject to treatment with an agent that inhibits PD-1.

It will be noted that embodiments of the method of treatment of cancer are also taken as embodiments of the ICOS binding protein and/or TIM-3 binding protein (and optionally PD-1 binding protein) for use in the treatment of cancer or use of an ICOS binding protein and/or TIM-3 binding protein (and optionally PD-1 binding protein) in the manufacture of a medicament for treating cancer and reciprocals thereof, in so far as it relates to dosages, treatment regimens and effects of said dosages and treatment regimens. It will also be noted that embodiments of the method of treatment of cancer, the ICOS binding protein and/or TIM-3 binding protein (and optionally PD-1 binding protein) for use in the treatment of cancer or use of an ICOS binding protein and/or TIM-3 binding protein (and optionally PD-1 binding protein) in the manufacture of a medicament for treating cancer are also taken as embodiments of the pharmaceutical composition, pharmaceutical formulation or pharmaceutical kit in so far as it relates to dosages, treatment regimens and effects of said dosages and treatment regimens.

### Pharmaceutical Compositions/Routes of Administration/Dosages

Antigen binding proteins as described herein may be incorporated into pharmaceutical compositions for use in the treatment of the human diseases described herein. In one embodiment, the pharmaceutical composition comprises an antigen binding protein in combination with one or more pharmaceutically acceptable carriers and/or excipients.

Such compositions comprise a pharmaceutically acceptable carrier as known and called for by acceptable pharmaceutical practice.

Pharmaceutical compositions may be administered by injection or continuous infusion (examples include, but are not limited to, intravenous, intraperitoneal, intradermal, subcutaneous, intramuscular, intraocular, and intraportal). In one embodiment, the composition is suitable for intravenous administration. Pharmaceutical compositions may be suitable for topical administration (which includes, but is not limited to, epicutaneous, inhaled, intranasal or ocular administration) or enteral administration (which includes, but is not limited to, oral, vaginal, or rectal administration).

Pharmaceutical formulations may be presented in unit dose forms containing a predetermined amount of active ingredient per unit dose. As is known to those skilled in the art, the amount of active ingredient per dose will depend on the condition being treated, the route of administration and the age, weight and condition of the patient.

It is especially advantageous to formulate parenteral compositions in dosage unit form for ease of administration and uniformity of dosage. Dosage unit form as used herein refers to physically discrete units suited as unitary dosages for the mammalian subjects to be treated; each unit containing a predetermined quantity of active compound calculated to produce the desired therapeutic effect in association with the required pharmaceutical carrier.

The pharmaceutical composition may be included in a kit containing the antigen binding proteins together with other medicaments, and/or with instructions for use. For convenience, the kit may comprise the reagents in predetermined amounts with instructions for use. The kit may also include devices used for administration of the pharmaceutical composition.

The terms “individual”, “subject” and “patient” are used herein interchangeably. In one embodiment, the subject is an animal. In another embodiment, the subject is a mammal, such as a primate, for example a marmoset or monkey. In another embodiment, the subject is a human (i.e. a human patient). “Subject” is defined broadly to include any patient in need of treatment, for example, a patient in need of cancer treatment. The subject in need of cancer treatment may include patients from a variety of stages including newly diagnosed, relapsed, refractory, progressive disease, remission, and others. The subject in need of cancer treatment may also include patients who have undergone stem cell transplant or who are considered transplant ineligible.

Subjects may be pre-screened in order to be selected for treatment with the combinations described herein. In one embodiment, a sample from the subject is tested for expression of TIM-3 prior to treatment with the combinations described herein.

### Kits

In some aspects, the invention provides a kit comprising:

(i) an ICOS binding protein comprising a heavy chain amino acid sequence comprising a CDRH1 of SEQ ID NO:1, a CDRH2 of SEQ ID NO:2, and a CDRH3 of SEQ ID NO:3, and a light chain amino acid sequence comprising a CDRL1 of SEQ ID NO:4, a CDRL2 of SEQ ID NO:5, and a CDRL3 of SEQ ID NO:6;

(ii) a TIM-3 binding protein comprising a heavy chain amino acid sequence comprising a CDRH1 of SEQ ID NO:13, a CDRH2 of SEQ ID NO:14, and a CDRH3 of SEQ ID NO:15, and a light chain amino acid sequence comprising a CDRL1 of SEQ ID NO:16, a CDRL2 of SEQ ID NO:17, and a CDRL3 of SEQ ID NO:18; and alternatively comprising

(iii) instructions for using (i) and (ii) in combination in the treatment of a cancer in a human.

In some aspects, the invention provides a kit comprising:

(i) an ICOS binding protein comprising a heavy chain amino acid sequence comprising a CDRH1 of SEQ ID NO:1, a CDRH2 of SEQ ID NO:2, and a CDRH3 of SEQ ID NO:3, and a light chain amino acid sequence comprising a CDRL1 of SEQ ID NO:4, a CDRL2 of SEQ ID NO:5, and a CDRL3 of SEQ ID NO:6;

(ii) a TIM-3 binding protein comprising a heavy chain amino acid sequence comprising a CDRH1 of SEQ ID NO:13, a CDRH2 of SEQ ID NO:14, and a CDRH3 of SEQ ID NO:15, and a light chain amino acid sequence comprising a CDRL1 of SEQ ID NO:16, a CDRL2 of SEQ ID NO:17, and a CDRL3 of SEQ ID NO:18;

(iii) a PD-1 binding protein comprising a heavy chain amino acid sequence comprising a CDRH1 of SEQ ID NO:24, a CDRH2 of SEQ ID NO:25, and a CDRH3 of SEQ ID NO:26, and a light chain amino acid sequence comprising a CDRL1 of SEQ ID NO:27, a CDRL2 of SEQ ID NO:28, and a CDRL3 of SEQ ID NO:29; and alternatively comprising

(iv) instructions for using (i) and (ii) in combination in the treatment of a cancer in a human.

In one embodiment, the kit comprises:

(i) an ICOS binding protein at a concentration of 10 mg/mL; and

(ii) a TIM-3 binding protein at a concentration of about 5 mg/mL to about 100 mg/mL, such as about 10 mg/mL to about 40 mg/mL, in particular 20 mg/mL.

In a further embodiment, the kit comprises:

(i) an ICOS binding protein at a concentration of 10 mg/mL;

(ii) a TIM-3 binding protein at a concentration of about 5 mg/mL to about 100 mg/mL, such as about 10 mg/mL to about 40 mg/mL, in particular 20 mg/mL; and

(iii) a PD-1 binding protein at a concentration of about 20 mg/mL to about 125 mg/mL, such as about 20 mg/mL to about 50 mg/mL, in particular 20 mg/mL or 50 mg/mL.

In some aspects, the kit is for use in the treatment of cancer.

In some embodiments, the ICOS binding protein and the TIM-3 binding protein are each individually formulated in their own pharmaceutical compositions with one or more pharmaceutically acceptable carriers. In further embodiments, the ICOS binding protein, PD-1 binding protein and TIM-3 binding protein are each individually formulated in their own pharmaceutical compositions with one or more pharmaceutically acceptable carriers.

In some aspects, the invention provides a kit for use in the treatment of cancer comprising:

(i) an ICOS binding protein comprising a heavy chain amino acid sequence comprising a CDRH1 of SEQ ID NO:1, a CDRH2 of SEQ ID NO:2, and a CDRH3 of SEQ ID NO:3, and a light chain amino acid sequence comprising a CDRL1 of SEQ ID NO:4, a CDRL2 of SEQ ID NO:5, and a CDRL3 of SEQ ID NO:6;

(ii) instructions for use in the treatment of cancer when combined with a TIM-3 binding protein. In further aspects, the invention provides a kit for use in the treatment of cancer comprising:

(i) an ICOS binding protein comprising a heavy chain amino acid sequence comprising a CDRH1 of SEQ ID NO:1, a CDRH2 of SEQ ID NO:2, and a CDRH3 of SEQ ID NO:3, and a light chain amino acid sequence comprising a CDRL1 of SEQ ID NO:4, a CDRL2 of SEQ ID NO:5, and a CDRL3 of SEQ ID NO:6;

(ii) instructions for use in the treatment of cancer when combined with a PD-1 binding protein and a TIM-3 binding protein.

In some aspects, the invention provides a kit for use in the treatment of cancer comprising:

(i) a TIM-3 binding protein comprising a heavy chain amino acid sequence comprising a CDRH1 of SEQ ID NO:13, a CDRH2 of SEQ ID NO:14, and a CDRH3 of SEQ ID NO:15, and a light chain amino acid sequence comprising a CDRL1 of SEQ ID NO:16, a CDRL2 of SEQ ID NO:17, and a CDRL3 of SEQ ID NO:18;

(ii) instructions for use in the treatment of cancer when combined with an ICOS binding protein.

In one embodiment, the kit for use in the treatment of cancer comprises:

(i) an ICOS binding protein at a concentration of 10 mg/mL; and

(ii) a TIM-3 binding protein at a concentration of about 5 mg/mL to about 100 mg/mL, such as about 10 mg/mL to about 40 mg/mL, in particular 20 mg/mL.

In a further embodiment, the kit for use in the treatment of cancer comprises:

(i) an ICOS binding protein at a concentration of 10 mg/mL;

(ii) a TIM-3 binding protein at a concentration of about 5 mg/mL to about 100 mg/mL, such as about 10 mg/mL to about 40 mg/mL, in particular 20 mg/mL; and

(iii) a PD-1 binding protein at a concentration of about 20 mg/mL to about 125 mg/mL, such as about 20 mg/mL to about 50 mg/mL, in particular 20 mg/mL or 50 mg/mL.

In particular embodiments of all of the above kit aspects, the ICOS binding protein is feladilimab. In particular embodiments of all of the above kit aspects, the TIM-3 binding protein is cobolimab. In particular embodiments of all of the above kit aspects, the PD-1 binding protein is dostalimab.

### EXAMPLES

### Example 1. In Vivo Efficacy Study of Test Antibodies in the Treatment of a Murine Tumor Model

This study was conducted under a protocol which was approved by the GSK Institutional Animal Care and Use Committee prior to commencement of the study. The objective of this study was to evaluate the therapeutic efficacy of anti-ICOS agonist antibody (clone murine ICOS IgG1 clone 7E. 17G9, mouse IgG1) alone and in combination with PD-1 (clone RMP1-14, rat IgG2a) and TIM-3 (clone RMT3-23, rat IgG2a) blockade in a syngeneic mouse tumor model, EMT-6 (mammary, BALB/c background).

### 1.1 Cell Culture

A breast cancer cell line (EMT-6) was maintained in vitro with DMEM+10% FBS at 37° C. in an atmosphere of 5% CO₂ in air. The tumor cells were routinely subcultured twice weekly. The cells in an exponential growth phase were harvested and counted for tumor inoculation.

### 1.2 Tumor Inoculation

BALB/C female mice were inoculated subcutaneously in the right lower flank with 5×10⁵ tumor cells in 0.1 ml of PBS for tumor development. The date of tumor cell inoculation was denoted as day 0.

### 1.3 Randomization

The randomization started when the mean tumor size reached approximately 95 mm³. 90 mice were enrolled in the study. All animals were randomly allocated to 9 study groups. Randomization was performed based on “Matched distribution” method (STUDYDIRECTOR software, version 3.1.399.19) randomized block design.

### 1.4 Observation and Data Collection

After tumor cell inoculation, the animals were checked daily for morbidity and mortality. During routine monitoring, the animals were checked for any effects of tumor growth and treatments on behavior such as mobility, food and water consumption, body weight gain/loss (Body weights were measured twice per week after randomization), eye/hair matting and any other abnormalities. Mortality and observed clinical signs were recorded for individual animals in detail.

### 1.5 Antibody Administration

Antibodies were administered in the study design described in Table 2. All antibodies were dosed concurrently, biweekly (BIW) for a total of 3 weeks. The treatment was initiated at the same day as grouping—day 6 for EMT-6 model.

TABLE 2
Antibody study design

| | | | Dose | Dosing | | |
|---|---|---|---|---|---|---|
| | | | level | Volume | | Dosing |
| | | | (μg/ | (μL/ | | Frequency |
| Group | No. | Treatment | mouse) | mouse) | ROA | & Duration |
| 1 | 10 | PBS | − | 100 | i.p. | BIW x 3 weeks |
| 2 | 10 | rIgG2a | 100 | 100 | i.p. | BIW x 3 weeks |
| | | mIgG1 | 10 | 100 | i.p. | BIW x 3 weeks |
| 3 | 10 | Anti-TIM3 | 100 | 100 | i.p. | BIW x 3 weeks |
| | | mIgG1 | 10 | 100 | i.p. | BIW x 3 weeks |
| 4 | 10 | Anti-PD1 | 100 | 100 | i.p. | BIW x 3 weeks |
| | | mIgG1 | 10 | 100 | i.p. | BIW x 3 weeks |
| 5 | 10 | rIgG2a | 100 | 100 | i.p. | BIW x 3 weeks |
| | | Anti-ICOS | 10 | 100 | i.p. | BIW x 3 weeks |
| 6 | 10 | Anti-TIM3 | 100 | 100 | i.p. | BIW x 3 weeks |
| | | Anti-PD1 | 100 | 100 | i.p. | BIW x 3 weeks |
| 7 | 10 | Anti-TIM3 | 100 | 100 | i.p. | BIW x 3 weeks |
| | | Anti-ICOS | 10 | 100 | i.p. | BIW x 3 weeks |
| 8 | 10 | Anti-PD1 | 100 | 100 | i.p. | BIW x 3 weeks |
| | | Anti-ICOS | 10 | 100 | i.p. | BIW x 3 weeks |
| 9 | 10 | Anti-TIM3 | 100 | 100 | i.p. | BIW x 3 weeks |
| | | Anti-PD1 | 100 | 100 | i.p. | BIW x 3 weeks |
| | | Anti-ICOS | 10 | 100 | i.p. | BIW x 3 weeks |

### 1.6 Statistical Analysis

The survival time was analyzed by Kaplan-Meier method. The event of interest was the animal death. The survival time was defined as the time from the day of tumor cell inoculation to the day when the tumor volume reached to 3000 mm³. For each group, the median survival time (MST), corresponding 95% confidence interval and the increased in life-span (ILS) were calculated. The Kaplan-Meier curves was constructed for each group and the log-rank test was used to compare survival curves between groups. All data were analyzed using SPSS 18.0. P<0.05 was considered to be statistically significant.

### 1.7 Results

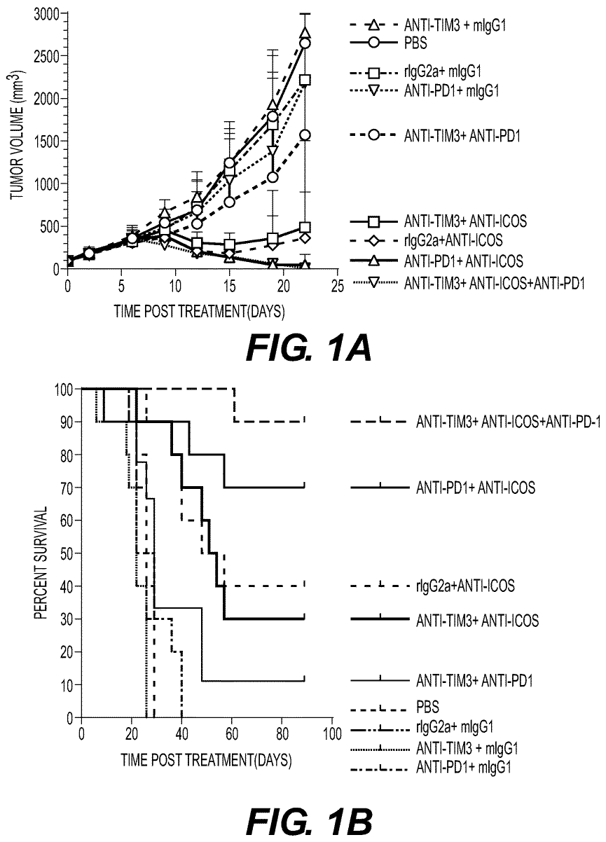

For the EMT-6 tumor model, anti-ICOS agonist monotherapy resulted in significant tumor growth inhibition (TGI, P<0.01) and tumor-free survival (P<0.01) relative to the isotype control group. Combination with anti-PD-1 led to a 30% increase of tumor-free survival relative to anti-ICOS agonist antibody alone and although not statistically significant, a trend in improved TGI and tumor-free survival was also observed on top of this doublet (anti-ICOS and anti-PD-1). Results are shown in FIG. 1.

A 20% increase in tumor-free survival was observed with the triple combination of anti-ICOS, anti-PD-1, and anti-TIM-3. Similarly, improved TGI was also observed in the triple combination group relative to the anti-ICOS and anti-PD-1 doublet, however, like the improvement in overall survival, the result was not significant, potentially due to the study size.

### Example 2. Combination Therapy Human Clinical Trial Protocol Development

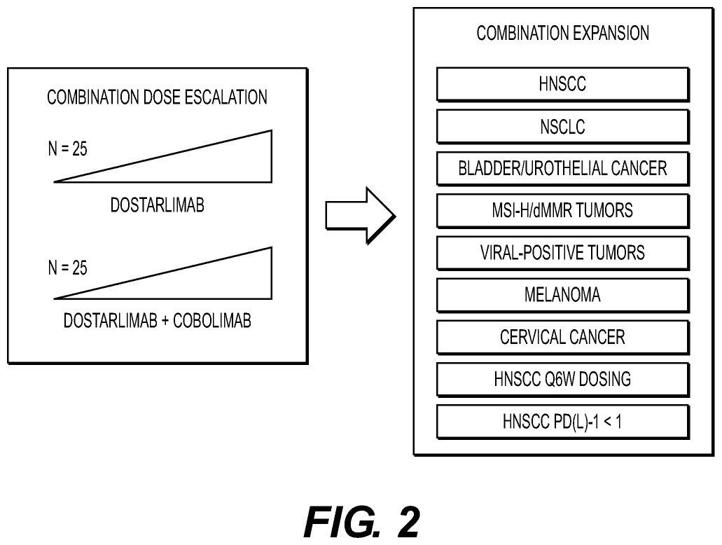

H2L5 hIgG4PE is an anti-Inducible T cell Co-Stimulator (ICOS) receptor agonist antibody intended for the treatment of cancers of different histology. It is expected to be active in combination with agents which prime or modulate tumor immunity. The study design as it relates to the combination of H2L5 hIgG4PE and dostarlimab+/−cobolimab is summarised in FIG. 2. H2L5 hIgG4PE comprises CDR sequences as set out in SEQ ID NOS: 1-6, variable heavy chain and variable light chain sequences as set out in SEQ ID NO:7 and SEQ ID NO: 8, respectively, and heavy chain and light chain sequences as set out in SEQ ID NO:9 and SEQ ID NO:10, respectively.

### 2.1 Study Design

H2L5 hIgG4PE will be tested in combination with dostarlimab. The study will investigate doses of 24 mg and 80 mg of H2L5 hIgG4PE and a fixed dose schedule of dostarlimab at 500 mg Q3W for 4 doses followed by 1000 mg Q6W thereafter.

H2L5 hIgG4PE will also be tested in combination with dostarlimab and cobolimab. The study will investigate doses of 24 mg and 80 mg of H2L5 hIgG4PE, dostarlimab at 500 mg Q3W for 4 doses followed by 1000 mg Q6W thereafter, and cobolimab at 300 mg Q3W.

These combinations evaluated will be investigated in subjects with selected, relapsed and/or refractory solid tumors. Approximately 25 subjects will be enrolled in each cohort.

In dose expansion phases, a Bayesian adaptive design with independent tumor type modeling will be implemented.

2.1.1 H2L5 hIgG4PE Combination with Dostarlimab or Dostarlimab Plus Cobolimab

The combination cohorts of dostarlimab and dostarlimab plus cobolimab will each have a dose escalation phase testing two different doses of H2L5 hIgG4PE, 24 mg (Dose Level 1) or 80 mg (Dose Level 2) with the combination partner at a fixed dose regimen for each Dose Level within each cohort of 25 subjects. The applicable combination partner dosing regimens are:
Dostarlimab combination therapy will begin with a fixed dose schedule of 500 mg Q3W administered intravenously for 4 doses, followed by 1000 mg Q6W administered intravenously thereafter;
Dostarlimab+cobolimab combination therapy will begin with a fixed dose of dostarlimab at 500 mg Q3W for 4 doses followed by 1000 mg Q6W thereafter administered intravenously and a fixed dose of cobolimab at 300 mg Q3W administered intravenously.

The goal for each cohort will be to determine the recommended Phase 2 dose (RP2D) based on a combination of safety and pharmacodynamic data including tissue level analysis based on biopsy samples. Alternate schedules or dose levels may be explored if data emerge supporting their investigation even after a RP2D is defined.

For each cohort of 25 total subjects, 3 subjects will be enrolled at the first dose level. If no dose-limiting toxicity (DLT) is observed among the 3 subjects, then a Dose Escalation discussion with the study investigators will occur. If DLT is observed among the 3 subjects, the cohort will be expanded to 6 subjects. If no further DLT is observed among the six subjects, then a Dose Escalation discussion with the study investigators will occur. If a second DLT is observed, then the H2L5 hIgG4PE dose will be de-escalated to a lower dose to be determined in discussion between the study team and investigators with a likely target of 0.1 mg/kg. The Dose Escalation Plan is summarized in Table 3.

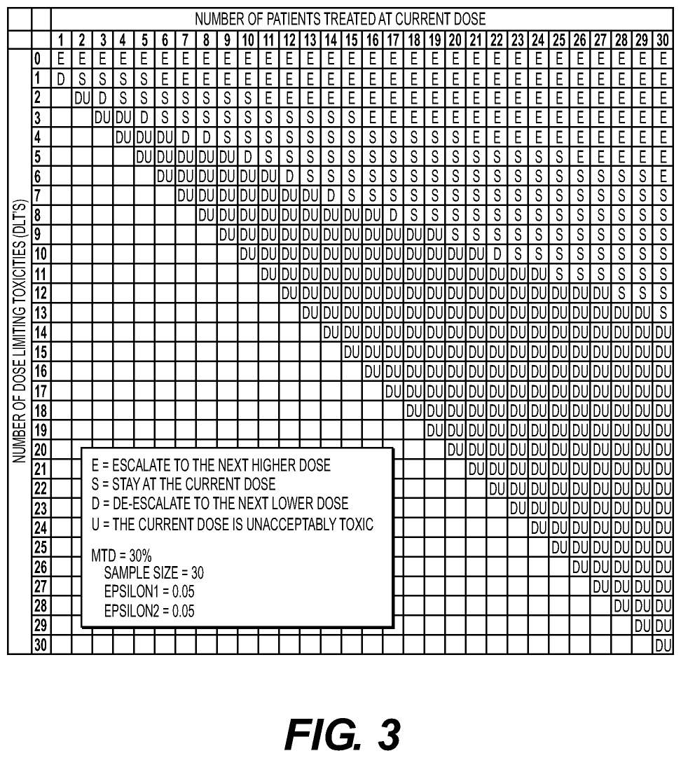

Dose decision rules will follow the modified Toxicity Probability Interval (mTPI) method with FIG. 3 depicting the dose-finding actions escalation decisions based on DLT observed within a cohort. Safety, tolerability, PK, pharmacodynamic measures, and anti-tumor activity will be considered in determining RP2D of H2L5 hIgG4PE in combination.

Because each cohort is limited to 25 subjects, the number enrolled in the PK/pharmacodynamic phase will be 25 minus the number of subjects enrolled in the dose escalation phase. For example, if a total of 3 subjects are enrolled at each of two dose levels, the total number of subjects in dose escalation is 6. Subtracting 6 from 25 will then allow up to 19 subjects to be enrolled in the PK/pharmacodynamic phase. Another scenario could be that the total number of subjects enrolled in dose escalation is 3 at one dose level and 6 at the second dose level, so the dose escalation total is 9, which would allow up to 16 subjects to be enrolled in the PK/pharmacodynamic phase.

TABLE 3
Dose Escalation Plan for Combination Therapies

| | | | | Additional |
|---|---|---|---|---|
| | | | | subjects for |
| | | | | PK/ |
| | | | | pharmacodynamics |
| | H2L5 | | N for | such that the total |
| Dose | hIgG4PE | Combination | safety | for all subjects |
| Level | dose | Partner | clearance | in the cohort is ≤25 |
| 1 | 24 mg | Fixed dose regimen | 3-6 | 6-19 |
| 2 | 80 mg | Fixed dose regimen | 3-6 | 6-19 |

If the combination doses in the starting dose cohort are not tolerable, lower doses of H2L5 hIgG4PE may be evaluated.

Additional subjects can be enrolled at one or both of the dose levels following safety clearance at that dose to generate PK/pharmacodynamic data to validate the dose at a tissue level. The PK/pharmacodynamic data will depend on obtaining evaluable tissue samples at baseline and on study at week 6. Based on prior experience, more subjects must be enrolled than samples required for analysis in order to account for non-evaluable or unobtainable tissue samples. All subjects in the PK/pharmacodynamic phase are also included in the anti-drug antibody (ADA) cohorts and assessed for anti-tumor activity based on imaging and immune-related Response Evaluation Criteria in Solid Tumors (irRECIST) criteria as anti-tumor activity is a pharmacodynamic outcome.

The study population in the dose escalation/safety run-in phases of the study are adults with advanced/recurrent solid tumors of the following type: bladder/urothelial cancer, cervical cancer, colorectal cancer (includes appendiceal carcinoma), esophageal cancer with squamous cell histology, head and neck cancer, melanoma, malignant pleural mesothelioma, non-small-cell lung cancer, and prostate cancer. Each cohort may enroll subjects with one specific tumor type selected from the aforementioned list at any time or enroll subjects based on additional features such as prior treatment history (i.e. anti-PD-1/L1 therapy), tumors exhibiting a specific molecular/genetic alteration (i.e. PD-L1 expression), or pathology (i.e. squamous).

2.1.2 Dose Limiting Toxicity

The severity of all toxicities will be graded using National Cancer Institute-Common Terminology Criteria for Adverse Events (NCI-CTCAE) (version 4.0) [NCI, 2010]. The DLT observation period is 28 days in length and begins on the day H2L5 hIgG4PE is first administrated to the subject.

A DLT is defined as an adverse event (AE) that meets at least one of the criteria listed in Table 4 and is considered by the investigator to be clinically relevant and attributed (probably, or possibly) to the study treatment during the 28-day DLT observation period. An AE considered related to the underlying disease under study it is not defined as a DLT.

TABLE 4
Dose-Limiting Toxicity Criteria

| Toxicity | DLT Definition |
|---|---|
| Hematologic | Febrile neutropenia as defined by CTCAE v4 |
| | Grade 4 neutropenia of >7 days in duration or requiring G-CSF |
| | Grade 4 anemia of any duration |
| | Grade 4 thrombocytopenia of any duration or Grade 3 thrombocytopenia |
| | with bleeding |
| Non- | Grade 4 toxicity |
| hematologic | Grade 3 pneumonitis of any duration |
| | Grade 3 toxicity that does not resolve to ≤ Grade 1 or baseline within 3 |
| | days despite optimal supportive care |
| | Any Grade 2 ocular toxicity requiring systemic steroids, or any ≥ Grade 3 |
| | ocular toxicity |
| | Following events are not considered DLTs |
| | Grade 3 and Grade 4 asymptomatic electrolyte abnormalities that |
| | are corrected within 24 hours without clinical sequelae |
| | Grade 3 nausea, vomiting, or fatigue that resolves to ≤ Grade 1 |
| | within 7 days with optimal supportive care |
| | Grade 3 and Grade 4 infusion reactions in subjects not receiving |
| | prophylaxis for infusion related reactions (IRRs) (refer to Section |
| | Error! Reference source not found. |
| | for details on IRR management) |
| Other | Toxicity that results in permanent discontinuation of H2L5 hIgG4PE |
| | monotherapy or H2L5 hIgG4PE and agent in combination during the first |
| | four weeks of treatment |
| | Grade 3/Grade 4 toxicity that results in a subject not receiving the |
| | expected doses of a regimen in Cycle 1, defined by 21 days |
| | Any other toxicity considered to be dose-limiting that occurs beyond four |
| | weeks will be considered in the selection of the dose to recommend for |
| | expansion cohorts |
| | Any other event which in the judgment of the investigator and GSK Medical |
| | Monitor is considered to be a DLT |

Note:
Suggested toxicity management guidelines may include systemic corticosteroids for immune-related toxicities; if systemic corticosteroids use delays administration of the second dose of study treatment and the event does not otherwise meet the DLT criteria for non-hematologic toxicity, the dose delay will not be considered a DLT.

If a subject experiences a DLT during the DLT observation period, the subject may resume dosing at the same or lower dose provided the toxicity did not meet study treatment discontinuation criteria and following approval by the Sponsor.

2.1.3 Intra-Subject Dose Escalation

Intra-subject dose escalations may be considered on a case-by-case basis provided the subject has completed at least one treatment cycle without the occurrence of drug-related Grade 2 AE or serious adverse events (SAEs) of any severity Grade in the first 28 days of treatment. For the expansion phases in which Week 6 on-treatment biopsy was mandatory, approval for intra-subject escalation also requires acquisition of this biopsy. Additionally, all subjects at the next higher dose level/levels must have completed the DLT observation period with maximum tolerated dose (MTD) not reached. Subjects may dose-escalate to the highest cleared dose. Individual subjects may dose-escalate multiple times provided that the above criteria are met at each intra-subject dose escalation step.

2.1.4 Dose Expansion Phase

Any dose level(s)/doses in the dose escalation phases may be selected for expansion in order to collect additional data on safety, PK, pharmacodynamic activity, and preliminary clinical activity.

Each expansion cohort will include subjects defined by a single tumor type as indicated in FIG. 2 or characterized by other features such as prior treatment with an immune checkpoint inhibitor, a molecular/genetic alteration (MSI-H/dMMR), or pathology. Subjects may be stratified by prior PD-1/L1 treatment history (i.e. naïve or experienced; best response).

The Steering Committee will review the totality of data available for the study to inform on the dose level indications for any of the expansion cohorts.

2.1.4.1. PK/Pharmacodynamic Dose Expansion Cohorts

Any dose level or levels may be expanded beyond the expected 3 subjects enrolled in dose escalation phase in order to collect additional data on safety, PK, pharmacodynamic activity, and preliminary efficacy. Subjects can only be enrolled at previously cleared dose levels. Subjects enrolled in PK/pharmacodynamic cohorts may have the dose escalated to a higher cleared dose level (i.e. not exceeding the MTD) once the necessary PK/pharmacodynamic procedures have been completed. Model-based designs may be employed for each PK/pharmacodynamic dose expansion cohort in order to sufficiently explore parameters critical (i.e. safety, tolerability, and efficacy) in establishing the biologically optimal doses of the agents in the combination.

2.1.5 Study Treatment and Duration

Each part and phase of the study includes a screening period, a treatment period, and a follow-up period. For subjects who meet all eligibility criteria and register into the study, the maximum duration of treatment with H2L5 hIgG4PE is expected to be two years, up to 35 cycles. The maximum follow-up period for safety assessments will be 90 days from the date of the last dose of study treatment. The expected maximum follow-up period for survival and subsequent anti-cancer therapy will be two years from the date of the last dose of study treatment. Subjects who discontinue study treatment due to achieving confirmed complete response (CR) (refer to Section 2.2.3 for additional requirements) will be followed for progression (refer to Section 2.2.3 for details on the frequency of these assessments).

Subjects participating in the dostarlimab combination cohort will receive H2L5 hIgG4PE 24 or 80 mg dose (refer to Table 5 for fixed doses) in combination with dostarlimab administered as an intravenous (IV) infusion at 500 mg Q3W for 4 cycles followed by 1000 mg Q6W.

Subjects participating in the dostarlimab+cobolimab combination cohort will receive H2L5 hIgG4PE 24 or 80 mg dose (refer to Table 5 for fixed doses) in combination with dostarlimab administered as an IV infusion at 500 mg Q3W for 4 cycles followed by 1000 mg Q6W plus cobolimab administered as an IV infusion at 300 mg Q3W.

2.1.6 Dose Justification

2.1.6.1 H2L5 hIgG4PE Starting Dose in Combination with Dostarlimab or Dostarlimab Plus Cobolimab

The H2L5 hIgG4PE doses of 24 mg and 80 mg were selected based on the preliminary ICOS receptor occupancy pharmacodynamic analysis in the periphery which showed high receptor occupancy levels on CD4 and CD8 T cells over the 21-day dosing cycle starting at 0.3 mg/kg (˜24 mg); close to total receptor saturation was observed at 1 mg/kg (˜80 mg) dose level. Based on prior clinical and non-clinical data, no overlapping toxicities are expected. Also, based on established pharmacology, no drug-drug interactions are expected.

2.1.6.2 H2L5 hIgG4PE Dosing Frequency

Since select partner agents may be dosed less frequently than every three weeks, alternative extended dosing schedules would provide additional convenience and flexibility to patients and clinicians beyond a Q3W option. Hence, a six-weekly (Q6W) dosing schedule for H2L5 hIgG4PE will be explored, specifically in randomized schedule optimization cohorts for subjects with PD-1/L1 Naive HNSCC. Two doses for initial Q6W schedule exploration, 48 and 160 mg, are selected to provide matching cumulative exposures corresponding to respective Q3W regimens in the Q3W HNSCC dose-randomized cohorts (0.3 and 1 mg/kg). Preliminary PK simulations suggest a doubling of dose and interval for H2L5 hIgG4PE (e.g. 0.3 mg/kg Q3W to 48 mg Q6W) is expected to provide similar cumulative AUC with an approximate doubling of end-of-infusion Cmax and marginally lower end-of-cycle trough concentrations (˜43% at steady-state). The typical Cmax for 160 mg Q6W will be maintained below thresholds established with the Q3W regimens.

2.1.6.3 H2L5 hIgG4PE Fixed Dose Rationale

Fixed doses may be tested in the dose escalation with dostarlimab and dostarlimab plus cobolimab, assuming a typical median weight of 80 kg.

Preliminary population PK simulations indicate that using fixed dosing would result in a similar range of exposures as that of body weight-based dosing. Also, fixed dosing offers the advantage of reduced dosing errors, reduced drug wastage, shorten preparation time, and improve ease of administration. Thus, switching to a fixed dose based on a reference body weight of 80 kg is reasonable and appropriate.

The fixed dose equivalents of the weight-based H2L5 hIgG4PE dose levels using 80 kg weight are presented in Table 5.

TABLE 5
H2L5 hIgG4PE Fixed Dose Calculations

| Dose Level | H2L5 hIgG4PE (mg/kg) | H2L5 hIgG4PE (mg) |
|---|---|---|
| 1 | 0.001 | 0.08 |
| 2 | 0.003 | 0.24 |
| 3 | 0.01 | 0.8 |
| 4 | 0.03 | 2.4 |
| 5 | 0.1 | 8.0 |
| 6 | 0.3 | 24.0 |
| 7 | 0.6 | 48.0 |
| 8 | 1.0 | 80.0 |
| 9 | 2.0 | 160.0 |
| 10 | 3.0 | 240.0 |

2.1.6.4 Dostarlimab (Anti-PD1) Dose Rationale

The recommended clinical dose and regimen of dostarlimab is 500 mg Q3W for 4 cycles followed by 1000 mg Q6W. This regimen was determined from the results of a corresponding Phase 1/2 study where the PK, efficacy and safety were evaluated over 3 parts within that study.

2.1.6.5 Cobolimab (Anti-TIM3) Dose Rationale

The recommended clinical dose and regimen of cobolimab is 300 mg Q3W. This regimen was determined from the results of a corresponding Phase 1 study, monotherapy and in combination with 500 mg dostarlimab Q3W.

Receptor Occupancy (RO) data suggests the potential for relatively higher target engagement at 900 mg, however, no improvement in clinical activity was observed with the 900 mg dose compared with the 300 mg dose (Caveat: Data from non-randomized sequential cohorts).

Together with the efficacy and safety data, these data support selection of a RP2D of 300 mg Q3W.

### 2.2 Selection of Study Population and Withdrawal Criteria

2.2.1 Inclusion Criteria

For a subject to be eligible for inclusion in this study all the following criteria must be fulfilled:

1. Capable of giving signed, written informed consent

2. Male or female, age 18 years (at the time consent is obtained).

3. Histological or cytological documentation of an invasive malignancy that was diagnosed as locally advanced/metastatic or relapsed/refractory and is of one of the following tumor types:
Bladder/urothelial cancer of the upper and lower urinary tract
Cervical
Colorectal (includes appendix)
Esophagus, squamous cell
Head and Neck Carcinoma
Melanoma
MPM
NSCLC
Prostate
MSI-H/dMMR tumor
HPV-positive or EBV-positive tumor

4. Disease that has progressed after standard therapy for the specific tumor type, or for which standard therapy has proven to be ineffective, intolerable, or is considered inappropriate, or if no further standard therapy exists.
Subjects must not have received more than 5 prior lines of therapy for advanced disease including both standards of care and investigational therapies.
Subjects who received prior anti-PD-1/L1 therapy must fulfill the following requirements:
   Have achieved a complete response [CR], partial response [PR]) and stable disease [SD] and subsequently had disease progression while still on PD 1/L1 therapy;
   Have received at least 2 doses of an approved PD-1/L1 inhibitor (by any regulatory authority);
   Have demonstrated disease progression as defined by RECIST v1.1 within 18 weeks from the last dose of the PD-1/L1 inhibitor. The initial evidence of disease progression is to be confirmed by a second assessment no less than four weeks from the date of the first documented PD (the confirmatory scan could be the baseline eligibility scan for this study).

5. Archival tumor tissue obtained at any time from the initial diagnosis to study entry; a fresh tumor biopsy using a procedure that is safe for the subject on a lesion not previously irradiated unless lesion progressed will be required if archival tissue is unavailable.

6. Agree to undergo a pre-treatment and on-treatment biopsy and have disease amenable to biopsy required in PK/pharmacodynamic, dose randomized HNSCC, Melanoma dose expansion and Biomarker cohorts.

7. Measurable disease per RECIST version 1.1 (refer to Section 2.6). Palpable lesions that are not measurable by radiographic or photographic evaluations may not be utilized as the only measurable lesion. Any measurable lesion biopsied at Screening cannot be followed as a target/index lesion unless agreed upon by GSK.

8. Eastern Cooperative Oncology Group (ECOG) performance status (PS) 0-1 (refer to Section 2.7).

9. Life expectancy of at least 12 weeks.

10. Adequate organ function as defined in Table 6:

TABLE 6
Definitions for Adequate Organ Function

| | |
|---|---|
| System | Laboratory Values |

Hematologic^{b}

| | |
|---|---|
| Absolute neutrophil count (ANC) | ≥1.5 × 10⁹/L |
| Hemoglobin | ≥29 g/dL |
| Platelets | ≥100 × 10⁹/L |

Hepatic

| | |
|---|---|
| Total bilirubin | ≤1.5 × upper limit of normal (ULN) |
| For subjects with Gilbert's Syndrome | ≤3.0 × ULN |
| (only if direct bilirubin ≤35%) | |
| Alanine aminotransferase (ALT) | ≤2.5 × ULN; |
| | or ≤5 × ULN for subjects with |
| | documented liver metastases |

Renal

| | |
|---|---|
| Calculated creatinine clearance^{c} | ≥30 mL/min |

Cardiac

| | |
|---|---|
| Ejection fraction | ≥50% by echocardiogram^{d} |

^{a}Absolute Lymphocyte Count will be included in the baseline assessment, but no range limit requirement for the eligibility.
^{b}Estimated CrCl should be calculated using the Chronic Kidney Disease Epidemiology Collaboration (CKD-EPI) formula.
^{c}Multigated acquisition scan (MUGA) is acceptable if ECHO is not available (refer to Echiocardiograms section, below)

11. QT duration corrected for heart rate by Fridericia's formula (QTcF)<450 milliseconds (msec) or QTcF <480 msec for subjects with bundle branch block. The QTcF is the QT interval corrected for heart rate according to Fridericia's formula, machine-read or manually over-read.

12. A female subject is eligible to participate if she is not pregnant (as confirmed by a negative serum beta-human chorionic gonadotrophin [β-hCG] test in females of reproductive potential) and not lactating, or at least one of the following conditions applies:

a) Non reproductive potential, defined as:
Pre-menopausal females with one of the following: Documented tubal ligation, Documented hysteroscopic tubal occlusion procedure with follow-up confirmation of bilateral tubal occlusion, Hysterectomy, Documented Bilateral Oophorectomy Postmenopausal defined as 12 months of spontaneous amenorrhea. Females on hormone replacement therapy (HRT) and whose menopausal status is in doubt will be required to use one of the highly effective contraception methods if they wish to continue their HRT during the study. Otherwise, they must discontinue HRT to allow confirmation of post-menopausal status prior to study enrolment.

b) Reproductive potential and agrees to follow highly effective methods for avoiding pregnancy from 30 days prior to the first dose of study medication and until 120 days after the last dose of study treatment.

13. Male subjects with female partners of child bearing potential must agree to use a highly effective method of contraception from time of first dose of study treatment until 120 days after the last dose of study treatment.

15. Documented Human Papilloma Virus (HPV)/Epstein-Barr (EBV)-positive tumor as determined by a local laboratory for viral-positive expansion cohorts only

16. Documented MSI-H or dMMR-positive tumor as determined by local laboratory for combination MSI-H/dMMR expansion cohorts only.

17. PD-L1 CPS<1 using the FDA approved PD-L1 IHC 22C3 pharmDx assay by central laboratory testing for HNSCC PD-L1 CPS<1 Cohort. Documented test result from FDA approved PD-L1 IHC 22C3 pharmDx assay in local laboratory, if available, may be accepted in lieu of the central laboratory test result.

18. Defined PD-L1 expression using the Ventana PD-L1 (SP263) IHC assay by central testing for enrollment in the PK/PD cohort with combination studies.

2.2.2 Exclusion Criteria

A subject will not be eligible for inclusion in this study if any of the following criteria apply:

1. Prior treatment with the following therapies:
Anti-cancer therapy within 30 days or 5 half-lives of the drug, whichever is shorter. At least 14 days must have elapsed between the last dose of prior anti-cancer agent and the first dose of study drug is administered.
Prior radiation therapy: permissible if at least one non-irradiated measurable lesion is available for assessment according to RECIST version 1.1 or if a solitary measurable lesion was irradiated, objective progression is documented. A wash out of at least two weeks before start of study drug for radiation of any intended use to the extremities for bone metastases and 4 weeks for radiation to the chest, brain, or visceral organs is required.
Investigational therapy within 30 days or 5 half-lives of the investigational product (whichever is shorter). At least 14 days must have elapsed between the last dose of investigational agent and the first dose of study drug is administered.

2. Prior allogeneic or autologous bone marrow transplantation or other solid organ transplantation.

3. Toxicity from previous anti-cancer treatment that includes:
≥Grade 3 toxicity considered related to prior immunotherapy and that led to treatment discontinuation.
Toxicity related to prior treatment that has not resolved to Grade 1 (except alopecia, endocrinopathy managed with replacement therapy, and peripheral neuropathy which must be Grade 2).

4. Invasive malignancy or history of invasive malignancy other than disease under study within the last two years, except as noted below:
Any other invasive malignancy for which the subject was definitively treated, has been disease-free for years and in the opinion of the principal investigator and GSK Medical Monitor will not affect the evaluation of the effects of the study treatment on the currently targeted malignancy, may be included in this clinical trial.
Curatively treated non-melanoma skin cancer.

5. Central nervous system (CNS) metastases, with the following exception:
Subjects who have previously-treated CNS metastases, are asymptomatic, and have no requirement for steroids at least 14 days prior to first dose of study drug. Note: Subjects with carcinomatous meningitis or leptomeningeal spread are excluded regardless of clinical stability.

6. Received transfusion of blood products (including platelets or red blood cells) or administration of colony stimulating factors (including granulocyte colony-stimulating factor [G-CSF], granulocyte-macrophage colony-stimulating factor, recombinant erythropoietin) within 14 days prior to the first dose of H2L5 hIgG4PE.

7. Major surgery weeks before the first dose of study treatment. Subjects must have also fully recovered from any surgery (major or minor) and/or its complications before initiating study treatment.

8. Active autoimmune disease that has required systemic treatment within the last two years (i.e. with use of disease modifying agents, corticosteroids or immunosuppressive drugs). Note: Replacement therapy (e.g. thyroxine or physiologic corticosteroid replacement therapy for adrenal or pituitary insufficiency, etc.) is not considered a form of systemic treatment.

9. Concurrent medical condition requiring the use of systemic immunosuppressive medications within 7 days before the first dose of study treatment. Physiologic doses of corticosteroids for treatment of endocrinopathies or steroids with minimal systemic absorption, including topical, inhaled, or intranasal corticosteroids may be continued if the subject is on a stable dose.

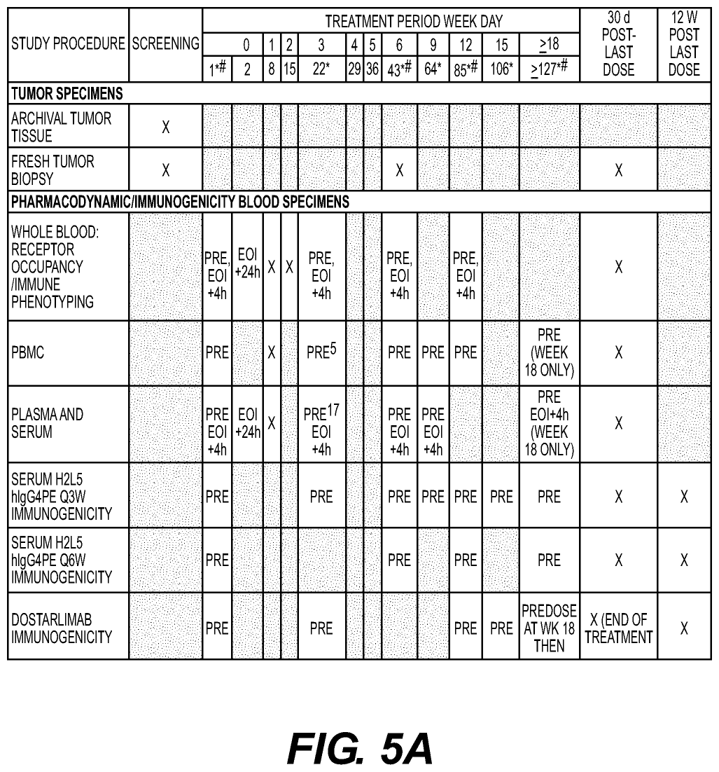

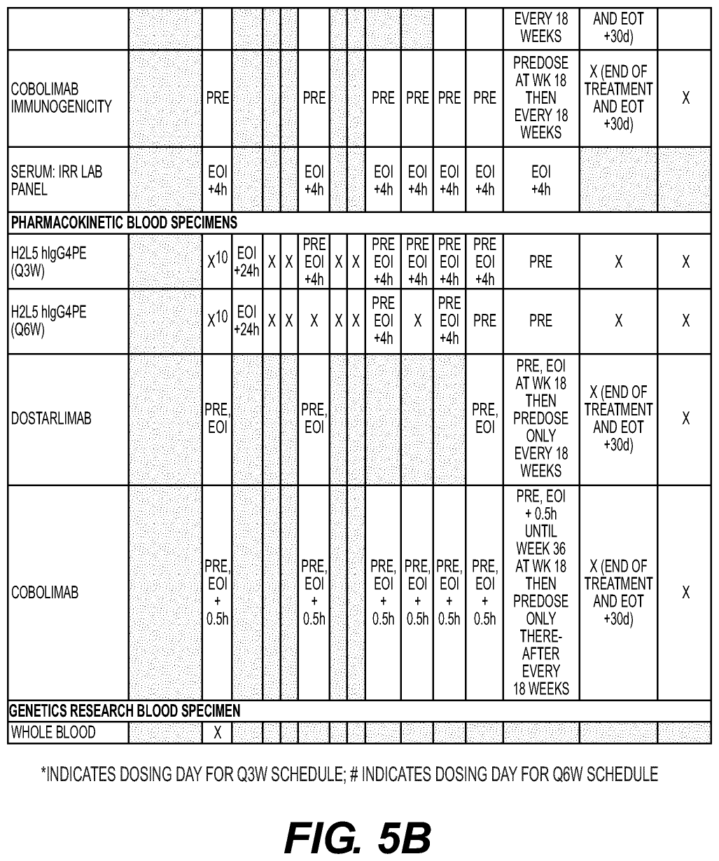

10. Active infection requiring systemic therapy, known human immunodeficiency virus infection, or positive test for hepatitis B active infection or hepatitis C active infection (refer to FIG. 5 for details).

11. Current active liver or biliary disease (with the exception of Gilbert's syndrome or asymptomatic gallstones, liver metastases, or otherwise stable chronic liver disease per investigator assessment). Note: Stable chronic liver disease should generally be defined by the absence of ascites, encephalopathy, coagulopathy, hypoalbuminemia, esophageal or gastric varices, persistent jaundice, or cirrhosis.

12. Recent history (within the past 6 months) of acute diverticulitis, inflammatory bowel disease, intra-abdominal abscess, or gastrointestinal obstruction that required surgery

13. Receipt of any live vaccine within 4 weeks prior to first dose of study treatment.

14. Recent history of allergen desensitization therapy within 4 weeks of starting study treatment.

15. History of severe hypersensitivity to monoclonal antibodies or to the chemotherapies under investigation including any ingredient used in the formulation.

16. History or evidence of cardiac abnormalities including any of the following:
Recent (within the past 6 months) history of serious uncontrolled cardiac arrhythmia or clinically significant electrocardiogram abnormalities including second degree (Type II) or third degree atrioventricular block.
Cardiomyopathy, myocardial infarction, acute coronary syndromes (including unstable angina pectoris), coronary angioplasty, stenting, or bypass grafting within the past 6 months before enrollment.
Congestive heart failure (Class II, III, or IV) as defined by the New York Heart Association functional classification system.
Recent (within the past 6 months) history of symptomatic pericarditis.

17. History (current and past) of idiopathic pulmonary fibrosis, pneumonitis (for past pneumonitis exclusion only if steroids were required for treatment), interstitial lung disease, or organizing pneumonia. Note: post-radiation changes in the lung related to prior radiotherapy and/or asymptomatic radiation-induced pneumonitis not requiring treatment may be permitted if agreed by the investigator and Medical Monitor.

18. Recent history (within 6 months) of uncontrolled symptomatic ascites or pleural effusions.

19. Any serious and/or unstable pre-existing medical, psychiatric disorder, or other condition that could interfere with the subject's safety, obtaining informed consent, or compliance to the study procedures.

20. Is or has an immediate family member (e.g. spouse, parent/legal guardian, sibling or child) who is an investigational site or sponsor staff directly involved with the trial, unless prospective IRB approval (by chair or designee) is given allowing exception to this criterion for a specific subject.

2.2.3 Withdrawal/Stopping Criteria

Subjects will receive study treatment for the scheduled time period, if applicable, unless one of the following events occurs earlier: disease progression (as determined by irRECIST), death, or unacceptable toxicity, including meeting stopping criteria for liver chemistry (refer to Section 2.2.3.1), or other criteria are met as defined in Section 2.2.3.2. Subjects with infusion delays >21 days due to toxicity should consider discontinuing study drug(s) unless the treating investigator and Sponsor/Medical Monitor agree there is strong evidence supporting continued treatment.

Subjects enrolled who require permanent discontinuation of one of the study agents in a given treatment combination due to toxicity must permanently discontinue both agents in that combination, unless continued treatment with the remaining agent is agreed upon by the treating investigator and Sponsor/Medical Monitor.

In addition, study treatment may be permanently discontinued for any of the following reasons:

a. Deviation(s) from the protocol

b. Request of the subject or proxy

c. Discretion of the investigator

d. Subject is lost to follow-up

e. Closure or termination of the study

The primary reason for discontinuation must be recorded in the subject's medical records and electronic case report form (eCRF).

If the subject voluntarily discontinues from treatment due to toxicity, ‘adverse event’ will be recorded as the primary reason for permanent discontinuation on the eCRF.

Once a subject has permanently discontinued from study treatment, the subject will not be allowed to be retreated.

The assessments required at the treatment discontinuation visit (TDV) must be completed within 30 days of the decision to permanently discontinue study drug(s) and prior to the start of subsequent anti-cancer therapy.

All subjects who discontinue from study treatment (early or permanent) for any reason will have safety assessments at the time of discontinuation and during post study treatment follow-up.

Subjects with a CR or PR require confirmation of response via imaging at least 4 weeks after the first imaging showed a CR or PR.

Early discontinuation of study treatment (early discontinuation of study treatment will not per se constitute permanent discontinuation) may be considered for subjects who have attained a confirmed complete response per RECIST 1.1 and who received study treatment for at least 24 weeks and had at least two treatments beyond the date when the initial CR was declared; these subjects will undergo disease assessments at a frequency of 12 weeks. These subjects may be permitted to resume study treatment upon disease progression; this retreatment is defined as a Second Course. In addition, subjects with RECISTv1.1 confirmed SD, PR, or CR who complete the 35 cycles of study treatment and study treatment is discontinued for this reason and not for other reasons such as disease progression or intolerability will undergo disease assessments at a frequency of 12 weeks: these subjects may be able to receive a second course of study treatment upon disease progression. For subjects to be eligible for a second course of study treatment, all following requirements must be met:
Experienced an investigator-determined radiographic disease progression by RECIST 1.1 after discontinuing the initial course of study treatment
No subsequent/new anti-cancer treatment was administered after the last dose of study treatment
Fulfilled all of the safety parameters listed in the inclusion criteria and none of the safety parameters listed in the exclusion criteria are met
The study is still ongoing

If study treatment is restarted, subjects will be required to resume assessments; in addition, limited PK and immunogenicity sampling is required.

All subjects who permanently discontinue study treatment for any reason will be followed for survival and new anti-cancer therapy (including radiotherapy) every 12 weeks until death, termination of the overall study or a cohort by the sponsor or until the subject has been followed for two years. If subjects are unable or unwilling to attend clinic visits during follow-up, contact to assess survival may be made via another form of communication (e.g. telephone, email, etc.).

All subjects who permanently discontinue study treatment for reasons other than disease progression or consent withdrawal will be followed for progression or until the start of anti-cancer therapy whichever comes first.

2.2.3.1 Liver Chemistry Stopping Criteria

Liver chemistry stopping and increased monitoring criteria have been designed to assure subject safety and evaluate liver event etiology (in alignment with the Food and Drug Administration (FDA) premarketing clinical liver safety guidance).

If any of the criteria in Table 7 are met, all study drugs must be discontinued.

TABLE 7
Liver Chemistry Stopping Criteria
Liver Stopping Event for the subjects with ALT ≤2.5 ULN at the baseline value

| | |
|---|---|
| ALT-Increase | ALT ≥5 × ULN |
| ALT Increase | ALT ≥3 × ULN but <5 × ULN persists for ≥4 weeks |
| Bilirubin^{a, b} | ALT ≥3 × ULN and bilirubin ≥2 × ULN (>35% direct bilirubin) |
| International Normalized | ALT ≥3 × ULN and INR >1.5 |
| Ratio (INR)^{b} | |
| Cannot Monitor | ALT ≥3 × ULN but <5 × ULN and cannot |
| | be monitored weekly for ≥4 weeks |
| Symptomatic^{c} | ALT ≥3 × ULN associated with symptoms (new or worsening) |
| | believed to be related to liver injury or hypersensitivity |

Liver Stopping Event for Subjects with ALT >2.5 or ≤5 × ULN at Baseline Value

| | |
|---|---|
| ALT absolute | Both ALT ≥5 × ULN and ≥2 × baseline value |
| ALT Increase | Both ALT ≥3 × ULN and ≥1.5 × baseline |
| | value that persists for ≥4 weeks |
| Bilirubin^{a, b} | ALT ≥3 × ULN and bilirubin ≥2 × ULN (>35% direct bilirubin) |
| INR^{b} | ALT ≥3 × ULN and INR >1.5 |
| Cannot Monitor | Both ALT ≥3 × ULN and ≥1.5 × baseline |
| | value that cannot be monitored for 4 weeks |
| Symptomatic | Both ALT ≥3 × ULN and ≥1.5 × baseline value associated with |
| | symptoms (new or worsening) believed to be related to liver injury |
| | or hypersensitivity |

^{a}Serum bilirubin fractionation should be performed if testing is available. If serum bilirubin fractionation is not immediately available, discontinue study treatment if ALT ≥3 × ULN and bilirubin ≥2 × ULN. Additionally, if serum bilirubin fractionation testing is unavailable, record presence of detectable urinary bilirubin on dipstick, indicating direct bilirubin elevations and suggesting liver injury.
^{b}All events of ALT ≥3 × ULN and bilirubin ≥2 × ULN (≥35% direct bilirubin) or ALT ≥3 × ULN and INR ≥1.5, which may indicate severe liver injury (possible ‘Hy's Law’), must be reported as an SAE (excluding studies of hepatic impairment or cirrhosis); INR measurement is not required and the threshold value stated will not apply to subjects receiving anticoagulants.
^{c}New or worsening symptoms believed to be related to liver injury (such as fatigue, nausea, vomiting, right upper quadrant pain or tenderness, or jaundice) or believed to be related to hypersensitivity (such as fever, rash or eosinophilia).

2.2.3.2 Stopping Rules for Clinical Deterioration

To adequately assess the anti-tumor effect of immunotherapeutic agents it is reasonable to allow subjects experiencing apparent progression as defined by RECIST 1.1 guidelines to continue to receive treatment until progression is confirmed at the next imaging assessment at least 4 weeks later as indicated by irRECIST guidelines. Nevertheless, these considerations should be balanced by clinical judgment as to whether the subject is clinically deteriorating and unlikely to receive any benefit from continued study treatment.

In cases where deterioration was assessed to have occurred after a clinical event that, in the investigator's opinion, is attributable to disease progression and is unlikely to reverse with continued study treatment or managed by supportive care (e.g. bisphosphonates and/or bone directed radiotherapy, thoracentesis, or paracentesis for accumulating effusions), study treatment should be discontinued. Examples of events that may, in the investigator's opinion, indicate a lack of clinical benefit include, but are not limited to, the following:
ECOG PS worsening of at least 2 points from baseline
Skeletal related events defined by the following: pathologic bone fracture in the region of cancer involvement; cancer related surgery to bone; and/or spinal cord or nerve root compression
Development of new CNS metastases
Any setting where the initiation of new antineoplastic therapy has been deemed beneficial to the subject even in the absence of any such documented clinical event.

2.2.4 Subject and Study Completion

For combinations with dostarlimab or dostarlimab plus cobolimab and the dose escalation phases of the study, subjects will be considered as completing the study if they complete screening assessments, receive at least two doses of study treatment or receive one dose but experience a DLT, are observed during the 28 day DLT observation period, and complete the treatment discontinuation visit and the follow-up visit for safety or have died while receiving study treatment or during post-study treatment follow-up period for safety.

### 2.3 Study Treatment

2.3.1 Investigational Product and Other Study Treatment

H2L5 hIgG4PE will be administered intravenously to subjects at each study site under medical supervision of an investigator or designee. When administered in combination, H2L5 hIgG4PE will be administered first. In the H2L5 hIgG4PE dostarlimab plus cobolimab cohort, H2L5 hIgG4PE will be administered first followed by cobolimab and dostarlimab last.

Dostarlimab or dostarlimab plus cobolimab (refer to Table 8) will be administered intravenously to subjects starting at least 30 minutes and no more than one hour following the end of the H2L5 hIgG4PE infusion under medical supervision of an investigator or designee.

All subjects are required to remain under observation at the study site for at least 1.5 hours post-infusion of the last study drug administered for the first two study treatment dosing visits. At subsequent study treatment dosing visits, for subjects who experience infusion-related reactions, the post-infusion observation time should remain as at least 1.5 hours; for subjects who do not experience infusion reactions, these subjects should remain under observation at the study site post-study treatment infusion for at least 30 minutes or as per the judgement of the investigator or as per institutional guidelines.

For drug administered by an investigator or designee, the dose of study treatment and study subject identification will be confirmed at the time of dosing by a member of the study site staff other than the person administering the study treatment. The specific time of study treatment administration (e.g. time of the week for first administration; time of the day for each administration) should take into consideration PK sampling time points, study visit procedures, and the post-infusion observation time interval. Infusions may be administered up to 72 hours before or after the planned date of treatment for administrative reasons only (e.g. scheduling an infusion around a holiday).

TABLE 8
Combination Study Products Description and Administration

| Product | Study Treatment | |
|---|---|---|
| Name: | Dostarlimab | Cobolimab |
| Product | PD-1 Inhibitor | Anti-TIM3 |
| Description | | |
| Dosage form/ | 50 mg/mL solution | 20 mg/mL solution |
| strength | | |
| Planned | 500-1000 mg (see protocol for | 300 mg |
| dosage | details) | |
| level(s) | | |
| Route of | IV infusion | IV infusion |
| Administration | | |
| Dosing | Administer diluted product/once | Administer diluted |
| instructions/ | Q3W for 4 doses followed by | product/once Q3W |
| Frequency | 1000 mg Q6W thereafter (refer to | (refer to SRM for |
| | SRM for infusion time) | infusion time) |
| Manufacturer | GSK | GSK |

2.3.2 Treatment Assignment

Subjects enrolled in the study will be assigned to a combination treatment in an open-label fashion and according to the combination treatment cohorts open for accrual. Other expansion cohorts may investigate more than one dose level of H2L5 hIgG4PE; if implemented, subjects in this cohort will be randomly assigned to the selected dose levels.

2.3.3 Blinding

This is an open-label study.

2.3.4 Concomitant Medications and Non-Drug Therapies

Subjects will be instructed to inform the investigator prior to starting any new medications from the time of first dose of study treatment until discontinuation of study treatment. Any permitted concomitant medication(s), including non-prescription medication(s) and herbal product(s), taken during the study will be recorded in the eCRF. The minimum requirement for reporting is drug name, dose, dates of administration, and the reason for medication.

2.3.4.1 Permitted Medications and Non-Drug Therapies Elective palliative surgery or radiation may be permitted on a case-by-case basis in consultation with GSK Medical Monitor.

The following medications are permitted as indicated:

a. Bisphosphonates and receptor activator of nuclear factor-kappaB ligand (RANKL) inhibitors (e.g. denosumab): subjects are required to have been on a stable dose for at least 4 weeks prior to receiving first dose of H2L5 hIgG4PE. Prophylactic use in subjects without evidence or history of bone metastasis is not permitted, except for the treatment of osteoporosis.

b. Growth factors: initiation of growth factors is not permitted during the first 4 weeks of study treatment, unless clinically indicated for toxicity management and agreed upon by the investigator and the GSK Medical Monitor.

c. Steroids: Subjects with pre-existing conditions requiring steroids are permitted to continue taking up to a maximum of 10 mg of prednisone or equivalent provided the subject has been on a stable dose for at least 28 days before first dose of H2L5 hIgG4PE; refer to exclusion criterion 9 in Section 2.2.2 for further requirements. Steroids used for chemotherapy premedication are permitted.

2.3.4.2. Prohibited Medications and Non-Drug Therapies

The following medications are prohibited before the first dose of study treatment (refer to Section 2.2.2 for specific time requirements) and while on treatment in this study:

a. Anti-cancer therapies (other than those used in this study) that include but are not limited to chemotherapy, immunotherapy, biologic therapy, hormonal therapy (other than physiologic replacement), surgery, and radiation therapy (other than palliative intervention as described in Section 2.3.4.1);

b. Any investigational drug (s) other than those referred to in this study;

c. Live vaccines such as intra-nasal flu vaccine.

### 2.4 Study Assessment and Procedures

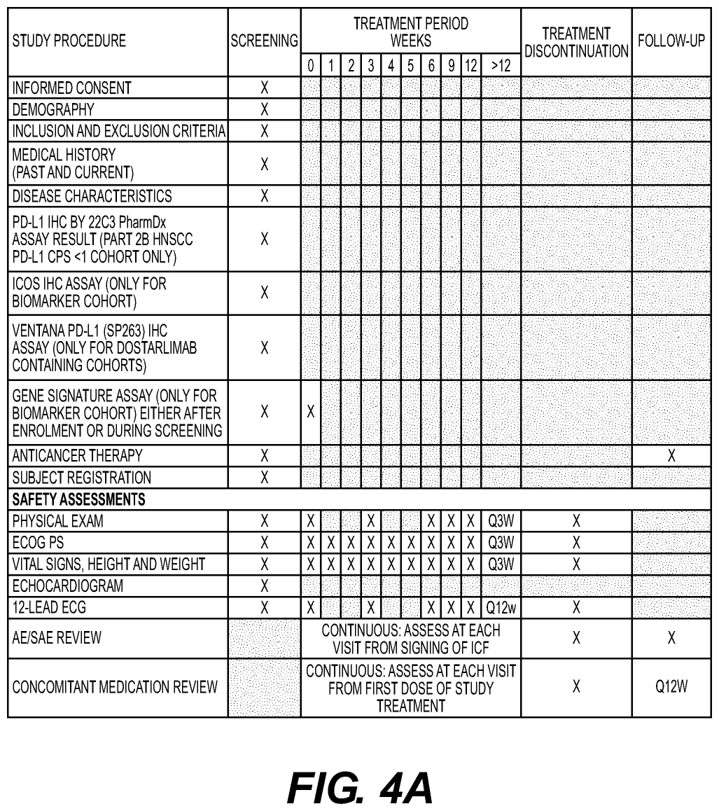

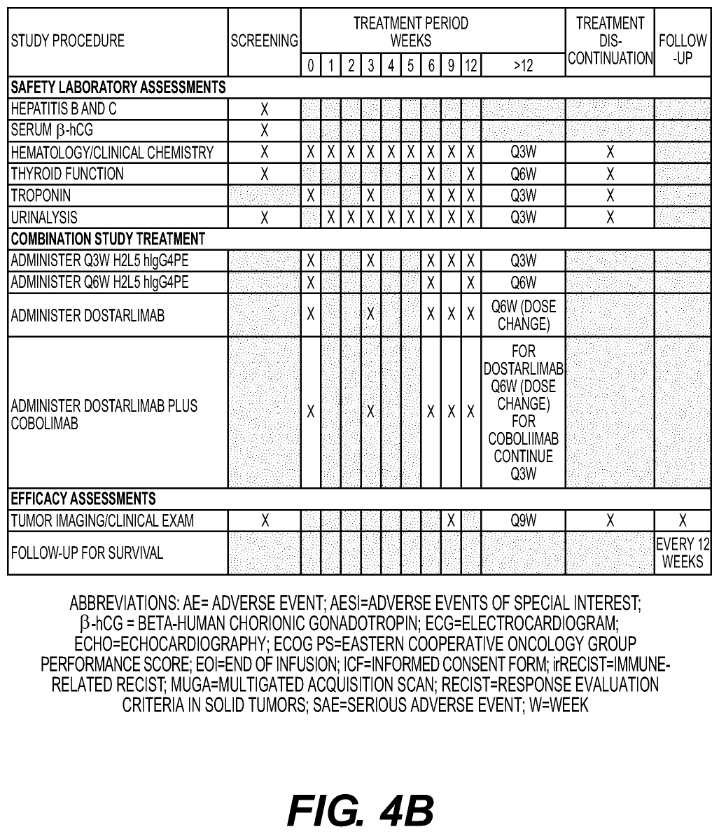

This section lists the procedures and parameters of each planned study assessment. The exact timing of each assessment is listed in the Time and Events Tables depicted in FIGS. 4 and 5.

The following points must be noted:
If assessments are scheduled for the same nominal time, then the assessments should occur in the following order:
1. 12-lead ECG
2. Vital signs
3. Blood draws (e.g. PK blood draws). Note: The timing of the assessments should allow the blood draw to occur at the exact nominal time.
The timing and number of planned study assessments, including safety, pharmacokinetic, pharmacodynamic/biomarker or others assessments may be altered during the course of the study based on emerging data (e.g. to obtain data closer to the time of peak plasma concentrations) to ensure appropriate monitoring.
No more than 500 mL of blood will be collected over the first four doses of study treatment.

2.4.1 Screening and Critical Baseline Assessments

The following demographic parameters will be captured: year of birth, sex, race and ethnicity.

Medical history including cardiovascular medical history/risk factors will be assessed as related to the inclusion/exclusion criteria listed in Section 2.2.1 and Section 2.2.2.

Disease characteristics including medical, surgical, and treatment history including radiotherapy, date of initial diagnosis, stage at initial diagnosis, histology, tumor genetic/genomic features, tumor viral status and current sites of disease will be taken as part of the medical history and disease status; scans from imaging studies performed prior to screening scans required for baseline lesion assessments may be requested. Details concerning prior anti-cancer therapy (e.g. systemic and radiation therapy) including best response to prior systemic therapy will be recorded for at least two prior lines of therapy (if available).

For subjects with PD-1/L1 treatment naïve HNSCC screening for enrollment to the HNSCC PD-L1 CPS<1 cohort only: PD-L1 protein expression using the PD-L1 IHC 22C3 pharmDx assay by local laboratory testing; if not available, central laboratory testing. An evaluable CPS score is required for eligibility; refer to Section 2.2.1 for CPS eligibility requirements.

Baseline lesion assessments required within 30 days prior to the first dose of H2L5 hIgG4PE include:
Computed Tomography (CT) scan with contrast of the chest, abdomen, and pelvis;
For subjects with head and neck cancer, a CT/Magnetic Resonance Imaging (MRI) of the head and neck area is required;
Clinical disease assessment for palpable/visible lesions;
Other areas as indicated by the subject's underlying disease present prior to screening.

Note: Although CT scan is preferred, MRI may be used as an alternative method of baseline disease assessment, especially for those subjects where a CT scan is contraindicated due to allergy to contrast, provided that the method used to document baseline status is used consistently throughout study treatment to facilitate direct comparison. Refer to RECIST version 1.1 guidelines for use of fluorodeoxyglucose-positron emission tomography (FDG-PET)/CT (Eisenhauer et al. Eur J Cancer. 2009; 45:228-247).

Refer to Section 2.4.2 for baseline documentation of target and non-target lesions.

Safety and laboratory assessments required at baseline include:
Physical examination
Performance Status
Vital Signs
Concomitant medication
   Recorded starting from screening through post-study follow-up.
   At a minimum, the drug name, route of administration, dose and frequency of dosing, along with start and stop dates should be recorded.
Electrocardiogram
Echocardiogram or MUGA
Laboratory assessments

Refer to Time and Events Tables in FIGS. 4 and 5 for additional details on assessments required at screening and prior to start of study treatment.

2.4.2 Evaluation of Anti-Cancer Activity

RECIST version 1.1 guidelines will be used to determine the overall tumor burden at screening, select target and non-target lesions, and in the disease assessments through the duration of the study (Eisenhauer, 2009).

As indicated in RECIST version 1.1 guidelines:
Lymph nodes that have a short axis of <10 mm are considered non-pathological and must not be recorded or followed.
Pathological lymph nodes with <15 mm, but 10 mm short axis are considered non-measurable.
Pathological lymph nodes with 15 mm short axis are considered measurable and can be selected as target lesions; however, lymph nodes should not be selected as target lesions when other suitable target lesions are available.
Measurable lesions up to a maximum of two lesions per organ and 5 lesions in total, representative of all involved organs, should be identified as target lesions, and recorded and measured at baseline. These lesions should be selected based on their size (lesions with the longest diameter) and their suitability for accurate repeated measurements (either by imaging techniques or clinically).

Note: Cystic lesions thought to represent cystic metastases must not be selected as target lesions when other suitable target lesions are available.

Note: Measurable lesions that have been previously irradiated and have not been shown to be progressing following irradiation must not be considered as target lesions.
Lytic bone lesions or mixed lytic-blastic lesions, with identifiable soft tissue components, that can be evaluated by CT or MRI) can be considered measurable. Bone scans, FDG-PET scans or X-rays are not considered adequate imaging techniques to measure bone lesions.
All other lesions (or sites of disease) must be identified as non-target and must also be recorded at baseline. Non-target lesions will be grouped by organ. Measurements of these lesions are not required, but the presence or absence of each must be noted throughout follow-up.

Disease assessment modalities may include imaging (e.g. CT scan, MRI, bone scan) and physical examination (as indicated for palpable/superficial lesions).

As indicated in Section 2.4.1, baseline disease assessment must be completed within 30 days prior to the first dose of H2L5 hIgG4PE. On-treatment disease assessments occur every 9 weeks until Week 54. After Week 54, disease assessments will be performed every 12 weeks then at the time of discontinuation of study treatment. At each post-baseline assessment, evaluation of the sites of disease (all target and non-target lesions) identified by the baseline scans is required. CT scans with contrast of the chest, abdomen, and pelvis, or if contra-indicated, MRI, is required at each post-baseline assessment. To ensure comparability between the baseline and subsequent assessments, the same method of assessment and the same technique will be used when assessing response.

For post-baseline assessments, a window of ±7 days is permitted to allow for flexible scheduling. If the last radiographic assessment was more than 9 weeks prior to the subject's discontinuation from study treatment, or >12 weeks if after Week 54, a disease assessment should be obtained.

Subjects with disease progression by RECIST version 1.1 guidelines are required to have a confirmatory disease assessment at least 4 weeks after the date disease progression was declared in order to confirm disease progression by irRECIST guidelines.

Subjects whose disease responds (either CR or PR) must have a confirmatory disease assessment performed at least 4 weeks after the date of assessment during which the response was demonstrated. More frequent disease assessments may be performed at the discretion of the investigator. In the subjects who attain a confirmed CR and fulfil the requirement for early discontinuation of study treatment (refer to Section 2.2.3), disease assessments at a frequency of will be performed every 12 weeks until progression. If study treatment is resumed upon disease progression and following consultation with the Investigator and GSK Medical Monitor, imaging scans which indicated progression will serve as the baseline scans.

The visit level responses and treatment-based decisions will incorporate irRECIST guidelines as described in Section 2.6.

2.4.3 Physical Examinations

A complete physical examination will include, at a minimum, assessment of the Cardiovascular, Respiratory, Gastrointestinal and Neurological systems. Height (at Screening only) and weight will also be measured and recorded.

A brief physical examination will include at a minimum, assessments of the skin, lungs, cardiovascular system, and abdomen (liver and spleen).

Investigators should pay special attention to clinical signs related to previous serious illnesses.

2.4.4 Performance Status

Performance status will be assessed using the ECOG scale as described in Section 2.7.

2.4.5 Vital Signs

Vital signs will be measured in semi-supine position after 5 minutes of rest and will include temperature, systolic and diastolic blood pressure and pulse rate. In the case of an abnormal first reading, three readings of blood pressure and/or pulse rate must be taken, whereby the first reading should be rejected and the second and third averaged to give the measurement to be recorded in the eCRF.

Vital signs will be measured more frequently if warranted by clinical condition of the subject.

On days where vital signs are measured multiple times, temperature does not need to be repeated unless clinically indicated.

If a subject develops fever, the subject will be managed using fever management guidelines.

2.4.6 Electrocardiogram

12-lead electrocardiograms will be obtained using an ECG machine that automatically calculates the heart rate and measures PR, QRS, QT, and QTcF intervals; manual calculation of QTcF is permitted.

2.4.7 Echocardiograms

Echocardiograms will be performed at baseline to assess cardiac ejection fraction and cardiac valve morphology for the purpose of study eligibility. Additional ECHO assessments may be performed if clinically warranted. The evaluation of the echocardiography must include an evaluation for left ventricular ejection fraction (LVEF) and both right and left-sided valvular lesions. MUGA can be used in lieu of ECHO (if not available) in the assessment of LVEF; the same modality should be used in any subsequent assessments.

2.4.8 Biomarkers/Pharmacodynamic Markers

2.4.8.1 Blood Biomarkers

Blood samples will be collected and analyzed by flow cytometry to evaluate the binding of H2L5 hIgG4PE to the ICOS receptor.

The numbers of T cells, B cell, natural killer (NK) cells as well as the subsets of T cells, activation and proliferation status of T cells will be simultaneously evaluated by flow cytometry in the same blood sample. Blood samples will be collected for isolation of PBMCs and plasma. Plasma and serum samples will be used for the analyses of circulating soluble factors in relation to T cell activation and may be utilized for analysis of soluble ICOS or soluble ICOS-drug complexes depending on the availability of the assays. Circulating factors to be analyzed may include but are not limited to the presences of IFNγ, TNFα, IL-2, IL-4, IL-6, IL-10, IL-8, IL-13, IL-12p70, IL-21, and chemokines as well as antibodies against the tumor, self or viral antigens. Plasma samples may also be analyzed for cell-free DNA (cfDNA) or exosomes (ribonucleic Acid [RNA]) for novel markers of immune activation or response to treatment with H2L5 hIgG4PE as a monotherapy or in combination.

PBMCs isolated from whole blood will be preserved and stored for flow cytometry of additional cells such as immune regulatory populations which may include but are not limited to myeloid derived suppressor cells, subsequent functional analyses, assessment of T cell repertoires, their relationship to clinical responses and changes in response to treatment with H2L5 hIgG4PE. The functional state of PBMCs may be analyzed for expression of cytokines which may include but not limited to IFNγ, IL-2, IL-10, TNFα, Granzyme B, PD-1, TIM3, and CD107a. PBMCs may also be evaluated for genomic (deoxyribonucleic acid [DNA]) and gene expression (RNA or protein) alterations to determine treatment-related changes in immune-related signatures.

2.4.8.2 Tumor Tissue

Archival tumor tissue, as well as, fresh pre- and on-treatment biopsies will be collected. The fresh biopsies samples are required in the pharmacodynamic/PK cohorts. Baseline tumor tissue at screening, either archival or fresh biopsy, and on-treatment fresh biosies at Week 6 are required for the HNSCC PD1/L1 treatment naïve PD-L1 CPS<1 and the HNSCC Q6W expansion cohorts.

Screening (archival or fresh) and on-treatment week 6 biopsy samples are required; required in subjects enrolled in the PK/pharmacodynamic cohort for the combination studies with dostarlimab and dostarlimab plus cobolimab.

Additionally, the following screening tests will be evaluated in cohorts specified below:
PD-L1 IHC 22C3 pharmDx assay for the enrolment into HNSCC PD-L1 CPS<1 cohort only.
Ventana PD-L1 (SP263) IHC assay for the enrolment into the expansion cohort with dostarlimab and dostarlimab plus cobolimab combination studies.

Tumor tissues collected at screening and on-treatment will also be evaluated by IHC, multiplex immunofluorescence technology or potentially other methods for expression of phenotypic and functional immune cell markers on tumor infiltrating lymphocytes (TIL) and other immune cells as well as immune signaling markers on tumor cells to understand the anti-tumor responses (including but not limited to PDL-1, ICOS, TIM-3, NY-ESO, TGF-beta) In addition, when possible, similar analyses will be performed on tumor tissue obtained upon progression. Additionally, tumor tissue may be sequenced to assess T cell receptor diversity (TCR diversity) as well as evaluated for any DNA/RNA/protein changes correlating with response.

### 2.5 Statistical Considerations and Data Analyses

2.5.1 Dose Escalation

Safety and tolerability of H2L5 hIgG4PE administered in combination with dostarlimab or dostarlimab plus cobolimab will be evaluated using an adaptive mTPI approach (shown in FIG. 3). The mTPI design is an extension of the toxicity probability interval method and employs a simple beta-binomial hierarchic model (Ji et al. Clin Trials. 2010; 7:653-663). Decision rules are based on calculating the unit probability mass (UPM) of three intervals corresponding to under dosing, proper dosing, and overdosing in terms of toxicity. Specifically, the under-dosing interval is defined as (0, pT−ε1), the overdosing interval as (pT+ε2, 1), and the proper dosing interval as (pT−ε1, pT+ε2), where ε1 and ε2 are small fractions, such as 0.05, to account for the uncertainty around the true target toxicity. A sensitivity analysis showed that the mTPI design is robust to the specification of ε values (Ji, 2010). In addition, ε1 and ε2 could take different values to reflect physician preference and the nature of the disease. For advanced diseases with few treatment options, higher toxicity rates might be considered acceptable, implying a specification of ε2>ε1. For less-advanced diseases, the two ε values could be identical or ε1>ε2. The three dosing intervals are associated with three different dose-escalation decisions. The under-dosing interval corresponds to a dose escalation (E), overdosing corresponds to a dose de-escalation (D), and proper dosing corresponds to staying at the current dose (S). Given an interval and a probability distribution, the UPM of that interval is defined as the probability of the interval divided by the length of the interval. The mTPI design calculates the UPMs for the three dosing intervals, and the one with the largest UPM implies the corresponding dose-finding decision. That decision provides the dose level to be used for future subjects. For example, if the under-dosing interval has the largest UPM, decision E, to escalate, will be executed, and the next cohort of subjects will be treated at the next higher dose level. Analyses showed that the decision based on the UPM is optimal in that it minimized a subsequent expected loss (Ji, 2010). Under the mTPI design, a trial is terminated when either the lowest dose is above the MTD or a pre-specified maximum sample size is reached.

2.5.2 Dose Expansion

In the expansion cohorts, after a minimum of 10 subjects have been enrolled in one dose/dose level in a cohort, the number of observed responses as well as other available date will be used for futility analysis.

If data permit, clinical activity of H2L5 hIgG4PE administered alone also may be evaluated using a Bayesian hierarchical modeling approach as an exploratory analysis. The design permits the trial to be frequently monitored for clinical activity with the constraint of both Type I and Type II error rates (Berry, 2013).

2.5.3 Sample Size Considerations

To complete dose escalation/safety run-ins for H2L5 hIgG4PE in combination with dostarlimab or dostarlimab plus cobolimab (refer to FIG. 2), it is estimated that approximately 241 subjects will be enrolled. Doses of H2L5 hIgG4PE to be studied will be guided by the mTPI design.

Simulations were conducted to determine the average sample size and percentage of times each dose would be selected as MTD under four different scenarios, considering the dose escalation phase of H2L5 hIgG4PE in combination with dostarlimab or dostarlimab plus cobolimab (guided by mTPI design). Cohort size of 3 subjects was used with a cap of 6 subjects at a dose level (the trial will stop recruitment if the next dose has already 6 subjects), the maximum sample size of 12 subjects for the dose escalation and 15 subjects at RP2D for further exploration. A safety rule for early termination was used where posterior probability exceeds target toxicity probability by 95%. 1000 simulated studies were used to derive the operating characteristics in FACTS version 6.1 software. The average sample sizes over the simulated clinical trials under four scenarios were 9.1, 9.3, 8.9 and 8.0 respectively, totaling approximately 25 subjects for each combination.

Details of the scenarios are provided in Table 9. The dose combinations in the table are the pre-selected dose combinations that are projected to be used in the trial.

TABLE 9
Simulation Results Under Various Scenarios

| | Scenario 1: | | Scenario 2: | | Scenario 3: | | Scenario 4: | |
|---|---|---|---|---|---|---|---|---|
| | Low Toxicity | | Low Toxicity | | Moderate Toxicity | | High Toxicity | |
| | | Per- | | Per- | | Per- | | Per- |
| | | cent | | cent | | cent | | cent |
| | | of | | of | | of | | of |
| | | Trials | | Trials | | Trials | | Trials |
| | | Se- | | Se- | | Se- | | Se- |
| Dose | | lect- | | lect- | | lect- | | lect- |
| H2L5 | | ing | | ing | | ing | | ing |
| hIgG4PE | | Dose | | Dose | | Dose | | Dose |
| (mg) in | True | as | True | as | True | as | True | as |
| com- | DLT | MTD | DLT | MTD | DLT | MTD | DLT | MTD |
| bination | Rate | (%) | Rate | (%) | Rate | (%) | Rate | (%) |
| 24 | 0.01 | <0.01% | 0.05 | 3% | 0.20 | 38% | 0.40 | 83% |
| 80 | 0.05 | 99.9% | 0.10 | 97% | 0.30 | 62% | 0.50 | 17% |

In the expansion phases, the sample size of a cohort or cohorts may target approximately 30 subjects per cohort. The condition by which the sample size will increase depends on the outcome from interim analysis of the null/alternative hypotheses that was determined for a tumor type.

For each tumor indication expansion cohort, an interim analysis will be conducted after efficacy data at any dose level are available on a minimum of subjects (refer to Section 2.5.5); a separate decision will be made for each disease cohort and dose. The trial may continue to enroll the maximum planned sample size to provide a better estimate on the distribution of the response rate in the different doses and target populations.

The trial is not designed to stop early for efficacy but is designed to assess futility if the predictive probability of success is 10% or less. The type I error rate, power, and predictive probability for assessing futility were determined from stating the minimum and maximum sample size, futility stopping rate, and the optimizing criterion as minimizing the sample size under null hypothesis. A very weak informative prior distribution with a mean response rate equal to the target response rate is assumed. Thus, the predictive probability for the response rate will be primarily driven by the data. The detailed decision criteria for all cohorts are documented in Section 2.5.5.

For any PD-1/L1 experienced combination therapy expansion cohorts starting with 10 subjects in each cohort and allowing for a maximum sample size of 30 for each cohort, this design will have an overall type I error rate (a) 5%. Under null hypotheses with 10% overall response rate (ORR), the expected sample size of the design is 15 subjects per cohort; and probability of early termination (PET) is 35% by 10 subjects evaluated and 80% by 20 subjects evaluated. Under the alternative hypothesis, if the true response rate is 30%, the probability of success is 83%; the expected sample size of the design is 28 subjects in total and PET is 3% by 10 subjects and 13% by 20 subjects.

For the PD-1/L1 naïve combination expansion cohorts including HNSCC, NSCLC with PD-L1<50%, bladder/urothelial cancer, cervical, and viral-positive cancers, starting with 10 subjects in each cohort and allowing for a maximum sample size of 30 for each cohort, this design will have an overall type I error rate (a) 9.8%. Under null hypotheses with 20% ORR, the expected sample size of the design is 16 subjects per cohort; and probability of early termination (PET) is 38% by 10 subjects evaluated and 72% by 20 subjects evaluated. Under the alternative hypothesis, if the true response rate is 40%, the probability of success is 83%; the expected sample size of the design is 28 subjects in total and PET is 5% by 10 subjects evaluated and 12% by 20 subjects evaluated.

For the biomarker positive cohort, starting with 12 subjects and allowing for a maximum sample size of 40, will have an overall type I error rate (a) of 4%. Under the null hypothesis of 10% ORR, the expected sample size of the design is 26 subjects; and the PET is 28% by 12 subjects evaluated and 55% by 30 subjects evaluated. Under the alternative hypothesis, if the true response rate is 25%, the power is 80%; the expected sample size of the design is 39 subjects in total and the PET is 3% by 12 subjects evaluated and 5% by 30 subjects evaluated. The biomarker negative group will similiarly allow for a maximum sample size of 40, and will follow enrolment/futility according to the biomarker positive group.

For the PD-1/L1 naïve combination expansion cohorts including NSCLC with PD-L150% and MSI-H/dMMR cancers, starting with 10 subjects in each cohort and allowing for a maximum sample size of 30 for each cohort, this design will have an overall type I error rate (a) 7.9%. Under null hypotheses with 30% ORR, the expected sample size of the design is 19 subjects per cohort; and probability of early termination (PET) is 15% by 10 subjects evaluated and 55% by 20 subjects evaluated. Under the alternative hypothesis, if the true response rate is 50%, the probability of success is 80%; the expected sample size of the design is 29 subjects in total and PET is 1.0% by 10 subjects evaluated and 6.2% by 20 subjects evaluated.

2.5.4 Data Analyses—xAnalysis Populations

All Treated Population will be defined as all subjects who receive at least one dose of H2L5 hIgG4PE. Safety and anti-cancer activity will be evaluated based on this analysis population.

Pharmacokinetic Population will be defined as all subjects from the All Treated Population for whom a PK sample is obtained and analyzed.

Pharmacodynamic Population will be defined as subjects in the All Treated Subjects Population for whom pre- and on-treatment paired and evaluable tumor biopsies or pre- and on-treatment blood samples were obtained and analyzed for biomarkers.

2.5.5 Interim Analysis

No formal interim analyses will be performed using the data generated during the dose escalation phases of the study. Available safety and PK/pharmacodynamic data will be reviewed after completion of each dose level. This review will support the decision to escalate to the dose level using the rules as described in Section 2.5.1. For dose expansion cohorts, continuous assessment of efficacy and safety will be performed after first interim analysis based upon a minimum of 10 subjects in at least one of the expansion cohort with available unconfirmed overall response data.

2.5.6 Pharmacokinetic Analyses

Validated analytical methods will be used to measure concentrations of dostarlimab and cobolimab. The following pharmacokinetic parameters will be determined using noncompartmental method, if data permit, and may include but not be limited to:
maximum observed plasma concentration (Cmax)
time to Cmax (tmax)
Cmin
area under the plasma concentration-time curve (AUC(0-t), AUC(0-∞)) and AUC(0-T))
apparent terminal phase elimination rate constant (λz) (single dose)
apparent terminal phase half-life (t½)
systemic clearance of parent drug (CL)
   2.6 Guidelines for Assessment of Disease, Disease Progression and Response Criteria—Adapted from RECIST Version 1.1

2.6.1 Assessment Guidelines

The same diagnostic method, including use of contrast when applicable, must be used throughout the study to evaluate a lesion. Contrast agents must be used in accordance with the Image Acquisition Guidelines.

All measurements must be taken and recorded in millimeters (mm), using a ruler or calipers.

Ultrasound is not a suitable modality of disease assessment. If new lesions are identified by ultrasound, confirmation by CT or MRI is required.

Fluorodeoxyglucose (FDG)-PET is generally not suitable for ongoing assessments of disease. However FDG-PET can be useful in confirming new sites of disease where a positive FDG-PET scans correlates with the new site of disease present on CT/MRI or when a baseline

FDG-PET was previously negative for the site of the new lesion. FDG-PET may also be used in lieu of a standard bone scan providing coverage allows interrogation of all likely sites of bone disease and FDG-PET is performed at all assessments.

If PET/CT is performed then the CT component can only be used for standard response assessments if performed to diagnostic quality, which includes the required anatomical coverage and prescribed use of contrast. The method of assessment must be noted as CT on the eCRF.

Clinical Examination: Clinically detected lesions will only be considered measurable when they are superficial (e.g. skin nodules). In the case of skin lesions, documentation by color photography, including a ruler/calipers to measure the size of the lesion, is required.

CT and MRI: Contrast enhanced CT with 5 mm contiguous slices is recommended. Minimum size of a measurable baseline lesion must be twice the slice thickness, with a minimum lesion size of 10 mm when the slice thickness is 5 mm. MRI is acceptable, but when used, the technical specification of the scanning sequences must be optimized for the evaluation of the type and site of disease and lesions must be measured in the same anatomic plane by use of the same imaging examinations. Whenever possible, the same scanner should be used.

X-ray: In general, X-ray should not be used for target lesion measurements owing to poor lesion definition. Lesions on chest X-ray may be considered measurable if they are clearly defined and surrounded by aerated lung; however, chest CT is preferred over chest X-ray.

Brain Scan: If brain scans are required, then contrast enhanced MRI is preferable to contrast enhanced CT.

2.6.2 Guidelines for Evaluation of Disease

Measurable and Non-Measurable Definitions are as follows:

Measurable lesion: A non-nodal lesion that can be accurately measured in at least one dimension (longest dimension) of:
≥10 mm with MRI or CT when the scan slice thickness is no greater than 5 mm. If the slice thickness is greater than 5 mm, the minimum size of a measurable lesion must be at least double the slice thickness (e.g., if the slice thickness is 10 mm, a measurable lesion must be 20 mm).
≥10 mm caliper/ruler measurement by clinical exam or medical photography.
≥20 mm by chest X-ray.
Additionally, lymph nodes can be considered pathologically enlarged and measurable if ≥15 mm in the short axis when assessed by CT or MRI (slice thickness recommended to be no more than 5 mm). At baseline and follow-up, only the short axis will be measured.

Non-measurable lesion: All other lesions including lesions too small to be considered measurable (longest diameter <10 mm or pathological lymph nodes with 10 mm and <15 mm short axis) as well as truly non-measurable lesions, which include: leptomeningeal disease, ascites, pleural or pericardial effusions, inflammatory breast disease, lymphangitic involvement of the skin or lung, abdominal masses/abdominal organomegaly identified by physical exam that is not measurable by reproducible imaging techniques.

Measurable disease: The presence of at least one measurable lesion. Palpable lesions that are not measurable by radiologic or photographic evaluations may not be utilized as the only measurable lesion.

Non-Measurable only disease: The presence of only non-measurable lesions. Note: non-measurable only disease is not allowed per protocol.

2.6.3 Immune-Related RECIST Response Criteria

Evaluation of target lesions are summarised in Table 10.

TABLE 10

| | |
|---|---|
| New, measurable^{a} lesions | Incorporated into tumor burden |
| New, non measurable | Do not define progression |
| lesions | (but preclude CR) |
| irCR | Disappearance of all lesions in two consecutive observations not less |
| | than 4 weeks apart. Any pathological lymph nodes (whether target |
| | or non-target) must have reduction in short axis to <10 mm. |
| irPR | >30% decrease in tumor burden compared with baseline in two |
| | observations at least 4 weeks apart |
| irSD | 30% decrease in tumor burden compared with baseline cannot be |
| | established nor 20% increase compared with nadir |
| irPD^{b} | At least 20% increase in tumor burden compared with nadir (at any |
| | single time point) in two consecutive observations at least 4 weeks |
| | apart. In addition to the relative increase of 20%, the sum must also |
| | demonstrate an absolute increase of at least 5 mm. |

^{a}Measureable per RECIST v1.1.
^{b}Treatment decisions will be based upon the immune-related RECIST guidelines.

2.6.3.1 Anti-Tumor Response Based on Total Measurable Tumor Burden

For Modified RECIST based on RECIST v1.1 and Immune-Related RECIST [Wolchok et al. Clin Cancer Res 2009; 15(23): 7412-20; Nishino et al. Clin Cancer Res. 2013; 19:3936-3943], the initial target (“index”) and measurable new lesions are taken into account. At the baseline tumor assessment, the sum of the diameters in the plane of measurement of all target lesions (maximum of five lesions in total and a maximum of two lesions per organ representative of all involved organs) is calculated.

Note: If pathological lymph nodes are included in the sum of diameters, the short axis of the lymph node(s) is added into the sum. The short axis is the longest perpendicular diameter to the longest diameter of a lymph node or nodal mass. At each subsequent tumor assessment, the sum of diameters of the baseline target lesions and of new, measurable nodal and non-nodal lesions (10 mm), up to 2 new lesions per organ are added together to provide the total tumor burden:

Tumor Burden=Sum of diameters_{target lesions}+sum of diameters_{new, measurable lesions}

2.6.3.2 Time-point response assessment using the Immune-Related RECIST criteria

Percentage changes in tumor burden per assessment time point describe the size and growth kinetics of both conventional and new, measurable lesions as they appear. At each tumor assessment, the response in index and new, measurable lesions is defined based on the change in tumor burden (after ruling out irPD). Decreases in tumor burden must be assessed relative to baseline measurements (i.e. the sum of diameters of all target lesions at screening).

2.6.3.3 Evaluation of Non-Target Lesions

Definitions for assessment of response for non-target lesions are as follows:
Complete Response (CR): The disappearance of all non-target lesions. All lymph nodes identified as a site of disease at baseline must be non-pathological (e.g. <10 mm short axis).
Non-CR/Non-PD: The persistence of 1 or more non-target lesion(s) or lymph nodes identified as a site of disease at baseline 10 mm short axis.
Progressive Disease (PD): Unequivocal progression of existing non-target lesions.
Not Applicable (NA): No non-target lesions at baseline.
Not Evaluable (NE): Cannot be classified by one of the four preceding definitions.

Note: In the presence of measurable disease, progression on the basis of solely non-target disease requires substantial worsening such that even in the presence of SD or PR in target disease, the overall tumor burden has increased sufficiently to merit discontinuation of therapy. Furthermore, sites of non-target lesions, which are not assessed at a time point based on the assessment schedule, should be excluded from the response determination (e.g. non-target response does not have to be “Not Evaluable”).

2.6.3.4 New Lesions

New malignancies denoting disease progression must be unequivocal. Lesions identified in follow-up in an anatomical location not scanned at baseline are considered new lesions.

Any equivocal new lesions must continue to be followed. Treatment can continue at the discretion of the investigator until the next scheduled assessment. If at the next assessment, the new lesion is considered to be unequivocal, progression would be declared.

2.6.3.5 Evaluation of Overall Response

Table 11 presents the overall response at an individual disease assessment time-point accounting for all possible combinations of responses in target and non-target lesions with or without the appearance of new lesions for subjects with measurable disease at baseline.

TABLE 11
Evaluation of Overall Response for Subjects with
Measurable Disease at Baseline

| Target | Non-Target | New | Overall |
|---|---|---|---|
| Lesions | Lesions | Lesions | Response |
| CR | CR or NA | No | CR |
| CR | Non-CR/Non-PD or NE | No | PR |
| PR | Non-PD or NA or NE | No | PR |
| SD | Non-PD or NA or NE | No | SD |
| NE | Non-PD or NA or NE | No | NE |
| PD | Any | Yes or No | PD |
| Any | PD | Yes or No | PD |
| Any | Any | Yes | PD |

Abbreviations: CR = Complete response, PR = Partial response, SD = Stable disease, PD = Progressive disease, NA = Not applicable, and NE = Not Evaluable

2.6.3.6 Evaluation of Best Overall Response

The best overall response is the best response recorded from the start of the treatment until disease progression/recurrence and will be determined programmatically by GSK based on the investigators assessment of response at each time point.

To be assigned a status of SD, follow-up disease assessment must have met the SD criteria at least once after the first dose at a minimum interval of days as defined in the RAP.

If the minimum time for SD is not met, best response will depend on the subsequent assessments. For example, if an assessment of PD follows the assessment of SD and SD does not meet the minimum time requirement the best response will be PD. Alternatively, subjects lost to follow-up after an SD assessment not meeting the minimum time criteria will be considered not evaluable.

2.6.3.7 Confirmation Criteria

To be assigned a status of PR or CR, a confirmatory disease assessment must be performed no less than 4 weeks (28 days) after the criteria for response are first met.

### 2.7 ECOG Performance Status

Summary presented in Table 12.

TABLE 12
ECOG Performance Status

| Grade | Descriptions |
|---|---|
| 0 | Normal activity. Fully active, able to carry on all pre-disease performance without |
| | restriction. |
| 1 | Symptoms, but ambulatory. Restricted in physically strenuous activity, but |
| | ambulatory and able to carry out work of a light or sedentary nature (e.g., light |
| | housework, office work). |
| 2 | In bed <50% of the time. Ambulatory and capable of all self-care, but unable to |
| | carry out any work activities. Up and about more than 50% of waking hours. |
| 3 | In bed >50% of the time. Capable of only limited self-care, confined to bed or chair |
| | more than 50% of waking hours. |
| 4 | 100% bedridden. Completely disabled. Cannot carry on any self-care. Totally |
| | confined to bed or chair. |
| 5 | Dead. |

Oken et al. Am J Clin Oncol. 1982; 5:649-655.

### 2.8 Events of Clinical Interest

These are selected events considered of clinical interest; they may be non-serious AEs or SAEs. Events of Clinical Interest are different from Adverse Events of Special Interest (AESI) in that an AESI is defined as an adverse event of potential immunologic etiology. Such events recently reported after treatment with other immune modulatory therapy include colitis, uveitis, hepatitis, pneumonitis, diarrhea, endocrine disorders, and specific cutaneous toxicities, as well as other events that may be immune mediated.

For the time period beginning with the administration of the first dose of study treatment through 30 days following discontinuation of study treatment, any ECI, or follow up to an ECI, whether or not related to the study drug(s), must be reported to the Sponsor. ECI include:

1. Overdose of study drug(s) that is not associated with clinical symptoms or abnormal laboratory results must be reported within 5 days.

2. An elevated aspartate aminotransferase (AST) or alanine aminotransferase (ALT) lab value that is greater than or equal to 3× the upper limit of normal and an elevated total bilirubin lab value that is greater than or equal to 2× the upper limit of normal and, at the same time, an alkaline phosphatase lab value that is less than 2× the upper limit of normal, as determined by way of protocol-specified laboratory testing or unscheduled laboratory testing. This ECI must be reported within 24 hours. These criteria are based upon available regulatory guidance documents. The purpose of the criteria is to specify a threshold of abnormal hepatic tests that may require an additional evaluation for an underlying etiology.

3. Infection with COVID-19 coronavirus, whether suspected based on exposure history and clinical signs and symptoms, or confirmed by laboratory test in the context of exposure history and clinical signs and symptoms. Reporting will follow WHO and GSK guidelines.

### 2.9 Genetic Research

2.9.1 Genetic Research Objectives and Analyses

The objectives of the genetic research are to investigate the relationship between genetic variants and:
Response to medicine, including H2L5 hIgG4PE, other immune therapy under investigation in this study, or any concomitant medicines;
Cancer susceptibility, severity, and progression and related conditions.

Genetic data may be generated while the study is underway or following completion of the study. Genetic evaluations may include focused candidate gene approaches and/or examination of a large number of genetic variants throughout the genome (whole genome analyses). Genetic analyses will utilize data collected in the study and will be limited to understanding the objectives highlighted above. Analyses may be performed using data from multiple clinical studies to investigate these research objectives.

Appropriate descriptive and/or statistical analysis methods will be used. A detailed description of any planned analyses will be documented in a Reporting and Analysis Plan (RAP) prior to initiation of the analysis. Planned analyses and results of genetic investigations will be reported either as part of the clinical RAP and study report, or in a separate genetics RAP and report, as appropriate.

2.9.2 Study Population

Any subject who is enrolled in the study can participate in genetic research. Any subject who has received an allogeneic bone marrow transplant must be excluded from the genetic research.

2.9.3 Study Assessments and Procedures

A key component of successful genetic research is the collection of samples during clinical studies. Collection of samples, even when no a priori hypothesis has been identified, may enable future genetic analyses to be conducted to help understand variability in disease and medicine response.

A 6 ml blood sample will be taken for DNA extraction. A blood sample is collected at the baseline visit, after the subject has been randomized and provided informed consent for genetic research. Instructions for collection and shipping of the genetic sample are described in the laboratory manual. The DNA from the blood sample may undergo quality control analyses to confirm the integrity of the sample. If there are concerns regarding the quality of the sample, then the sample may be destroyed. The blood sample is taken on a single occasion unless a duplicate sample is required due to an inability to utilize the original sample.

The genetic sample is labelled (or “coded”) with the same study specific number used to label other samples and data in the study. This number can be traced or linked back to the subject by the investigator or site staff. Coded samples do not carry personal identifiers (such as name or social security number).

### Example 3. Combination Therapy Human Clinical Trial Protocol Development

### 3.1 Study Design

A combination of H2L5 hIgG4PE and dostarlimab plus cobolimab in a platform NSCLC study and compared with the current standard of care, docetaxel. Docetaxel as the current standard of care for NSCLC is administered at a dose of 75 mg/m² via IV infusion once every three weeks for 6 cycles and may be discontinued after 6 cycles at the discretion of the investigator. The study will investigate H2L5 hIgG4PE at a dose of 24 mg Q3W, dostarlimab at a dose of Q3W and cobolimab at a dose of Q3W. Part 1 will be a non-randomized, safety and PK/PD evaluation, Part 2 will be a randomized, Phase II comparing the efficacy and safety of the combination.

The combination evaluated will be investigated in patients with relapsed/refractory advanced NSCLC who have failed prior platinum-containing chemotherapy regimen and an agent targeting PD-1/PD-L1—either in combination or as separate lines.

3.1.1 H2L5 hIgG4PE and Dostarlimab Plus Cobolimab

24 mg H2L5 hIgG4PE will be administered first as a 30-minute IV infusion (infusion time may be adjusted in the event an infusion related reaction occurs) under medical supervision of an investigator or designee. 500 mg dostarlimab will be administered via IV infusion at least 30 minutes and no longer than one hour following H2L5 hIgG4PE EOI (end of infusion) Q3W administered intravenously and 300 mg cobolimab Q3W at least 30 minutes and no longer than one hour after dostarlimab EOI. The study design is shown in Table 13.

TABLE 13
Description and administration of H2L5 hIgG4PE and dostarlimab plus
cobolimab

| | | | H2L5 hIgG4PE |
|---|---|---|---|
| Name | Dostarlimab | Cobolimab | (ICOS Agonist) |
| Description | PD-1 Inhibitor | Anti-Tim3 | Humanized anti- |
| | | | ICOS IgG4 mAb |
| Dosage form/ | 50 m² mg/ml | 20 mg/ml solution | Solution for |
| strength | solution | | injection/ |
| | | | 10 mg/mL |
| Dosage | 500 mg (see | 300 mg | 24 mg |
| | protocol for details) | | |
| Route of administration | IV infusion | IV infusion | IV infusion |
| Dosing | Administer | Administer | Administer |
| instructions^{a}/ | diluted | diluted | diluted |
| frequency | product/once | product/once | product/once |
| | Q3W | Q3W | Q3W |
| | (refer to SRM for | (refer to SRM for | |
| | infusion time) | infusion time) | |

3.1.2 Dose Limiting Toxicity

The severity of all toxicities will be graded using National Cancer Institute-Common Terminology Criteria for Adverse Events (NCI-CTCAE) (version 5.0) [NCI, 2017]. The DLT observation period is 21 days in length and begins on the day H2L5 hIgG4PE is first administrated to the participant.

A DLT is defined as an adverse event (AE) that meets at least one of the criteria listed in Table 4 and is considered by the investigator to be clinically relevant and attributed (probably, or possibly) to the study treatment during the 28-day DLT observation period. An AE considered related to the underlying disease under study it is not defined as a DLT.

TABLE 14
Dose-Limiting Toxicity Criteria

| Toxicity | DLT Definition |
|---|---|
| Hematologic | Febrile neutropenia as defined by CTCAE v4 |
| | Grade 4 neutropenia of >7 days in duration or requiring G-CSF |
| | Grade 4 anemia of any duration |
| | Grade 4 thrombocytopenia of any duration or Grade 3 thrombocytopenia |
| | with bleeding |
| Non- | Grade 4 toxicity |
| hematologic | Grade 3 pneumonitis of any duration |
| | Grade 3 toxicity that does not resolve to ≤ Grade 1 or baseline within 3 |
| | days despite optimal supportive care |
| | Any Grade 2 ocular toxicity requiring systemic steroids, or any ≥ Grade 3 |
| | ocular toxicity |
| | Following events are not considered DLTs |
| | Grade 3 and Grade 4 asymptomatic electrolyte abnormalities that |
| | are corrected within 24 hours without clinical sequelae |
| | Grade 3 nausea, vomiting, or fatigue that resolves to ≤Grade 1 |
| | within 7 days with optimal supportive care |
| | Grade 3 and Grade 4 infusion reactions in participants not |
| | receiving prophylaxis for infusion related reactions (IRRs) |
| Other | Toxicity that results in permanent discontinuation of H2L5 hIgG4PE |
| | monotherapy or H2L5 hIgG4PE and agent in combination during the first |
| | four weeks of treatment |
| | Grade 3/Grade 4 toxicity that results in a participant not receiving the |
| | expected doses of a regimen in Cycle 1, defined by 21 days |
| | Any other event which in the judgment of the investigator and GSK Medical |
| | Monitor is considered to be a DLT |

Note:
Suggested toxicity management guidelines may include systemic corticosteroids for immune-related toxicities; if systemic corticosteroids use delays administration of the second dose of study treatment and the event does not otherwise meet the DLT criteria for non-hematologic toxicity, the dose delay will not be considered a DLT.

If a participant experiences a DLT during the DLT observation period, the participant may resume dosing provided the toxicity did not meet study treatment discontinuation criteria and following approval by the Sponsor.

3.1.3 Study Treatment and Duration

Participants enrolled will be treated until disease progression, intolerable toxicity, informed consent withdrawal or death. Combination study treatment will continue to be administered at the indicated schedule for a maximum duration of approximately 2 years or up to 35 treatment visits, whichever comes first.

After study treatment is permanently discontinued, participants will be followed for AEs. Additionally, participants will be followed for survival and subsequent anticancer therapy every 12 weeks via telephone contact until death or participant's withdrawal from further contact.

iRECIST is based on RECIST 1.1 but adapted to account for the unique tumor response seen with immunotherapeutic drugs (Seymour, 2017). Participants who attain a confirmed CR per iRECIST, have received at least 2 additional doses of study treatment beyond the date the initial CR was declared, and have been treated for a minimum of 6 months, may discontinue study treatment; and these participants will continue with the scheduled disease assessments. Participants may be permitted to resume study treatment upon disease progression following consultation between the treating investigator and the Sponsor/Medical Monitor, and upon written consent by the participant.

Participants who permanently discontinue study treatment will enter the survival follow-up period of the study and undergo assessments.

3.1.4 Dose Justification

The dose rational for of H2L5 hIgG4PE and dostarlimab plus cobolimab are as set out in 2.1.6. The recommended clinical dose and regimen of dostarlimab is 500 mg Q3W for 4 cycles followed by 1000 mg Q6W. For this study, dostarlimab will be dosed at 500 mg Q3W in order to match the schedule of the other drugs (cobolimab and H2L5 hIgG4PE) that are also administered Q3W and to reduce patient burden.

### 3.2 Selection of Study Population and Withdrawal Criteria

3.2.1 Inclusion Criteria

For a participant to be eligible for inclusion in this study all the following criteria must be fulfilled:
1. Capable of giving signed informed consent/assent
2. Male or female, aged 18 years or older at the time consent is obtained
3. Histologically or cytologically confirmed diagnosis of NSCLC (squamous or non-squamous) and:
   a. Documented disease progression based on radiographic imaging, during or after a maximum of 2 lines of systemic treatment for locally/regionally advanced recurrent, Stage IIIb/Stage IIIc/Stage IV or metastatic disease
   Two components of treatment must have been received in the same line or as separate lines of therapy:
      i. no more than or less than 1 line of platinum-containing chemotherapy regimen, and;
      ii. no more than or less than 1 line of PD(L)1 mAb containing regimen.
   b. Participants with known BRAF molecular alterations must have had disease progression after receiving the locally available standard of care treatment for the molecular alteration.
4. Measurable disease, presenting with at least 1 measurable lesion per RECIST 1.1 (Section 3.6 for definition of a measurable lesion)
5. Eastern Cooperative Oncology Group (ECOG) Performance Status (PS) score of 0 or 1 (refer to Section 3.7)
6. A tumor tissue sample obtained at any time from the initial diagnosis of NSCLC to time of study entry is mandatory. Although a fresh tumor tissue sample obtained during screening is preferred, archival tumor specimen is acceptable.
7. Adequate organ function as defined in Table 15:

TABLE 15
Definitions of Adequate Organ Function

| | |
|---|---|
| System | Laboratory Values |

Hematologic^{a}

| | |
|---|---|
| Absolute neutrophil count (ANC) | ≥1.5 × 10⁹/L (≥21500/μL) |
| Hemoglobin | ≥9 g/dL or ≥5.6 mmol/L |
| Platelets | ≥100 × 10⁹/L (≥100 000/μL) |

Hepatic

| | |
|---|---|
| Albumin | ≥2.5 g/dL |
| Total bilirubin | ≤1.5 × upper limit of normal (ULN) |
| For participants with | (isolated bilirubin >1.5 × ULN is |
| Gilbert's Syndrome (only | acceptable if bilirubin is fractionated |
| if direct bilirubin <35%) | and direct bilirubin <35%) |
| | ≤3.0 × ULN |
| Alanine aminotransferase (ALT) | ≤2.5 × ULN; |
| | or ≤5 × ULN for participants with |
| | documented liver metastases |

Renal

| | |
|---|---|
| Calculated creatinine clearance^{b} | >30 mL/min |

^{a}Participants may be transfused or receive growth factor treatment to meet minimum hematologic values up to 7 days prior to determining eligibility. Absolute Lymphocyte Count will be included in the baseline assessment, but no range limit requirement for the eligibility.
^{b}Calculated creatinine clearance is required to be calculated using the Chronic Kidney Disease Epidemiology Collaboration (CKD EPI) or Cockcroft-Gault formula. Either formula is acceptable and must be consistently utilized for each participant throughout the study.

8. A male participant must agree to use a highly effective contraception during the treatment period and for at least 120 days after the last dose of study treatment and refrain from donating sperm during this period.
9. A female participant is eligible to participate if she is not pregnant, not breastfeeding, and at least 1 of the following conditions apply:
   i. Not a woman of childbearing potential (WOCBP)
      or
   ii. A WOCBP who agrees to follow the contraceptive guidance during the treatment period and for at least 120 days after the last dose of study treatment.

3.2.2 Exclusion Criteria

A participant will not be eligible for inclusion in this study if any of the following criteria apply:
1. Received prior treatment with the following therapies (calculation is based on date of last therapy to date of first dose of study treatment):
   a. Docetaxel at any time
   b. Any of the investigational agents being tested in the study, including experimental ICOS agonist
   c. Systemic approved or investigational anticancer therapy within 30 days or 5 half-lives of the drug, whichever is shorter. At least 14 days must have elapsed between the last dose of prior anticancer agent and the first dose of study drug is administered.
   d. Prior radiation therapy: permissible if at least one non-irradiated measurable lesion is available for assessment per RECIST version 1.1 or if a solitary measurable lesion was irradiated, objective progression is documented. A wash out of at least 2 weeks before start of study drug for radiation of any intended use is required.
2. Received >2 prior lines of therapy for NSCLC, including participants with BRAF molecular alterations.
Note: Patients with known EGFR/ALK/ROS1 molecular alterations are excluded from participation in this study however patients with known exon 20 EGFR molecular alteration may be considered for inclusion in this study, if no other therapeutic options are available locally.
3. Invasive malignancy or history of invasive malignancy other than disease under study within the last 2 years, except as noted below:
   Any other invasive malignancy for which the participant was definitively treated, has been disease-free for at least 2 years and in the opinion of the principal investigator and GSK Medical Monitor will not affect the evaluation of the effects of the study treatment on the currently targeted malignancy, may be included in this clinical trial.
   Curatively treated non-melanoma skin cancer or successfully treated in situ carcinoma.
4. Central nervous system (CNS) metastases, with the following exception: Participants with asymptomatic CNS metastases who are clinically stable and have no requirement for steroids for at least 14 days prior to first dose of study treatment.
   Note: Participants with carcinomatous meningitis or leptomeningeal spread are excluded regardless of clinical stability.
5. Major surgery 28 days of first dose of study treatment.
6. Autoimmune disease (current or history) or syndrome that required systemic treatment within the past 2 years. Replacement therapies which include physiological doses of corticosteroids for treatment of endocrinopathies (for example, adrenal insufficiency) are not considered systemic treatments.
   Note: Participants with controlled Type 1 diabetes mellitus (T1DM) are eligible.
7. Receiving systemic steroids (>10 mg oral prednisone or equivalent) or other immunosuppressive agents within 7 days prior to first dose of study treatment.
Note: Steroids as premedication for hypersensitivity reactions (e.g., computed tomography (CT) scan premedication) are permitted.
8. Prior allogeneic/autologous bone marrow or solid organ transplantation.
9. Receipt of any live vaccine within 30 days prior to first dose of study treatment. Examples of live vaccines include, but are not limited to the following: measles, mumps, rubella, varicella/zoster (chicken pox), yellow fever, rabies, Bacillus Calmette-Guérin (BCG), and typhoid vaccine. Seasonal influenza vaccines for injection are generally killed virus vaccines and are allowed; however, intranasal influenza vaccines (for example, FluMist) are live attenuated vaccines and are not allowed.
10. Toxicity from previous anticancer treatment that includes:
   a. ≥Grade 3 toxicity considered related to prior immunotherapy and that led to treatment discontinuation.
   b. Toxicity related to prior treatment that has not resolved to Grade 1 (except alopecia, hearing loss, endocrinopathy managed with replacement therapy, and peripheral neuropathy which must be Grade 2).
11. History (current and past) of idiopathic pulmonary fibrosis, pneumonitis (for past pneumonitis exclusion only if steroids were required for treatment), interstitial lung disease, or organizing pneumonia.
Note: post-radiation changes in the lung related to prior radiotherapy and/or asymptomatic radiation-induced pneumonitis not requiring treatment may be permitted if agreed upon by the investigator and Medical Monitor.
12. Recent history (within the past 6 months) of uncontrolled symptomatic ascites, pleural or pericardial effusions
13. Recent history (within the past 6 months) of gastrointestinal obstruction that required surgery, acute diverticulitis, inflammatory bowel disease, or intra-abdominal abscess
14. History or evidence of cardiac abnormalities within the 6 months prior to enrollment which include:
   a. Serious, uncontrolled cardiac arrhythmia or clinically significant electrocardiogram abnormalities including second degree (Type II) or third degree atrioventricular block.
   b. Cardiomyopathy, myocardial infarction, acute coronary syndromes (including unstable angina pectoris), coronary angioplasty, stenting or bypass grafting
   c. Symptomatic pericarditis.
15. Current unstable liver or biliary disease per investigator assessment defined by the presence of ascites, encephalopathy, coagulopathy, hypoalbuminemia, esophageal or gastric varices, persistent jaundice, or cirrhosis.
Note: Stable chronic liver disease (including Gilbert's syndrome or asymptomatic gallstones) is acceptable if participant otherwise meets entry criteria.
16. Active infection requiring systemic therapy.
17. Known human immunodeficiency virus infection
18. History of severe hypersensitivity to monoclonal antibodies or hypersensitivity to ingredients used in the formulation of docetaxel.
19. Any serious and/or unstable pre-existing medical (aside from malignancy), psychiatric disorder, or other condition that could interfere with participant's safety, obtaining informed consent, or compliance to the study procedures in the opinion of the investigator
20. Pregnant or lactating female participants
21. Is currently participating in or has participated in a study of an investigational device within 4 weeks prior to the first dose of study treatment.
22. Presence of hepatitis B surface antigen (HBsAg) at screening or within 3 months prior to first dose of study intervention
23. Positive hepatitis C antibody test result at screening or within 3 months prior to first dose of study intervention.
Note: Participants with positive Hepatitis C antibody due to prior resolved disease can be enrolled, only if a confirmatory negative Hepatitis C RNA test is obtained.
24. Positive hepatitis C RNA test result at screening or within 3 months prior to first dose of study treatment.
Note: Test is optional and participants with negative Hepatitis C antibody test are not required to also undergo Hepatitis C RNA testing.
25. Known hypersensitivity to dostarlimab components or excipients.
26. Known hypersensitivity to dostarlimab components or excipients

### 3.3 Study Treatment

3.3.1 Method of Treatment Assignment

Once determined to be eligible for the study, all participants will be centrally randomized using an Interactive Web Response System (IWRS).

3.3.2 Blinding

This is an open-label study.

3.3.3 Concomitant Medications and Non-Drug Therapies

Participants will be instructed to inform the investigator prior to starting any new medications from the time of first dose of study treatment until discontinuation of study treatment. Any permitted concomitant medication(s), including non-prescription medication(s) and herbal product(s), taken during the study will be recorded in the eCRF. The minimum requirement for reporting is drug name, dose, dates of administration, and the reason for medication.

3.3.3.1 Permitted Medications and Non-Drug Therapies

All participants should receive full supportive care during the treatment course of the study, including transfusion of blood and blood products, and treatment with antibiotics, antiemetics, antidiarrheals, and analgesics, as appropriate. Seasonal flu vaccine is permitted as an injection only, that is, intra-nasal flu vaccine is not permitted. Elective palliative surgery or radiation may be permitted on a case-by-case basis in consultation with GSK Medical Monitor.

The following medications are permitted as indicated:

a. Bisphosphonates and receptor activator of nuclear factor-kappaB ligand (RANKL) inhibitors (e.g. denosumab): participants are required to have been on a stable dose for at least 4 weeks prior to receiving first dose of H2L5 hIgG4PE. Prophylactic use in participants without evidence or history of bone metastasis is not permitted, except for the treatment of osteoporosis.

b. Growth factors: Prophylactic use of growth factors is not permitted during study treatment, unless clinically indicated for toxicity management.

c. Steroids: Participants with pre-existing conditions requiring steroids are permitted to continue taking up to a maximum of 10 mg of prednisone or equivalent provided the participant has been on a stable dose for at least 28 days before first dose of study treatment; refer to exclusion criteria in Section 3.2.2 for further requirements. Steroids used for chemotherapy premedication are permitted.

d. Prescribed medicinal cannabinoids are permitted during the study as palliative therapy

3.3.3.2. Prohibited Medications and Non-Drug Therapies

The following medications are prohibited before the first dose of study treatment (refer to Section 3.2.2 for specific time requirements) and while on treatment in this study:

a. Anti-cancer therapies (other than those used in this study) that include but are not limited to chemotherapy, immunotherapy, biologic therapy, hormonal therapy (other than physiologic replacement), surgery, and radiation therapy (other than palliative intervention as described in Section 2.3.4.1);

b. Any investigational drug(s) other than those referred to in this study;

c. Live vaccines such as intra-nasal flu vaccine.

### 3.4 Study Assessment and Procedures

The following points must be noted:
Informed consent must be signed by a participant before any study required procedures are performed. However, procedures conducted as part of the routine clinical management (e.g., imaging studies) and conducted prior to signing of the study informed consent may be used for screening/baseline assessments.
If assessments are scheduled for the same nominal time, then the assessments should occur in the following order:
   1. 12-lead ECG
   2. Vital signs
   3. Blood draws Note: The timing of the assessments must allow the blood draw to occur at the exact nominal time.
The timing and number of planned study assessments, including safety, biomarker or others assessments may be altered during the course of the study based on emerging data to ensure appropriate monitoring.
No more than 900 mL of blood will be collected from each participant over the full course of study treatment (2 years).

3.4.1 Screening and Critical Baseline Assessments

Demographic parameters such as year of birth and sex will be captured.

Medical history including cardiovascular medical history, tobacco use, and other risk factors will be assessed as related to the inclusion/exclusion criteria.

Disease characteristics including medical, surgical, and treatment history including radiotherapy, date of initial diagnosis, stage at initial diagnosis according to the 8th Edition of TNM for Lung Cancer by the Union for International Cancer Control (UICC), histology, tumor genetic/genomic features sand current sites of disease will be taken as part of the medical history and disease status. Scans from imaging studies performed prior to screening may be requested for assessment of baseline lesions. Details concerning prior anticancer therapy (for example, systemic and radiation therapy), including best response to prior systemic therapy will be recorded.

If available, any antibiotic use within 90 days prior to the first dose of study should ideally be recorded to help inform the effect of antibiotics on clinical outcome through its manipulation of the immune system.

Baseline lesion assessments required within 28 days prior to the first dose of study treatment include: Computed tomography (CT) scan with contrast of the chest and abdomen

Note: Although a CT scan is preferred, magnetic resonance imaging (MRI) may be used as an alternative method of baseline disease assessment, especially for those participants where a CT scan is contraindicated due to allergy to contrast, provided the method used to document baseline status is used consistently throughout study treatment to facilitate direct comparison. When MRI is used for disease assessment, a non-contrast CT of the chest should also be performed, to evaluate the lungs. Refer to RECIST 1.1 guidelines for use of fluorodeoxyglucose-positron emission tomography (FDG-PET)/CT (Eisenhauer, 2009; Seymour, 2017).
MRI of brain with and without IV gadolinium (if clinically indicated)
Bone scan (if clinically indicated)
Clinical disease assessment for palpable/visible lesions
Other areas as indicated by the participant's underlying disease present prior to screening

Refer to Section 3.4.2 for baseline documentation of target and non-target lesions.

Safety and laboratory assessments required at baseline include:
Physical examination
ECOG Performance Status
Vital Signs
Concomitant medication
   Recorded starting from screening through post-study follow-up.
   Record all medications the participant is taking including prescription medications, over-the-counter (OTC) drugs or preparations, and herbal preparations including any cannabinoids and/or recreational drugs used.
   At a minimum, the drug name, route of administration, dose and frequency of dosing, along with start and stop dates should be recorded.
Electrocardiogram
Echocardiogram
Laboratory assessments

3.4.2 Evaluation of Anti-Cancer Activity

RECIST version 1.1 guidelines will be used to determine the overall tumor burden at screening, select target and non-target lesions, and in the disease assessments through the duration of the study (Eisenhauer, 2009).

As indicated in RECIST version 1.1 guidelines:
Lymph nodes that have a short axis of <10 mm are considered non-pathological and must not be recorded or followed.
Pathological lymph nodes with <15 mm, but 10 mm short axis are considered non-measurable.
Pathological lymph nodes with 15 mm short axis are considered measurable and can be selected as target lesions; however, lymph nodes should not be selected as target lesions when other suitable target lesions are available.
Measurable lesions up to a maximum of two lesions per organ and 5 lesions in total, representative of all involved organs, should be identified as target lesions, and recorded and measured at baseline. These lesions should be selected based on their size (lesions with the longest diameter) and their suitability for accurate repeated measurements (either by imaging techniques or clinically).

Note: Cystic lesions thought to represent cystic metastases must not be selected as target lesions when other suitable target lesions are available.

Note: Measurable lesions that have been previously irradiated and have not been shown to be progressing following irradiation must not be considered as target lesions.
Lytic bone lesions or mixed lytic-blastic lesions, with identifiable soft tissue components, that can be evaluated by CT or MRI) can be considered measurable. Bone scans, FDG-PET scans or X-rays are not considered adequate imaging techniques to measure bone lesions.
All other lesions (or sites of disease) must be identified as non-target and must also be recorded at baseline. Non-target lesions will be grouped by organ. Measurements of these lesions are not required, but the presence or absence of each must be noted throughout follow-up.

3.4.3 Physical Examinations

A complete physical examination performed at screening will include, at a minimum, assessment of the cardiovascular, respiratory, gastrointestinal, and neurological systems.

A brief physical examination performed at each subsequent visit will include, at a minimum, assessments of the skin, lungs, cardiovascular system, and abdomen (liver and spleen).

Investigators should pay special attention to clinical signs related to previous serious illnesses.

Physical examinations may be performed within one day of dosing (i.e., as opposed to the day of dosing), if necessary.

3.4.4 Performance Status

Performance status will be assessed using the ECOG scale at each visit and on the day of treatment.

3.4.5 Vital Signs

Vital signs will be measured after 5 minutes of rest and will include temperature, systolic and diastolic blood pressure, pulse rate, respiratory rate, and oxygen saturation (measured by pulse oximetry). Blood pressure should be taken in the same position throughout the study and captured in the eCRF.

Vital signs will be measured more frequently if warranted by clinical condition of the participant.

On days where vital signs are measured multiple times, temperature does not need to be repeated unless clinically indicated.

If a participant develops fever and infusion related reaction or cytokine release syndrome is suspected, the participant will be managed using management guidelines.

Height will be recorded at screening only.

Weight will be measured and recorded (in kilograms) at baseline and every other treatment visit.

Vital signs must be recorded prior to dosing on treatment days.

3.4.6 Electrocardiogram

A 12-lead ECG will be obtained at screening using an ECG machine that automatically calculates the heart rate and measures PR, QRS, QT, and QTcF intervals; manual calculation of QTcF is permitted. ECGs may be repeated during the study as clinically indicated.

3.4.7 Echocardiograms

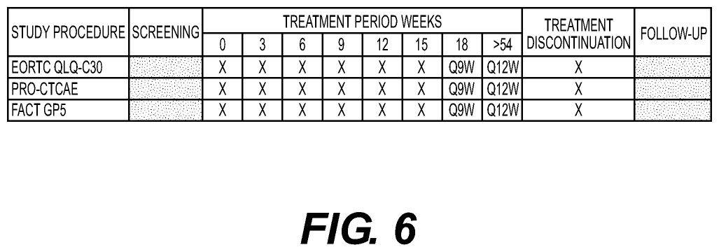

Echocardiograms (ECHO) will be performed locally at baseline to assess cardiac ejection fraction for study eligibility, as specified in the schedule of activities (FIGS. 4-6). Additional ECHO assessments may be performed if clinically warranted. The evaluation of the echocardiography should include an evaluation for left ventricular ejection fraction (LVEF) and both right and left-sided valvular lesions. Multigated Acquisition Scan (MUGA) can be used in lieu of ECHO (if not feasible) in the assessment of LVEF; the same modality should be used in any subsequent assessments.

3.4.8 Biomarkers

3.4.8.1 Blood Biomarkers

Blood samples will also be collected for isolation of PBMC, plasma, and serum. Whole blood samples will be collected and may be utilized to assess immune cell number, phenotype, activation, and function. Plasma and serum samples will be used for an analysis of circulating soluble factors in relation to T-cell activation, cfDNA, exosomes circulating proteins, and may be analyzed for soluble H2L5 hIgG4PE (ICOS Agonist) depending on the availability of the assays. Factors to be analyzed may include but are not limited to: the presence of IFN-γ, TNF-α, IL-2, IL-4, IL-6, IL 10, IL-8, IL-12p70 and IL-13, as well as antibodies against tumor, self-tumor mutations, gene expression (RNA or protein), genetic analysis (DNA) or viral antigens.

PBMCs isolated from whole blood will be preserved and stored for flow cytometry of additional cell types such as immune regulatory populations which may include but are not limited to myeloid derived suppressor cells, subsequent functional analysis or genetic analysis for assessment of the diversity of the T-cell repertoire, its relationship to clinical responses, and changes in response to treatment. The functional state of PBMCs may be analyzed for expression of cytokines which may include but not limited to IFN-γ, IL-2, TNF-α, IL-17, Granzyme B, and CD107a. PBMCs may also be evaluated for genomic (DNA) and gene expression (RNA or protein) alterations to determine treatment-related changes in immune-related signatures.

3.4.8.2 Tumor Tissue

For Part 1, all participants are required to have tumor tissue available (either archival or fresh biopsy) prior to start of study treatment. A fresh biopsy is required if archival tissue is unavailable. Following Part 1 initial safety evaluation (up to first 10 participants), for the additional participants enrolled to assess further safety as well as PK/PD, fresh tumor tissue and archival tumor tissue samples at screening are required prior to start of study treatment.

For Part 2, tumor tissue at screening (either archival or fresh biopsy if archival tissue is unavailable) is required for all participants. Fresh tumor tissue and an archival tissue sample obtained during screening are required for at least 20 participants.

For Part 1, participants in the initial safety evaluation, fresh biopsy at week 7 (±8 days) is optional. Following Part 1 initial safety evaluation (up to first 10 participants), additional participants enrolled to assess further safety and PK/PD are required to provide paired fresh biopsies collected at screening and at week 7 (±8 days).

Fresh biopsy collected at week 7 (±8 days) is optional for participants in Part 2, if tumor is amenable to biopsy and upon participant's consent. However, paired fresh tumor biopsies collected at screening and week 7 (±8 days) is required for at least 20 participants.

Additional optional fresh tumor tissue sample will be collected at Week 19 (±8 days) at the time of imaging assessment and/or at the time of confirmed PR or PD, upon participant consent (±8 days).

When feasible, tumor imaging should be completed prior to tissue collection to avoid potential radiographic alterations due to the biopsy procedure.

These tissues will be evaluated by IHC or other potential methods for expression of phenotypic and functional immune cell markers on tumor infiltrating lymphocytes (TIL) and other immune cells as well as immune signaling markers on tumor cells to understand the anti-tumor responses. In addition, when possible, similar analyses will be performed on tumor tissue obtained upon progression. Additionally, tumor tissue may be sequenced to assess T-cell diversity (TCR diversity) as well as evaluated for any DNA/RNA/protein changes correlating with response, including tumor mutational load assessments. These samples may also be evaluated for predictive measures of response to include in the biomarker selected population.

### 3.5 Statistical Considerations and Data Analyses

3.5.1 Part 1

The primary objective of Part 1 is to establish the safety and tolerability of experimental regimens prior to transition to Part 2 of the study.

Safety and tolerability will be guided using a mTPI approach with some additional modifications due to only one dose level being evaluated. The mTPI design is an extension of the toxicity probability interval method and employs a simple beta-binomial hierarchic model (Ji, 2010). Decision rules are based on calculating the unit probability mass (UPM) of three intervals corresponding to under dosing, proper dosing, and overdosing in terms of toxicity. Specifically, the under-dosing interval is defined as (0, pT−ε1), the overdosing interval as (pT+ε2, 1), and the proper dosing interval as (pT−ε1, pT+ε2), where pT is the target toxicity rate, ε1 and ε2 are small fractions, such as 0.05, to account for the uncertainty around the true target toxicity. The three dosing intervals are associated with three different dose decisions. Given an interval and a probability distribution, the UPM of that interval is defined as the probability of the interval divided by the length of the interval. The mTPI design calculates the UPMs for the three dosing intervals, and the one with the largest UPM implies the corresponding dose-finding decision. For example, if the over-dosing interval has the largest UPM, decision will be to stop further evaluation.

A futility analysis of ORR will be conducted after 10 participants have had at least two post baseline RECIST assessments. A maximum of 15 participants will be enrolled to allow for 10 evaluable participants to be assessed for futility. If at least one objective response is observed in 10 evaluable participants, the experimental regimen may proceed to Part 2 of the study. If no objective responses are observed in 10 evaluable participants, development of the experimental regimen may be stopped. Decisions will be made after evaluation of other endpoints and will be based on the totality of data, including the Disease Control Rate endpoint.

3.5.2 Part 2

The primary endpoint for Part 2 is OS. The primary analysis is based on the predictive probability of Phase III study success comparing the combination with the standard of care.

The predictive probability of success in a future Phase III study will be calculated using the posterior distribution of log(HR in Phase 3), given log(HR from Phase 2), assuming that both the observed log(HR) and prior for mean of log(HR) follow normal distributions.

The total number of events in phase 3 is assumed to be 210, and a cutoff of 70% or greater for the predictive probability of phase 3 trial success is used to define success for the combination.

3.5.3 Sample Size Determination

A maximum of 15 participants will be enrolled in Part 1.

For Part 2, sample size and associated operating characteristics were evaluated via simulation.

A maximum sample size of 70 participants in the combination treatment and a minimum of 35 participants in the control arm will be enrolled.

3.5.4 Population for Analyses

Intent to Treat Population (ITT) is defined as all participants who were randomized to treatment regardless of whether the participants actually received study treatment. All efficacy endpoints will be evaluated based on this population.

Safety Population is defined as all participants who receive at least 1 dose of standard of care or experimental regimen (i.e. H2L5 IgG4PE and dostarlimab plus cobolimab) based on actual treatment received. All safety endpoints will be evaluated based on this population.

PK Population will consist of all participants from the ITT Population from whom a blood sample is obtained and analyzed for PK concentration.

3.5.5 Interim Analysis (Part 2)

Interim analyses will be performed approximately every 3 to 6 months depending on the amount of additional data accrued.

An initial interim analysis based on OS will be performed.

3.5.6 Pharmacokinetic/Pharmacodynamic Analyses

If deemed appropriate and if data permit, exploratory pharmacokinetic/pharmacodynamic analyses such as exposure-response relationships between exposure (e.g., dose, Cₘₐₓ or Cₘᵢₙ) and clinical endpoints (e.g., anti-tumor response, biomarkers) may be conducted.

3.6 Guidelines for Assessment of Disease, Disease Progression and Response Criteria—Adapted from RECIST Version 1.1

3.6.1 Assessment Guidelines

The assessment guidelines are as set out in section 2.6.1.

3.6.2 Guidelines for Evaluation of Disease

The guidelines for evaluation of disease are as set out in section 2.6.2

### 3.7 ECOG Performance Status

The summary as presentation in section 2.7

SEQUENCE LISTINGS

| SEQ ID NO. | Sequence | Description |
|---|---|---|
| 1 | DYAMH | ICOS binding protein CDRH1 |
| 2 | LISIYSDHTNYNQKFQG | ICOS binding protein CDRH2 |
| 3 | NNYGNYGWYFDV | ICOS binding protein CDRH3 |
| 4 | SASSSVSYMH | ICOS binding protein CDRL1 |
| 5 | DTSKLAS | ICOS binding protein CDRL2 |
| 6 | FQGSGYPYT | ICOS binding protein CDRL3 |
| 7 | QVQLVQSGAEVKKPGSSVKVSCKASGYTFTDYAMHWVRQAPGQGLEWM | ICOS humanized heavy chain |
| | GLISIYSDHTNYNQKFQGRVTITADKSTSTAYMELSSLRSEDTAVYYC | variable region (H2) |
| | GRNNYGNYGWYFDVWGQGTTVTVSS | |
| 8 | EIVLTQSPATLSLSPGERATLSCSASSSVSYMHWYQQKPGQAPRLLIY | ICOS humanized light chain |
| | DTSKLASGIPARFSGSGSGTDYTLTISSLEPEDFAVYYCFQGSGYPYT | variable region (L5) |
| | FGQGTKLEIK | |
| 9 | QVQLVQSGAEVKKPGSSVKVSCKASGYTFTDYAMHWVRQAPGQGLEWM | ICOS humanized monoclonal |
| | GLISIYSDHTNYNQKFQGRVTITADKSTSTAYMELSSLRSEDTAVYYC | antibody heavy chain |
| | GRNNYGNYGWYFDVWGQGTTVTVSSASTKGPSVFPLAPCSRSTSESTA | |
| | ALGCLVKDYFPEPVTVSWNSGALTSGVHTFPAVLQSSGLYSLSSVVTV | |
| | PSSSLGTKTYTCNVDHKPSNTKVDKRVESKYGPPCPPCPAPEFEGGPS | |
| | VFLFPPKPKDTLMISRTPEVTCVVVDVSQEDPEVQFNWYVDGVEVHNA | |
| | KTKPREEQFNSTYRVVSVLTVLHQDWLNGKEYKCKVSNKGLPSSIEKT | |
| | ISKAKGQPREPQVYTLPPSQEEMTKNQVSLTCLVKGFYPSDIAVEWES | |
| | NGQPENNYKTTPPVLDSDGSFFLYSRLTVDKSRWQEGNVFSCSVMHEA | |
| | LHNHYTQKSLSLSLGK | |
| 10 | EIVLTQSPATLSLSPGERATLSCSASSSVSYMHWYQQKPGQAPRLLIY | ICOS humanized monoclonal |
| | DTSKLASGIPARFSGSGSGTDYTLTISSLEPEDFAVYYCFQGSGYPYT | antibody light chain |
| | FGQGTKLEIKRTVAAPSVFIFPPSDEQLKSGTASWVCLLNNFYPREAK | |
| | VQWKVDNALQSGNSQESVTEQDSKDSTYSLSSTLTLSKADYEKHKVYA | |
| | CEVTHQGLSSPVTKSFNRGEC | |
| 11 | MKSGLWYFFLFCLRIKVLTGEINGSANYEMFIFHNGGVQILCKYPDIV | Human ICOS (isoform 2) |
| | QQFKMQLLKGGQILCDLTKTKGSGNTVSIKSLKFCHSQLSNNSVSFFL | |
| | YNLDHSHANYYFCNLSIFDPPPFKVTLTGGYLHIYESQLCCQLKFWLP | |
| | IGCAAFVVVCILGCILICWLTKKM | |
| 12 | MKSGLWYFFLFCLRIKVLTGEINGSANYEMFIFHNGGVQILCKYPDIV | Human ICOS (isoform 1) |
| | QQFKMQLLKGGQILCDLTKTKGSGNTVSIKSLKFCHSQLSNNSVSFFL | |
| | YNLDHSHANYYFCNLSIFDPPPFKVTLTGGYLHIYESQLCCQLKFWLP | |
| | IGCAAFVVVCILGCILICWLTKKKYSSSVHDPNGEYMFMRAVNTAKKS | |
| | RLTDVTL | |
| 13 | SYDMS | TIM-3 binding protein CDRH1 |
| 14 | TISGGGTYTYYQDSVKG | TIM-3 binding protein CDRH2 |
| 15 | MDY | TIM-3 binding protein CDRH3 |
| 16 | RASQSIRRYLN | TIM-3 binding protein CDRL1 |
| 17 | GASTLQS | TIM-3 binding protein CDRL2 |
| 18 | QQSHSAPLT | TIM-3 binding protein CDRL3 |
| 19 | EVQLLESGGGLVQPGGSLRLSCAAASGFTFSSYDMSWVRQAPGKGLDW | TIM-3 heavy chain variable |
| | VSTISGGGTYTYYQDSVKGRFTISRDNSKNTLYLQMNSLRAEDTAVYY | region |
| | CASMDYWGQGTTVTVSS | |
| 20 | DIQMTQSPSSLSASVGDRVTITCRASQSIRRYLNWYHQKPGKAPKLLI | TIM-3 light chain variable |
| | YGASTLQSGVPSRFSGSGSGTDFTLTISSLQPEDFAVYYCQQSHSAPL | region |
| | TFGGGTKVEIK | |
| 21 | EVQLLESGGGLVQPGGSLRLSCAAASGFTFSSYDMSWVRQAPGKGLDW | TIM-3 monoclonal antibody |
| | VSTISGGGTYTYYQDSVKGRFTISRDNSKNTLYLQMNSLRAEDTAVYY | heavy chain |
| | CASMDYWGQGTTVTVSSASTKGPSVFPLAPCSRSTSESTAALGCLVKD | |
| | YFPEPVTVSWNSGALTSGVHTFPAVLQSSGLYSLSSWTVPSSSLGTKT | |
| | YTCNVDHKPSNTKVDKRVESKYGPPCPPCPAPEFLGGPSVFLFPPKPK | |
| | DTLMISRTPEVTCVWVDVSQEDPEVQFNWYVDGVEVHNAKTKPREEQF | |
| | NSTYRVVSVLTVLHQDWLNGKEYKCKVSNKGLPSSIEKTISKAKGQPR | |
| | EPQVYTLPPSQEEMTKNQVSLTCLVKGFYPSDIAVEWESNGQPENNYK | |
| | TTPPVLDSDGSFFLYSRLTVDKSRWQEGNVFSCSVMHEALHNHYTQKS | |
| | LSLSLGK | |
| 22 | DIQMTQSPSSLSASVGDRVTITCRASQSIRRYLNWYHQKPGKAPKLLI | TIM-3 monoclonal antibody |
| | YGASTLQSGVPSRFSGSGSGIDFILIISSLQPEDFAVYYCQQSHSAPL | light chain |
| | TFGGGTKVEIKRTVAAPSVFIFPPSDEQLKSGTASVVCLLNNFYPREA | |
| | KVQWKVDNALQSGNSQESVTEQDSKDSTYSLSSTLTLSKADYEKHKVY | |
| | ACEVTHQGLSSPVTKSFNRGEC | |
| 23 | MFSHLPFDCVLLLLLLLLTRSSEVEYRAEVGQNAYLPCFYTPAAPGNL | Human TIM-3 |
| | VPVCWGKGACPVFECGNVVLRTDERDVNYWTSRYWLNGDFRKGDVSLT | |
| | IENVTLADSGIYCCRIQIPGIMNDEKFNLKLVIKPAKVTPAPTRQRDF | |
| | TAAFPRMLTTRGHGPAETQTLGSLPDINLTQISTLANELRDSRLANDL | |
| | RDSGATIRIGIYIGAGICAGLALALIFGALIFKWYSHSKEKIQNLSLI | |
| | SLANLPPSGLANAVAEGIRSEENIYTIEENVYEVEEPNEYYCYVSSRQ | |
| | QPSQPLGCRFAMP | |
| 24 | SYDMS | PD-1 binding protein CDRH1 |
| 25 | TISGGGSYTYYQDSVKG | PD-1 binding protein CDRH2 |
| 26 | PYYAMDY | PD-1 binding protein CDRH3 |
| 27 | KASQDVGTAVA | PD-1 binding protein CDRL1 |
| 28 | WASTLHT | PD-1 binding protein CDRL2 |
| 29 | QHYSSYPWT | PD-1 binding protein CDRL3 |
| 30 | EVQLLESGGGLVQPGGSLRLSCAASGFTFSSYDMSWVRQAPGKGLEWV | PD-1 heavy chain variable |
| | STISGGGSYTYYQDSVKGRFTISRDNSKNTLYLQMNSLRAEDTAVYYC | region |
| | ASPYYAMDYWGQGTTVTVSS | |
| 31 | DIQLTQSPSFLSAYVGDRVTITCKASQDVGTAVAWYQQKPGKAPKLLI | PD-1 light chain variable |
| | YWASTLHTGVPSRFSGSGSGTEFTLTISSLQPEDFATYYCQHYSSYPW | region |
| | TFGQGTKLEIK | |
| 32 | EVQLLESGGGLVQPGGSLRLSCAASGFTFSSYDMSWVRQAPGKGLEWV | PD-1 monoclonal antibody |
| | STISGGGSYTYYQDSVKGRFTISRDNSKNTLYLQMNSLRAEDTAVYYC | heavy chain |
| | ASPYYAMDYWGQGTTVTVSSASTKGPSVFPLAPCSRSTSESTAALGCL | |
| | VKDYFPEPVTVSWNSGALTSGVHTFPAVLQSSGLYSLSSVVTVPSSSL | |
| | GTKTYTCNVDHKPSNTKVDKRVESKYGPPCPPCPAPEFLGGPSVFLFP | |
| | PKPKDTLMISRTPEVTCVVVDVSQEDPEVQFNWYVDGVEVHNAKTKPR | |
| | EEQFNSTYRVVSVLTVLHQDWLNGKEYKCKVSNKGLPSSIEKTISKAK | |
| | GQPREPQVYTLPPSQEEMTKNQVSLTCLVKGFYPSDIAVEWESNGQPE | |
| | NNYKTTPPVLDSDGSFFLYSRLTVDKSRWQEGNVFSCSVMHEALHNHY | |
| | TQKSLSLSLGK | |
| 33 | DIQLTQSPSFLSAYVGDRVTITCKASQDVGTAVAWYQQKPGKAPKLLI | PD-1 monoclonal antibody |
| | YWASTLHTGVPSRFSGSGSGTEFTLTISSLQPEDFATYYCQHYSSYPW | light chain |
| | TFGQGTKLEIKRTVAAPSVFIFPPSDEQLKSGTASVVCLLNNFYPREA | |
| | KVQWKVDNALQSGNSQESVTEQDSKDSTYSLSSTLTLSKADYEKHKVY | |
| | ACEVTHQGLSSPVTKSFNRGEC | |
| 34 | EVQLLESGGGLVQPGGSLRLSCAASGFTFSSYDMSWVRQAPGKGLEWV | PD-1 heavy chain sequence |
| | STISGGGSYTYYQDSVKGRFTISRDNSKNTLYLQMNSLRAEDTAVYYC | with N380D modification |
| | ASPYYAMDYWGQGTTVTVSSASTKGPSVFPLAPCSRSTSESTAALGCL | |
| | VKDYFPEPVTVSWNSGALTSGVHTFPAVLQSSGLYSLSSVVTVPSSSL | |
| | GTKTYTCNVDHKPSNTKVDKRVESKYGPPCPPCPAPEFLGGPSVFLFP | |
| | PKPKDTLMISRTPEVTCVVDVSQEDPEVQFNWYVDGVEVHNAKTKPRE | |
| | EQFNSTYRVVSVLTVLHQDWLNGKEYKCKVSNKGLPSSIEKTISKAKG | |
| | APREPQVYTLPPSQEEMTKNQVSLTCLVKGFYPSDIAVEWESDGQPEN | |
| | NYKTTPPVLDSDGSFFLYSRLTVDKSRWQEGNVFSCSVMHEALHNHYT | |
| | QKSLSLSLGK | |
| 35 | EVQLLESGGGLVQPGGSLRLSCAASGFTFSSYDMSWVRQAPGKGLEWV | PD-1 heavy chain sequence |
| | STISGGGSYTYYQDSVKGRFTISRDNSKNTLYLQMNSLRAEDTAVYYC | with N385D modification |
| | ASPYYAMDYWGQGTTVTVSSASTKGPSVFPLAPCSRSTSESTAALGCL | |
| | VKDYFPEPVTVSWNSGALTSGVHTFPAVLQSSGLYSLSSVVTVPSSSL | |
| | GTKTYTCNVDHKPSNTKVDKRVESKYGPPCPPCPAPEFLGGPSVFLFP | |
| | PKPKDTLMISRTPEVTCVVDVSQEDPEVQFNWYVDGVEVHNAKTKPRE | |
| | EQFNSTYRVVSVLTVLHQDWLNGKEYKCKVSNKGLPSSIEKTISKAKG | |
| | QPREPQVYTLPPSQEEMTKNQVSLTCLVKGFYPSDIAVEWESNGQPED | |
| | NYKTTPPVLDSDGSFFLYSRLTVDKSRWQEGNVFSCSVMHEALHNHYT | |
| | QKSLSLSLGK | |
| 36 | EVQLLESGGGLVQPGGSLRLSCAASGFTFSSYDMSWVRQAPGKGLEWV | PD-1 heavy chain sequence |
| | STISGGGSYTYYQDSVKGRFTISRDNSKNTLYLQMNSLRAEDTAVYYC | with N380D and N385D |
| | ASPYYAMDYWGQGTTVTVSSASTKGPSVFPLAPCSRSTSESTAALGCL | modifications |
| | VKDYFPEPVTVSWNSGALTSGVHTFPAVLQSSGLYSLSSWTVPSSSLG | |
| | TKTYTCNVDHKPSNTKVDKRVESKYGPPCPPCPAPEFLGGPSVFLFPP | |
| | KPKDTLMISRTPEVTCVVDVSQEDPEVQFNWYVDGVEVHNAKTKPREE | |
| | QFNSTYRWVSVLTVLHQDWLNGKEYKCKVSNKGLPSSIEKTISKAKGQ | |
| | PREPQVYTLPPSQEEMTKNQVSLTCLVKGFYPSDIAVEWESDGQPEDN | |
| | YKTTPPVLDSDGSFFLYSRLTVDKSRWQEGNVFSCSVMHEALHNHYTQ | |
| | KSLSLSLGK | |
| 37 | QHYNSYPWT | PD-1 binding protein CDRL3 |
| | | Alternative |
| 38 | MQIPQAPWPVVWAVLQLGWRPGWFLDSPDRPWNPPTFSPALLVVTEGD | Human PD-1 amino acid |
| | NATFTCSFSNTSESFVLNWYRMSPSNQTDKLAAFPEDRSQPGQDCRFR | sequence in NCBI Locus No.: |
| | VTQLPNGRDFHMSVVRARRNDSGTYLCGAISLAPKAQIKESLRAELRV | NP_005009 |
| | TERRAEVPTAHPSPSPRPAGQFQTLVVGVVGGLLGSLVLLVWVLAVIC | |
| | SRAARGTIGARRTGQPLKEDPSAVPVFSVDYGELDFQWREKTPEPPVP | |
| | CVPEQTEYATIVFPSGMGTSSPARRGSADGPRSAQPLRPEDGHCSWPL | |
| 39 | MRIFAVFIFMTYWHLLNAFTVTVPKDLYWEYGSNMTIECKFPVEKQLD | Human PD-L1 amino acid |
| | LAALIVYWEMEDKNIIQFVHGEEDLKVQHSSYRQRARLLKDQLSLGNA | sequence in NCBI Locus No.: |
| | ALQITDVKLQDAGVYRCMISYGGADYKRITVKVNAPYNKINQRILVVD | NP_054862 |
| | PVTSEHELTCQAEGYPKAEVIWTSSDHQVLSGKTTTTNSKREEKLFNV | |
| | TSTLRINTTTNEIFYCTFRRLDPEENHTAELVIPELPLAHPPNERTHL | |
| | VILGAILLCLGVALTFIFRLRKGRMMDVKKCGIQDTNSKKQSDTHLEE | |
| | T | |
| 40 | MIFLLLMLSLELQLHQIAALFTVTVPKELYIIEHGSNVTLECNFDTGS | Human PD-L2 amino acid |
| | HVNLGAUASLQKVENDTSPHRERATLLEEQLPLGKASFHIPQVQVRDE | sequence in NCBI Locus No.: |
| | GQYQCIIIYGVAWDYKYLTLKVKASYRKINTHILKVPETDEVELTCQA | NP_079515 |
| | TGYPLAEVSWPNVSVPANTSHSRTPEGLYQVTSVLRLKPPPGRNFSCV | |
| | FWNTHVRELTLASIDLQSQMEPRTHPTWLLHIFIPFCIIAFIFIATVI | |
| | ALRKQLCQKLYSSKDTTKRPVTTTKREVNSAI | |
| 41 | GFTFSDYWMD | 37A10S713 VH CDR1 |
| 42 | NIDEDGSITEYSPFVKG | 37A10S713 VH CDR2 |
| 43 | WGRFGFDS | 37A10S713 VH CDR3 |
| 44 | KSSQSLLSGSFNYLT | 37A10S713 VL CDR1 |
| 45 | YASTRHT | 37A10S713 VL CDR2 |
| 46 | HHHYNAPPT | 37A10S713 VL CDR3 |
| 47 | EVQLVESGGLVQPGGSLRLSCAASGFTFSDYWMDWVRQAPGKGLVWVS | 37A10S713 heavy chain |
| | NIDEDGSrTEYSPFVKGRFTISRDNAKNTLYLQMNSLRAEDTAVYYCT | variable region |
| | RWGRFGFDSWGQGTLVTVSS | |
| 48 | DIVMTQSPDSLAVSLGERATINCKSSQSLLSGSFNYLTWYQQKPGQPP | 37A10S713 light chain variable |
| | KLLIFYASTRHTGVPDRFSGSGSGTDFTLTISSLQAEDVAVYYCHHHY | region |
| | NAPPTFGPGTKVDIK | |
| 49 | EVQLVESGGGLVKPGGSLRLSCAASGFTFSDYFMHWVRQAPGKGLEWV | ICOS.33 IgGlf S267E heavy |
| | GVIDTKSFNYATYYSDLVKGRFTISRDDSKNTLYLQMNSLKTEDTAVY | chain variable region |
| | YCTATIAVPYYFDYWGQGTLVTVSS | |
| 50 | DIQMTQSPSSLSASVGDRVTITCQASQDISNYLSWYQQKPGKAPKLLI | ICOS.33 IgGlf S267E light |
| | YYTNLLAEGVPSRFSGSGSGTDFTFTISSLQPEDIATYYCQQYYNYRT | chain variable region |
| | FGPGTKVDIK | |
| 51 | EVQLVESGGGVVRPGGSLRLSCVASGVTFDDYGMSWVRQAPGKGLEWV | STIM003 heavy chain variable |
| | SGINWNGGDTDYSDSVKGRFTISRDNAKNSLYLQMNSLRAEDTALYYC | region |
| | ARDFYGSGSYYHVPFDYWGQGILVTVSS | |
| 52 | EIVLTQSPGTLSLSPGERATLSCRASQSVSRSYLAWYQQKRGQAPRLL | STIM003 light chain variable |
| | IYGASSRATGIPDRFSGDGSGTDFTLSISRLEPEDFAVYYCHQYDMSP | region |
| | FTFGPGTKVDIK | |
| 53 | QVQLVQSGAEVKKPGASVKVSCKASGYTFTGYYMHWVRQAPGQGLEWM | XENP23104 [ICOS]_H0.66_L0 |
| | GWINPHSGETIYAQKFQGRVTMTRDTSISTAYMELSSLRSEDTAVYYC | heavy chain variable region |
| | ARTYYYDTSGYYHDAFDVWGQGTMVTVSS | |
| 54 | GYYMH | XENP23104 [ICOS]_H0.66_L0 |
| | | CDRH1 |
| 55 | WINPHSGETIYAQKFQG | XENP23104 [ICOS]_H0.66_L0 |
| | | CDRH2 |
| 56 | TYYYDTSGYYHDAFDV | XENP23104 [ICOS]_H0.66_L0 |
| | | CDRH3 |
| 57 | DIQMTQSPSSVSASVGDRVTITCRASQGISRLLAWYQQKPGKAPKLLI | XENP23104 [ICOS]_H0.66_L0 |
| | YVASSLQSGVPSRFSGSGSG IUFILIISSLQPEDFATYYCQQANSFP | light chain variable region |
| | WTFGQGTKVEIK | |
| 58 | RASQGISRLLA | XENP23104 [ICOS]_H0.66_L0 |
| | | CDRL1 |
| 59 | VASSLQS | XENP23104 [ICOS]_H0.66_L0 |
| | | CDRL2 |
| 60 | QQANSFPWT | XENP23104 [ICOS]_H0.66_L0 |
| | | CDRL3 |

1-30. (canceled)

31. A method for the treatment of cancer in a subject in need thereof comprising administering a therapeutically effective amount of a combination comprising an ICOS binding protein comprising a heavy chain amino acid sequence comprising a CDRH1 of SEQ ID NO:1, a CDRH2 of SEQ ID NO:2, and a CDRH3 of SEQ ID NO:3, and a light chain amino acid sequence comprising a CDRL1 of SEQ ID NO:4, a CDRL2 of SEQ ID NO:5, and a CDRL3 of SEQ ID NO:6, and a TIM-3 binding protein comprising a heavy chain amino acid sequence comprising a CDRH1 of SEQ ID NO:13, a CDRH2 of SEQ ID NO:14, and a CDRH3 of SEQ ID NO:15, and a light chain amino acid sequence comprising a CDRL1 of SEQ ID NO:16, a CDRL2 of SEQ ID NO:17, and a CDRL3 of SEQ ID NO:18, to the subject.

32. The method of claim 31, further comprising administering a therapeutically effective amount of a PD-1 binding protein comprising a heavy chain amino acid sequence comprising a CDRH1 of SEQ ID NO:24, a CDRH2 of SEQ ID NO:25, and a CDRH3 of SEQ ID NO:26, and a light chain amino acid sequence comprising a CDRL1 of SEQ ID NO:27, a CDRL2 of SEQ ID NO:28, and a CDRL3 of SEQ ID NO:29.

33. The method of claim 31, wherein the ICOS binding protein comprises a heavy chain variable region (V_{H}) at least about 90% identical to the amino acid sequence of SEQ ID NO:7 and/or a light chain variable region (V_{L}) at least about 90% identical to the amino acid sequence of SEQ ID NO:8.

34. The method of claim 33, wherein the ICOS binding protein comprises a V_{H} comprising an amino acid sequence of SEQ ID NO:7 and a V_{L} comprising an amino acid sequence of SEQ ID NO:8.

35. The method of claim 31, wherein the ICOS binding protein comprises a heavy chain amino acid sequence at least about 90% identical to the amino acid sequence of SEQ ID NO:9 and/or a light chain amino acid sequence at least about 90% identical to the amino acid sequence of SEQ ID NO:10.

36. The method of claim 35, wherein the ICOS binding protein comprises a heavy chain amino acid sequence of SEQ ID NO:9 and a light chain amino acid sequence of SEQ ID NO:10.

37. The method of claim 31, wherein the TIM-3 binding protein comprises a heavy chain variable region (V_{H}) at least about 90% identical to the amino acid sequence of SEQ ID NO:19 and/or a light chain variable region (V_{L}) at least about 90% identical to the amino acid sequence of SEQ ID NO:20.

38. The method of claim 37, wherein the TIM-3 binding protein comprises a V_{H} comprising an amino acid sequence of SEQ ID NO:19 and a V_{L} comprising an amino acid sequence of SEQ ID NO:20.

39. The method of claim 31, wherein the TIM-3 binding protein comprises a heavy chain amino acid sequence at least about 90% identical to the amino acid sequence of SEQ ID NO:21 and/or a light chain amino acid sequence at least about 90% identical to the amino acid sequence of SEQ ID NO:22.

40. The method of claim 39, wherein the TIM-3 binding protein comprises a heavy chain amino acid sequence of SEQ ID NO:21 and a light chain amino acid sequence of SEQ ID NO:22.

41. The method of claim 32, wherein the PD-1 binding protein comprises a heavy chain variable region (V_{H}) at least about 90% identical to the amino acid sequence of SEQ ID NO:30 and/or a light chain variable region (V_{L}) at least about 90% identical to the amino acid sequence of SEQ ID NO:31.

42. The combination of claim 41, wherein the PD-1 binding protein comprises a V_{H} comprising an amino acid sequence of SEQ ID NO:30 and a V_{L} comprising an amino acid sequence of SEQ ID NO:31.

43. The combination of claim 32, wherein the PD-1 binding protein comprises a heavy chain amino acid sequence at least about 90% identical to the amino acid sequence of SEQ ID NO:32 and/or a light chain amino acid sequence at least about 90% identical to the amino acid sequence of SEQ ID NO:33.

44. The combination of claim 43, wherein the PD-1 binding protein comprises a heavy chain amino acid sequence of SEQ ID NO:32 and a light chain amino acid sequence of SEQ ID NO:33.

45. The method of claim 31, wherein the ICOS binding protein or the TIM-3 binding protein is a monoclonal antibody or antigen binding fragment thereof.

46. The method of claim 31, wherein the ICOS binding protein or the TIM-3 binding protein is an IgG4 monoclonal antibody.

47. (canceled)

48. (canceled)

49. The combination of claim 32, wherein the PD-1 binding protein is a monoclonal antibody or antigen binding fragment thereof.

50. The combination of claim 32, wherein the PD-1 binding protein is an IgG4 monoclonal antibody.

51. The method of claim 31, wherein the subject is a human.

52. The method of claim 31, wherein the cancer is selected from appendiceal cancer, bladder cancer, breast cancer, cervical cancer, colorectal cancer, endometrial cancer, esophageal cancer, fallopian tube cancer, gastric cancer, glioma (such as diffuse intrinsic pontine glioma), head and neck cancer (in particular head and neck squamous cell carcinoma and oropharyngeal cancer), leukemia (in particular acute lymphoblastic leukemia, acute myeloid leukemia) lung cancer (in particular non small cell lung cancer), lymphoma (in particular Hodgkin's lymphoma, non-Hodgkin's lymphoma), mesothelioma (in particular malignant pleural mesothelioma), melanoma, Merkel cell carcinoma, neuroblastoma, oral cancer, osteosarcoma, ovarian cancer, prostate cancer, renal cancer, salivary gland tumor, sarcoma (in particular Ewing's sarcoma or rhabdomyosarcoma) squamous cell carcinoma, soft tissue sarcoma, thymoma, thyroid cancer, urothelial cancer, uterine cancer, vaginal cancer, vulvar cancer or Wilms tumor.

53. (canceled)

54. (canceled)

55. The method of claim 31, wherein the binding proteins are administered simultaneously.

56. The method of claim 31, wherein the binding proteins are administered sequentially.

57. The method as of claim 31, wherein the ICOS binding protein is administered at a dose of about 0.08 mg to about 240 mg.

58. The method of claim 31, wherein the ICOS binding protein is administered at a dose of 8 mg, 24 mg, 48 mg, 80 mg, 160 mg or 240 mg.

59. The method of claim 31, wherein the ICOS binding protein is administered at a dose of about 24 mg or about 80 mg every three weeks or a dose of about 48 mg or about 160 mg every six weeks.

60. The method of claim 31, wherein the TIM-3 binding protein is administered at a dose of about 100 mg to about 1000 mg.

61. The method of claim 31, wherein the TIM-3 binding protein is administered at a dose of 100 mg, 300 mg or 900 mg.

62. The method of claim 31, wherein the TIM-3 binding protein is administered at a dose of 300 mg.

63. The method of claim 31, wherein the TIM-3 binding protein is administered at a dose of about 300 mg every three weeks.

64. The method of claim 32, wherein the PD-1 binding protein is administered at a dose of about 100 mg to about 2000 mg.

65. The method of claim 32, wherein the PD-1 binding protein is administered at a dose of 500 mg or 1000 mg.

66. The method of claim 32, wherein the PD-1 binding protein is administered at a dose of 500 mg every 3 weeks or a dose of 1000 mg every six weeks.

67. The method of claim 32, wherein the PD-1 binding protein is administered at a first dose of 500 mg once every three weeks (Q3W) for 4 cycles, followed by a second dose of 1000 mg once every six weeks (Q6W).

68-70. (canceled)

71. A kit comprising:
(i) an ICOS binding protein comprising a heavy chain amino acid sequence comprising a CDRH1 of SEQ ID NO:1, a CDRH2 of SEQ ID NO:2, and a CDRH3 of SEQ ID NO:3, and a light chain amino acid sequence comprising a CDRL1 of SEQ ID NO:4, a CDRL2 of SEQ ID NO:5, and a CDRL3 of SEQ ID NO:6;
(ii) a TIM-3 binding protein comprising a heavy chain amino acid sequence comprising a CDRH1 of SEQ ID NO:13, a CDRH2 of SEQ ID NO:14, and a CDRH3 of SEQ ID NO:15, and a light chain amino acid sequence comprising a CDRL1 of SEQ ID NO:16, a CDRL2 of SEQ ID NO:17, and a CDRL3 of SEQ ID NO:18; and
(iii) instructions for using (i) and (ii) in combination in the treatment of a cancer in a human.

72. A kit comprising:
(i) an ICOS binding protein comprising a heavy chain amino acid sequence comprising a CDRH1 of SEQ ID NO:1, a CDRH2 of SEQ ID NO:2, and a CDRH3 of SEQ ID NO:3, and a light chain amino acid sequence comprising a CDRL1 of SEQ ID NO:4, a CDRL2 of SEQ ID NO:5, and a CDRL3 of SEQ ID NO:6;
(ii) a TIM-3 binding protein comprising a heavy chain amino acid sequence comprising a CDRH1 of SEQ ID NO:13, a CDRH2 of SEQ ID NO:14, and a CDRH3 of SEQ ID NO:15, and a light chain amino acid sequence comprising a CDRL1 of SEQ ID NO:16, a CDRL2 of SEQ ID NO:17, and a CDRL3 of SEQ ID NO:18;
(iii) a PD-1 binding protein comprising a heavy chain amino acid sequence comprising a CDRH1 of SEQ ID NO:24, a CDRH2 of SEQ ID NO:25, and a CDRH3 of SEQ ID NO:26, and a light chain amino acid sequence comprising a CDRL1 of SEQ ID NO:27, a CDRL2 of SEQ ID NO:28, and a CDRL3 of SEQ ID NO:29; and
(iv) instructions for using (i) and (ii) in combination in the treatment of a cancer in a human.

73-74. (canceled)


SEQUENCE LISTING (60 sequences)

SEQ ID NO: 1 - protein, 5 aa, Artificial Sequence
  Artificially Synthesized Sequence
DYAMH

SEQ ID NO: 2 - protein, 17 aa, Artificial Sequence
  Artificially Synthesized Sequence
LISIYSDHTNYNQKFQG

SEQ ID NO: 3 - protein, 12 aa, Artificial Sequence
  Artificially Synthesized Sequence
NNYGNYGWYFDV

SEQ ID NO: 4 - protein, 10 aa, Artificial Sequence
  Artificially Synthesized Sequence
SASSSVSYMH

SEQ ID NO: 5 - protein, 7 aa, Artificial Sequence
  Artificially Synthesized Sequence
DTSKLAS

SEQ ID NO: 6 - protein, 9 aa, Artificial Sequence
  Artificially Synthesized Sequence
FQGSGYPYT

SEQ ID NO: 7 - protein, 121 aa, Artificial Sequence
  Artificially Synthesized Sequence
QVQLVQSGAEVKKPGSSVKVSCKASGYTFTDYAMHWVRQAPGQGLEWMGLISIYSDHTNYNQKFQGRVTITADKSTSTAYMELSSLRSEDTAVYYCGRNNYGNYGWYFDVWGQGTTVTVSS

SEQ ID NO: 8 - protein, 106 aa, Artificial Sequence
  Artificially Synthesized Sequence
EIVLTQSPATLSLSPGERATLSCSASSSVSYMHWYQQKPGQAPRLLIYDTSKLASGIPARFSGSGSGTDYTLTISSLEPEDFAVYYCFQGSGYPYTFGQGTKLEIK

SEQ ID NO: 9 - protein, 448 aa, Artificial Sequence
  Artificially Synthesized Sequence
QVQLVQSGAEVKKPGSSVKVSCKASGYTFTDYAMHWVRQAPGQGLEWMGLISIYSDHTNYNQKFQGRVTITADKSTSTAYMELSSLRSEDTAVYYCGRNNYGNYGWYFDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 10 - protein, 213 aa, Artificial Sequence
  Artificially Synthesized Sequence
EIVLTQSPATLSLSPGERATLSCSASSSVSYMHWYQQKPGQAPRLLIYDTSKLASGIPARFSGSGSGTDYTLTISSLEPEDFAVYYCFQGSGYPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 168 aa, Artificial Sequence
  Artificially Synthesized Sequence
MKSGLWYFFLFCLRIKVLTGEINGSANYEMFIFHNGGVQILCKYPDIVQQFKMQLLKGGQILCDLTKTKGSGNTVSIKSLKFCHSQLSNNSVSFFLYNLDHSHANYYFCNLSIFDPPPFKVTLTGGYLHIYESQLCCQLKFWLPIGCAAFVVVCILGCILICWLTKKM

SEQ ID NO: 12 - protein, 199 aa, Artificial Sequence
  Artificially Synthesized Sequence
MKSGLWYFFLFCLRIKVLTGEINGSANYEMFIFHNGGVQILCKYPDIVQQFKMQLLKGGQILCDLTKTKGSGNTVSIKSLKFCHSQLSNNSVSFFLYNLDHSHANYYFCNLSIFDPPPFKVTLTGGYLHIYESQLCCQLKFWLPIGCAAFVVVCILGCILICWLTKKKYSSSVHDPNGEYMFMRAVNTAKKSRLTDVTL

SEQ ID NO: 13 - protein, 5 aa, Artificial Sequence
  Artificially Synthesized Sequence
SYDMS

SEQ ID NO: 14 - protein, 17 aa, Artificial Sequence
  Artificially Synthesized Sequence
TISGGGTYTYYQDSVKG

SEQ ID NO: 15 - protein, 3 aa, Artificial Sequence
  Artificially Synthesized Sequence
MDY

SEQ ID NO: 16 - protein, 11 aa, Artificial Sequence
  Artificially Synthesized Sequence
RASQSIRRYLN

SEQ ID NO: 17 - protein, 7 aa, Artificial Sequence
  Artificially Synthesized Sequence
GASTLQS

SEQ ID NO: 18 - protein, 9 aa, Artificial Sequence
  Artificially Synthesized Sequence
QQSHSAPLT

SEQ ID NO: 19 - protein, 113 aa, Artificial Sequence
  Artificially Synthesized Sequence
EVQLLESGGGLVQPGGSLRLSCAAASGFTFSSYDMSWVRQAPGKGLDWVSTISGGGTYTYYQDSVKGRFTISRDNSKNTLYLQMNSLRAEDTAVYYCASMDYWGQGTTVTVSS

SEQ ID NO: 20 - protein, 107 aa, Artificial Sequence
  Artificially Synthesized Sequence
DIQMTQSPSSLSASVGDRVTITCRASQSIRRYLNWYHQKPGKAPKLLIYGASTLQSGVPSRFSGSGSGTDFTLTISSLQPEDFAVYYCQQSHSAPLTFGGGTKVEIK

SEQ ID NO: 21 - protein, 440 aa, Artificial Sequence
  Artificially Synthesized Sequence
EVQLLESGGGLVQPGGSLRLSCAAASGFTFSSYDMSWVRQAPGKGLDWVSTISGGGTYTYYQDSVKGRFTISRDNSKNTLYLQMNSLRAEDTAVYYCASMDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 22 - protein, 214 aa, Artificial Sequence
  Artificially Synthesized Sequence
DIQMTQSPSSLSASVGDRVTITCRASQSIRRYLNWYHQKPGKAPKLLIYGASTLQSGVPSRFSGSGSGTDFTLTISSLQPEDFAVYYCQQSHSAPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 23 - protein, 301 aa, Homo sapiens
MFSHLPFDCVLLLLLLLLTRSSEVEYRAEVGQNAYLPCFYTPAAPGNLVPVCWGKGACPVFECGNVVLRTDERDVNYWTSRYWLNGDFRKGDVSLTIENVTLADSGIYCCRIQIPGIMNDEKFNLKLVIKPAKVTPAPTRQRDFTAAFPRMLTTRGHGPAETQTLGSLPDINLTQISTLANELRDSRLANDLRDSGATIRIGIYIGAGICAGLALALIFGALIFKWYSHSKEKIQNLSLISLANLPPSGLANAVAEGIRSEENIYTIEENVYEVEEPNEYYCYVSSRQQPSQPLGCRFAMP

SEQ ID NO: 24 - protein, 5 aa, Artificial Sequence
  Artificially Synthesized Sequence
SYDMS

SEQ ID NO: 25 - protein, 17 aa, Artificial Sequence
  Artificially Synthesized Sequence
TISGGGSYTYYQDSVKG

SEQ ID NO: 26 - protein, 7 aa, Artificial Sequence
  Artificially Synthesized Sequence
PYYAMDY

SEQ ID NO: 27 - protein, 11 aa, Artificial Sequence
  Artificially Synthesized Sequence
KASQDVGTAVA

SEQ ID NO: 28 - protein, 7 aa, Artificial Sequence
  Artificially Synthesized Sequence
WASTLHT

SEQ ID NO: 29 - protein, 9 aa, Artificial Sequence
  Artificially Synthesized Sequence
QHYSSYPWT

SEQ ID NO: 30 - protein, 116 aa, Artificial Sequence
  Artificially Synthesized Sequence
EVQLLESGGGLVQPGGSLRLSCAASGFTFSSYDMSWVRQAPGKGLEWVSTISGGGSYTYYQDSVKGRFTISRDNSKNTLYLQMNSLRAEDTAVYYCASPYYAMDYWGQGTTVTVSS

SEQ ID NO: 31 - protein, 107 aa, Artificial Sequence
  Artificially Synthesized Sequence
DIQLTQSPSFLSAYVGDRVTITCKASQDVGTAVAWYQQKPGKAPKLLIYWASTLHTGVPSRFSGSGSGTEFTLTISSLQPEDFATYYCQHYSSYPWTFGQGTKLEIK

SEQ ID NO: 32 - protein, 443 aa, Artificial Sequence
  Artificially Synthesized Sequence
EVQLLESGGGLVQPGGSLRLSCAASGFTFSSYDMSWVRQAPGKGLEWVSTISGGGSYTYYQDSVKGRFTISRDNSKNTLYLQMNSLRAEDTAVYYCASPYYAMDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 33 - protein, 214 aa, Artificial Sequence
  Artificially Synthesized Sequence
DIQLTQSPSFLSAYVGDRVTITCKASQDVGTAVAWYQQKPGKAPKLLIYWASTLHTGVPSRFSGSGSGTEFTLTISSLQPEDFATYYCQHYSSYPWTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 34 - protein, 443 aa, Artificial Sequence
  Artificially Synthesized Sequence
EVQLLESGGGLVQPGGSLRLSCAASGFTFSSYDMSWVRQAPGKGLEWVSTISGGGSYTYYQDSVKGRFTISRDNSKNTLYLQMNSLRAEDTAVYYCASPYYAMDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESDGQPENNYKTTPPVLDSDGSFFLYSRLTVDKSRWQEGNVFSCSVMHEALHNHYTQKSLSLSLGK

SEQ ID NO: 35 - protein, 443 aa, Artificial Sequence
  Artificially Synthesized Sequence
EVQLLESGGGLVQPGGSLRLSCAASGFTFSSYDMSWVRQAPGKGLEWVSTISGGGSYTYYQDSVKGRFTISRDNSKNTLYLQMNSLRAEDTAVYYCASPYYAMDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPEDNYKTTPPVLDSDGSFFLYSRLTVDKSRWQEGNVFSCSVMHEALHNHYTQKSLSLSLGK

SEQ ID NO: 36 - protein, 443 aa, Artificial Sequence
  Artificially Synthesized Sequence
EVQLLESGGGLVQPGGSLRLSCAASGFTFSSYDMSWVRQAPGKGLEWVSTISGGGSYTYYQDSVKGRFTISRDNSKNTLYLQMNSLRAEDTAVYYCASPYYAMDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESDGQPEDNYKTTPPVLDSDGSFFLYSRLTVDKSRWQEGNVFSCSVMHEALHNHYTQKSLSLSLGK

SEQ ID NO: 37 - protein, 9 aa, Artificial Sequence
  Artificially Synthesized Sequence
QHYNSYPWT

SEQ ID NO: 38 - protein, 288 aa, Homo sapiens
MQIPQAPWPVVWAVLQLGWRPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVGVVGGLLGSLVLLVWVLAVICSRAARGTIGARRTGQPLKEDPSAVPVFSVDYGELDFQWREKTPEPPVPCVPEQTEYATIVFPSGMGTSSPARRGSADGPRSAQPLRPEDGHCSWPL

SEQ ID NO: 39 - protein, 290 aa, Homo sapiens
MRIFAVFIFMTYWHLLNAFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTHLVILGAILLCLGVALTFIFRLRKGRMMDVKKCGIQDTNSKKQSDTHLEET

SEQ ID NO: 40 - protein, 273 aa, Homo sapiens
MIFLLLMLSLELQLHQIAALFTVTVPKELYIIEHGSNVTLECNFDTGSHVNLGAITASLQKVENDTSPHRERATLLEEQLPLGKASFHIPQVQVRDEGQYQCIIIYGVAWDYKYLTLKVKASYRKINTHILKVPETDEVELTCQATGYPLAEVSWPNVSVPANTSHSRTPEGLYQVTSVLRLKPPPGRNFSCVFWNTHVRELTLASIDLQSQMEPRTHPTWLLHIFIPFCIIAFIFIATVIALRKQLCQKLYSSKDTTKRPVTTTKREVNSAI

SEQ ID NO: 41 - protein, 10 aa, Artificial Sequence
  Artificially Synthesized Sequence
GFTFSDYWMD

SEQ ID NO: 42 - protein, 17 aa, Artificial Sequence
  Artificially Synthesized Sequence
NIDEDGSITEYSPFVKG

SEQ ID NO: 43 - protein, 8 aa, Artificial Sequence
  Artificially Synthesized Sequence
WGRFGFDS

SEQ ID NO: 44 - protein, 15 aa, Artificial Sequence
  Artificially Synthesized Sequence
KSSQSLLSGSFNYLT

SEQ ID NO: 45 - protein, 7 aa, Artificial Sequence
  Artificially Synthesized Sequence
YASTRHT

SEQ ID NO: 46 - protein, 9 aa, Artificial Sequence
  Artificially Synthesized Sequence
HHHYNAPPT

SEQ ID NO: 47 - protein, 116 aa, Artificial Sequence
  Artificially Synthesized Sequence
EVQLVESGGLVQPGGSLRLSCAASGFTFSDYWMDWVRQAPGKGLVWVSNIDEDGSITEYSPFVKGRFTISRDNAKNTLYLQMNSLRAEDTAVYYCTRWGRFGFDSWGQGTLVTVSS

SEQ ID NO: 48 - protein, 111 aa, Artificial Sequence
  Artificially Synthesized Sequence
DIVMTQSPDSLAVSLGERATINCKSSQSLLSGSFNYLTWYQQKPGQPPKLLIFYASTRHTGVPDRFSGSGSGTDFTLTISSLQAEDVAVYYCHHHYNAPPTFGPGTKVDIK

SEQ ID NO: 49 - protein, 121 aa, Artificial Sequence
  Artificially Synthesized Sequence
EVQLVESGGGLVKPGGSLRLSCAASGFTFSDYFMHWVRQAPGKGLEWVGVIDTKSFNYATYYSDLVKGRFTISRDDSKNTLYLQMNSLKTEDTAVYYCTATIAVPYYFDYWGQGTLVTVSS

SEQ ID NO: 50 - protein, 106 aa, Artificial Sequence
  Artificially Synthesized Sequence
DIQMTQSPSSLSASVGDRVTITCQASQDISNYLSWYQQKPGKAPKLLIYYTNLLAEGVPSRFSGSGSGTDFTFTISSLQPEDIATYYCQQYYNYRTFGPGTKVDIK

SEQ ID NO: 51 - protein, 124 aa, Artificial Sequence
  Artificially Synthesized Sequence
EVQLVESGGGVVRPGGSLRLSCVASGVTFDDYGMSWVRQAPGKGLEWVSGINWNGGDTDYSDSVKGRFTISRDNAKNSLYLQMNSLRAEDTALYYCARDFYGSGSYYHVPFDYWGQGILVTVSS

SEQ ID NO: 52 - protein, 108 aa, Artificial Sequence
  Artificially Synthesized Sequence
EIVLTQSPGTLSLSPGERATLSCRASQSVSRSYLAWYQQKRGQAPRLLIYGASSRATGIPDRFSGDGSGTDFTLSISRLEPEDFAVYYCHQYDMSPFTFGPGTKVDIK

SEQ ID NO: 53 - protein, 125 aa, Artificial Sequence
  Artificially Synthesized Sequence
QVQLVQSGAEVKKPGASVKVSCKASGYTFTGYYMHWVRQAPGQGLEWMGWINPHSGETIYAQKFQGRVTMTRDTSISTAYMELSSLRSEDTAVYYCARTYYYDTSGYYHDAFDVWGQGTMVTVSS

SEQ ID NO: 54 - protein, 5 aa, Artificial Sequence
  Artificially Synthesized Sequence
GYYMH

SEQ ID NO: 55 - protein, 17 aa, Artificial Sequence
  Artificially Synthesized Sequence
WINPHSGETIYAQKFQG

SEQ ID NO: 56 - protein, 16 aa, Artificial Sequence
  Artificially Synthesized Sequence
TYYYDTSGYYHDAFDV

SEQ ID NO: 57 - protein, 107 aa, Artificial Sequence
  Artificially Synthesized Sequence
DIQMTQSPSSVSASVGDRVTITCRASQGISRLLAWYQQKPGKAPKLLIYVASSLQSGVPSRFSGSGSGTDFTLTISSLQPEDFATYYCQQANSFPWTFGQGTKVEIK

SEQ ID NO: 58 - protein, 11 aa, Artificial Sequence
  Artificially Synthesized Sequence
RASQGISRLLA

SEQ ID NO: 59 - protein, 7 aa, Artificial Sequence
  Artificially Synthesized Sequence
VASSLQS

SEQ ID NO: 60 - protein, 9 aa, Artificial Sequence
  Artificially Synthesized Sequence
QQANSFPWT